(19) United States
(12) Patent Application Publication
(10) Pub. No.: US 2025/0019450 A1
(43) Pub. Date: Jan. 16, 2025

(54) BISPECIFIC AGONISTIC ANTIBODIES TO IL12 RECEPTOR

(71) Applicant: DIAGONAL THERAPEUTICS INC., Cambridge, MA (US)

(72) Inventors: Alexey Alexandrovich Lugovskoy, Belmont, MA (US); Jean-Christophe Hus, Concord, MA (US); Melissa Geddie, Arlington, MA (US)

(21) Appl. No.: 18/667,584

(22) Filed: May 17, 2024

Related U.S. Application Data
(60) Provisional application No. 63/467,832, filed on May 19, 2023.

(51) Int. Cl. C07K 16/28

(52) U.S. Cl. CPC C07K 16/2866; C07K 2317/31; C07K 2317/52; C07K 2317/55; C07K 2317/565; C07K 2317/569; C07K 2317/622; C07K 2317/75; C07K 2317/92

(57) ABSTRACT

Bispecific agonistic antibodies that bind to IL-12 receptor, and methods of using the same, are provided.

### RELATED APPLICATIONS

This application claims priority to U.S. Provisional Patent Application Ser. No. 63/467,832, filed May 19, 2023, the entire disclosure of which is hereby incorporated herein by reference.

### SEQUENCE LISTING

The instant application contains a Sequence Listing which has been submitted electronically in XML format and is hereby incorporated by reference in its entirety. Said XML file, created on Sep. 24, 2024, is named 750632_DGT9-004_ST26.xml and is 915,653 bytes in size.

### BACKGROUND

Interleukin-12 (IL-12) has emerged as one of the most potent cytokines in mediating antitumor activity in a variety of preclinical models. It is known as a T-cell stimulating factor, which can stimulate the growth and function of T cells. Through pleiotropic effects on different immune cells that form the tumor microenvironment, IL-12 establishes a link between innate and adaptive immunity that involves different immune effector cells and cytokines depending on the type of tumor or the affected tissue. IL-12 is a heterodimeric pro-inflammatory cytokine that regulates T-cell and natural killer (NK) cell responses. IL-12 is also known to induce the production of interferon-γ (IFN-γ) and favors the differentiation of T helper 1 (T_{H}1) cells. IL-12 binds to the IL-12 receptor, which is a heterodimeric receptor formed by IL-12Rβ 1 and IL-12Rβ2. IL-12Rβ2 is considered to play a key role in IL-12 function, as it is found on activated T cells. Upon binding, IL-12Rβ2 becomes tyrosine phosphorylated and provides binding sites for Tyk2 and Jak2 kinases. These are important in activating critical transcription factor proteins such as STAT4 that are implicated in IL-12 signaling in T cells and NK cells.

IL-12 has been considered a strong candidate for immunotherapy-based interventions in the treatment of cancer, as it potentiates tumor-specific cytotoxic NK and CD8+ T cells that are largely responsible for tumor killing. However, systemic administration of IL-12 is quite toxic, therefore, alternative methods of inducing signaling through the IL-12 receptor are needed.

### SUMMARY

The present disclosure improves upon the prior art by providing heteromeric antibodies which can effectively crosslink the IL-12Rβ1 and the IL-12Rβ2 subunits of the IL-12 receptor and thereby activate IL-12R-mediated cell signaling.

In one aspect, provided herein is a multi-specific binding protein comprising at least a first binding moiety which binds specifically to a human interleukin-12 receptor 31 (IL-12Rβ 1) subunit, and at least a second binding moiety which binds specifically to a human IL-12 receptor β2 (IL-12Rβ2) subunit, wherein the second binding moiety exhibits higher binding affinity (KD) for the human IL-12Rβ2 subunit than the first binding moiety exhibits for the human IL-12Rβ 1 subunit, and is capable of inducing IL-12 receptor signaling by inducing proximity between the IL-12Rβ 1 and IL-12Rβ2 subunits of human IL-12 receptor.

In some embodiments, the binding affinity of the second binding moiety for the IL-12Rβ2 subunit is at least 5-fold greater than the binding affinity of the first binding moiety for the IL-12Rβ1 subunit. In some embodiments, the binding affinity of the second binding moiety for the IL-12Rβ2 subunit is at least 10-fold greater than the binding affinity of the first binding moiety for the IL-12Rβ1 subunit. In some embodiments, the binding affinity of the second binding moiety for the IL-12Rβ2 subunit is at least 20-fold greater than the binding affinity of the first binding moiety for the IL-12Rβ 1 subunit. In some embodiments, the binding affinity of the second binding moiety for the IL-12Rβ2 subunit is at least 40-fold greater than the binding affinity of the first binding moiety for the IL-12Rβ 1 subunit. In some embodiments, the binding affinity of the second binding moiety for the IL-12Rβ2 subunit is at least 100-fold greater than the binding affinity of the first binding moiety for the IL-12Rβ 1 subunit.

In some embodiments, the binding affinity of the second binding moiety for the IL-12Rβ2 subunit promotes selective binding to activated T cells and/or NK cells.

In some embodiments, the first binding moiety comprises an IL-12Rβ 1 VHH domain and the second binding moiety comprises an IL-12Rβ2 VHH domain. In some embodiments, the first binding moiety comprises an IL-12Rβ1 Fab domain and the second binding moiety comprises an IL-12Rβ2 Fab domain. In some embodiments, the first binding moiety comprises an IL-12Rβ 1 VHH domain and the second binding moiety comprises an IL-12Rβ2 Fab domain. In some embodiments, the first binding moiety comprises an IL-12Rβ 1 Fab domain and the second binding moiety comprises an IL-12Rβ2 VHH domain. In some embodiments, the first binding moiety comprises an IL-12Rβ1 Fab domain and the second binding moiety comprises an IL-12Rβ2 scFv domain. In some embodiments, the first binding moiety comprises an IL-12Rβ 1 scFv domain and the second binding moiety comprises an IL-12Rβ2 Fab domain. In some embodiments, the first binding moiety comprises an IL-12Rβ1 scFv domain and the second binding moiety comprises an IL-12Rβ2 scFv domain. In some embodiments, the first binding moiety comprises an IL-12Rβ 1 scFv domain and the second binding moiety comprises an IL-12Rβ2 VHH domain. In some embodiments, the first binding moiety comprises an IL-12Rβ1 VHH domain and the second binding moiety comprises an IL-12Rβ2 scFv domain.

In some embodiments, the first binding moiety comprises a IL-12Rβ1 VHH domain. In some embodiments, the IL-12Rβ1 VHH domain comprises a HCDR1 sequence, a HCDR2 sequence, and a HCDR3 sequence as found in Table 4. In some embodiments, the IL-12Rβ 1 VHH domain comprises a sequence that is at least about 90% identical, at least about 95%, identical, at least about 96%, at least about 97%, at least about 98%, or at least about 99% identical to the amino acid sequence of any one of the amino acid sequences in Table 5.

In some embodiments, the second binding moiety comprises an IL-12Rβ2 VHH domain. In some embodiments, the IL-12Rβ2 VHH domain comprises a HCDR1 sequence, a HCDR2 sequence, and a HCDR3 sequence as found in Table 9. In some embodiments, the IL-12Rβ2 VHH domain comprises a sequence that is at least about 90% identical, at least about 95%, identical, at least about 96%, at least about 97%, at least about 98%, or at least about 99% identical to the amino acid sequence of any one of the amino acid sequences in Table 10.

In some embodiments, the IL-12Rβ1 VHH domain comprises the amino acid sequence of any one of the amino acid sequences in Table 5. In some embodiments, the IL-12Rβ2 VHH domain comprises the amino acid sequence of any one of the amino acid sequences in Table 10.

In some embodiments, the VHH domain comprises a P14A amino acid substitution according to Kabat numbering.

In some embodiments, the P14A amino acid substitution further stabilizes the multispecific binding protein.

In some embodiments, the P14A amino acid substitution increases the agonist properties of the multispecific binding protein.

In some embodiments, the first binding moiety or second binding moiety is a Fab or an scFv. In some embodiments, the Fab or scFv comprises a variable heavy chain region (VH) and a variable light chain region (VL). In some embodiments, the VH comprises a HCDR1 sequence, a HCDR2 sequence, and a HCDR3 sequence as found in Table 1. In some embodiments, the VL comprises a LCDR1 sequence, a LCDR2 sequence, and a LCDR3 sequence as found in Table 2. In some embodiments, the VH of the Fab or scFv of the first binding moiety comprises a sequence that is at least 90% identical, at least about 95% identical, at least about 96% identical, at least about 97% identical, at least about 98% identical, or at least about 99% identical to an amino acid sequence of Table 3. In some embodiments, the VL of the Fab or scFv of the first binding moiety comprises a sequence that is at least 90% identical, at least about 95% identical, at least about 96% identical, at least about 97% identical, at least about 98% identical, or at least about 99% identical to an amino acid sequence of Table 3. In some embodiments, the VH of the Fab or scFv of the first binding moiety comprises the amino acid sequence of Table 3. In some embodiments, the VL of the Fab or scFv of the first binding moiety comprises the amino acid sequence of Table 3. In some embodiments, the VH of the Fab or scFv of the second binding moiety comprises a sequence that is at least 90% identical, at least about 95% identical, at least about 96% identical, at least about 97% identical, at least about 98% identical, or at least about 99% identical to an amino acid sequence of Table 8. In some embodiments, the VL of the Fab or scFv of the second binding moiety comprises a sequence that is at least 90% identical, at least about 95% identical, at least about 96% identical, at least about 97% identical, at least about 98% identical, or at least about 99% identical to an amino acid sequence of Table 8.

In some embodiments, the first binding moiety comprises a IL-12Rβ1 VHH domain comprising a HCDR1 sequence of SEQ ID NO: 293, a HCDR2 sequence of SEQ ID NO: 294, and a HCDR3 sequence of SEQ ID NO: 295, and the second binding moiety comprises a IL-12Rβ2 VH domain and VL domain, the VH domain comprising a HCDR1 sequence of SEQ ID NO: 456, a HCDR2 sequence of SEQ ID NO: 457, and a HCDR3 sequence of SEQ ID NO: 458, and the VL domain comprising a LCDR1 sequence of SEQ ID NO: 532, a LCDR2 sequence of SEQ ID NO: 533, and a LCDR3 sequence of SEQ ID NO: 534.

In some embodiments, the first binding moiety comprises a IL-12Rβ1 VHH domain comprising SEQ ID NO: 369, and the second binding moiety comprises a IL-12Rβ2 VH domain and VL domain, the VH domain comprising SEQ ID NO: 586, and the VL domain comprising SEQ ID NO: 587.

In some embodiments, the multi-specific binding protein comprises a first heavy chain polypeptide (HC1) of SEQ ID NO: 736, a second heavy chain polypeptide (HC2) of SEQ ID NO: 737, and a light chain polypeptide (LC) of SEQ ID NO: 738.

In some embodiments, the first binding moiety comprises a IL-12Rβ1 VHH domain comprising a HCDR1 sequence of SEQ ID NO: 344, a HCDR2 sequence of SEQ ID NO: 345, and a HCDR3 sequence of SEQ ID NO: 346, and the second binding moiety comprises a IL-12Rβ2 VHH domain comprising a HCDR1 sequence of SEQ ID NO: 600, a HCDR2 sequence of SEQ ID NO: 601, and a HCDR3 sequence of SEQ ID NO: 602.

In some embodiments, the first binding moiety comprises a IL-12Rβ1 VHH domain comprising SEQ ID NO: 389, and the second binding moiety comprises a IL-12Rβ2 VHH domain comprising SEQ ID NO: 674.

In some embodiments, the multi-specific binding protein comprises a first heavy chain polypeptide (HC1) of SEQ ID NO: 859, and a second heavy chain polypeptide (HC2) of SEQ ID NO: 860.

In some embodiments, the first binding moiety comprises a IL-12Rβ 1 VH domain and VL domain, the VH domain comprising a HCDR1 sequence of SEQ ID NO: 70, a HCDR2 sequence of SEQ ID NO: 71, and a HCDR3 sequence of SEQ ID NO: 72, and the VL domain comprising a LCDR1 sequence of SEQ ID NO: 175, a LCDR2 sequence of SEQ ID NO: 176, and a LCDR3 sequence of SEQ ID NO: 177, and the second binding moiety comprises a IL-12Rβ2 VHH domain comprising a HCDR1 sequence of SEQ ID NO: 594, a HCDR2 sequence of SEQ ID NO: 595, and a HCDR3 sequence of SEQ ID NO: 596.

In some embodiments, the first binding moiety comprises a IL-12Rβ 1 VH domain and VL domain, the VH domain comprising SEQ ID NO: 257, and the VL domain comprising SEQ ID NO: 258, and the second binding moiety comprises a IL-12Rβ2 a VHH domain comprising SEQ ID NO: 672.

In some embodiments, the multi-specific binding protein comprises a first heavy chain polypeptide (HC1) of SEQ ID NO: 724, a second heavy chain polypeptide (HC2) of SEQ ID NO: 725, and a light chain polypeptide (LC) of SEQ ID NO: 726.

In some embodiments, the first binding moiety comprises a IL-12Rβ1 VHH domain comprising a HCDR1 sequence of SEQ ID NO: 293, a HCDR2 sequence of SEQ ID NO: 294, and a HCDR3 sequence of SEQ ID NO: 295, and the second binding moiety comprises a IL-12Rβ2 VH domain and VL domain, the VH domain comprising a HCDR1 sequence of SEQ ID NO: 411, a HCDR2 sequence of SEQ ID NO: 412, and a HCDR3 sequence of SEQ ID NO: 413, and the VL domain comprising a LCDR1 sequence of SEQ ID NO: 486, a LCDR2 sequence of SEQ ID NO: 487, and a LCDR3 sequence of SEQ ID NO: 488.

In some embodiments, the first binding moiety comprises a IL-12Rβ1 VHH domain comprising SEQ ID NO: 369, and the second binding moiety comprises a IL-12Rβ2 VH domain and VL domain, the VH domain comprising SEQ ID NO: 556, and the VL domain comprising SEQ ID NO: 557.

In some embodiments, the multi-specific binding protein comprises a first heavy chain polypeptide (HC1) of SEQ ID NO: 778, and a second heavy chain polypeptide (HC2) of SEQ ID NO: 779.

In some embodiments, the first binding moiety comprises a IL-12Rβ1 VHH domain comprising a HCDR1 sequence of SEQ ID NO: 344, a HCDR2 sequence of SEQ ID NO: 345, and a HCDR3 sequence of SEQ ID NO: 346, and the second binding moiety comprises a IL-12Rβ2 VHH domain comprising a HCDR1 sequence of SEQ ID NO: 597, a HCDR2 sequence of SEQ ID NO: 598, and a HCDR3 sequence of SEQ ID NO: 599.

In some embodiments, the first binding moiety comprises a IL-12Rβ1 VHH domain comprising SEQ ID NO: 389, and the second binding moiety comprises a IL-12Rβ2 VHH domain comprising SEQ ID NO: 673.

In some embodiments, the multi-specific binding protein comprises a first heavy chain polypeptide (HC1) of SEQ ID NO: 857, and a second heavy chain polypeptide (HC2) of SEQ ID NO: 858.

In some embodiments, the first binding moiety comprises a IL-12Rβ1 VHH domain comprising a HCDR1 sequence of SEQ ID NO: 344, a HCDR2 sequence of SEQ ID NO: 345, and a HCDR3 sequence of SEQ ID NO: 346, and the second binding moiety comprises a IL-12Rβ2 VHH domain comprising a HCDR1 sequence of SEQ ID NO: 612, a HCDR2 sequence of SEQ ID NO: 613, and a HCDR3 sequence of SEQ ID NO: 614.

In some embodiments, the first binding moiety comprises a IL-12Rβ1 VHH domain comprising SEQ ID NO: 389, and the second binding moiety comprises a IL-12Rβ2 VHH domain comprising SEQ ID NO: 678.

In some embodiments, the multi-specific binding protein comprises a first heavy chain polypeptide (HC1) of SEQ ID NO: 863, and a second heavy chain polypeptide (HC2) of SEQ ID NO: 864.

In some embodiments, the first binding moiety comprises a IL-12Rβ1 VHH domain comprising a HCDR1 sequence of SEQ ID NO: 308, a HCDR2 sequence of SEQ ID NO: 309, and a HCDR3 sequence of SEQ ID NO: 310, and the second binding moiety comprises a IL-12Rβ2 VHH domain comprising a HCDR1 sequence of SEQ ID NO: 612, a HCDR2 sequence of SEQ ID NO: 613, and a HCDR3 sequence of SEQ ID NO: 614.

In some embodiments, the first binding moiety comprises a IL-12Rβ 1 VHH domain comprising SEQ ID NO: 374, and the second binding moiety comprises a IL-12Rβ2 VHH domain comprising SEQ ID NO: 678.

In some embodiments, the multi-specific binding protein comprises a first heavy chain polypeptide (HC1) of SEQ ID NO: 828, and a second heavy chain polypeptide (HC2) of SEQ ID NO: 829.

In some embodiments, the first binding moiety comprises a IL-12Rβ 1 VHH domain comprising a HCDR1 sequence of SEQ ID NO: 308, a HCDR2 sequence of SEQ ID NO: 309, and a HCDR3 sequence of SEQ ID NO: 310, and the second binding moiety comprises a IL-12Rβ2 VH domain and VL domain, the VH domain comprising a HCDR1 sequence of SEQ ID NO: 447, a HCDR2 sequence of SEQ ID NO: 448, and a HCDR3 sequence of SEQ ID NO: 449, and the VL domain comprising a LCDR1 sequence of SEQ ID NO: 523, a LCDR2 sequence of SEQ ID NO: 524, and a LCDR3 sequence of SEQ ID NO: 525.

In some embodiments, the first binding moiety comprises a IL-12Rβ 1 VHH domain comprising SEQ ID NO: 374, and the second binding moiety comprises a IL-12Rβ2 VH domain and VL domain, the VH domain comprising SEQ ID NO: 580, and the VL domain comprising SEQ ID NO: 581.

In some embodiments, the multi-specific binding protein comprises a first heavy chain polypeptide (HC1) of SEQ ID NO: 745, a second heavy chain polypeptide (HC2) of SEQ ID NO: 746, and a light chain polypeptide (LC) of SEQ ID NO: 747.

In some embodiments, the first binding moiety comprises a IL-12Rβ 1 VHH domain comprising a HCDR1 sequence of SEQ ID NO: 302, a HCDR2 sequence of SEQ ID NO: 303, and a HCDR3 sequence of SEQ ID NO: 304, and the second binding moiety comprises a IL-12Rβ2 VHH domain comprising a HCDR1 sequence of SEQ ID NO: 597, a HCDR2 sequence of SEQ ID NO: 598, and a HCDR3 sequence of SEQ ID NO: 599.

In some embodiments, the first binding moiety comprises a IL-12Rβ 1 VHH domain comprising SEQ ID NO: 372, and the second binding moiety comprises a IL-12Rβ2 VHH domain comprising SEQ ID NO: 673.

In some embodiments, the multi-specific binding protein is capable of inducing IL-12 receptor signaling in the presence of IL-12.

In some embodiments, induction of IL-12 receptor signaling is detected via a surface plasmon resonance (SPR) assay.

In some embodiments, the SPR assay comprises the following steps: 1) contacting the first binding moiety and/or the second binding moiety with an extracellular domain (ECD) of one or both of IL-12R β1 and IL-12R β2 and isolated IL-12; and 2) detecting binding of the first binding moiety and/or the second binding moiety with the ECD of one or both of IL-12R β1 and IL-12R β2, wherein detection of binding indicates that the multi-specific binding protein is capable of inducing IL-12 receptor signaling in the presence of IL-12.

In some embodiments, the multi-specific binding protein is capable of binding specifically to human IL-12Rβ1 subunit and human IL-12Rβ2 subunit in the presence of IL-12.

In some embodiments, binding specifically to human IL-12Rβ1 subunit and human IL-12Rβ2 subunit is detected via a surface plasmon resonance (SPR) assay.

In some embodiments, the SPR assay comprises the following steps:
1) contacting the first binding moiety and/or the second binding moiety with an extracellular domain (ECD) of one or both of IL-12R β1 and IL12R β2 and isolated IL-12; and
2) detecting binding of the first binding moiety and/or the second binding moiety with the ECD of one or both of IL-12R β1 and IL-12R β2,
wherein detection of binding indicates that the multi-specific binding protein is capable of binding specifically to human IL-12Rβ 1 subunit and human IL-12Rβ2 subunit in the presence of IL-12.

In some embodiments, the first binding moiety designated 97B1 and 263B1 of the multi-specific binding protein compete for binding to IL-12Rβ 1.

In some embodiments, the first binding moiety designated 115B1, 258B1, 32B1, and 72B1 of the multi-specific binding protein compete for binding to IL-12Rβ 1.

In some embodiments, the first binding moiety designated 202B1 of the multi-specific binding protein competes for binding to IL-12Rβ 1 with one or more anti-IL-12 Rβ1 binding moieties disclosed herein.

In some embodiments, the first binding moiety designated 233B1 of the multi-specific binding protein competes for binding to IL-12Rβ 1 with one or more anti-IL-12 Rβ1 binding moieties disclosed herein.

In some embodiments, the first binding moiety designated 245B1v2 of the multi-specific binding protein competes for binding to IL-12Rβ 1 with one or more anti-IL-12 Rβ1 binding moieties disclosed herein.

In some embodiments, the first binding moiety designated 187B1 of the multi-specific binding protein competes for binding to IL-12Rβ 1 with one or more anti-IL-12 Rβ1 binding moieties disclosed herein.

In some embodiments, the second binding moiety designated 13B2 and 64B2 of the multi-specific binding protein compete for binding to IL-12Rβ2.

In some embodiments, the second binding moiety designated 185B2 and 219B2 of the multi-specific binding protein compete for binding to IL-12Rβ2.

In some embodiments, the second binding moiety designated 85B2 of the multi-specific binding protein competes for binding to IL-12Rβ2 with one or more anti-IL-12 Rβ2 binding moieties disclosed herein.

In some embodiments, the second binding moiety designated 19B2 of the multi-specific binding protein competes for binding to IL-12Rβ2 with one or more anti-IL-12 Rβ2 binding moieties disclosed herein.

In some embodiments, the second binding moiety designated 230B2 of the multi-specific binding protein competes for binding to IL-12Rβ2 with one or more anti-IL-12 Rβ2 binding moieties disclosed herein.

In some embodiments, the multi-specific binding protein further comprises all or part of an immunoglobulin Fc domain or variant thereof. In some embodiments, the Fc domain or variant thereof comprises a first Fc heavy chain and a second Fc heavy chain.

In some embodiments, the multi-specific binding protein further comprises a variant Fc domain with reduced effector function. In some embodiments, at least one Fc heavy chain comprises a substitution at amino acid position 234, according to EU numbering. In some embodiments, the substitution at amino acid position 234 is an alanine (A). In some embodiments, at least one Fc heavy chain comprises a substitution at amino acid position 235, according to EU numbering. In some embodiments, the substitution at amino acid position 235 is an alanine (A). In some embodiments, at least one Fc heavy chain comprises a substitution at amino acid position 237, according to EU numbering. In some embodiments, wherein the substitution at amino acid position 237 is an alanine (A). In some embodiments, at least one Fc heavy chain comprises one or more substitutions at amino acid positions 234, 235, or 237, according to EU numbering. In some embodiments, the substitution at amino acid position 234 is an alanine (A), the substitution at amino acid position 235 is an alanine (A), and the substitution at amino acid position 237 is an alanine (A).

In some embodiments, the Fc domain comprises heterodimerization mutations to promote heterodimerization of the first binding moiety with the second binding moiety. In some embodiments, wherein the heterodimerization mutations are Knob-in-Hole (KIH) mutations. In some embodiments, the first Fc heavy chain comprises an amino acid substitution at position 366, 368, or 407 which produced a hole, and the second Fc heavy chain comprises an amino acid substitution at position 366 which produced a knob. In some embodiments, the first Fc heavy chain comprises the amino acid substitution T366S, L368A, or Y407V, and the second Fc heavy chain comprises the amino acid substitution T366W.

In some embodiments, the heterodimerization mutations are charge stabilization mutations. In some embodiments, the first Fc heavy chain comprises the amino acid substitution N297K, and the second Fc heavy chain comprises the amino acid substitution N297D. In some embodiments, the first Fc heavy chain comprises the amino acid substitution T299K, and the second Fc heavy chain comprises the amino acid substitution T299D.

In some embodiments, the heterodimerization mutations comprise an engineered disulfide bond. In some embodiments, the engineered disulfide bond is formed by a first Fc heavy chain comprising the amino acid substitution Y349C, and a second Fc heavy chain comprising the amino acid substitution S354C. In some embodiments, the engineered disulfide bond is formed by a C-terminal extension peptide fused to the C-terminus of each of the first Fc heavy chain and the second Fc heavy chain. In some embodiments, the first Fc heavy chain C-terminal extension comprises the amino acid sequence GEC, and the second Fc heavy chain C-terminal extension comprises the amino acid sequence SCDKT(SEQ ID NO:951).

In some embodiments, at least one Fc domain comprises one or more mutations to promote increased half-life. In some embodiments, at least one Fc heavy chain comprises one or more substitutions at amino acid positions 252, 254, or 256, according to EU numbering.

In some embodiments, the substitution at amino acid position 252 is a tyrosine (Y), the substitution at amino acid position 254 is a threonine (T), and the substitution at amino acid position 236 is a glutamic acid (E).

In some embodiments, at least one Fc heavy chain comprises one or more substitutions at amino acid positions 428 or 434, according to EU numbering. In some embodiments, the substitution at amino acid 428 is a leucine (L), and the substitution at amino acid position 434 is a serine (S).

In some embodiments, the first binding moiety which specifically binds to human IL-12Rβ1 comprises heavy chain domain comprising an amino acid sequence set forth in any one of the sequences of Table 11. In some embodiments, the first binding moiety which specifically binds to human IL-12Rβ1 comprises a light chain domain comprising an amino acid sequence set forth in any one of the sequences of Table 11. In some embodiments, the second binding moiety which specifically binds to human IL-12Rβ2 comprises a heavy chain domain comprising an amino acid sequence set forth in any one of the sequences of Table 11. IN some embodiments, the second binding moiety which specifically binds to human IL-12Rβ2 comprises a light chain domain comprising an amino acid sequence set forth in any one of the sequences of Table 11.

In some embodiments, the multi-specific binding protein comprises the HC and LC of any of the antibodies of Table 11.

In another aspect, provided herein is a pharmaceutical composition comprising the multi-specific binding protein of any one of the preceding claims and a pharmaceutically acceptable carrier.

In another aspect, provided herein is an isolated nucleic acid molecule encoding the multi-specific binding protein as disclosed herein. In some embodiments, an expression comprises the nucleic acid encoding the multi-specific binding protein as disclosed herein. In some embodiments, an expression vector comprises the nucleic molecule encoding the multi-specific binding protein as disclosed herein. In some embodiments, a host cell comprises the expression vector disclosed herein.

In an aspect, provided herein is a method for treating a disease or disorder in a subject, comprising administering to a subject in need thereof the multi-specific binding protein as disclosed herein. In some embodiments, the disease or disorder is a cancer.

In some embodiments, the multi-specific binding protein as disclosed herein is for use as a medicament.

In yet another aspect, provided herein is a multi-specific binding protein comprising at least a first binding moiety which binds specifically to a human interleukin-12 receptor 1 (IL-12Rβ1) subunit, and at least a second binding moiety which binds specifically to a human IL-12 receptor β2 (IL-12Rβ2) subunit, wherein:
a) the first binding moiety comprises:
i) a HCDR1 amino acid sequence, a HCDR2 amino acid sequence, and a HCDR3 amino acid sequence disclosed in Tables 1, 3, 4, 5, or 11;
ii) a LCDR1 amino acid sequence, a LCDR1 amino acid sequence, and a LCDR3 amino acid sequence disclosed in Tables 2, 3, or 11; and
b) the second binding moiety comprises:
i) a HCDR1 amino acid sequence, a HCDR2 amino acid sequence, and a HCDR3 amino acid sequence disclosed in Tables 6, 8, 9, 10, or 11;
ii) a LCDR1 amino acid sequence, a LCDR1 amino acid sequence, and a LCDR3 amino acid sequence disclosed in Tables 7, 8, or 11.

### DETAILED DESCRIPTION

Before the present disclosure is described, it is to be understood that this disclosure is not limited to particular methods and experimental conditions described, as such methods and conditions may vary. It is also understood that the terminology used herein is for the purpose of describing particular embodiments only, and it is not intended to be limiting, since the scope of the present disclosure will be limited only by the appended claims.

Unless otherwise defined, all technical and scientific terms used herein have the same meaning as commonly understood by one of ordinary skill in the art to which this disclosure belongs.

Although the methods and materials similar or equivalent to those described herein can be used in the practice of the present disclosure, exemplary methods and materials are now described. All publications mentioned herein are incorporated herein by reference to describe in their entirety.

As used herein, the term “interleukin-12” or “IL-12” refers to the heterodimeric cytokine comprising a p40 subunit and a p35 subunit. IL-12 is considered a largely pro-inflammatory cytokine, and is produced by antigen-presenting cells, such as dendritic cells and macrophages, and is crucial for the recruitment and effector functions of CD8+ T and NK cells. IL-12 binds through the IL-12 receptor.

As used herein, the term “interleukin-12 receptor” or “IL-12 receptor” or “IL-12R” refers to the type I cytokine receptor, which binds to the IL-12 cytokine. The IL-12 receptor is a heterodimeric receptor comprising an IL-12Rβ 1 subunit and an IL-12Rβ2 subunit. Binding of IL-12 to the IL-12 receptor is initiated by anchoring of the p40 subunit on IL-12Rβ1. The IL-12/IL-12 receptor interaction mediates signaling through the Jak/STAT pathway. Following binding, Jak kinases are activated, leading to the phosphorylation of the IL-12Rβ2 subunit, and recruits STAT4 proteins. STAT4 is phosphorylated to induce homodimerization and translocation to the nucleus where they bind to specific sequences and regulate IFN-γ gene transcription.

As used herein, the term “antigen-binding moiety” or “binding domain” or “binding specificity” refers to a molecule that specifically binds to an antigen as such binding is understood by one skilled in the art. For example, an antigen-binding moiety that specifically binds to an antigen binds to other molecules, generally with lower affinity as determined by, e.g., immunoassays, BIAcore®, KinExA 3000 instrument (Sapidyne Instruments, Boise, ID), or other assays known in the art. In certain embodiments, an antigen-binding moiety that specifically binds to an antigen binds to the antigen with a Ka that is at least 2 logs (e.g., factors of 10), 2.5 logs, 3 logs, 4 logs or greater than the Ka when the molecule binds non-specifically to another antigen. As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multi-specific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, common light chain antibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof.

As used herein, the terms “VH” and “VL” refer to antibody heavy and light chain variable domain, respectively, as described in Kabat et al., (1991) Sequences of Proteins of Immunological Interest (NIH Publication No. 91-3242, Bethesda), which is herein incorporated by reference in its entirety.

As used herein, the term “VHH” refers to the heavy chain variable domain of a camelid heavy chain-only antibody (HCAb) and humanized variants thereof, as described in Hamers-Casterman C. et al., Nature (1993) 363:446-8.10.1038/363446a0, which is incorporated by reference herein in its entirety.

As used herein, the term “VH/VL Pair” refers to a combination of a VH and a VL that together form the binding site for an antigen.

As used herein, the term “heavy chain” when used in reference to an antibody can refer to any distinct type, e.g., alpha (a), delta (6), epsilon (F), gamma (γ), and mu (p), based on the amino acid sequence of the constant domain, which give rise to IgA, IgD, IgE, IgG, and IgM classes of antibodies, respectively, including subclasses of IgG, e.g., IgG1, IgG2, IgG3, and IgG4.

As used herein, the term “full-length antibody heavy chain” refers to an antibody heavy chain comprising, from N to C terminal, a VH, a CH1 region, a hinge region, a CH2 domain and a CH3 domain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain. As used herein, the term “complementarity determining region” or “CDR” refers to sequences of amino acids within antibody variable regions, which confer antigen specificity and binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” or “FR” are known in the art to refer to the non-CDR portions of the variable regions of the heavy and light chains. In general, there are four FRs in each heavy chain variable region (FR-H1, FR-H2, FR-H3, and FR-H4), and four FRs in each light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M. P. et al., “IMGT unique numbering for immunoglobulin and T cell receptor variable domains and Ig superfamily V-like domains,” Dev. Comp. Immunol., 2003 January; 27(1):55-77 (“IMGT” numbering scheme), and Honegger A. and Pluckthun A., “Yet another numbering scheme for immunoglobulin variable domains: an automatic modeling and analysis tool,” J. Mol. Biol., 2001 Jun. 8; 309(3):657-70, (AHo numbering scheme).

The boundaries of a given CDR or FR may vary depending on the scheme used for identification. For example, the Kabat scheme is based on sequence alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

As used herein, the term “single chain variable fragment” (scFv) refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The term “human antibody,” as used herein, is intended to include antibodies having variable and constant regions derived from human germline immunoglobulin sequences. The human mAbs of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include mAbs in which CDR sequences derived from the germline of another mammalian species (e.g., mouse), have been grafted onto human FR sequences. The term includes antibodies recombinantly produced in a non-human mammal, or in cells of a non-human mammal. The term is not intended to include antibodies isolated from or generated in a human subject.

The term “multi-specific antigen-binding molecules,” as used herein refers to bispecific, tri-specific or multi-specific antigen-binding molecules, and antigen-binding fragments thereof. Multi-specific antigen-binding molecules may be specific for different epitopes of one target polypeptide or may contain antigen-binding domains specific for epitopes of more than one target polypeptide. In certain embodiment, the multi-specific antigen binding molecules of the disclosure comprises at least a first binding specificity for the IL-12Rβ 1 subunit and at least a second binding specificity for the IL-12Rβ2 subunit. A multi-specific antigen-binding molecule can be a single multifunctional polypeptide, or it can be a multimeric complex of two or more polypeptides that are covalently or non-covalently associated with one another. The term “multi-specific antigen-binding molecules” includes antibodies of the present disclosure that may be linked to or co-expressed with another functional molecule, e.g., another peptide or protein. For example, an antibody or fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity. According to the present disclosure, the term “multi-specific antigen-binding molecules” also includes bispecific, trispecific or multi-specific antibodies or antigen-binding fragments thereof. In certain exemplary embodiments, an antibody of the present disclosure is functionally linked to another antibody or antigen-binding fragment thereof to produce a bispecific antibody with a second binding specificity.

In exemplary embodiments, the heteromeric antibodies of the present disclosure are bispecific antibodies. Bispecific antibodies can be monoclonal, e.g., human or humanized, antibodies that have binding specificities for at least two different antigens. In certain embodiments, the bispecific antibodies of the disclosure comprises at least a first binding domain for the IL-12Rβ1 subunit and at least a second binding domain for the IL-12Rβ2 subunit.

Methods for making bispecific antibodies are well-known. Traditionally, the recombinant production of bispecific antibodies was based on the co-expression of two immunoglobulin heavy chain/light chain pairs, where the two heavy chains have different specificities (Milstein et al., Nature 305:537 (1983)). Because of the random assortment of immunoglobulin heavy and light chains, the hybridomas (quadromas) produce a potential mixture of ten different antibody molecules, of which only one has the correct bispecific structure. The purification of the correct molecule is usually accomplished by affinity chromatography steps. More modern techniques for generating bispecific antibodies employ heterodimerization domains that favor desired pairing of heavy chain from the antibody with a first specificity to the heavy chain of an antibody with a second specificity.

Antibody variable domains with the desired binding specificities can be fused to immunoglobulin constant domain sequences. The fusion typically is with an immunoglobulin heavy chain constant domain, comprising at least part of the hinge, CH2, and CH3 regions. It may have the first heavy chain constant region (CH1) containing the site necessary for light chain binding present in at least one of the fusions. DNAs encoding the immunoglobulin heavy chain fusions and, if desired, the immunoglobulin light chain, are inserted into separate expression vectors, and are co-transformed into a suitable host organism. For further details of generating bispecific antibodies see, for example Suresh et al., Meth. Enzymol. 121:210 (1986).

As used herein, the term “Fc” refers to a polypeptide comprising a CH2 domain and a CH3 domain, wherein the C-terminus of the CH2 domain is linked (directly or indirectly) to the N-terminus of the CH3 domain. The term “Fc polypeptide” includes an antibody heavy chain linked to an antibody light chain by disulfide bonds (e.g., to form a half-antibody).

As used herein, the term “CH1 domain” refers to the first constant domain of an antibody heavy chain (e.g., amino acid positions 118-215 of human IgG1, according to the EU index). The term includes naturally occurring CH1 domains and engineered variants of naturally occurring CH1 domains (e.g., CH1 domains comprising one or more amino acid insertions, deletions, substitutions, or modifications relative to a naturally occurring CH1 domain).

As used herein, the term “CH2 domain” refers to the second constant domain of an antibody heavy chain (e.g., amino acid positions 231-340 of human IgG1, according to the EU index). The term includes naturally occurring CH2 domains and engineered variants of naturally occurring CH2 domains (e.g., CH2 domains comprising one or more amino acid insertions, deletions, substitutions, or modifications relative to a naturally occurring CH2 domain).

As used herein, the term “CH3 domain” refers to the third constant domain of an antibody heavy chain (e.g., amino acid positions 341-447 of human IgG1, according to the EU index). The term includes naturally occurring CH3 domains and engineered variants of naturally occurring CH3 domains (e.g., CH3 domains comprising one or more amino acid insertions, deletions, substitutions, or modifications relative to a naturally occurring CH3 domain).

As used herein, the term “hinge region” refers to the portion of an antibody heavy chain comprising the cysteine residues (e.g., the cysteine residues at amino acid positions 226 and 229 of human IgG1, according to the EU index) that mediate disulfide bonding between two heavy chains in an intact antibody. The term includes naturally occurring hinge regions and engineered variants of naturally occurring hinge regions (e.g., hinge regions comprising one or more amino acid insertions, deletions, substitutions, or modifications relative to a naturally occurring hinge regions). An exemplary full-length IgG1 hinge region comprises amino acid positions 216-230 of human IgG1, according to the EU index. A hinge region may consist of at least 2 (e.g., 5, 10, 15, 20, 40, 60 or more) amino acids which result in a flexible or semi-flexible linkage between adjacent variable regions and/or constant domains in a single polypeptide molecule. In some embodiments, the hinge region is an immunoglobulin-like hinge region. In some embodiments, the immunoglobulin-like hinge region can be from or derived from any IgG1, IgG2, IgG3, or IgG4 subtype, or from IgA, IgE, IgD or IgM, including chimeric forms thereof, e.g., a chimeric IgG1/2 hinge region.

In some embodiments, the hinge region can be from the human IgG1 subtype extending from amino acid 216 to amino acid 230 according to the numbering system of the EU index, or from amino acid 226 to amino acid 243 according to the numbering system of Kabat. Those skilled in the art may differ in their understanding of the exact amino acids corresponding to the various domains of the IgG molecule. Thus, the N-terminal or C-terminal of the domains outlined above may extend or be shortened by 1, 2, 3, 4, 5, 6, 7, 8, 9, or even 10 amino acids.

The term “upper hinge” as used herein typically refers to the last residue of the CH1 domain up to but not including the first inter-heavy chain cysteine. The upper hinge can sometimes be defined as the N-terminal sequence from position 216 to position 225 according to the Kabat EU numbering system of an IgG1 antibody (Kabat et al., Sequences of Proteins of Immunological Interest, 5th Ed. Public Health Service, National Institute of Health, Bethesda, Md., 1991). The term “middle hinge” refers to the region extending from the first inter-heavy chain cysteine to a proline residue adjacent to the carboxyl-end of the last middle hinge cysteine. The middle hinge can be the N-terminal sequence from position 226 to position 230 according to the Kabat EU numbering system. The term “lower hinge” refers to a highly conserved 7-8 amino acids. The lower hinge can be defined as the sequence from position 231 to 238 according the Kabat EU numbering system of an IgG1 antibody. In some embodiments, the antibody according to the present invention effectively comprises an upper, a middle, and a lower hinge.

As used herein, the term “a modified hinge region” refers to a hinge region in which alterations are made in one or more of the characteristics of the hinge, including, but not limited to, flexibility, length, conformation, charge and hydrophobicity relative to a wild-type hinge. The modified hinge regions disclosed herein may be generated by methods well known in the art, such as, for example introducing a modification into a wild-type hinge. In some embodiments, the hinge region may be modified by one or more amino acids. Modifications which may be utilized to generate a modified hinge region include, but are not limited to, amino acid insertions, deletions, substitutions, and rearrangements. Said modifications of the hinge and the modified hinge regions disclosed are referred to herein jointly as “hinge modifications of the invention”, “modified hinge(s) of the invention” or simply “hinge modifications” or “modified hinge(s).” The modified hinge regions disclosed herein may be incorporated into a molecule of choice including, but not limited to, antibodies and fragments thereof. In some embodiments, the hinge region may be truncated and contain only a portion of the full hinge region.

As demonstrated herein, molecules comprising a modified hinge may exhibit altered (e.g., enhanced) agonistic activity when compared to a molecule having the same amino acid sequence except for the modified hinge, such as, for example, a molecule having the same amino acid sequence except comprising a wild type hinge. In some embodiments, the antibody comprises a modified hinge region wherein the upper hinge region is up to 7 amino acids in length. In some embodiments, the upper hinge region is absent. In some embodiments, the modified hinge is a modified IgG1 linker. In some embodiments, the modified IgG1 hinge is derived from the sequence PLAPDKTHT (SEQ ID NO: 910). In some embodiments, the modified IgG1 hinge comprises the sequence PLAP (SEQ ID NO: 911). In some embodiments, the modified IgG1 hinge comprises the sequence DKTHT (SEQ ID NO: 912). In some embodiments, the modified hinge is a modified IgG4 hinge. In some embodiments, the modified IgG1 hinge comprises the sequence EKSYGPP (SEQ ID NO: 913). In some embodiments, the modified hinge is a Gly/Ser hinge. In some embodiments, the Gly/Ser hinge comprises the sequence GGGGSGGGGSGGGGSGGGGS (SEQ ID NO: 914). In some embodiments, the C-terminal residues of the variable domain adjacent to the upper hinge are truncated. In some embodiments, at least one residue of the variable domain adjacent to the upper hinge is truncated. In some embodiments, at least two residues of the variable domain adjacent to the upper hinge are truncated.

The modified hinge region of the disclosure may be used as a linker to attach one or more binding moieties (e.g., antigen binding domains) of the disclosure. In certain embodiments, a first binding moiety is linked to a second binding moiety via at least one modified hinge region. In certain embodiments, a first variable heavy chain domain (VH1) linked to a second variable heavy chain domain (VH2) via at least one modified hinge region. In certain embodiments, a first variable light chain domain (VL1) linked to a second variable light chain domain (VL2) via at least one modified hinge region. The VH1 and VL1 associate to form a first antigen binding domain and the VH2 and VL2 associate to form a second antigen binding domain. In other embodiments, a first scFv is linked to a second scFv via at least one modified hinge region.

In certain embodiments, the multispecific binding proteins of the disclosure (i.e., multispecific binding proteins having at least a first binding moiety and a second binding moiety) have greater agonist activity compared to a multispecific binding protein that lacks at least one modified hinge region. For example, but in no way limiting, a multispecific binding protein having a VH1 linked to a VH2 via at least one modified hinge region and/or a VL1 linked to a VL2 via at least one modified hinge region may possess greater agonist activity of a target receptor pair (e.g., a human interleukin-12 receptor R 1 (IL-12Rβ 1) subunit and a human IL-12 receptor 32 (IL-12Rβ2) subunit), than the same multispecific binding protein that does not have the at least one modified hinge region.

As used herein, the term “EU index” refers to the EU numbering convention for the constant regions of an antibody, as described in Edelman, G M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety. All numbering of amino acid positions of the Fc polypeptides, or fragments thereof, used herein is according to the EU index. As used herein, the term “linker” refers to 0-100 contiguous amino acid residues. The linkers are, present or absent, and same or different. Linkers comprised in a protein or a polypeptide may all have the same amino acid sequence or may have different amino acid sequences.

In some embodiments, the term “linker” refers to 1-100 contiguous amino acid residues. Typically, a linker provides flexibility and spatial separation between two amino acids or between two polypeptide domains. A linker may be inserted between VH, VL, CH and/or CL domains to provide sufficient flexibility and mobility for the domains of the light and heavy chains depending on the format of the molecule. A linker is typically inserted at the transition between variable domains between variable and constant domain, or between constant and constant domains, respectively, at the amino sequence level. The transition between domains can be identified because the approximate sizes of the immunoglobulin domains are well understood. The precise location of a domain transition can be determined by locating peptide stretches that do not form secondary structural elements such as beta-sheets or alpha-helices as demonstrated by experimental data or as can be determined by techniques of modeling or secondary structure prediction.

As used herein, the term “specifically binds,” “specifically binding,” “binding specificity” or “specifically recognized” refers that an antigen binding protein or antigen-binding fragment thereof that exhibits appreciable affinity for an antigen (e.g., an IL-12R antigen) and does not exhibit significant cross reactivity to a target that is not an IL-12R protein. As used herein, the term “affinity” refers to the strength of the interaction between an antigen binding protein or antigen-binding fragment thereof antigen binding site and the epitope to which it binds. In certain exemplary embodiments, affinity is measured by surface plasmon resonance (SPR), e.g., in a BIAcore® instrument. As readily understood by those skilled in the art, an antigen binding protein affinity may be reported as a dissociation constant (KD) in molarity (M). The antigen binding protein or antigen-binding fragment thereof of the disclosure have KD values in the range of about 10-5 M to about 10-12 M (i.e., low micromolar to picomolar range), about 10-7 M to 10-11 M, about 10-8 M to about 10-10 M, about 10-9 M. In certain embodiments, the antigen binding protein or antigen-binding fragment thereof has a binding affinity of about 10-5 M, 10-6 M, 10-7 M, 10-8 M, 10-9 M, 10-10 M, 10-11 M, or 10-12 M. In certain embodiments, the antigen binding protein or antigen-binding fragment thereof has a binding affinity of about 10-7 M to about 10-9 M (nanomolar range).

Specific binding can be determined according to any art-recognized means for determining such binding. In some embodiments, specific binding is determined by competitive binding assays (e.g., ELISA) or BIAcore® assays. In certain embodiments, the assay is conducted at about 20° C., 25° C., 30° C., or 37° C.

As used herein, “administer” or “administration” refers to the act of injecting or otherwise physically delivering a substance as it exists outside the body (e.g., an isolated binding polypeptide provided herein) into a patient, such as by, but not limited to, pulmonary (e.g., inhalation), mucosal (e.g., intranasal), intradermal, intravenous, intramuscular delivery and/or any other method of physical delivery described herein or known in the art. When a disease, or a symptom thereof, is being managed or treated, administration of the substance typically occurs after the onset of the disease or symptoms thereof. When a disease, or symptom thereof, is being prevented, administration of the substance typically occurs before the onset of the disease or symptoms thereof and may be continued chronically to defer or reduce the appearance or magnitude of disease-associated symptoms.

As used herein, the term “composition” is intended to encompass a product containing the specified ingredients (e.g., an isolated binding polypeptide provided herein) in, optionally, the specified amounts, as well as any product which results, directly or indirectly, from combination of the specified ingredients in, optionally, the specified amounts.

“Effective amount” means the amount of active pharmaceutical agent (e.g., an isolated binding polypeptide of the present disclosure) sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

As used herein, the terms “subject” and “patient” are used interchangeably. As used herein, a subject can be a mammal, such as a non-primate (e.g., cows, pigs, horses, cats, dogs, rats, mice, etc.) or a primate (e.g., monkey and human). In certain embodiments, the term “subject,” as used herein, refers to a vertebrate, such as a mammal. Mammals include, without limitation, humans, non-human primates, wild animals, feral animals, farm animals, sport animals, and pets.

As used herein, the term “therapy” refers to any protocol, method and/or agent that can be used in the prevention, management, treatment and/or amelioration of a disease or a symptom related thereto. In some embodiments, the term “therapy” refers to any protocol, method and/or agent that can be used in the modulation of an immune response to an infection in a subject or a symptom related thereto. In some embodiments, the terms “therapies” and “therapy” refer to a biological therapy, supportive therapy, and/or other therapies useful in the prevention, management, treatment and/or amelioration of a disease or a symptom related thereto, known to one of skill in the art such as medical personnel. In other embodiments, the terms “therapies” and “therapy” refer to a biological therapy, supportive therapy, and/or other therapies useful in the modulation of an immune response to an infection in a subject or a symptom related thereto known to one of skill in the art such as medical personnel.

As used herein, the terms “treat,” “treatment” and “treating” refer to the reduction or amelioration of the progression, severity, and/or duration of a disease or a symptom related thereto, resulting from the administration of one or more therapies (including, but not limited to, the administration of one or more prophylactic or therapeutic agents, such as an isolated binding polypeptide provided herein). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

The term “about” or “approximately” means within about 20%, such as within about 10%, within about 5%, or within about 1% or less of a given value or range.

### The IL-12R Signaling Pathway

IL-12 was identified in the late 1980s from the culture media of Epstein-Barr virus-transformed lymphoblastoid B cell lines, which was able to stimulate natural killer (NK) cell activity, generate lymphokine-activated killer cells, and induce the production of IFN-7 from NK and T-cells (Kobayashi et al. J Exp Med. 1989; 170:827-45). It was found to be a heterodimeric molecule, consisting of a p35 subunit and a p40 subunit, which is formed via disulfide bonds. It has been noted that a number of cell types, including B lymphocytes, macrophages, and dendritic cells, produce IL-12.

The IL-12 receptor is primarily expressed on activated T and NK cells (Desai et al., J Immunol. 1992; 148:3125-32), and comprises the IL-12Rβ 1 subunit and the IL-12Rβ2 subunit. IFN-γ, and to a lesser extent, IL-2 positively affect expression of the receptor. Upon binding of IL-12, heterodimerization of the two subunits occurs. This dimerization induces activation of the receptor-associated JAK molecules JAK2 and Tyk2, which trans-phosphorylate one another as well as tyrosine residues in the intracellular domain of IL-12Rβ2, which serve as docking sites for the SH2-containing STAT4 (Yao et al., Arch. Biochem. Biophys. 1999; 368: 147-155). Receptor-associated STAT4 proteins are phosphorylated before translocating to the nucleus, promoting expression of IFN-7 and the polarization of CD4+ T cells toward a T helper (Th1) phenotype (Trinchieri, Nat. Rev. Immunol. 2003; 3: 133-146). In CD4+ T cells, STAT4 activation by IL-12 is required for the transcription of T-bet, a positive regulator of Th1 differentiation. T-bet enhances the expression of Th1-specific cytokines, chemokines, and Th1's associated receptors. T-bet in combination with STAT4 enhances transcription of CXCR3, IL-12Rβ 1, CCL3, and CCL4. CCL3 and CCL4 are required for the intra-tumoral recruitment of cytotoxic NK cells and CD8+ T cells (Vilgelm et al., Front. Immunol. 2019; 10:333. doi: 10.3389/fimmu.2019.00333; Allen et al., Oncoimmunology. 2018; 7:e1393598. doi: 10.1080/2162402X.2017.1393598; and Allen et al., Front. Immunol. 2017; 8:1390. doi: 10.3389/fimmu.2017.01390).

In the presence of IL-12, NK cells are activated, express CD69 and CD25, and can further proliferate in the tumor microenvironment. Activated Th1 and NK cells proliferate and can infiltrate into the tumor, where Th1 cells support the effector functions of tumor-specific cytotoxic T cells. The IFN-γ, granzyme, and perforin secreted by cytotoxic NK cells and CD8+ T cells can induce apoptosis of cancer cells and control tumor growth. Further, IL-12 has been shown to facilitate antigen presentation by upregulating major histocompatibility complex (MHC) I on tumor cells, favoring polarization of M1 macrophages and attracting effector immune cells by enhancing production of chemokines (e.g., CXCL9, CXCL10, and CXCL11). IL-12 can also neutralize signaling by negative regulatory receptors on CD8+ T cells, such as PD-1 and IFNγR2, protecting tumor infiltrating CD8+ T cells from IFN-γ-induced cell death. IL-12 also inhibits tumor-induced Treg cell proliferation.

Cytokine-based immunotherapy has been shown to be effective for a number of different cancers. Because of its effects on cytotoxic T cells and NK cells, IL-12 has been considered a candidate for immunotherapy-based interventions. However, systemic administration of IL-12 has been shown to be quite toxic, therefore alternative methods of delivering IL-12 or inducing signaling via the IL-12 receptor have been investigated. Instead of systemic administration, localized delivery of IL-12 has been the subject of cancer immunotherapy (Nguyen et al., Front. Immunol. 2020; 11:575597. doi: 10.3389/fimmu.2020.575597.). Others have reported systemic delivery using an oncolytic adenovirus encoding IL-12 lacking a signal peptide to reduce the toxic side effects seen with IL-12 administration (Wang et al., Nat. Commun. 2017; 8:1-15). Other strategies employed have utilized nanoparticle-mediated delivery of IL-12. These findings suggest that increasing signaling via the IL-12 receptor, can be an effective cancer immunotherapy.

### Anti-IL-12Rβ1 Binding Domains

In one aspect of the multi-specific binding protein of the present disclosure is a binding moiety, binding domain, or binding specificity which binds the IL-12Rβ 1 receptor subunit of the IL-12 receptor (e.g., human IL-12R). Any type of binding moiety that specifically binds the IL-12Rβ1 receptor subunit can be employed in the multi-specific binding proteins disclosed herein. In certain embodiments, the binding moiety comprises an antibody variable domain. Exemplary binding moieties comprising an antibody variable domain include, without limitation: a heavy chain variable domain (VH), a light chain variable domain (VL), a VHH, a VH/VL pair, a single-chain variable fragment (scFv), a diabody, or a fragment antigen binding region (Fab). Other suitable binding moiety formats include, without limitation, lipocalins (see e.g., Gebauer M. et al., 2012, Method Enzymol. 503:157-188, which is incorporated by reference herein in its entirety), adnectins (see e.g., Lipovsek D., 2011, Protein Eng. Des. Sel. 24:3-9, which is incorporated by reference herein in its entirety), avimers (see e.g., Silverman J, et al., 2005, Nat. Biotechnol. 23:1556-1561, which is incorporated by reference herein in its entirety), fynomers (see e.g., Schlatter D, et al., 2012, mAbs 4:497-508, which is incorporated by reference herein in its entirety), kunitz domains (see e.g., Hosse R. J. et al., 2006, Protein Sci. 15:14-27, which is incorporated by reference herein in its entirety), knottins (see e.g., Kintzing J. R. et al., 2016, Curr. Opin. Chem. Biol. 34:143-150, which is incorporated by reference herein in its entirety), affibodies (see e.g., Feldwisch J. et al., 2010 J. Mol. Biol. 398:232-247, which is incorporated by reference herein in its entirety), and DARPins (see e.g., Pluckthun A., 2015, Annu. Rev. Pharmacol. Toxicol. 55:489-511, which is incorporated by reference herein in its entirety).

In certain embodiments, the binding domain comprises the heavy and/or light chain variable regions of a conventional antibody or antigen binding fragment thereof (e.g., a Fab or scFv), wherein the term “conventional antibody” is used herein to describe heterotetrameric antibodies containing heavy and light immunoglobulin chains arranged according to the “Y” configuration. Such conventional antibodies may derive from any suitable species including but not limited to antibodies of llama, alpaca, camel, mouse, rat, rabbit, goat, hamster, chicken, monkey, or human origin. In certain exemplary embodiments, the conventional antibody comprises a VH and a VL wherein the VH and/or VL domains or one or more complementarity determining regions (CDRs) thereof are derived from the same antibodies. In certain embodiments, the conventional antibody antigen binding region may be referred to as a “Fab” (Fragment antigen-binding). The Fab comprises one constant and one variable domain from each of heavy chain and light chain. The variable heavy and light chains contain the CDRs responsible for antigen binding.

In some embodiments, the IL-12Rβ1 receptor subunit binding domain comprises the exemplary CDRs as provided in Table 1 and Table 2. In some embodiments, the IL-12Rβ1 receptor subunit binding domain comprises the sequences as provided in Table 3.

TABLE 1
IL-12Rβ1 heavy variable chain (VH) CDRs (HCDR1, HCDR2, and HCDR3)

| Heavy Chain | | | |
|---|---|---|---|
| Hybridoma ID | HCDR1 | HCDR2 | HCDR3 |
| 85801_01D14A | NAWMN | RIKSKTDGGTTDYAAPVKG | DPGWEFDY |
| | (SEQ ID NO: 1) | (SEQ ID NO: 2) | (SEQ ID NO: 3) |
| 85801_01J03A | NYYWS | YIFYNGITDYNPSLKS | ESGMGATLMAFDI |
| | (SEQ ID NO: 4) | (SEQ ID NO: 5) | (SEQ ID NO: 6) |
| 85801_01K04A | NARMGVS | HIFSNDEKSYSTSLKS | IGSGTYYGWFDP |
| | (SEQ ID NO: 7) | (SEQ ID NO: 8) | (SEQ ID NO: 9) |
| 85801_01L02A | DYYMH | WINPDSGGTNYAQKFQG | EGATSNFDY |
| | (SEQ ID NO: 10) | (SEQ ID NO: 11) | (SEQ ID NO: 12) |
| 85801_01M07A | DDYMH | WINPNSGGTNYAQKFQG | GGLNWGWDWFDP |
| | (SEQ ID NO: 13) | (SEQ ID NO: 14) | (SEQ ID NO: 15) |
| 85801_01O02A | DYYMH | WINPDNGGANYAQRFLG | EGSGSPYAFDL |
| | (SEQ ID NO: 16) | (SEQ ID NO: 17) | (SEQ ID NO: 18) |
| 85801_02G10A | SYYIH | IINPSGGITSYAQQFQG | EGDWYFDL |
| | (SEQ ID NO: 19) | (SEQ ID NO: 20) | (SEQ ID NO: 21) |
| 85801_02K24A | SYDWS | RIYTSGSTIYNPSLKS | DRGNYPHDAFDI |
| | (SEQ ID NO: 22) | (SEQ ID NO: 23) | (SEQ ID NO: 24) |
| 85801_02N03A | DYYIH | WINPNSGGTHYAQKFQG | EGGWNYFSGMDV |
| | (SEQ ID NO: 25) | (SEQ ID NO: 26) | (SEQ ID NO: 27) |
| 85801_02O11A | SYDMN | YINSRSSTIYYADSVKG | EIEDSMDV |
| | (SEQ ID NO: 28) | (SEQ ID NO: 29) | (SEQ ID NO: 30) |
| 85801_03P23A | SYWMS | NIKQDGSEKYYVDSVKG | GSIAAPFDY |
| | (SEQ ID NO: 31) | (SEQ ID NO: 32) | (SEQ ID NO: 33) |
| 85801_04D10A | SYAMS | GISGSGGGIYYADSVKG | RLIGYWYFDL |
| | (SEQ ID NO: 34) | (SEQ ID NO: 35) | (SEQ ID NO: 36) |
| 85801_04D14A | DYYMH | WINPNSGGTHYSQKFQG | EGDYISSSAFDY |
| | (SEQ ID NO: 37) | (SEQ ID NO: 38) | (SEQ ID NO: 39) |
| 85801_04E12A | GYYMH | WINPNSSGTNYAQKFQG | MKRWLQGIDF |
| | (SEQ ID NO: 40) | (SEQ ID NO: 41) | (SEQ ID NO: 42) |
| 85801_04G19A | SYYWS | RIYTSGSTIYNPSLKS | SRSYPHDAFDI |
| | (SEQ ID NO: 43) | (SEQ ID NO: 44) | (SEQ ID NO: 45) |
| 85801_04G23A | SYGMH | VIWNDGSNKYYADSVKG | GELFFDY |
| | (SEQ ID NO: 46) | (SEQ ID NO: 47) | (SEQ ID NO: 48) |
| 85801_04M04A | SYYIH | IINPSGGSTSYAQKFQG | SNWAYAFDI |
| | (SEQ ID NO: 49) | (SEQ ID NO: 50) | (SEQ ID NO: 51) |
| 85801_04M10A | SYYWS | RFYTSGSTIYNPSLES | ERDYPHDAFDI |
| | (SEQ ID NO: 52) | (SEQ ID NO: 53) | (SEQ ID NO: 54) |
| 85801_04M12A | SHGMH | IIWYAGNKKYYADSVKG | ESLLNSASDI |
| | (SEQ ID NO: 55) | (SEQ ID NO: 56) | (SEQ ID NO: 57) |
| 85801_04N20A | SYWMS | NIKQDGSEKYYVDSVKG | EELTGLYWYFDL |
| | (SEQ ID NO: 58) | (SEQ ID NO: 59) | (SEQ ID NO: 60) |
| 85801_05E17A | NYYMH | IITSSGGSTSYAQKFQG | EDLYYFDY |
| | (SEQ ID NO: 61) | (SEQ ID NO: 62) | (SEQ ID NO: 63) |
| 85801_05F05A | GYYWN | EINHSGSTHYNPSLKS | ALTGYFDY |
| | (SEQ ID NO: 64) | (SEQ ID NO: 65) | (SEQ ID NO: 66) |
| 85801_05F12A | DYYMH | WINPNSGGTHYAQKFQG | EGETVAGYFDY |
| | (SEQ ID NO: 67) | (SEQ ID NO: 68) | (SEQ ID NO: 69) |
| 85801_05G01A | DYYLH | WINPNNGGTHYAQKFQG | EMGGTTAFDY |
| | (SEQ ID NO: 70) | (SEQ ID NO: 71) | (SEQ ID NO: 72) |
| 85801_06L19A | DYYIH | WINPDSGGTHYAQKFQG | EGEGLASFDS |
| | (SEQ ID NO: 73) | (SEQ ID NO: 74) | (SEQ ID NO: 75) |
| 85801_07A01A | SYWMS | NIKQDGSEKYYVDSVKG | EGLQLGIFDY |
| | (SEQ ID NO: 76) | (SEQ ID NO: 77) | (SEQ ID NO: 78) |
| 85801_07A17A | SYFWS | YLDYRGSTSYNPSVKS | DLEDAFDV |
| | (SEQ ID NO: 79) | (SEQ ID NO: 80) | (SEQ ID NO: 81) |
| 85801_07A19A | DYYMH | WINPNSGGTNYAQKFQG | EVGDIAVAGVFDY |
| | (SEQ ID NO: 82) | (SEQ ID NO: 83) | (SEQ ID NO: 84) |
| 85801_07H04A | SYSMN | YFSSRSGTKYYADSVKG | EPHWDAFDL |
| | (SEQ ID NO: 85) | (SEQ ID NO: 86) | (SEQ ID NO: 87) |
| 85801_07L08A | SYYMH | IINPSGGITSYAQKFQG | DPDWGFFDY |
| | (SEQ ID NO: 88) | (SEQ ID NO: 89) | (SEQ ID NO: 90) |
| 85801_07O09A | SDYWI | RIYTSGSTNYNPSLKS | EVTGEFDY |
| | (SEQ ID NO: 91) | (SEQ ID NO: 92) | (SEQ ID NO: 93) |
| 85801_08M02A | DYYIH | WINPDSGGTTYAQKFQG | EGSGSSSFDY |
| | (SEQ ID NO: 94) | (SEQ ID NO: 95) | (SEQ ID NO: 96) |
| 85801_10F19A | DYYMH | WINPNSGGTHYAQKFQG | EGATTVYFDY |
| | (SEQ ID NO: 97) | (SEQ ID NO: 98) | (SEQ ID NO: 99) |
| 85801_10K15A | TGGVGVG | LIYWNDDKRYSPSLKS | TLRWYHGFDI |
| | (SEQ ID NO: 100) | (SEQ ID NO: 101) | (SEQ ID NO: 102) |
| 85801_10M19A | SYDMH | VIGTAGDTYYPGSVKG | SRGETDWYFDL |
| | (SEQ ID NO: 103) | (SEQ ID NO: 104) | (SEQ ID NO: 105) |

TABLE 2
IL-12Rβ1 light variable chain (VL) CDRs (LCDR1, LCDR2, and LCDR3)

| Light Chain | | | |
|---|---|---|---|
| Hybridoma | | | |
| ID | LCDR1 | LCDR2 | LCDR3 |
| 85801_01D14A | QASQDISNFLN | DASDLET | QQYDNLPLT |
| | (SEQ ID NO: 106) | (SEQ ID NO: 107) | (SEQ ID NO: 108) |
| 85801_01J03A | KSSQSVLYSSNNKNYLA | WASTRES | QQYYSSPLT |
| | (SEQ ID NO: 109) | (SEQ ID NO: 110) | (SEQ ID NO: 111) |
| 85801_01K04A | TGTSSDIGSYNLVS | EGSKRPS | CSYAGTSTFV |
| | (SEQ ID NO: 112) | (SEQ ID NO: 113) | (SEQ ID NO: 114) |
| 85801_01L02A | RASQGISNYLA | AASNLQG | QQYRYYPFT |
| | (SEQ ID NO: 115) | (SEQ ID NO: 116) | (SEQ ID NO: 117) |
| 85801_01M07A | RASQNINNNYLA | GASSRAT | QQYGSSLT |
| | (SEQ ID NO: 118) | (SEQ ID NO: 119) | (SEQ ID NO: 120) |
| 85801_01O02A | RASQGISSYLA | AASTLQS | QQLNRYPFT |
| | (SEQ ID NO: 121) | (SEQ ID NO: 122) | (SEQ ID NO: 123) |
| 85801_02G10A | RASQSVSSSYLA | GASSRAT | QQYGSSYT |
| | (SEQ ID NO: 124) | (SEQ ID NO: 125) | (SEQ ID NO: 126) |
| 85801_02K24A | RASQGISSFLA | AASTLQS | QQLNGYPFT |
| | (SEQ ID NO: 127) | (SEQ ID NO: 128) | (SEQ ID NO: 129) |
| 85801_02N03A | RASQGISSYLA | AASTLHS | QQLNRYPYT |
| | (SEQ ID NO: 130) | (SEQ ID NO: 131) | (SEQ ID NO: 132) |
| 85801_02O11A | SGSSSNIGNNYVS | DNNERPS | GTWDSSLSAGV |
| | (SEQ ID NO: 133) | (SEQ ID NO: 134) | (SEQ ID NO: 135) |
| 85801_03P23A | QASQDISNYLN | DASNLET | QQYDNLPIT |
| | (SEQ ID NO: 136) | (SEQ ID NO: 137) | (SEQ ID NO: 138) |
| 85801_04D10A | RASQGISNYLA | AASSLQS | QQYNSYPPT |
| | (SEQ ID NO: 139) | (SEQ ID NO: 140) | (SEQ ID NO: 141) |
| 85801_04D14A | RASQGISSYLA | AASTLQS | QQLHRYPYT |
| | (SEQ ID NO: 142) | (SEQ ID NO: 143) | (SEQ ID NO: 144) |
| 85801_04E12A | RSSQSLLDSNNGNTYLD | TLSYRAS | MQRIDFPLT |
| | (SEQ ID NO: 145) | (SEQ ID NO: 146) | (SEQ ID NO: 147) |
| 85801_04G19A | RASQGISSYLA | AASTLQS | QQLNGYPFT |
| | (SEQ ID NO: 148) | (SEQ ID NO: 149) | (SEQ ID NO: 150) |
| 85801_04G23A | QASQDISNYLN | DASNLET | QQYDNLFT |
| | (SEQ ID NO: 151) | (SEQ ID NO: 152) | (SEQ ID NO: 153) |
| 85801_04M04A | QGDSLSSYYAG | GKNNRPS | NSRDSSDNHLV |
| | (SEQ ID NO: 154) | (SEQ ID NO: 155) | (SEQ ID NO: 156) |
| 85801_04M10A | RASQDISSFLA | AASTLQS | QQLNGYPLT |
| | (SEQ ID NO: 157) | (SEQ ID NO: 158) | (SEQ ID NO: 159) |
| 85801_04M12A | SGSSSNIGNNYVS | DNNKRPS | GTWDSSLSAVV |
| | (SEQ ID NO: 160) | (SEQ ID NO: 161) | (SEQ ID NO: 162) |
| 85801_04N20A | RASQSINSNYLA | GASSRAT | QQYDS |
| | (SEQ ID NO: 163) | (SEQ ID NO: 164) | (SEQ ID NO: 165) |
| 85801_05E17A | TGTSSDVGSYNLVS | EGSKRPS | CSYAGSSTFYV |
| | (SEQ ID NO: 166) | (SEQ ID NO: 167) | (SEQ ID NO: 168) |
| 85801_05F05A | SGSSSNIGSNYVY | SNNQRPS | AAWDDSLSGPVV |
| | (SEQ ID NO: 169) | (SEQ ID NO: 170) | (SEQ ID NO: 171) |
| 85801_05F12A | RASQGISSYLA | AASTLQN | QQHNSYPYT |
| | (SEQ ID NO: 172) | (SEQ ID NO: 173) | (SEQ ID NO: 174) |
| 85801_05G01A | RASQGIGNYLA | AASSLQS | QQYNIYPYT |
| | (SEQ ID NO: 175) | (SEQ ID NO: 176) | (SEQ ID NO: 177) |
| 85801_06L19A | RASQGINNYLA | AASSLQS | QQYNNYPFT |
| | (SEQ ID NO: 178) | (SEQ ID NO: 179) | (SEQ ID NO: 180) |
| 85801_07A01A | QASQDISNYLN | DASNLET | QQYDNLPFT |
| | (SEQ ID NO: 181) | (SEQ ID NO: 182) | (SEQ ID NO: 183) |
| 85801_07A17A | SGSSSNIGNNYVS | DNNERPS | GTWDSNLSAGV |
| | (SEQ ID NO: 184) | (SEQ ID NO: 185) | (SEQ ID NO: 186) |
| 85801_07A19A | RASQGISSYLA | AASTLQS | QQHSRYPYT |
| | (SEQ ID NO: 187) | (SEQ ID NO: 188) | (SEQ ID NO: 189) |
| 85801_07H04A | SGSSSNIGSNTVN | SNNQRPS | AAWDDSLNGHV |
| | (SEQ ID NO: 190) | (SEQ ID NO: 191) | (SEQ ID NO: 192) |
| 85801_07L08A | RASQSISSWLA | KASSLES | QQYTSYMYT |
| | (SEQ ID NO: 193) | (SEQ ID NO: 194) | (SEQ ID NO: 195) |
| 85801_07O09A | QASQDISNYLN | DASNLET | QQYDNLYT |
| | (SEQ ID NO: 196) | (SEQ ID NO: 197) | (SEQ ID NO: 198) |
| 85801_08M02A | RASQAISNYLA | TASSLQS | QQYRFYPYT |
| | (SEQ ID NO: 199) | (SEQ ID NO: 200) | (SEQ ID NO: 201) |
| 85801_10F19A | RASQGINNYLA | DASTLQS | QQLNMYPYT |
| | (SEQ ID NO: 202) | (SEQ ID NO: 203) | (SEQ ID NO: 204) |
| 85801_10K15A | GLTSGSVSTSYYPS | STYTRSS | VLYMGSGIWV |
| | (SEQ ID NO: 205) | (SEQ ID NO: 206) | (SEQ ID NO: 207) |
| 85801_10M19A | RASQSVSRLLA | DASNRAT | QQRRNWLT |
| | (SEQ ID NO: 208) | (SEQ ID NO: 209) | (SEQ ID NO: 210) |

TABLE 3
IL-12Rβ1 VH and VL sequences.

| Hybridoma ID | VH | VL |
|---|---|---|
| 85801_01D14A | EVQLVESGGGLVKPGGSLRLSC | DIQMTQSPSSLSASVGDRVTITCQ |
| | AASGFTFSNAWMNWVRQAPG | ASQDISNFLNWYQQKPGKAPNLL |
| | KGLEWVGRIKSKTDGGTTDYA | IYDASDLETGVPSRFSGSGSGTDF |
| | APVKGRFTVSRDDPKNTLYLQ | TFTISSLQPEDIATYYCQQYDNLP |
| | MNSLKTEDTAVYYCTTDPGWE | LTFGGGTKVEIK (SEQ ID NO: 212) |
| | FDYWGQGILVTVSS (SEQ ID NO: | |
| | 211) | |
| 85801_01J03A | QVQLQESGPGLVKPSETLSLTC | DIVMTQSPDSLAVSLGERATINCK |
| | TVSGGSISNYYWSWIRQPPGKG | SSQSVLYSSNNKNYLAWYQQKP |
| | LEWIGYIFYNGITDYNPSLKSR | GQPPKMVIYWASTRESGVPDRFS |
| | VTISVDTSKNQFSLKLSSVTAA | GSGSGTDFTLTISSLQAEDVAVYY |
| | DTAMYFCVRESGMGATLMAF | CQQYYSSPLTFGGGTKVEIK (SEQ |
| | DIWGQGTMVTVSS (SEQ ID NO: | ID NO: 214) |
| | 213) | |
| 85801_01K04A | QVTLKESGPVLVKPTETLTLTC | QSALTQPASVSGSPGQSITISCTGT |
| | TVSGFSLTNARMGVSWIRQPPG | SSDIGSYNLVSWYQQHPGKAPKL |
| | KALEWLAHIFSNDEKSYSTSLK | LIYEGSKRPSGVSNRFSGSKSGTT |
| | SRFTISKDTSKSQVVLTMTNLD | ASLTISGLQAEDEADYYCCSYAG |
| | PVDTATYYCARIGSGTYYGWF | TSTFVFGTGTKVTVL (SEQ ID NO: |
| | DPWGQGTLVTVSS (SEQ ID NO: | 216) |
| | 215) | |
| 85801_01L02A | QVQLVHSGPEVKKPGASVKVS | DIQMTQSPSSLSASVGDRVTITCR |
| | CKASGYTFTDYYMHWVRQAP | ASQGISNYLAWFQQKPGKAPTSLI |
| | GQGLEWMGWINPDSGGTNYA | YAASNLQGGVPSKFSGSGSGTDF |
| | QKFQGRVTMTRDTSISTAYME | TLTISSLQPEDFATYYCQQYRYYP |
| | LSRLRSDDTAVYHCAREGATS | FTFGPGTKVDIK (SEQ ID NO: 218) |
| | NFDYWGQGTLVTVSS (SEQ ID | |
| | NO: 217) | |
| 85801_01M07A | QVQLVQSGAEVKKPGASVKVS | EIVLTQSPGTLSLSPGERATLSCRA |
| | CKASEYTFTDDYMHWVRQAP | SQNINNNYLAWYQRKPGQAPRLL |
| | GQGLVWMGWINPNSGGTNYA | IYGASSRATGIPDRFSGSGSGTDFT |
| | QKFQGRVTMTRDTSISTAYME | LTISRLEPEDFAMYYCQQYGSSLT |
| | LSRLRSDDTAVYYCARGGLNW | FGGGTKVEIK (SEQ ID NO: 220) |
| | GWDWFDPWGQGTLVTVSS | |
| | (SEQ ID NO: 219) | |
| 85801_01O02A | QVQLVQSGAEVRKPGASVKVS | DIQLTQSPSFLSASVGDRVTITCRA |
| | CKASGYTFTDYYMHWVRQAP | SQGISSYLAWYQQKPGKAPKLLI |
| | GQGLEWMGWINPDNGGANYA | YAASTLQSGVPSRFSGSGSGTEFT |
| | QRFLGRVTMTRDTSISTAYMEL | LTISSLQPEDFATYCCQQLNRYPF |
| | SRLRSDDTAVYYCAREGSGSP | TFGQGTKLEIK (SEQ ID NO: 222) |
| | YAFDLWGLGTVVTVSS (SEQ ID | |
| | NO: 221) | |
| 85801_02G10A | QVQLVQSGAEVKKPGASVKVS | EIVLTQSPGTLSLSPGERATLSCRA |
| | CKTSGYTFTSYYIHWVRQAPG | SQSVSSSYLAWYQQKPGQAPRLL |
| | QGLEWMGIINPSGGITSYAQQF | IYGASSRATGIPDRESGSGSGTDFT |
| | QGRVTMTRDTSTSTVYMELSS | LTISRLEPEDFAVYYCQQYGSSYT |
| | LRSEDTAVYYCAREGDWYFDL | FGQGTKLEIK (SEQ ID NO: 224) |
| | WGRGTLVTVSS (SEQ ID NO: | |
| | 223) | |
| 85801_02K24A | QVQLQESGPGLVKPSETLSLTC | DIQLTQSPSFLSASVGDRVTITCRA |
| | TVSGGSISSYDWSWIRQSAGKG | SQGISSFLAWYQQKPGKAPKLLIY |
| | LEWIGRIYTSGSTIYNPSLKSRV | AASTLQSGVPSRFSGSGSGTEFTL |
| | TMSVDTSKNEISLKLSSVTAAD | TISSLQPEDFATYYCQQLNGYPFT |
| | TAFYYCAKDRGNYPHDAFDIW | FGPGTKVDIK (SEQ ID NO: 226) |
| | GQGTMITVSS (SEQ ID NO: | |
| | 225) | |
| 85801_02N03A | QVHLVQSGAEVKKPGASVKVS | DIQLTQSPSFLSASVGDRVTITCRA |
| | CEASGYTFTDYYIHWLRQAPG | SQGISSYLAWYQQQPGKPPKLLIY |
| | QGLEWMGWINPNSGGTHYAQ | AASTLHSGVPSRFSGSGSGTEFTL |
| | KFQGRVTMTRDTSISTAYMELS | TISSLQPEDFATYYCQQLNRYPYT |
| | RLSSDDTAVYYCAREGGWNYF | CGQGTKLEIK (SEQ ID NO: 228) |
| | SGMDVWGQGTTVTVSS (SEQ ID | |
| | NO: 227) | |
| 85801_02O11A | EVQLVESGGGLVQPGGSLRLSC | QSVLTQPPSVSAAPGQKVTISCSG |
| | AASGFTFSSYDMNWVRQAPGK | SSSNIGNNYVSWYQQLPGTAPKL |
| | GLEWISYINSRSSTIYYADSVKG | LIYDNNERPSGIPDRFSGSKSGTSA |
| | RFTISRDNAKNSLYLQMNSLRD | TLGITGLQPGDEADYYCGTWDSS |
| | EDTALYYCAREIEDSMDVWGQ | LSAGVFGGGTKLTVL (SEQ ID NO: |
| | GTTVTVSS (SEQ ID NO: 229) | 230) |
| 85801_03P23A | EVQLVESGGGLVQPGGSLRLSC | DIQMTQSPSSLSASVGDRVTITCQ |
| | AASGFTFSSYWMSWVRQAPGK | ASQDISNYLNWYQQKPGKAPKLL |
| | GLEWVANIKQDGSEKYYVDSV | IYDASNLETGVPSRFSGSGSGTDF |
| | KGRFTISRDNAKNSLYLQMNSL | TFTISSLQPEDIATYYCQQYDNLPI |
| | RAEDTAVYYCARGSIAAPFDY | TFGQGTRLEIK (SEQ ID NO: 232) |
| | WGQGTLVTVSS (SEQ ID NO: | |
| | 231) | |
| 85801_04D10A | EVQLLESGGGLVQPGGSLRLSC | DIQMTQSPSSLSASVGDRVTITCR |
| | AASGFTFSSYAMSWVRQAPGK | ASQGISNYLAWFQQKPGKAPKSLI |
| | GLEWVAGISGSGGGIYYADSV | YAASSLQSGVPSKFSGSGSGTDFT |
| | KGRFTISRDNSKNTLYLQLNSL | LTISSLQPEDFATYYCQQYNSYPP |
| | RADDTAVYYCARRLIGYWYFD | TFGGGTKVEIK (SEQ ID NO: 234) |
| | LWGRGTLVPVSS (SEQ ID NO: | |
| | 233) | |
| 85801_04D14A | QVHLVQSGAEVKKPGASVKVS | DIQLTQSPSFLSASVGDRVTITCRA |
| | CKASGYTFIDYYMHWVRQAPG | SQGISSYLAWYQQKPGKAPKVLI |
| | QGLEWMGWINPNSGGTHYSQ | YAASTLQSGVPSRFSGSGSGTEFT |
| | KFQGRVTMTWDTSISTAYMEL | LTISSLLPEDFATYFCQQLHRYPY |
| | SRLTSDDTAVYYCAREGDYISS | TFGQGAKLEIK (SEQ ID NO: 236) |
| | SAFDYWGQGTLVTVSS (SEQ ID | |
| | NO: 235) | |
| 85801_04E12A | QVQLVQSGAEVMKPGASVKVS | DIVMTQTPLSLPVTPGEPASISCRS |
| | CKASGYTFTGYYMHWIRQAPG | SQSLLDSNNGNTYLDWYLQKPG |
| | QGLEWMGWINPNSSGTNYAQ | QSPHLLIYTLSYRASGVPDRFSGS |
| | KFQGRVTMTRDTSISTAYMELS | GSGTDFTLKISRVEAEDVGVYYC |
| | RLRSDDTAVYYCARMKRWLQ | MQRIDFPLTFGGGTKVEIK (SEQ ID |
| | GIDFWGQGILVTVSS (SEQ ID | NO: 238) |
| | NO: 237) | |
| 85801_04G19A | QVQLQESGPGLVKPSETLSLTC | DIQLTQSPSFLSASVGYRVTITCRA |
| | TVSGGSISSYYWSWIRQSAGKE | SQGISSYLAWYQQKPGKAPQLLI |
| | LEWIGRIYTSGSTIYNPSLKSRV | YAASTLQSGVPSRFSGRGSGTEFT |
| | TMSVDPSKNQFSLKLSSVTAAD | LTISSLQPEDFATYYCQQLNGYPF |
| | TAVYYCANSRSYPHDAFDIWG | TFGPGTKVNIK (SEQ ID NO: 240) |
| | QGTMVTVSS (SEQ ID NO: 239) | |
| 85801_04G23A | QVQLVESGGGVVQPGRSLRLS | DIQMTQSPSSLSASVGDRVTITCQ |
| | CAASGFTFSSYGMHWVRQAPG | ASQDISNYLNWYQQKPGKAPKLL |
| | KGLEWVAVIWNDGSNKYYAD | IYDASNLETGVPSRFSGSGSGTDF |
| | SVKGRFTISRDNSKNTLYLQMN | TFTISSLQPEDIATYYCQQYDNLF |
| | SLRAEDTAVYYCARGELFFDY | TFGGGTKVEIK (SEQ ID NO: 242) |
| | WGQGTLVTVSS (SEQ ID NO: | |
| | 241) | |
| 85801_04M04A | QVQLVQSGAEVKKPGASVRVS | SSELTQDPAVSVALGQTVRITCQG |
| | CKASGYTFTSYYIHWVRQAPG | DSLSSYYAGWYQQKPGQAPVLVI |
| | QGLEWMGIINPSGGSTSYAQKF | YGKNNRPSGIPDRFSGSSSEDTAS |
| | QGRVTMTRDTSTTTVYMELSS | LTITGAQAEDEADYYCNSRDSSD |
| | LRSEDTAVYYCAKSNWAYAFD | NHLVFGGGTKLTVL (SEQ ID NO: |
| | IWGQGTMVTVSS (SEQ ID NO: | 244) |
| | 243) | |
| 85801_04M10A | QVQLQESGPGLVKPSETLSLTC | DIQLTQSPSFLSASVGDRVTIPCRA |
| | TVSGGSISSYYWSWIRQPAGTG | SQDISSFLAWYQQKPGKAPNLLIY |
| | LEWIGRFYTSGSTIYNPSLESRV | AASTLQSGVPSRFSGSGSGTEFTL |
| | TMSVDTSKNQFSLKLSSVTAA | TISSLQPEDFATYYCQQLNGYPLT |
| | DTAVYYCATERDYPHDAFDIW | FGGGTKVEIK (SEQ ID NO: 246) |
| | GQGTMVTVSS (SEQ ID NO: | |
| | 245) | |
| 85801_04M12A | QVQLVESGGGVVQPGRSLRLS | QSVLTQPPSVSAAPGQTVTISCSG |
| | CAASGFTFSSHGMHWVRQAPG | SSSNIGNNYVSWYQQLPGTAPKL |
| | KGLEWVAIIWYAGNKKYYADS | LIYDNNKRPSGIPDRFSGSKSGTS |
| | VKGRFTISRDNSKNTLYLQMNS | ATLGITGLQTGDEADYYCGTWDS |
| | LRAEDTAVYYCARESLLNSAS | SLSAVVFGGGTKLTVL (SEQ ID |
| | DIWGQGTMVTVSS (SEQ ID NO: | NO: 248) |
| | 247) | |
| 85801_04N20A | EVQLVESGGGLVQPGGSLRLSC | EIVLTQSPGTLSLSPGERATLSCRA |
| | AASGFTFSSYWMSWVRQAPGK | SQSINSNYLAWYQQKPGQAPRLLI |
| | GLEWVANIKQDGSEKYYVDSV | YGASSRATGIPDRFSGSGSGTDFT |
| | KGRFTISRDNAKNSLYLQMNSL | LTISRLEPEDFAVYYCQQYDSFGG |
| | RAEDTAVYYCAREELTGLYWY | GTKVEIK (SEQ ID NO: 250) |
| | FDLWGRGTLVTVSS (SEQ ID NO: | |
| | 249) | |
| 85801_05E17A | QVQLVQSGAEVKKPGASVKVS | QSALTQPASVSGSPGQSITISCTGT |
| | CKASGYTFTNYYMHWVRQAP | SSDVGSYNLVSWYQQHPGKAPK |
| | GQGLEWMGIITSSGGSTSYAQK | LMIYEGSKRPSGVSNRFSGSKSGN |
| | FQGRVTMTRDTSTSTVYMELS | TASLTISGLQAEDEADYYCCSYA |
| | SLKSEDTAVYYCAREDLYYFD | GSSTFYVFGTGTKVTVL (SEQ ID |
| | YWGQGTLVTVSS (SEQ ID NO: | NO: 252) |
| | 251) | |
| 85801_05F05A | QVQLQQWGAGLLKPSETLSLT | QSVLTQPPSASGTPGQRVTISCSG |
| | CAVYGGSFSGYYWNWIRQPPG | SSSNIGSNYVYWYQQLPGTAPKL |
| | KGLEWIGEINHSGSTHYNPSLK | LIYSNNQRPSGVPDRFSGSKSGTS |
| | SRVTISVDTSKNQFSLKLSSVTA | ASLAISGLRSEDEADYYCAAWDD |
| | ADTALYYCARALTGYFDYWG | SLSGPVVFGGGTKLTVL (SEQ ID |
| | QGTLVTVSS (SEQ ID NO: 253) | NO: 254) |
| 85801_05F12A | QVQLVQSGAEVKKPGASVKVS | DIQLTQSPSFLSASVGDRVTITSRA |
| | CKASGYTFTDYYMHWVRQAP | SQGISSYLAWYQQKPGKAPKLLI |
| | GQGLEWMGWINPNSGGTHYA | YAASTLQNGVPSRFSGSGSGTEFT |
| | QKFQGRVTMTKDTSISTAYME | LTISSLQPEDFATYYCQQHNSYPY |
| | LSRLRSDDTAVYYCAREGETV | TFGQGTKLEIK (SEQ ID NO: 256) |
| | AGYFDYWGQGILVTVSS (SEQ | |
| | ID NO: 255) | |
| 85801_05G01A | QVQLVQSGAEVKKPGASAKVS | DIQMTQSPSSLSASVGDRVTITCR |
| | CKASGYTFTDYYLHWVRQAPG | ASQGIGNYLAWFQQRPGKAPKSL |
| | QGLEWMGWINPNNGGTHYAQ | IYAASSLQSGVPSKFSGSGSGTDF |
| | KFQGRVTMTRDTSISTAYMELS | TLTISSLQPADFAIYYCQQYNIYPY |
| | RLRSDDTAVYYCAREMGGTTA | TFGPGTRLEIK (SEQ ID NO: 258) |
| | FDYWGQGTLVTVSS (SEQ ID | |
| | NO: 257) | |
| 85801_06L19A | QVQLVQSGAEVKKPGASVKVS | DIQMTQSPSSLSASVGNRVTITCR |
| | CKASGYTFTDYYIHWVRQAPG | ASQGINNYLAWFQQKPGKAPKSL |
| | QGLEWMGWINPDSGGTHYAQ | IYAASSLQSGVPSKFSGSGSGTDF |
| | KFQGRVTMTRDTSISTVYMDL | TLTISSLQPEDFATYYCQQYNNYP |
| | SRLRSDDTAVYYCAREGEGLA | FTFGPGTKVDIK (SEQ ID NO: 260) |
| | SFDSWGQGTLVTVSS (SEQ ID | |
| | NO: 259) | |
| 85801_07A01A | EVQLVESGGGLVQPGGSLRLSC | DIQMTQSPSSLSASVGDRVTITCQ |
| | AASGFTFSSYWMSWVRQAPGK | ASQDISNYLNWYQQKPGKAPKLL |
| | GLEWVANIKQDGSEKYYVDSV | IYDASNLETGVPSRFSGSGSGTDF |
| | KGRFTISRDNAKNSLYLQMNSL | TFTISSLQPEDIATYYCQQYDNLPF |
| | RAEDTAVYYCAREGLQLGIFD | TFGPGTKVDIK (SEQ ID NO: 262) |
| | YWGQGTLVTVSS (SEQ ID NO: | |
| | 261) | |
| 85801_07A17A | QVQLQESGPGLVKPSETLSLTC | QSVLTQPPSVSAAPGQKVTISCSG |
| | TVSGVSISSYFWSWIRQPPGKG | SSSNIGNNYVSWYQQLPGTAPKL |
| | LEWIGYLDYRGSTSYNPSVKSR | LIYDNNERPSGIPDRFSGSKSGTSA |
| | VTISLDTSKNQFSLKLTSVTAA | TLGITGLQTGDEADYYCGTWDSN |
| | DTAAYYCARDLEDAFDVWGQ | LSAGVFGGGTRLTVL (SEQ ID NO: |
| | GTMVTVSS (SEQ ID NO: 263) | 264) |
| 85801_07A19A | QVQLVQSGAEVKKPGASVKVS | DIQLTQSPSFLSASVGDRVTITCRA |
| | CKASGYTFTDYYMHWVRQAP | SQGISSYLAWYQQKPGKAPKLLI |
| | GQGLEWMGWINPNSGGTNYA | YAASTLQSGVPSRFSGSGSGTEFT |
| | QKFQGRVTMTRDTSISTAYME | VTISSLQPEDFATYYCQQHSRYPY |
| | LSRLRSDDTAVYYCAREVGDI | TFGQGTKVEIK (SEQ ID NO: 266) |
| | AVAGVFDYWGQGTLVTVSS | |
| | (SEQ ID NO: 265) | |
| 85801_07H04A | EVQLVESGGGLVQPGGSLRLSC | QSVLTQPPSASGTPGQRVTISCSG |
| | AASGFTFSSYSMNWVRQAPGK | SSSNIGSNTVNWYQQLPGTAPKL |
| | GLEWLSYFSSRSGTKYYADSV | LIYSNNQRPSGVPDRESGSKSGTS |
| | KGRFTISRDNAKNSLYLQMNSL | ASLAISGLQSEDEADYYCAAWDD |
| | RDEDTAVYYCAREPHWDAFDL | SLNGHVFGTGTKVTVL (SEQ ID |
| | WGQGTMVTVSS (SEQ ID NO: | NO: 268) |
| | 267) | |
| 85801_07L08A | QVQLVQSGAEVKKPGASVKVS | DIQMTQSPSTLSASVGDRVTITCR |
| | CKPSGYTFTSYYMHWVRQAPG | ASQSISSWLAWYQQKPGKAPKLL |
| | QGLEWMGIINPSGGITSYAQKF | IYKASSLESGVPSRFSGSGSGTEFT |
| | QGRVTMTRDTSTSTVYMELSS | LTISSLQPDDFATYYCQQYTSYM |
| | LRSEDTAVYYCARDPDWGFFD | YTFGQGTKLEIK (SEQ ID NO: 270) |
| | YWGQGSLVTVSS (SEQ ID NO: | |
| | 269) | |
| 85801_07O09A | QVHLQESGPGLVKPSETLSLTC | DIQMTQSPSSLSASVGDRVTITCQ |
| | TVSGVSISSDYWIWIRQPAGKG | ASQDISNYLNWYQQKPGKAPKLL |
| | LEWIGRIYTSGSTNYNPSLKSR | IYDASNLETGVPSRFSGSGSGTDF |
| | VTMSVDTSKNQFSLKLSSVTA | TFTISSLQPEDIATYYCQQYDNLY |
| | ADTAVYYCAREVTGEFDYWG | TFGQGTKLEIK (SEQ ID NO: 272) |
| | QGTLVTVSS (SEQ ID NO: 271) | |
| 85801_08M02A | QVQLVQTGAEVKKPGASVKVS | DIQMTQSPSSLSASVGDRVTITCR |
| | CKASRYTFTDYYIHWVRQAPG | ASQAISNYLAWFQQKPGKAPKSLI |
| | QGLEWMGWINPDSGGTTYAQ | YTASSLQSGVPSKFSGSGSGTDFT |
| | KFQGRVTMTRDTSINTAYMEL | LTISSLHPEDFATYYCQQYRFYPY |
| | SRLRSDDTAVYFCAREGSGSSS | TFGLGTKLEIK (SEQ ID NO: 274) |
| | FDYWGQGTLVTVSS (SEQ ID | |
| | NO: 273) | |
| 85801_10F19A | QVQLVQSGAEVRKPGASVKVS | DIQLTQSPSFLSASVGDRVTLTCR |
| | CKASGYTFTDYYMHWVRQAP | ASQGINNYLAWYQEKPGKAPNPL |
| | GQGREWMGWINPNSGGTHYA | IYDASTLQSGVPSRFSGSGSGTEFT |
| | QKFQGRVTMTWDTSISTAYME | LTISSLQPEDFATYYCQQLNMYPY |
| | LSRLRSDDTAVYYCAREGATT | TFGQGTKLEIK (SEQ ID NO: 276) |
| | VYFDYWGQGTLVTVSS (SEQ ID | |
| | NO: 275) | |
| 85801_10K15A | QITLKESGPTLVKPTQTLTLTCT | QTVVTQEPSFSVSPGGTVTLTCGL |
| | FFGFSLNTGGVGVGWIRQPPGK | TSGSVSTSYYPSWYQQTPGQAPR |
| | ALEWLTLIYWNDDKRYSPSLK | TLIYSTYTRSSGVPDRFSGSILGNK |
| | SRLTITKDTSKNQVVLTMTNM | AALTITGAQADDESDYYCVLYM |
| | DPVDTATYYCAHTLRWYHGF | GSGIWVFGGGTKLTVL (SEQ ID |
| | DIWGQGTMVTVSS (SEQ ID NO: | NO: 278) |
| | 277) | |
| 85801_10M19A | EVQLVESGGGLVQPGGSLRLSC | EIVLTQSPATLSLSPGERATLSCRA |
| | AASGFTFSSYDMHWVRQATGK | SQSVSRLLAWYQQKPGQAPRLLI |
| | GLEWVSVIGTAGDTYYPGSVK | YDASNRATGIPARFSGSGSGTDFT |
| | GRFTISRENAKNSLYLQMNSLR | LTISSLEPEDFAVYYCQQRRNWLT |
| | AGDTAVYYCARSRGETDWYF | FGGGTKVEIK (SEQ ID NO: 280) |
| | DLWGRGTLVTVSS (SEQ ID NO: | |
| | 279) | |

In other embodiments, the IL-12Rβ1 receptor subunit binding domain comprises at least the CDRs or a VHH domain of a VHH antibody or Nanobody. VHH antibodies, which are camelid-derived heavy chain antibodies, are composed of two heavy chains and are devoid of light chains (Hamers-Casterman, et al. Nature. 1993; 363; 446-8). Each heavy chain of the VHH antibody has a variable domain at the N-terminus, and these variable domains are referred to in the art as “VHH” domains in order to distinguish them from the variable domains of the heavy chains of the conventional antibodies i.e., the VH domains. Similar to conventional antibodies, the VHH domains of the molecule comprise HCDR1, HCDR2 and HCDR3 regions which confer antigen binding specificity and therefore VHH antibodies or fragments such as isolated VHH domains, are suitable as components of the multi-specific binding proteins of the present disclosure.

In some embodiments, the IL-12Rβ1 receptor subunit binding domain comprises the exemplary VHH CDRs as provided in Table 4. In some embodiments, the IL-12Rβ1 receptor subunit binding domain comprises the VHH sequences as provided in Table 5.

TABLE 4
IL-12Rβ1 receptor subunit VHH CDR sequences.

| Clone | | | |
|---|---|---|---|
| number | HCDR1 | HCDR2 | HCDR3 |
| VHH25B1 | YFAIG | CIGSSDGSTIYSDSVKG | DRGRRTIIGYACYATMKY |
| | (SEQ ID | (SEQ ID NO: 282) | (SEQ ID NO: 283) |
| | NO: 281) | | |
| VHH32B1 | NYAMS | VINSGGGSTLYADSVKG | RRDDSTFGSLLYTH |
| | (SEQ ID | (SEQ ID NO: 285) | (SEQ ID NO: 286) |
| | NO: 284) | | |
| VHH41B1 | TYAMA | HILWSGGSTVYPDSVRG | KSGRQVYRIDSWDSFDY |
| | (SEQ ID | (SEQ ID NO: 288) | (SEQ ID NO: 289) |
| | NO: 287) | | |
| VHH50B1 | RYSMG | ANYWSNGWTDYADTVKG | RSSSIPSADVVGYDY |
| | (SEQ ID | (SEQ ID NO: 291) | (SEQ ID NO: 292) |
| | NO: 290) | | |
| VHH54B1 | GYVMG | GITGISSTYFADSVKG | VNSFGVIPTSPNGMDY |
| | (SEQ ID | (SEQ ID NO: 294) | (SEQ ID NO: 295) |
| | NO: 293) | | |
| VHH58B1 | YAVG | VAAITVSGGSKYYEDSVKG | RTPKFGSGWYNRRGEYDY |
| | (SEQ ID | (SEQ ID NO: 297) | (SEQ ID NO: 298) |
| | NO: 296) | | |
| VHH67B1 | SYAVA | SISWSGGSTNYADSVKG | DRNYYPSVLGKYTY |
| | (SEQ ID | (SEQ ID NO: 300) | (SEQ ID NO: 301) |
| | NO: 299) | | |
| VHH72B1 | NYAMA | GITWGGGLTHYVDSVKG | KTQGSTWYHLTPDGYDY |
| | (SEQ ID | (SEQ ID NO: 303) | (SEQ ID NO: 304) |
| | NO: 302) | | |
| VHH86B1 | AYAMG | GISRSGSTTDYVDTAKG | VTGTFPSSTYHNANAYHH |
| | (SEQ ID | (SEQ ID NO: 306) | (SEQ ID NO: 307) |
| | NO: 305) | | |
| VHH97B1 | NDDMA | HIDRNAASTNYADSVKG | DPDHFGTTWYQQYTY |
| | (SEQ ID | (SEQ ID NO: 309) | (SEQ ID NO: 310) |
| | NO: 308) | | |
| VHH104B1 | INIMG | SITSGGSTFYADSVKD | YRSVPLNTVYP |
| | (SEQ ID | (SEQ ID NO: 312) | (SEQ ID NO: 313) |
| | NO: 311) | | |
| VHH108B1 | DRSMG | AITVGGSTNYADSVKG | RYPPDDY |
| | (SEQ ID | (SEQ ID NO: 315) | (SEQ ID NO: 316) |
| | NO: 314) | | |
| VHH115B1 | YFMG | AISRSGGGTWYADSVKG | DRRTGTSVYTVDEYDY |
| | (SEQ ID | (SEQ ID NO: 318) | (SEQ ID NO: 319) |
| | NO: 317) | | |
| VHH136B1 | YAVG | VAAITVSGGSKYYEDSVKG | RTPKFGSGWYNRRGEYDY |
| | (SEQ ID | (SEQ ID NO: 321) | (SEQ ID NO: 322) |
| | NO: 320) | | |
| VHH137B1 | TYAMG | IAAWKVASTYYVDYGDSVRG | GAFRTDWRSYAY |
| | (SEQ ID | (SEQ ID NO: 324) | (SEQ ID NO: 325) |
| | NO: 323) | | |
| VHH172B1 | YAVG | VAAITVSGGSKYYEDSVKG | RTPKFGSGWYNRRGEYDY |
| | (SEQ ID | (SEQ ID NO: 327) | (SEQ ID NO: 328) |
| | NO: 326) | | |
| VHH154B1 | VNTRA | QISSGGNINYADSVKG | HRQHPYADV |
| | (SEQ ID | (SEQ ID NO: 330) | (SEQ ID NO: 331) |
| | NO: 329) | | |
| VHH174B1 | HYVMA | SIWWGGSTYYADSVKD | GTKIGTMGPRYDY |
| | (SEQ ID | (SEQ ID NO: 333) | (SEQ ID NO: 334) |
| | NO: 332) | | |
| VHH187B1 | YYAIG | CISSSDRSTWYADSVKG | PCYSDYDPEGYEYDY |
| | (SEQ ID | (SEQ ID NO: 336) | (SEQ ID NO: 337) |
| | NO: 335) | | |
| VHH195B1 | TYAMG | RISNSGAFTHYADSVKG | TRGTNSAYYTRSTMYEY |
| | (SEQ ID | (SEQ ID NO: 339) | (SEQ ID NO: 340) |
| | NO: 338) | | |
| VHH202B1 | IDVMA | AISSGGSLNYRDSVKG | NIRTSPVTTRPMGNY |
| | (SEQ ID | (SEQ ID NO: 342) | (SEQ ID NO: 343) |
| | NO: 341) | | |
| VHH233B1 | FNEMA | TIVSTVGFTNYADSVKG | RRISTDY |
| | (SEQ ID | (SEQ ID NO: 345) | (SEQ ID NO: 346) |
| | NO: 344) | | |
| VHH238B1 | SYGMS | YISAGGGTTTYVDFVKG | AADGS |
| | (SEQ ID | (SEQ ID NO: 348) | (SEQ ID NO: 349) |
| | NO: 347) | | |
| VHH245B1 | INAMG | IQLSGQTSYAHSVRG | SNTGSLYGLRNY |
| | (SEQ ID | (SEQ ID NO: 351) | (SEQ ID NO: 352) |
| | NO: 350) | | |
| VHH258B1 | SYVMG | AIMRNGIMTYYADSVKG | ASRVGVSISQSSSYTS |
| | (SEQ ID | (SEQ ID NO: 354) | (SEQ ID NO: 355) |
| | NO: 353) | | |
| VHH263B1 | FNAMG | IIINGDMINYADSVKG | TRYVYDY |
| | (SEQ ID | (SEQ ID NO: 357) | (SEQ ID NO: 358) |
| | NO: 356) | | |
| VHH276B1 | SYAMG | AISGGTPNNIYYTNYKDAVRG | TMGRYWNTGGYDY |
| | (SEQ ID | (SEQ ID NO: 360) | (SEQ ID NO: 361) |
| | NO: 359) | | |
| VHH321B1 | DYAMK | VIDSEGPTIKYSDAVRG | SNGGSAFSSVLYTD |
| | (SEQ ID | (SEQ ID NO: 363) | (SEQ ID NO: 364) |
| | NO: 362) | | |

TABLE 5
IL-12Rβ1 receptor subunit VHH sequences.

| Clone Number | VHH Sequence |
|---|---|
| VHH25B1 | QVQLQESGGALVQPGGSLRLSCVVSGFT |
| | SDYFAIGWFRQAPGKEREGVSCIGSSDG |
| | STIYSDSVKGRFTISRDNAKNTVYLQMN |
| | SLKPEDTGVYYCALDRGRRTIIGYACYA |
| | TMKYWGKGTPVTVSS (SEQ ID NO: 365) |
| VHH32B1 | EVQLVESGGGLVQPGGSLRLSCAASGFT |
| | FQNYAMSWLRKAPGEGLEWVSVINSGG |
| | GSTLYADSVKGRFTISRDNAKNTLYLQM |
| | HSLKSEDTAVYFCAKRRDDSTFGSLLYT |
| | HRGQGTQVTVSS (SEQ ID NO: 366) |
| VHH41B1 | EVQLVESGGGLVQAGGSLRVACAASGR |
| | TFSTYAMAWFRQAPGKEREFVAHILWS |
| | GGSTVYPDSVRGRFTISRDNAKNTVYLQ |
| | MNSLKPEDTAVYYCAQKSGRQVYRIDS |
| | WDSFDYWGQGTSVTVSS (SEQ ID NO: |
| | 367) |
| VHH50B1 | QVQLVESGGGLVQAGGSLRLSCAASGR |
| | AFSRYSMGWFRQAPGKEREFVAANYWS |
| | NGWTDYADTVKGRFSISRDNAKNTVYL |
| | QMNSLKPEDTAVYYCAGRSSSIPSADVV |
| | GYDYWGQGTQVTVSS (SEQ ID NO: 368) |
| VHH54B1 | QVQLVESGGGLVQAGGSLRLSCAASGG |
| | PFSGYVMGWFRQVSGKEREFVAGITGIS |
| | STYFADSVKGRFTISRDNAKNMVYLQM |
| | NSLKPEDTAVYYCAAVNSFGVIPTSPNG |
| | MDYWGKGTLVTVSS (SEQ ID NO: 369) |
| VHH58B1 | QVQLVESGGGLVQAGGSLRLSCAAPGR |
| | YAVGWFRQAPGKEYEFVAAITVSGGSK |
| | YYEDSVKGRFAISRDNAKNTVYLQMNS |
| | LKAEDTAVYYCAARTPKFGSGWYNRRG |
| | EYDYWGQGTQVTVSS (SEQ ID NO: 370) |
| VHH67B1 | QVQLVESGGGLVQAGGSLRLSCAASARI |
| | FNSYAVAWFRQTPGKEREFVASISWSGG |
| | STNYADSVKGRFTISRDNAKNTVFLQM |
| | NSLKPADTAVYYCAADRNYYPSVLGKY |
| | TYWGQGTQVTVSS (SEQ ID NO: 371) |
| VHH72B1 | EVQLVESGGGLVQAGGSLRLSCAASGLT |
| | FRNYAMAWFRQAPGKEREFVAGITWGG |
| | GLTHYVDSVKGRFTISRDNGKNTVYLQ |
| | MNSLKSEDTAVYYCAGKTQGSTWYHLT |
| | PDGYDYWGQGTQVTVSS (SEQ ID NO: |
| | 372) |
| VHH86B1 | QVQLVESGGGLMQAGGSLRLSCALSRGI |
| | FSAYAMGWFRQVQGKEREFVSGISRSGS |
| | TTDYVDTAKGRFTISRDNARDTVYLQM |
| | NSLKPEDTAVYYCAAVTGTFPSSTYHNA |
| | NAYHHWGQGTQVTVSS (SEQ ID NO: |
| | 373) |
| VHH97B1 | QVQLVESGGGLAQAGGSLRLSCAASGR |
| | TFSNDDMAWFRQAPGKEREFVAHIDRN |
| | AASTNYADSVKGRFTISRDNAKNTVYL |
| | QMTRLKPEDTAVYYCAADPDHFGTTW |
| | YQQYTYWGPGTQVTVSS (SEQ ID NO: |
| | 374) |
| VHH104B1 | EVQLVESGGGLVQPGGSLRLFCAASRSIF |
| | NINIMGWYRQAPGKQREWLASITSGGST |
| | FYADSVKDRFTISRDSVKNTVYLQMNSL |
| | KPEDTAAYYCNRYRSVPLNTVYPWGQG |
| | TLVTVSS (SEQ ID NO: 375) |
| VHH108B1 | EVQLVESGGGLVQPGGSLTLSCTASGFR |
| | FSDRSMGWYRQAPGKQRELVAAITVGG |
| | STNYADSVKGRFTISRDNAKNTVYLQM |
| | NSLKPEDTALYYCNRRYPPDDYWGQGT |
| | QVTVSS (SEQ ID NO: 376) |
| VHH115B1 | QVQLVESGGGLVQAGGSLRLSCLASGR |
| | TSNYFMGWFRQAPGKEREFVAAISRSGG |
| | GTWYADSVKGRFAISRDNAKNTVNLQM |
| | NSLKREDTAVYYCAADRRTGTSVYTVD |
| | EYDYWGQGTQVTVSS (SEQ ID NO: 377) |
| VHH136B1 | QVQLQESGGGLVQAGGSLRLSCAAPGR |
| | YAVGWFRQAPGKEYEFVAAITVSGGSK |
| | YYEDSVKGRFTISRDNAKNTVYLEMNSL |
| | KAEDTAVYYCAARTPKFGSGWYNRRGE |
| | YDYWGQGTQVTVSS (SEQ ID NO: 378) |
| VHH137B1 | QVQLVESGGGLVQAGGSLRLSCAASGR |
| | TFRTYAMGWFRQAPGKDREVVGIAAW |
| | KVASTYYVDYGDSVRGRFTISRDDAKST |
| | VYLQMNNLMPEDTAVYYCAAGAFRTD |
| | WRSYAYWGQGTQVTVSS (SEQ ID NO: |
| | 379) |
| VHH172B1 | QVQLVESGGGLVQAGGSLRLSCAAPGR |
| | YAVGWFRQAPGKEYEFVAAITVSGGSK |
| | YYEDSVKGRFTISRDNAKNTVYLEMNSL |
| | KAEDTAVYYCAARTPKFGSGWYNRRGE |
| | YDYWGQGTQVAVSS (SEQ ID NO: 380) |
| VHH154B1 | QVQLQESGGGLVQVGDSLRLSCVASGRI |
| | FGVNTRAWYRQAPGNQRELVAQISSGG |
| | NINYADSVKGRFTISMDKFTDTVYLQM |
| | NSLKPEDTAIYRCAAHRQHPYADVWGQ |
| | GTQVSVSS (SEQ ID NO: 381) |
| VHH174B1 | QVQLVESGGGAVQAGGSLRLSCAASGL |
| | TSRHYVMAWFRQAPGIEREFVASIWWG |
| | GSTYYADSVKDRFTISRDNAENTVYLQ |
| | MNSLKPEDTAVYRCAVGTKIGTMGPRY |
| | DYWGQGTQVTVSS (SEQ ID NO: 382) |
| VHH187B1 | EVQLVESGGGLVQPGGSLRLSCAASGFT |
| | LDYYAIGWFRQAPGKEREGVSCISSSDR |
| | STWYADSVKGRFTISRDNAKNTVYLQM |
| | HSLKPEDTAVYYCATPCYSDYDPEGYE |
| | YDYWGQGTQVTVSS (SEQ ID NO: 383) |
| VHH195B1 | QVQLVESGGGLVQAGGSLRLSCAASGR |
| | TFGTYAMGWFRQAPGKERDFVARISNS |
| | GAFTHYADSVKGRFTISRDNAKNTVYL |
| | HMNSLENGDTAAYYCVATRGTNSAYYT |
| | RSTMYEYWGQGTQVTVSS (SEQ ID NO: |
| | 384) |
| VHH202B1 | QVQLQESGGGLVQAGGSLRLSCAASGTI |
| | FSIDVMAYYRQAPGKQRELVAAISSGGS |
| | LNYRDSVKGRFTISRDNAKNAVYLQMN |
| | SLKPDDTAVYYCYANIRTSPVTTRPMGN |
| | YWGQGTQVTVSS (SEQ ID NO: 385) |
| VHH233B1 | QVQLVESGGGLVQAGGSLRLSCAASRSI |
| | SSFNEMAWYRQAPGEQRELVATIVSTV |
| | GFTNYADSVKGRFTISRDNAKNTVYLQ |
| | MNSLKAEDTAVYYCNARRISTDYWGQG |
| | TQVTVSS (SEQ ID NO: 386) |
| VHH238B1 | QLQLVESGGGLVQPGGSLRLSCAASGFT |
| | FSSYGMSWVRQAPGKGLEWVSYISAGG |
| | GTTTYVDFVKGRFTISRDNAKNMLYLQ |
| | MNSLKPEDTAVYYCVHAADGSRGQGT |
| | QVTVSS (SEQ ID NO: 387) |
| VHH245B1 | QVQLQESGGGLVQAGGSLRLSCAASGSI |
| | ASINAMGWYRQAPGKQRDFVARIQLSG |
| | QTSYAHSVRGRFTISRDNAKNTVYLQM |
| | NNLKPEDTAVYVCAASNTGSLYGLRNY |
| | WGQGTQVTVSS (SEQ ID NO: 388) |
| VHH258B1 | QVQLVESGGGLVQAGGSLRLSCAASGR |
| | TFSSYVMGWFRQAPGQEREFVAAIMRN |
| | GIMTYYADSVKGRFTISRDNAKNTVYLQ |
| | MNTLKPDDTAVYYCAAASRVGVSISQSS |
| | SYTSWGQGTQVTVSS (SEQ ID NO: 389) |
| VHH263B1 | EVQLVESGGGLVQAGGSLRLSCAASGS |
| | KFSFNAMGWYRQTPGKQRELVAIIINGD |
| | MINYADSVKGRFTISRDNAKNTMYLQM |
| | NSLKPEDTAVYYCKMTRYVYDYWGQG |
| | TQVTVSS (SEQ ID NO: 390) |
| VHH276B1 | QVQLVESGGGLVQAGASLRLSCAASGR |
| | TFSSYAMGWFRQAPGKEREFVAAISGGT |
| | PNNIYYTNYKDAVRGRFTISRDNAKNTV |
| | YLQMSSLKPEDTAVYYCGATMGRYWN |
| | TGGYDYWGQGTQVTVSS (SEQ ID NO: |
| | 391) |
| VHH321B1 | QVQLVESGGGLVQPGGSLRLSCIASGFT |
| | FSDYAMKWVRRPPGKGLEWVSVIDSEG |
| | PTIKYSDAVRGRFTISRDNAKNTLYLQM |
| | NSLKPEDTAVYYCAKSNGGSAFSSVLYT |
| | DRGQGTQVTVSS (SEQ ID NO: 392) |

### Anti-IL-12Rβ2 Binding Domains

In another aspect of the multi-specific binding protein of the present disclosure is a binding domain or binding specificity which binds to the IL-12Rβ2 receptor subunit of the IL-12R receptor (e.g., human IL-12).

In certain embodiments, the binding domain comprises the heavy and/or light chain variable regions or a conventional antibody or antigen binding fragment thereof. In certain embodiments, the binding domain is a Fab. In certain embodiments, the binding domain is a scFv. In certain embodiments, the IL-12Rβ2 binding domain is a Fab or a scFv and is paired with an IL-12Rβ2 binding domain that is a Fab, scFv, or a VHH domain. In some embodiments, the IL-12Rβ2 binding domain is a Fab that shares a common light chain with a Fab of the IL-12Rβ1 binding domain.

Exemplary binding moieties comprising an antibody variable domain include, without limitation, a VH, a VL, a VHH, a VH/VL pair, an scFv, a diabody, or a Fab. Other suitable binding moiety formats, include, without limitation, lipocalins (see e.g., Gebauer M. et al., 2012, Method Enzymol. 503:157-188, which is incorporated by reference herein in its entirety), adnectins (see e.g., Lipovsek D., 2011, Protein Eng. Des. Sel. 24:3-9, which is incorporated by reference herein in its entirety), avimers (see e.g., Silverman J, et al., 2005, Nat. Biotechnol. 23:1556-1561, which is incorporated by reference herein in its entirety), fynomers (see e.g., Schlatter D, et al., 2012, mAbs 4:497-508, which is incorporated by reference herein in its entirety), kunitz domains (see e.g., Hosse R. J. et al., 2006, Protein Sci. 15:14-27, which is incorporated by reference herein in its entirety), knottins (see e.g., Kintzing J. R. et al., 2016, Curr. Opin. Chem. Biol. 34:143-150, which is incorporated by reference herein in its entirety), affibodies (see e.g., Feldwisch J. et al., 2010 J. Mol. Biol. 398:232-247, which is incorporated by reference herein in its entirety), and DARPins (see e.g., Pluckthun A., 2015, Annu. Rev. Pharmacol. Toxicol. 55:489-511, which is incorporated by reference herein in its entirety).

In some embodiments, the IL-12Rβ2 receptor subunit binding domain comprises the exemplary CDRs as provided in Table 6 and Table 7. In some embodiments, the IL-12Rβ2 receptor subunit binding domain comprises the sequences as provided in Table 8. In some embodiments, the IL-12Rβ2 receptor subunit binding domain binds to the IL-12Rβ2 receptor subunit more tightly than the IL-12Rβ1 receptor subunit binding domain to the IL-12Rβ1 receptor subunit.

TABLE 6
IL-12Rβ2 heavy variable chain (VH) CDRs (HCDR1, HCDR2, and HCDR3)

| Heavy Chain | | | |
|---|---|---|---|
| Hybridoma ID | HCDR1 | HCDR2 | HCDR3 |
| 85802_02K15A | SYWIG | NIYPGDSDTRYSPSFQG | RGGGY |
| | (SEQ ID | (SEQ ID NO: 394) | (SEQ ID NO: 395) |
| | NO: 393) | | |
| 85802_06A17A | SYWIG | IIYPGDSDTRYSPSFQG | HEGAYWYFDL |
| | (SEQ ID | (SEQ ID NO: 397) | (SEQ ID NO: 398) |
| | NO: 396) | | |
| 85802_06F06A | SYYWS | YIYDSGSTNYNPSLKS | GSYFWYFDL |
| | (SEQ ID | (SEQ ID NO: 400) | (SEQ ID NO: 401) |
| | NO: 399) | | |
| 85802_06H06A | YYYWN | SIYNSGNINYNPSLKS | GGYSYGYFDN |
| | (SEQ ID | (SEQ ID NO: 403) | (SEQ ID NO: 404) |
| | NO: 402) | | |
| 85802_08F04A | SYAMS | AISGSGGSTYYADSVKG | DGGYFDY |
| | (SEQ ID | (SEQ ID NO: 406) | (SEQ ID NO: 407) |
| | NO: 405) | | |
| 85802_08F06A | GYYMH | WINPKSGGTNYAQKFQG | DQGYGDYVLKH |
| | (SEQ ID | (SEQ ID NO: 409) | (SEQ ID NO: 410) |
| | NO: 408) | | |
| 85802_08F20A | GYYIH | WINPHSGGTNYAQKFQG | EANNWGYAFDI |
| | (SEQ ID | (SEQ ID NO: 412) | (SEQ ID NO: 413) |
| | NO: 411) | | |
| 85802_08G15A | NYYMH | IINPSGGSTSYSQKFQG | DLGTTTDY |
| | (SEQ ID | (SEQ ID NO: 415) | (SEQ ID NO: 416) |
| | NO: 414) | | |
| 85802_08G21A | TYDMH | LIDTAGDTYYPDSVKG | EGWGYFDY |
| | (SEQ ID | (SEQ ID NO: 418) | (SEQ ID NO: 419) |
| | NO: 417) | | |
| 85802_08G21A2 | DYYWS | YIYYSGSTTYSPSLKS | DNGDYGFDY |
| | (SEQ ID | (SEQ ID NO: 421) | (SEQ ID NO: 422) |
| | NO: 420) | | |
| 85802_08J15A | DYHWS | EINHSGSTIYKSSLKS | VDWANAFDI |
| | (SEQ ID | (SEQ ID NO: 424) | (SEQ ID NO: 425) |
| | NO: 423) | | |
| 85802_08K10A | GSVMH | RIRSKANSYATAFPASVKG | GSLDVFDF |
| | (SEQ ID | (SEQ ID NO: 427) | (SEQ ID NO: 428) |
| | NO: 426) | | |
| 85802_08K16A | TSGMCVN | LIDWDDDKYYSTSLKT | GIAAAGSNWFDP |
| | (SEQ ID | (SEQ ID NO: 430) | (SEQ ID NO: 431) |
| | NO: 429) | | |
| 85802_09B21A | GYWIG | IIYPGDSDTRYSPSFQG | HPRGWWYFDL |
| | (SEQ ID | (SEQ ID NO: 433) | (SEQ ID NO: 434) |
| | NO: 432) | | |
| 85802_09K20A | DSSMH | RIRDKANTYATAYAASVKG | HEWDLDAFDI |
| | (SEQ ID | (SEQ ID NO: 436) | (SEQ ID NO: 437) |
| | NO: 435) | | |
| 85802_09M19A | SYAMS | AINDRGNSPYYADSVKG | MGATSNWFDP |
| | (SEQ ID | (SEQ ID NO: 439) | (SEQ ID NO: 440) |
| | NO: 438) | | |
| 85802_09N16A | GSVMH | RIRSKANSYATAYAASVTG | GSYDYFDY |
| | (SEQ ID | (SEQ ID NO: 442) | (SEQ ID NO: 443) |
| | NO: 441) | | |
| 85802_09N23A | GSSMH | RIRDKANSYATAYAASVKG | HEWDLDAFDI |
| | (SEQ ID | (SEQ ID NO: 445) | (SEQ ID NO: 446) |
| | NO: 444) | | |
| 85802_10D03A | SYYWS | RIYTSGSTNYNPSLKS | AGSSGSSYYYYGMDV |
| | (SEQ ID | (SEQ ID NO: 448) | (SEQ ID NO: 449) |
| | NO: 447) | | |
| 85802_10G07A | SYYMH | IINPSGGSTSYAQKFQG | GEEWELLGDY |
| | (SEQ ID | (SEQ ID NO: 451) | (SEQ ID NO: 452) |
| | NO: 450) | | |
| 85802_10K02A | TSGMCVS | LIYWDDDKYYSTSLKT | MRRGIEAFDI |
| | (SEQ ID | (SEQ ID NO: 454) | (SEQ ID NO: 455) |
| | NO: 453) | | |
| 85802_10K12A | TSGMCVS | LIYWDDDKYYSTSLKT | MRRGIEAFDI |
| | (SEQ ID | (SEQ ID NO: 457) | (SEQ ID NO: 458) |
| | NO: 456) | | |
| 85802_10M17A | SYSMN | SISSSSDYIHYADSVRG | SELGFDY |
| | (SEQ ID | (SEQ ID NO: 460) | (SEQ ID NO: 461) |
| | NO: 459) | | |
| 85802_10N05A | TSGMCVS | LIYWDDDKYYSTSLKT | MRRGIEAFDI |
| | (SEQ ID | (SEQ ID NO: 463) | (SEQ ID NO: 464) |
| | NO: 462) | | |
| 85802_10O18A | DYYWT | YIYYSGNTNYNPSLKS | EGWMGVTPIAFDF |
| | (SEQ ID | (SEQ ID NO: 466) | (SEQ ID NO: 467) |
| | NO: 465) | | |

TABLE 7
IL-12Rβ2 light variable chain (VL) CDRs (LCDR1, LCDR2, and LCDR3)

| Light Chain | | | |
|---|---|---|---|
| Hybridoma ID | LCDR1 | LCDR2 | LCDR3 |
| 85802_02K15A | RSSQSLVYSDGNTYLN | KVSNRDS | MQGTHWPIT |
| | (SEQ ID NO: 468) | (SEQ ID NO: 469) | (SEQ ID NO: 470) |
| 85802_06A17A | TGSSSNIGAGYDVH | GNSNRPS | QSLDTSHVV |
| | (SEQ ID NO: 471) | (SEQ ID NO: 472) | (SEQ ID NO: 473) |
| 85802_06F06A | SGDKLGAKYAS | QDTKRPS | QAWDSSTVV |
| | (SEQ ID NO: 474) | (SEQ ID NO: 475) | (SEQ ID NO: 476) |
| 85802_06H06A | SGRELGNKYAY | EDYKRPS | QAWDSSTVI |
| | (SEQ ID NO: 477) | (SEQ ID NO: 478) | (SEQ ID NO: 479) |
| 85802_08F04A | RASQSISSWLA | KASSLES | QQYNSYSPT |
| | (SEQ ID NO: 480) | (SEQ ID NO: 481) | (SEQ ID NO: 482) |
| 85802_08F06A | GGDNIGSKSVH | DDSDRPS | QVWDSSSDHVV |
| | (SEQ ID NO: 483) | (SEQ ID NO: 484) | (SEQ ID NO: 485) |
| 85802_08F20A | SGGSSNIGTNSVN | YNNQRPS | AAWDDSLNGWV |
| | (SEQ ID NO: 486) | (SEQ ID NO: 487) | (SEQ ID NO: 488) |
| 85802_08G15A | RASQAIRNDLG | AASSFQS | LQDYRYPYT |
| | (SEQ ID NO: 489) | (SEQ ID NO: 490) | (SEQ ID NO: 491) |
| 85802_08G21A | TGTSSDVGSYNRVS | EVSNRPS | SLYTGSSTVV |
| | (SEQ ID NO: 492) | (SEQ ID NO: 493) | (SEQ ID NO: 494) |
| 85802_08G21A2 | TGTSSDVGSYNRVS | EVSNRPS | SLYTGSSTVV |
| | (SEQ ID NO: 495) | (SEQ ID NO: 496) | (SEQ ID NO: 497) |
| 85802_08J15A | TGSSSNIGAGYDVH | ANSNRPS | QSYDSSLSVV |
| | (SEQ ID NO: 498) | (SEQ ID NO: 499) | (SEQ ID NO: 500) |
| 85802_08K10A | QASQDISNYLN | DASNLEA | QQYDNLLT |
| | (SEQ ID NO: 501) | (SEQ ID NO: 502) | (SEQ ID NO: 503) |
| 85802_08K16A | SGSSSNIGSHAVN | VNNQRPS | AAWDDSLNGPV |
| | (SEQ ID NO: 504) | (SEQ ID NO: 505) | (SEQ ID NO: 506) |
| 85802_09B21A | TGSSSNIGAGYDVD | GNSNRPS | QSYDNSLSGSV |
| | (SEQ ID NO: 507) | (SEQ ID NO: 508) | (SEQ ID NO: 509) |
| 85802_09K20A | RASQGISSYLV | AASNLQS | QQFNSYPFT |
| | (SEQ ID NO: 510) | (SEQ ID NO: 511) | (SEQ ID NO: 512) |
| 85802_09M19A | TLSSGYSHYKVD | VGTGGIVGSKGD | GADYGSGSHFVYV |
| | (SEQ ID NO: 513) | (SEQ ID NO: 514) | (SEQ ID NO: 515) |
| 85802_09N16A | QASQDISNYLN | DASNLET | QQYDDLLIT |
| | (SEQ ID NO: 516) | (SEQ ID NO: 517) | (SEQ ID NO: 518) |
| 85802_09N23A | RASQGISSYLA | AASTLQS | QQFNSYPFT |
| | (SEQ ID NO: 519) | (SEQ ID NO: 521) | (SEQ ID NO: 522) |
| 85802_10D03A | SGDKLGDKYAC | QDSKRPS | QAWDSSTVV |
| | (SEQ ID NO: 523) | (SEQ ID NO: 524) | (SEQ ID NO: 525) |
| 85802_10G07A | RASQVISNYLA | AASSLQS | LQHNDYPFT |
| | (SEQ ID NO: 526) | (SEQ ID NO: 527) | (SEQ ID NO: 528) |
| 85802_10K02A | TGSSSNIGAGYDVH | GYSNRPS | QSFDISLTGWV |
| | (SEQ ID NO: 529) | (SEQ ID NO: 530) | (SEQ ID NO: 531) |
| 85802_10K12A | TGSSSNIGAGYDVH | GYSNRPS | QSFDISLTGWV |
| | (SEQ ID NO: 532) | (SEQ ID NO: 533) | (SEQ ID NO: 534) |
| 85802_10M17A | RASQGISIWLA | AASSLQS | QQANSFPLT |
| | (SEQ ID NO: 535) | (SEQ ID NO: 536) | (SEQ ID NO: 537) |
| 85802_10N05A | TGSSSNIGAGYDVH | GYSNRPS | QSFDISLTGWV |
| | (SEQ ID NO: 538) | (SEQ ID NO: 539) | (SEQ ID NO: 540) |
| 85802_10O18A | SGSSSNIGSNTVN | NNDQRPS | AAWDDSLNGWV |
| | (SEQ ID NO: 541) | (SEQ ID NO: 542) | (SEQ ID NO: 543) |

TABLE 8
IL-12Rβ2 VH and VL sequences.

| Hybridoma ID | VH | VL |
|---|---|---|
| 85802_02K15A | EVQLVQSGTEVKKPGESLKIS | DVVMTQSPLSLPVTLGQPASISC |
| | CKGSGYSFTSYWIGWVRQMP | RSSQSLVYSDGNTYLNWFQQRP |
| | GKGLEWMGNIYPGDSDTRYS | GQSPRRLIYKVSNRDSGVPDRFS |
| | PSFQGHVTISADKSISTAYLQW | GSGSGTDFTLKISRVEADDVGV |
| | SSLKASDTAMYYCARRGGGY | YYCMQGTHWPITFGQGTRLEIK |
| | WGQGTLVTVSS (SEQ ID NO: | (SEQ ID NO: 545) |
| | 544) | |
| 85802_06A17A | EVQLVQSGAEVKKPGESLKIS | QSVLTQPPSVSGAPGQRVTISCT |
| | CKGSGYSFTSYWIGWVRQMP | GSSSNIGAGYDVHWYQQLPGT |
| | GKGLECMGIIYPGDSDTRYSPS | APKVLIYGNSNRPSGVPDRFSGS |
| | FQGQVTISADKSISTAYLQWSS | KSGTSASLAITGLQAEDEADYY |
| | LKASDTAMYYCARHEGAYW | CQSLDTSHVVFGGGTKLTVL |
| | YFDLWGRGTLVTVSS (SEQ ID | (SEQ ID NO: 547) |
| | NO: 546) | |
| 85802_06F06A | QVQLQESGPGLVKPSETLSLT | SYELTQPPSVSVSPGQTASITCS |
| | CTVSGGSISSYYWSWIRQPPG | GDKLGAKYASWYHQKPGQSPV |
| | KGLEWFGYIYDSGSTNYNPSL | LVIYQDTKRPSGIPERFSGSNSG |
| | KSRVTISVDTSKNQFSLKLSSV | NTATLTISGTQAMDEADYYCQA |
| | TAADTAVYYCARGSYFWYFD | WDSSTVVFGGGTKLTVL (SEQ |
| | LWGRGTLVTVSS (SEQ ID | ID NO: 549) |
| | NO: 548) | |
| 85802_06H06A | QVHLQESGPGLVKPSETLSLT | SYELTQPPSVSVSPGQTASITCS |
| | CTVSGGSISYYYWNWIRQPPG | GRELGNKYAYWYQQKPGQSPV |
| | KGLEWIASIYNSGNINYNPSLK | LVIYEDYKRPSGIPERFSGSNSG |
| | SRVTISVDTSKNQFSLKLTSVT | NTATLTISGTQAMDEADYYCQA |
| | AADTAVYYCARGGYSYGYFD | WDSSTVIFGGGTKLTVL (SEQ ID |
| | NWGQGILVTVSS (SEQ ID | NO: 551) |
| | NO: 550) | |
| 85802_08F04A | EVQLLESGGGLVQPGGSLRLS | DIQMTQSPSTLSASVGDRVTITC |
| | CAASGFTFSSYAMSWVRQAP | RASQSISSWLAWYQQKPGKAPK |
| | GKGLEWVSAISGSGGSTYYAD | LLIYKASSLESGVPSRFSGSGSG |
| | SVKGRFTISRDNSKNTLYLQM | TEFTLTISSLQPDDFATYYCQQY |
| | NSLRAEDTAVYYCAKDGGYF | NSYSPTFGQGTKVEIK (SEQ ID |
| | DYWGQGTLVTVSS (SEQ ID | NO: 553) |
| | NO: 552) | |
| 85802_08F06A | QVQLVQPGAEVKKPGASVKV | SYVLTQPPSVSVAPGQTARITCG |
| | SCKASGYTFTGYYMHWVRQA | GDNIGSKSVHWYQQKPGQAPV |
| | PGQGLEWMGWINPKSGGTNY | PVVYDDSDRPSGIPERFSGSNSG |
| | AQKFQGRVTMTRDTSISTAYM | NTATLTISRVEAGDEADYYCQV |
| | ELTRLRSDDTAVYYCARDQG | WDSSSDHVVFGGGTKLTVL |
| | YGDYVLKHWGQGTLVTVSS | (SEQ ID NO: 555) |
| | (SEQ ID NO: 554) | |
| 85802_08F20A | QVQLVQSGAEVKKPGASVKV | QSVLTQPPSASGTPGQRVTISCS |
| | SCKASGYTFIGYYIHWVRQAP | GGSSNIGTNSVNWYQQLPGTAP |
| | GQGLEWMGWINPHSGGTNYA | KLLIYYNNQRPSGVPDRFSGSKS |
| | QKFQGRVTMTRDTSISTAYME | GTSASLAISGLQSEDEADYYCA |
| | LSRLRSDDTAIYYCTKEANNW | AWDDSLNGWVFGGGTKLTVL |
| | GYAFDIWGQGTMVTVSS (SEQ | (SEQ ID NO: 557) |
| | ID NO: 556) | |
| 85802_08G15A | QVQLVQSGAEVKKPGASVKV | AIQMTQSPSSLSASVGDRVTITC |
| | SCKSSGYTFTNYYMHWVRQA | RASQAIRNDLGWFQQKPGKAPR |
| | PGQGLEWMGIINPSGGSTSYS | LLIYAASSFQSGVPSRFSGSGSG |
| | QKFQGRVTMTRDTSTSTDYM | TDFTLTISSLQPEDFATYYCLQD |
| | ELSSLRSDDTAVYYCARDLGT | YRYPYTFGQGTKLEIK (SEQ ID |
| | TTDYWGQGTLVTVSS (SEQ ID | NO: 559) |
| | NO: 558) | |
| 85802_08G21A | EVQLVESGGGLVQPGGSLRLS | QSALTQPPSVSGSPGQSVTISCT |
| | CAASGFTFSTYDMHWVRQAT | GTSSDVGSYNRVSWYQQPPGT |
| | GKGLEWVSLIDTAGDTYYPDS | APKLMIYEVSNRPSGVPDRFSGS |
| | VKGRFTISRENAKNSLYLQMN | KSGNTASLTISGLQAEDEADYY |
| | SLRAGDTAVYYCAREGWGYF | CSLYTGSSTVVFGGGTKVTVL |
| | DYWGQGALVTVSS (SEQ ID | (SEQ ID NO: 561) |
| | NO: 560) | |
| 85802_08G21A2 | QVQLLESGPGLVKPSETLSLTC | QSALTQPPSVSGSPGQSVTISCT |
| | TVSGGSISDYYWSWIRQSPGK | GTSSDVGSYNRVSWYQQPPGT |
| | GLEWIGYIYYSGSTTYSPSLKS | APKLMIYEVSNRPSGVPDRFSGS |
| | RVTISVDTSKNQFSLKLGSVTA | KSGNTASLTISGLQAEDEADYY |
| | ADTAVYYCARDNGDYGFDY | CSLYTGSSTVVFGGGTKVTVL |
| | WGQGTLVTVSS (SEQ ID NO: | (SEQ ID NO: 563) |
| | 562) | |
| 85802_08J15A | QVQLQQWGAGLLKPSETLSLT | QSVLTQPPSVSGAPGQRVTISCT |
| | CAVYGGSFSDYHWSWIRQPP | GSSSNIGAGYDVHWYQQLPGT |
| | GKGLEWIGEINHSGSTIYKSSL | APKLLIYANSNRPSGVPDRFSGS |
| | KSRVTISVDTSKNQFSLKLSSV | KSGTSASLAITGLQAEDEADYY |
| | TAADTAVYYCARVDWANAF | CQSYDSSLSVVFGGGTKLTVL |
| | DIWGQGTMVTVSS (SEQ ID | (SEQ ID NO: 565) |
| | NO: 564) | |
| 85802_08K10A | EVQLVESGGGLVQPGGSLKLS | DIQMTQSPSSLSASVGDRVTITC |
| | CAGSGFTFSGSVMHWVRQAS | QASQDISNYLNWYQQKPGKAP |
| | GKGLEWVGRIRSKANSYATAF | KLLIYDASNLEAGVPSRFSGSGF |
| | PASVKGRFTISRDDSKNTAYL | GTDFTFTISSLHPEDFATYYCQQ |
| | QMNSLKTEDTAVYYCTSGSL | YDNLLTFGGGTKVEIK (SEQ ID |
| | DVFDFWGQGTMVTVSS (SEQ | NO: 567) |
| | ID NO: 566) | |
| 85802_08K16A | QVTLRESGPALVKPTQTLTLT | QSVLTQPPSASGTPGQRVTISCS |
| | CTFSGFSLSTSGMCVNWIRQPP | GSSSNIGSHAVNWYQQLPGTAP |
| | GKALEWLALIDWDDDKYYST | KLLIYVNNQRPSGVPDRFSGSKS |
| | SLKTRLTISKDTSKNQVVLTM | GTSASLAISGLQSEDEADYYCA |
| | TNMDPVDTATYYCTRGIAAA | AWDDSLNGPVFGGGTKLTVL |
| | GSNWFDPWGQGTLVTVSS | (SEQ ID NO: 569) |
| | (SEQ ID NO: 568) | |
| 85802_09B21A | EVQLVQSGAEVKKPGESLKIS | QSVLTQPPSVSGAPGQRVTISCT |
| | CKGSGYSFTGYWIGWVRQMP | GSSSNIGAGYDVDWYQQLPGT |
| | GKGLEWMGIIYPGDSDTRYSP | APKVLIHGNSNRPSGVPDRFSGS |
| | SFQGQVTISADKSISTAYLQWS | KSGTSASLAITGLQAEDEADYY |
| | SLKASDTAMYYCVRHPRGW | CQSYDNSLSGSVFGGGTKLTVL |
| | WYFDLWGRGTLVTVSS (SEQ | (SEQ ID NO: 571) |
| | ID NO: 570) | |
| 85802_09K20A | EVQLVESGGGLVQPGGSLKLS | DIQLTQSPSFLSASVGDRVTITC |
| | CAASGFTFSDSSMHWVRQAS | RASQGISSYLVWYQQNPGKAPK |
| | GKGLEWVGRIRDKANTYATA | LLIYAASNLQSGVPSRFSGSGSG |
| | YAASVKGRFTISRDDSKNTAY | TEFTLTISSLQPEDFATYYCQQF |
| | LQMNSLKTEDTAVYYCTRHE | NSYPFTFGPGTKVDVK (SEQ ID |
| | WDLDAFDIWGQGTMVTVSS | NO: 573) |
| | (SEQ ID NO: 572) | |
| 85802_09M19A | EVQLLESGGDLIQPGGSLRLSC | QPVLTQPPSASASLGASVTLTCT |
| | AASGFTFSSYAMSWVRQAPG | LSSGYSHYKVDWYQQRPGKGP |
| | KGLEWVSAINDRGNSPYYADS | RFVMRVGTGGIVGSKGDGIPDR |
| | VKGRFTISRDNSKNTLFLQMIS | FSVLGSGLNRYLTIKNIQEEDES |
| | LRAEDTAVYYCARMGATSNW | DYHCGADYGSGSHFVYVFGGG |
| | FDPWGQGTLVTVSS (SEQ ID | TKLTVL (SEQ ID NO: 575) |
| | NO: 574) | |
| 85802_09N16A | EVQLVESGGGLVQPGGSLKLS | DIQMTQSPSSLSASVGDRVTITC |
| | CAASGFTFSGSVMHWVRQAS | QASQDISNYLNWYQQKPGKAP |
| | GKGLEWVGRIRSKANSYATA | KLLIYDASNLETGVPSRFSGSGS |
| | YAASVTGRFTISRDDSKNTAF | GTDFTFTISSLQPEDIATYYCQQ |
| | LQMNSLKTEDTAVYYCSSGSY | YDDLLITFGQGTRLEIK (SEQ ID |
| | DYFDYWGQGTLVTVSS (SEQ | NO: 577) |
| | ID NO: 576) | |
| 85802_09N23A | EVQLVESGGGLVQPGGSLKLS | DIQLTQSPSFLSASVGDRVTITC |
| | CAASGFSISGSSMHWVRQASG | RASQGISSYLAWYQQKPGKAPK |
| | KGLEWVGRIRDKANSYATAY | LLIYAASTLQSGVPSRFSGSGSG |
| | AASVKGRFTVSRDDSKNTAYL | TEFTLTISSLQPEDFATYYCQQF |
| | QMTSLKTEDTAVYFCTRHEW | NSYPFTFGPGTKVDIK (SEQ ID |
| | DLDAFDIWGQGTMVTVSS | NO: 579) |
| | (SEQ ID NO: 578) | |
| 85802_10D03A | QVQLQESGPGLVKPSETLSLT | SYELTQPPSVSVSPGQTASITCS |
| | CTVSGGSISSYYWSWIRQPAG | GDKLGDKYACWYQQKPGQSPV |
| | KGLEWIGRIYTSGSTNYNPSLK | LVIYQDSKRPSGIPERFSGSNSG |
| | SRVTMSVDTSKNQFSLKLSSV | NTATLTISGTQAMDEADYYCQA |
| | TAADTAEYYCARAGSSGSSYY | WDSSTVVFGGGTKLTVL (SEQ |
| | YYGMDVWGQGTTVTVSS | ID NO: 581) |
| | (SEQ ID NO: 580) | |
| 85802_10G07A | QVQLVQSGAEVKKPGASVKV | DIQMTQSPSAMSASVGDRVTIT |
| | SCKASGYTFTSYYMHWVRQA | CRASQVISNYLAWFQQKPGKVP |
| | PGQGLEWMGIINPSGGSTSYA | KRLIYAASSLQSGVPSRFSGSGS |
| | QKFQGRVTMTRDTSTSTVYM | GTEFTLTISSLQPEDFATYYCLQ |
| | ELSSLRSEDTAVYYCARGEEW | HNDYPFTFGPGTKVDIK (SEQ ID |
| | ELLGDYWGQGTLVTVSS (SEQ | NO: 583) |
| | ID NO: 582) | |
| 85802_10K02A | QVTLRESGPALVKPTQTLTLT | QSVLTQPPSVSGAPGQRVTISCT |
| | CTFSGFSLSTSGMCVSWIRQPP | GSSSNIGAGYDVHWYQQLPGT |
| | GKALEWLALIYWDDDKYYST | APKLLIFGYSNRPSGVPVRFSGS |
| | SLKTRLTISKDTSKNQVVLTM | KSGTSASLAITGLLAEDEADFYC |
| | TNMDPVDTATYYCARMRRGI | QSFDISLTGWVFGGGTKLTVL |
| | EAFDIWGQGTMVTVFS (SEQ | (SEQ ID NO: 585) |
| | ID NO: 584) | |
| 85802_10K12A | QVTLRESGPALVKPTQTLTLT | QSVLTQPPSVSGAPGQRVTISCT |
| | CTFSGFSLSTSGMCVSWIRQPP | GSSSNIGAGYDVHWYQQLPGT |
| | GKALEWLALIYWDDDKYYST | APKLLIFGYSNRPSGVPVRFSGS |
| | SLKTRLTISKDTSKNQVVLTM | KSGTSASLAITGLLAEDEADFYC |
| | TNMDPVDTATYYCARMRRGI | QSFDISLTGWVFGGGTKLTVL |
| | EAFDIWGQGTMVTVFS (SEQ | (SEQ ID NO: 587) |
| | ID NO: 586) | |
| 85802_10M17A | EVLLVESGGGLVKPGGSLRLS | DIQMTQSPSSVSASVGDRVTITC |
| | CAASGFTFSSYSMNWVRQAP | RASQGISIWLAWYQQKPGKAPK |
| | GKGLEWVSSISSSSDYIHYADS | PLIYAASSLQSGVPSRFSGSGSG |
| | VRGRFTISRDNAKNSLYLQMN | TDFTLTISSLQPEDFATYYCQQA |
| | SLRAEDTAVYYCARSELGFDY | NSFPLTFGGGTKVEIK (SEQ ID |
| | WGQGTLVTVSS (SEQ ID NO: | NO: 589) |
| | 588) | |
| 85802_10N05A | QVTLRESGPALVKPTQTLTLT | QSVLTQPPSVSGAPGQRVTISCT |
| | CTFSGFSLSTSGMCVSWIRQPP | GSSSNIGAGYDVHWYQQLPGT |
| | GKALEWLALIYWDDDKYYST | APKLLIFGYSNRPSGVPVRFSGS |
| | SLKTRLTISKDTSKNQVVLTM | KSGTSASLAITGLLAEDEADFYC |
| | TNMDPVDTATYYCARMRRGI | QSFDISLTGWVFGGGTKLTVL |
| | EAFDIWGQGTMVTVFS (SEQ | (SEQ ID NO: 591) |
| | ID NO: 590) | |
| 85802_10O18A | QVQLQESGPGLVKPSETLSLT | QSVLTQPPSASGTPGQRVTISCS |
| | CTVSGDSISDYYWTWIRQSPG | GSSSNIGSNTVNWYQQLPGTAP |
| | KGLEWIGYIYYSGNTNYNPSL | KLLIFNNDQRPSGVPDRFSGSRS |
| | KSRVTISVDTSKNQFSLKLSSV | GTSASLAISGLQSEDEADYYCA |
| | TAADTAVYYCTREGWMGVTP | AWDDSLNGWVFGGGTKLTVL |
| | IAFDFWGQGTMVTVSS (SEQ | (SEQ ID NO: 593) |
| | ID NO: 592) | |

In other embodiments, the IL-12Rβ2 receptor subunit binding domain comprises at least the CDRs or a VHH domain of a VHH antibody or Nanobody. In some embodiments, the IL-12Rβ2 VHH binding subunit is paired with a Fab, scFv domain that specifically binds to IL-12Rβ1.

In some embodiments, the IL-12R(S2 V binding domain is paired with an IL-12Rβ1/VHH binding domain.

In some embodiments, the IL-12Rβ2 receptor subunit binding domain comprises the exemplary VHH CDRs as provided in Table 9. In some embodiments, the IL-12Rβ2 receptor subunit binding domain comprises the VHH sequences as provided in Table 10.

TABLE 9
IL-12Rβ2 receptor subunit VHH CDR sequences.

| Clone | | | |
|---|---|---|---|
| number | HCDR1 | HCDR2 | HCDR3 |
| VHH13B2 | SNGMH | SIGSGGTSIYYADSVKG | EVTGAT |
| | (SEQ ID | (SEQ ID NO: 595) | (SEQ ID NO: 596) |
| | NO: 594) | | |
| VHH19B2 | NYAMS | AINSDGTSTSYADSVKG | WEGDTDSDYKFKDY |
| | (SEQ ID | (SEQ ID NO: 598) | (SEQ ID NO: 599) |
| | NO: 597) | | |
| VHH64B2 | SKAMH | SINSGGAITYYKDSVKG | DTDGRT |
| | (SEQ ID | (SEQ ID NO: 601) | (SEQ ID NO: 602) |
| | NO: 600) | | |
| VHH67B2 | NYAMS | RINSGGGTTSYADSVKG | HSHPDYSSNYY |
| | (SEQ ID | (SEQ ID NO: 604) | (SEQ ID NO: 605) |
| | NO: 603) | | |
| VHH68B2 | SYAMG | AIMRSGDTTVYVDSVKG | GRPVTRTYYSTYTYPHDYDY |
| | (SEQ ID | (SEQ ID NO: 607) | (SEQ ID NO: 608) |
| | NO: 606) | | |
| VHH85B2 | LNTMA | FITNGGTTNYVDSVEG | QISQYPYNY |
| | (SEQ ID | (SEQ ID NO: 610) | (SEQ ID NO: 611) |
| | NO: 609) | | |
| VHH93B2 | PYDMS | AIDKGGGAGATYYADSVKG | TAGPYSDSTPRVAY |
| | (SEQ ID | (SEQ ID NO: 613) | (SEQ ID NO: 614) |
| | NO: 612) | | |
| VHH94B2 | SYAMS | AINSGGDSTSYADSVEG | WSDCSEYGCYDQT |
| | (SEQ ID | (SEQ ID NO: 616) | (SEQ ID NO: 617) |
| | NO: 615) | | |
| VHH185B2 | RYRMG | AITKSGATTYDADSVKG | SYHSNWWPKNADEYAY |
| | (SEQ ID | (SEQ ID NO: 619) | (SEQ ID NO: 620) |
| | NO: 618) | | |
| VHH190B2 | TNYYYWS | AIAYSGSTYYSPSLKS | GINFYSNYPTLNEYDY |
| | (SEQ ID | (SEQ ID NO: 622) | (SEQ ID NO: 623) |
| | NO: 621) | | |
| VHH194B2 | GYTVG | AIQWSSAYTYSEFVKG | RRSRTSWYASTAEAYDY |
| | (SEQ ID | (SEQ ID NO: 625) | (SEQ ID NO: 626) |
| | NO: 624) | | |
| VHH211B2 | SYVVG | RISWSGARTYYADSVKG | DLKWEVLLSEHFEY |
| | (SEQ ID | (SEQ ID NO: 628) | (SEQ ID NO: 629) |
| | NO: 627) | | |
| VHH216B2 | TYRMG | GISWTGQTTYYAASVKG | ETNNLGRWDRPSEYDY |
| | (SEQ ID | (SEQ ID NO: 631) | (SEQ ID NO: 632) |
| | NO: 630) | | |
| VHH219B2 | SYVVG | RISWSGARTYYADSVEG | DLKWEVLLSEHYEY |
| | (SEQ ID | (SEQ ID NO: 634) | (SEQ ID NO: 635) |
| | NO: 633) | | |
| VHH222B2 | AYRMG | GITWRGQTTYYAASVKG | ETNGLGRWDRPSEYDY |
| | (SEQ ID | (SEQ ID NO: 637) | (SEQ ID NO: 638) |
| | NO: 636) | | |
| VHH229B2 | SYAMG | AISWNGGSRYYVESVEG | SRIYTNYVARSYEYDD |
| | (SEQ ID | (SEQ ID NO: 640) | (SEQ ID NO: 641) |
| | NO: 639) | | |
| VHH230B2 | SYAMG | AISWSGLSTYYVESVQG | SRIYTNYVARSYEYDD |
| | (SEQ ID | (SEQ ID NO: 643) | (SEQ ID NO: 644 ) |
| | NO: 642) | | |
| VHH241B2 | SWTMA | AITATGARTSSAESEG | TGIASWDPGMADKYTY |
| | (SEQ ID | (SEQ ID NO: 646) | (SEQ ID NO: 647) |
| | NO: 645) | | |
| VHH277B2 | SWTMA | AITATGARTSSAESEG | TEIAGWDPGMGDKYPY |
| | (SEQ ID | (SEQ ID NO: 649) | (SEQ ID NO: 650) |
| | NO: 648) | | |
| VHH278B2 | SYAMG | VIMRSGDTTAYVDSVKG | GRPVTRTYYHTYTYPHDYDY |
| | (SEQ ID | (SEQ ID NO: 652) | (SEQ ID NO: 653) |
| | NO: 651) | | |
| VHH289B2 | SYAMG | AIMRSGGTTVYVNSVKG | GRPVSRTSYYTYTYPHDYDY |
| | (SEQ ID | (SEQ ID NO: 655) | (SEQ ID NO: 656) |
| | NO: 654) | | |
| VHH294B2 | GYTVG | AIQWSSAYTYSEFVKG | QRSRTSWYASTAEAYDY |
| | (SEQ ID | (SEQ ID NO: 658) | (SEQ ID NO: 659) |
| | NO: 657) | | |
| VHH307B2 | RYIMG | AISWSGLSTHYADSVQG | DRHWETRLLPREYDY |
| | (SEQ ID | (SEQ ID NO: 661) | (SEQ ID NO: 662) |
| | NO: 660) | | |
| VHH322B2 | IHGMG | AIRWSDGTTIHADSVKG | TSNPQFGRNWYTVAAGVEYGY |
| | (SEQ ID | (SEQ ID NO: 664) | (SEQ ID NO: 665) |
| | NO: 663) | | |
| VHH356B2 | INAMG | AITSGGSTAYADSVKG | DRDPITSWYVRGDY |
| | (SEQ ID | (SEQ ID NO: 667) | (SEQ ID NO: 668) |
| | NO: 666) | | |
| VHH361B2 | RYEMA | HISSSGLTTTYADSVKG | NVDGSAWSTLGAGMDY |
| | (SEQ ID | (SEQ ID NO: 670) | (SEQ ID NO: 671) |
| | NO: 669) | | |

TABLE 10
IL-12Rβ2 receptor subunit VHH sequences.

| Clone | |
|---|---|
| Number | VHH Sequence |
| VHH13B2 | QVQLVESGGGLVRPGGSLRLSCAASGFT |
| | FSSNGMHWFRQAPGKGLEWVSSIGSGG |
| | TSIYYADSVKGRFTISRDNAKNTLYLQM |
| | NSLKSDDTAVYYCSKEVTGATRGQGTQ |
| | VTVSS (SEQ ID NO: 672) |
| VHH19B2 | QVQLVESGGGLVQPGGSLRLSCAASGFT |
| | FSNYAMSWVRQAPGKGLEWVSAINSDG |
| | TSTSYADSVKGQFTISRDNAKNTLYLQM |
| | NSLKPEDTAVYYCTKWEGDTDSDYKFK |
| | DYWGQGTQVTVSS (SEQ ID NO: 673) |
| VHH64B2 | QVQLVESGGGLVQPGGSLRLSCAASGFT |
| | FSSKAMHWFRQAPGKGLEWVSSINSGG |
| | AITYYKDSVKGRFTVSRDNAKNILYLEM |
| | NSLKPEDTALYYCATDTDGRTRGQGTQ |
| | VTVSS (SEQ ID NO: 674) |
| VHH67B2 | QVQLVESGGGLVQPGGSLRLSCAASGFT |
| | FSNYAMSWIRQGPERGFEWVSRINSGGG |
| | TTSYADSVKGRFTISRDNAKNTLYLQMN |
| | SLKPEDTAVYYCAKHSHPDYSSNYYRP |
| | GQGTQVTVSS (SEQ ID NO: 675) |
| VHH68B2 | EVQLVESGGGLVQAGGSLKLSCAASGG |
| | TFSSYAMGWFRQAPGKEREFVAAIMRS |
| | GDTTVYVDSVKGRFTVSRDNAKNTVYL |
| | QMNSLKPEDAAVYYCAAGRPVTRTYYS |
| | TYTYPHDYDYWGQGTQVTVSS (SEQ ID |
| | NO: 676) |
| VHH85B2 | QVQLVESGGGLVQAGGSLRVSCAASGRI |
| | PSLNTMAWYRQAPGKQREFVAFITNGG |
| | TTNYVDSVEGRFSISRDNTKNAVDLQM |
| | NSLKPEDTGVYYCNVQISQYPYNYWGQ |
| | GTQVTVSS (SEQ ID NO: 677) |
| VHH93B2 | QVQLVESGGGLVQPGGSLRLSCAASGFT |
| | FEPYDMSWFRQAPGKGPEWVSAIDKGG |
| | GAGATYYADSVKGRFTISRDNAKKTLY |
| | LQMKSLKPEDTAVYYCAITAGPYSDSTP |
| | RVAYRGEGTQVTVSS (SEQ ID NO: 678) |
| VHH94B2 | EVQLVESGGGLVQPGGSLRLSCAASGFT |
| | FSSYAMSWVRQAPGKGLEWVSAINSGG |
| | DSTSYADSVEGRFTISRDNAKNTLYLQM |
| | NSLKPDDTAVYYCAKWSDCSEYGCYDQ |
| | TTTEGTQVTVSS (SEQ ID NO: 679) |
| VHH185B2 | QVQLVESGGGLVQAGGSLRLSCAASGR |
| | TFSRYRMGWFRQAPGKEREFVAAITKSG |
| | ATTYDADSVKGRFTISRDNAKNTLYLQ |
| | MNSLKPEDTAVYYCAASYHSNWWPKN |
| | ADEYAYWGQGTQVTVSS (SEQ ID NO: |
| | 680) |
| VHH190B2 | QVQLQESGPGLVKPSQTLSLTCTVSGGSI |
| | TTNYYYWSWIRQPPGKGLEWMGAIAYS |
| | GSTYYSPSLKSRTSISRDTSKNQFTLQLSS |
| | VTPEDTAVYYCASGINFYSNYPTLNEYD |
| | YWGQGTQVTVSS (SEQ ID NO: 681) |
| VHH194B2 | QLQLVESGGGLVQAGGSLRLSCAASGLT |
| | FSGYTVGWFRQAPGKEREFVAAINWSS |
| | AYTYSEFVKGRFTISRDNAKNTVYLQMS |
| | SLKPEDTAVYYCAARRSRTSWYASTAE |
| | AYDYWGQGTQVTVSS (SEQ ID NO: 682) |
| VHH211B2 | QVQLVESGGGLVQAGNSLRLSCAASGR |
| | TFSSYVVGWFRQAPGKEREFVGRISWSG |
| | ARTYYADSVKGRFTISRDNAKNTVYLQ |
| | MNSLKPEDTAVYYCAADLKWEVLLSEH |
| | FEYWGQGTQVTVSS (SEQ ID NO: 683) |
| VHH216B2 | QVQLVESGGGLVQPGGSLRLSCATSEHT |
| | FGTYRMGWFRQAPGKEREFVAGISWTG |
| | QTTYYAASVKGRFAISKDNAKNTVYLQ |
| | MNTLKSDDTAVYYCAVETNNLGRWDR |
| | PSEYDYWGQGTQVTVSS (SEQ ID NO: |
| | 684) |
| VHH219B2 | QVQLEESGGGLVQAGGSLRLSCAASGR |
| | TFSSYVVGWFRQAPGKEREFVGRISWSG |
| | ARTYYADSVEGRFTISRDNAKNTVYLQ |
| | MNSLKPEDTAVYYCAADLKWEVLLSEH |
| | YEYWGQGTQVTVSS (SEQ ID NO: 685) |
| VHH222B2 | QVQLVESGGGLVQAGGSLRLSCAASGRI |
| | FSAYRMGWFRQAPGKEREFVAGITWRG |
| | QTTYYAASVKGRFTISKDNAKKTVYLQ |
| | MNSLKPEETAVYYCAVETNGLGRWDRP |
| | SEYDYWGQGTQVTVSS (SEQ ID NO: 686) |
| VHH229B2 | QVQLVESGGGLVQAGGSLRLSCAASGR |
| | TFSSYAMGWFRQAPGKEREFVSAISWN |
| | GGSRYYVESVEGRFTISRDNAKNTVYLQ |
| | MNSLKPEDTAVYYCAASRIYTNYVARS |
| | YEYDDWGQGTQVTVSS (SEQ ID NO: |
| | 687) |
| VHH230B2 | QVQLVESGGGLVQAGGSLRLSCAASGR |
| | TFSSYAMGWFRQAPGKEREFVSAISWSG |
| | LSTYYVESVQGRFTISRDNAKNTVYLQM |
| | NSLKPEDTAVYYCAASRIYTNYVARSYE |
| | YDDWGQGTQVTVSS (SEQ ID NO: 688) |
| VHH241B2 | QVQLVESGGGLVQLGDSLRLSCAASGR |
| | GFSSWTMAWFRQALGKRREFVAAITAT |
| | GARTCSAESEGRFTISRDNAKNMVFLQM |
| | NSLKAEDTAVYYCAATGIASWDPGMAD |
| | KYTYWGQGTQVTVSS (SEQ ID NO: 689) |
| VHH277B2 | QVQLVESGGGLVQLGDSLRLSCAASGR |
| | GFSSWTMAWFRQALGKRREFVAAITAT |
| | GARTCSAESEGRFTISRDNAKNMVFLQM |
| | NSLKAEDTAVYYCAATEIAGWDPGMGD |
| | KYPYWGQGTQVTVSS (SEQ ID NO: 690) |
| VHH278B2 | QVQLVESGGGLVQAGGSLKLSCAASGG |
| | TFSSYAMGWFRQAPGKEREFVAVIMRS |
| | GDTTAYVDSVKGRFTVSRDNAKNTVYL |
| | QMNSLKPEDAAVYFCAAGRPVTRTYYH |
| | TYTYPHDYDYWGQGTQVTVSS (SEQ ID |
| | NO: 691) |
| VHH289B2 | QVQLVESGGGLVQAGGSLKLSCAASGG |
| | TFSSYAMGWFRQAPGKEREFVAAIMRS |
| | GGTTVYVNSVKGRFTVSRDNAKNTVYL |
| | QMNSLKPEDAAVYYCAAGRPVSRTSYY |
| | TYTYPHDYDYWGQGTQVSVSS (SEQ ID |
| | NO: 692) |
| VHH294B2 | QLQLVESGGGLVQAGGSLRLSCAASGLT |
| | FSGYTVGWFRQAPGKEREFVAAINWSS |
| | AYTYSEFVKGRFTISRDNAKNTVYLQMS |
| | SLKPEDTAVYYCAAQRSRTSWYASTAE |
| | AYDYWGQGTQVTVSS (SEQ ID NO: 693) |
| VHH307B2 | QVQLVESGGGLVQAGGSLRLSCAASGR |
| | TFSRYIMGWFRQATGKEREFVAAISWSG |
| | LSTHYADSVQGRFTISRDNAENTVYLQM |
| | NSLKPEDTAVYYCAADRHWETRLLPRE |
| | YDYWGQGTQVTVSS (SEQ ID NO: 694) |
| VHH322B2 | QLQLVESGGGLVQAGGSLRLSCAASGR |
| | TFGIHGMGWFRQAPGKEREFAAAIRWS |
| | DGTTIHADSVKGRLTISRDNAKNTVYLQ |
| | MNSLKPEDTAVYYCAATSNPQFGRNWY |
| | TVAAGVEYGYWGQGTQVTVSS (SEQ ID |
| | NO: 695) |
| VHH356B2 | QLQLVESGGGLVQPGGSLRLSCAASGSI |
| | FSINAMGWYRQAPGKQRELVAAITSGGS |
| | TAYADSVKGRFTISRDNAKNTVYLQMN |
| | SLKPEDTAVYYCNADRDPITSWYVRGD |
| | YWGQGTQVTVSS (SEQ ID NO: 696) |
| VHH361B2 | QVQLVESGGGLVQTGASLRLSCAASGR |
| | TFGRYEMAWFRQAPGKEREFVAHISSSG |
| | LTTTYADSVKGRFTISRGNALNTVYLQM |
| | NSLKPEDTAVYYCAANVDGSAWSTLGA |
| | GMDYWGKGTLVTISS (SEQ ID NO: 697) |

### VHH or VH P14A Substitutions

It was previously observed that a proline in position 14 (P14) could be destabilizing the molecule in the context of a rigidified hinge (no upper hinge region). Reverting this mutation back to alanine, which is found in the germline gene sequence, improved both the affinity and the activity of the bispecific. Accordingly, an alanine substitution at position 14 of a VHH or a VH domain, according to Kabat, enhances agonist activity. This is further described in PCT/US2024/023406, filed Apr. 5, 2024, and incorporated herein by reference.

Thus, in some embodiments, the VHH domains disclosed herein comprise a P14A amino acid substitution according to Kabat numbering. In other embodiments, VH domains disclosed herein comprise a P14A amino acid substitution according to Kabat numbering.

In some embodiments, the P14A amino acid substitution further stabilizes the multispecific binding protein.

In some embodiments, the P14A amino acid substitution increases the agonist properties of the multispecific binding protein.

### Multi-Specific IL-12R Binding Proteins

In some embodiments, the IL-12Rβ1 and IL-12Rβ2 binding domains disclosed herein can be paired together or operatively linked to generate a multi-specific binding protein which is capable of cross-linking the IL-12Rβ1 and IL-12Rβ2 subunits of the IL-12 receptor (e.g., the human IL-12 receptor). In some embodiments, the IL-12Rβ1 binding domain (e.g., VHH) is operatively linked (directly or indirectly) to the N- and/or C-terminus of a first Fc domain or polypeptide, and the IL-12Rβ2 binding domain is operatively linked to the N- and/or C-terminus of a second Fc domain or polypeptide, such that the first Fc domain and the second Fc domain facilitate heterodimerization of the IL-12Rβ1 and the IL-12Rβ2 binding domains.

In certain exemplary embodiments, the multi-specific binding protein comprises a first heavy chain (HC1), a second heavy chain (HC2), and a light chain (LC) of an antibody. In some embodiments, the multi-specific binding protein lacks a LC sequence. In some embodiments, the multi-specific binding protein comprises any one of the sequences of Table 11.

TABLE 11
Heavy chain and light chain amino acid sequences of IL-12
receptor binders.

| Antibody | | | |
|---|---|---|---|
| ID | HC1 | HC2 | LC1 |
| DGL393 | QVQLVHSGPEVKKPGASVK | QVQLVESGGGLVQAGGSLR | |
| | VSCKASGYTFTDYYMHWV | LSCAASGRTFSRYRMGWFR | |
| | RQAPGQGLEWMGWINPDS | QAPGKEREFVAAITKSGAT | |
| | GGTNYAQKFQGRVTMTRD | TYDADSVKGRFTISRDNAK | |
| | TSISTAYMELSRLRSDDTAV | NTLYLQMNSLKPEDTAVYY | |
| | YHCAREGATSNFDYWGQG | CAASYHSNWWPKNADEYA | |
| | TLVTVSSASTKGPSVFPLAP | YWGQGTQVTVEPKSSDKT | |
| | SSKSTSGGTAALGCLVKDY | HTCPPCPAPEAAGAPSVFLF | |
| | FPEPVTVSWNSGALTSGVH | PPKPKDTLMISRTPEVTCVV | |
| | TFPAVLQSSGLYSLSSVVTV | VDVSHEDPEVKFNWYVDG | |
| | PSSSLGTQTYICNVNHKPSN | VEVHNAKTKPREEQYNSTY | |
| | TKVDKKVEPKSCDKTHTCP | RVVSVLTVLHQDWLNGKE | |
| | PCPAPEAAGAPSVFLFPPKP | YKCKVSNKALPAPIEKTISK | |
| | KDTLMISRTPEVTCVVVDV | AKGQPREPQVCTLPPSRDEL | |
| | SHEDPEVKFNWYVDGVEV | TKNQVSLSCAVKGFYPSDI | |
| | HNAKTKPREEQYNSTYRVV | AVEWESNGQPENNYKTTPP | |
| | SVLTVLHQDWLNGKEYKC | VLDSDGSFFLVSKLTVDKS | |
| | KVSNKALPAPIEKTISKAKG | RWQQGNVFSCSVMHEALH | |
| | QPREPQVYTLPPCRDELTK | NRFTQKSLSLSPG | |
| | NQVSLWCLVKGFYPSDIAV | (SEQ ID NO: 699) | |
| | EWESNGQPENNYKTTPPVL | | |
| | DSDGSFFLYSKLTVDKSRW | | |
| | QQGNVFSCSVMHEALHNH | | |
| | YTQKSLSLSPG | | |
| | (SEQ ID NO: 698) | | |
| DGL395 | QVQLQESGPGLVKPSETLSL | QVQLVESGGGLVQAGGSLR | DIQLTQSPSFLSAS |
| | TCTVSGGSISSYDWSWIRQS | LSCAASGRTFSSYAMGWFR | VGDRVTITCRAS |
| | AGKGLEWIGRIYTSGSTIYN | QAPGKEREFVSAISWSGLST | QGISSFLAWYQQ |
| | PSLKSRVTMSVDTSKNEISL | YYVESVQGRFTISRDNAKN | KPGKAPKLLIYA |
| | KLSSVTAADTAFYYCAKDR | TVYLQMNSLKPEDTAVYY | ASTLQSGVPSRFS |
| | GNYPHDAFDIWGQGTMITV | CAASRIYTNYVARSYEYDD | GSGSGTEFTLTISS |
| | SSASTKGPSVFPLAPSSKSTS | WGQGTQVTVEPKSSDKTHT | LQPEDFATYYCQ |
| | GGTAALGCLVKDYFPEPVT | CPPCPAPEAAGAPSVFLFPP | QLNGYPFTFGPGT |
| | VSWNSGALTSGVHTFPAVL | KPKDTLMISRTPEVTCVVV | KVDIKRTVAAPS |
| | QSSGLYSLSSVVTVPSSSLG | DVSHEDPEVKFNWYVDGV | VFIFPPSDEQLKS |
| | TQTYICNVNHKPSNTKVDK | EVHNAKTKPREEQYNSTYR | GTASVVCLLNNF |
| | KVEPKSCDKTHTCPPCPAPE | VVSVLTVLHQDWLNGKEY | YPREAKVQWKV |
| | AAGAPSVFLFPPKPKDTLMI | KCKVSNKALPAPIEKTISKA | DNALQSGNSQES |
| | SRTPEVTCVVVDVSHEDPE | KGQPREPQVCTLPPSRDELT | VTEQDSKDSTYS |
| | VKFNWYVDGVEVHNAKTK | KNQVSLSCAVKGFYPSDIA | LSSTLTLSKADYE |
| | PREEQYNSTYRVVSVLTVL | VEWESNGQPENNYKTTPPV | KHKVYACEVTHQ |
| | HQDWLNGKEYKCKVSNKA | LDSDGSFFLVSKLTVDKSR | GLSSPVTKSFNRG |
| | LPAPIEKTISKAKGQPREPQ | WQQGNVFSCSVMHEALHN | EC |
| | VYTLPPCRDELTKNQVSLW | RFTQKSLSLSPG | (SEQ ID |
| | CLVKGFYPSDIAVEWESNG | (SEQ ID NO: 701) | NO: 702) |
| | QPENNYKTTPPVLDSDGSFF | | |
| | LYSKLTVDKSRWQQGNVFS | | |
| | CSVMHEALHNHYTQKSLSL | | |
| | SPG | | |
| | (SEQ ID NO: 700) | | |
| DGL397 | QVHLVQSGAEVKKPGASV | QVQLVESGGGLVQAGGSLR | DIQLTQSPSFLSAS |
| | KVSCEASGYTFTDYYIHWL | LSCAASGRIFSAYRMGWFR | VGDRVTITCRAS |
| | RQAPGQGLEWMGWINPNS | QAPGKEREFVAGITWRGQT | QGISSYLAWYQQ |
| | GGTHYAQKFQGRVTMTRD | TYYAASVKGRFTISKDNAK | QPGKPPKLLIYAA |
| | TSISTAYMELSRLSSDDTAV | KTVYLQMNSLKPEETAVYY | STLHSGVPSRFSG |
| | YYCAREGGWNYFSGMDV | CAVETNGLGRWDRPSEYD | SGSGTEFTLTISSL |
| | WGQGTTVTVSSASTKGPSV | YWGQGTQVTVEPKSSDKT | QPEDFATYYCQQ |
| | FPLAPSSKSTSGGTAALGCL | HTCPPCPAPEAAGAPSVFLF | LNRYPYTCGQGT |
| | VKDYFPEPVTVSWNSGALT | PPKPKDTLMISRTPEVTCVV | KLEIKRTVAAPSV |
| | SGVHTFPAVLQSSGLYSLSS | VDVSHEDPEVKFNWYVDG | FIFPPSDEQLKSGT |
| | VVTVPSSSLGTQTYICNVNH | VEVHNAKTKPREEQYNSTY | ASVVCLLNNFYP |
| | KPSNTKVDKKVEPKSCDKT | RVVSVLTVLHQDWLNGKE | REAKVQWKVDN |
| | HTCPPCPAPEAAGAPSVFLF | YKCKVSNKALPAPIEKTISK | ALQSGNSQESVT |
| | PPKPKDTLMISRTPEVTCVV | AKGQPREPQVCTLPPSRDEL | EQDSKDSTYSLSS |
| | VDVSHEDPEVKFNWYVDG | TKNQVSLSCAVKGFYPSDI | TLTLSKADYEKH |
| | VEVHNAKTKPREEQYNSTY | AVEWESNGQPENNYKTTPP | KVYACEVTHQGL |
| | RVVSVLTVLHQDWLNGKE | VLDSDGSFFLVSKLTVDKS | SSPVTKSFNRGEC |
| | YKCKVSNKALPAPIEKTISK | RWQQGNVFSCSVMHEALH | (SEQ ID |
| | AKGQPREPQVYTLPPCRDE | NRFTQKSLSLSPG | NO: 705) |
| | LTKNQVSLWCLVKGFYPSD | (SEQ ID NO: 704) | |
| | IAVEWESNGQPENNYKTTP | | |
| | PVLDSDGSFFLYSKLTVDKS | | |
| | RWQQGNVFSCSVMHEALH | | |
| | NHYTQKSLSLSPG | | |
| | (SEQ ID NO: 703) | | |
| DGL398 | QVHLVQSGAEVKKPGASV | QVQLVESGGGLVQAGGSLR | DIQLTQSPSFLSAS |
| | KVSCEASGYTFTDYYIHWL | LSCAASGRTFSSYAMGWFR | VGDRVTITCRAS |
| | RQAPGQGLEWMGWINPNS | QAPGKEREFVSAISWSGLST | QGISSYLAWYQQ |
| | GGTHYAQKFQGRVTMTRD | YYVESVQGRFTISRDNAKN | QPGKPPKLLIYAA |
| | TSISTAYMELSRLSSDDTAV | TVYLQMNSLKPEDTAVYY | STLHSGVPSRFSG |
| | YYCAREGGWNYFSGMDV | CAASRIYTNYVARSYEYDD | SGSGTEFTLTISSL |
| | WGQGTTVTVSSASTKGPSV | WGQGTQVTVEPKSSDKTHT | QPEDFATYYCQQ |
| | FPLAPSSKSTSGGTAALGCL | CPPCPAPEAAGAPSVFLFPP | LNRYPYTCGQGT |
| | VKDYFPEPVTVSWNSGALT | KPKDTLMISRTPEVTCVVV | KLEIKRTVAAPSV |
| | SGVHTFPAVLQSSGLYSLSS | DVSHEDPEVKFNWYVDGV | FIFPPSDEQLKSGT |
| | VVTVPSSSLGTQTYICNVNH | EVHNAKTKPREEQYNSTYR | ASVVCLLNNFYP |
| | KPSNTKVDKKVEPKSCDKT | VVSVLTVLHQDWLNGKEY | REAKVQWKVDN |
| | HTCPPCPAPEAAGAPSVFLF | KCKVSNKALPAPIEKTISKA | ALQSGNSQESVT |
| | PPKPKDTLMISRTPEVTCVV | KGQPREPQVCTLPPSRDELT | EQDSKDSTYSLSS |
| | VDVSHEDPEVKFNWYVDG | KNQVSLSCAVKGFYPSDIA | TLTLSKADYEKH |
| | VEVHNAKTKPREEQYNSTY | VEWESNGQPENNYKTTPPV | KVYACEVTHQGL |
| | RVVSVLTVLHQDWLNGKE | LDSDGSFFLVSKLTVDKSR | SSPVTKSFNRGEC |
| | YKCKVSNKALPAPIEKTISK | WQQGNVFSCSVMHEALHN | (SEQ ID |
| | AKGQPREPQVYTLPPCRDE | RFTQKSLSLSPG | NO: 708) |
| | LTKNQVSLWCLVKGFYPSD | (SEQ ID NO: 707) | |
| | IAVEWESNGQPENNYKTTP | | |
| | PVLDSDGSFFLYSKLTVDKS | | |
| | RWQQGNVFSCSVMHEALH | | |
| | NHYTQKSLSLSPG | | |
| | (SEQ ID NO: 706) | | |
| DGL400 | QVHLVQSGAEVKKPGASV | QVQLVESGGGLVQAGGSLR | DIQLTQSPSFLSAS |
| | KVSCKASGYTFIDYYMHW | LSCAASGRTFSRYRMGWFR | VGDRVTITCRAS |
| | VRQAPGQGLEWMGWINPN | QAPGKEREFVAAITKSGAT | QGISSYLAWYQQ |
| | SGGTHYSQKFQGRVTMTW | TYDADSVKGRFTISRDNAK | KPGKAPKVLIYA |
| | DTSISTAYMELSRLTSDDTA | NTLYLQMNSLKPEDTAVYY | ASTLQSGVPSRFS |
| | VYYCAREGDYISSSAFDYW | CAASYHSNWWPKNADEYA | GSGSGTEFTLTISS |
| | GQGTLVTVSSASTKGPSVFP | YWGQGTQVTVEPKSSDKT | LLPEDFATYFCQQ |
| | LAPSSKSTSGGTAALGCLV | HTCPPCPAPEAAGAPSVFLF | LHRYPYTFGQGA |
| | KDYFPEPVTVSWNSGALTS | PPKPKDTLMISRTPEVTCVV | KLEIKRTVAAPSV |
| | GVHTFPAVLQSSGLYSLSSV | VDVSHEDPEVKFNWYVDG | FIFPPSDEQLKSGT |
| | VTVPSSSLGTQTYICNVNHK | VEVHNAKTKPREEQYNSTY | ASVVCLLNNFYP |
| | PSNTKVDKKVEPKSCDKTH | RVVSVLTVLHQDWLNGKE | REAKVQWKVDN |
| | TCPPCPAPEAAGAPSVFLFP | YKCKVSNKALPAPIEKTISK | ALQSGNSQESVT |
| | PKPKDTLMISRTPEVTCVVV | AKGQPREPQVCTLPPSRDEL | EQDSKDSTYSLSS |
| | DVSHEDPEVKFNWYVDGV | TKNQVSLSCAVKGFYPSDI | TLTLSKADYEKH |
| | EVHNAKTKPREEQYNSTYR | AVEWESNGQPENNYKTTPP | KVYACEVTHQGL |
| | VVSVLTVLHQDWLNGKEY | VLDSDGSFFLVSKLTVDKS | SSPVTKSFNRGEC |
| | KCKVSNKALPAPIEKTISKA | RWQQGNVFSCSVMHEALH | (SEQ ID |
| | KGQPREPQVYTLPPCRDEL | NRFTQKSLSLSPG | NO: 711) |
| | TKNQVSLWCLVKGFYPSDI | (SEQ ID NO: 710) | |
| | AVEWESNGQPENNYKTTPP | | |
| | VLDSDGSFFLYSKLTVDKS | | |
| | RWQQGNVFSCSVMHEALH | | |
| | NHYTQKSLSLSPG | | |
| | (SEQ ID NO: 709) | | |
| DGL405 | QVQLQESGPGLVKPSETLSL | QVQLVESGGGLVQAGGSLR | DIQLTQSPSFLSAS |
| | TCTVSGGSISSYYWSWIRQS | LSCAASGRIFSAYRMGWFR | VGYRVTITCRAS |
| | AGKELEWIGRIYTSGSTIYN | QAPGKEREFVAGITWRGQT | QGISSYLAWYQQ |
| | PSLKSRVTMSVDPSKNQFSL | TYYAASVKGRFTISKDNAK | KPGKAPQLLIYA |
| | KLSSVTAADTAVYYCANSR | KTVYLQMNSLKPEETAVYY | ASTLQSGVPSRFS |
| | SYPHDAFDIWGQGTMVTVS | CAVETNGLGRWDRPSEYD | GRGSGTEFTLTIS |
| | SASTKGPSVFPLAPSSKSTS | YWGQGTQVTVEPKSSDKT | SLQPEDFATYYC |
| | GGTAALGCLVKDYFPEPVT | HTCPPCPAPEAAGAPSVFLF | QQLNGYPFTFGP |
| | VSWNSGALTSGVHTFPAVL | PPKPKDTLMISRTPEVTCVV | GTKVNIKRTVAA |
| | QSSGLYSLSSVVTVPSSSLG | VDVSHEDPEVKFNWYVDG | PSVFIFPPSDEQLK |
| | TQTYICNVNHKPSNTKVDK | VEVHNAKTKPREEQYNSTY | SGTASVVCLLNN |
| | KVEPKSCDKTHTCPPCPAPE | RVVSVLTVLHQDWLNGKE | FYPREAKVQWKV |
| | AAGAPSVFLFPPKPKDTLMI | YKCKVSNKALPAPIEKTISK | DNALQSGNSQES |
| | SRTPEVTCVVVDVSHEDPE | AKGQPREPQVCTLPPSRDEL | VTEQDSKDSTYS |
| | VKFNWYVDGVEVHNAKTK | TKNQVSLSCAVKGFYPSDI | LSSTLTLSKADYE |
| | PREEQYNSTYRVVSVLTVL | AVEWESNGQPENNYKTTPP | KHKVYACEVTHQ |
| | HQDWLNGKEYKCKVSNKA | VLDSDGSFFLVSKLTVDKS | GLSSPVTKSFNRG |
| | LPAPIEKTISKAKGQPREPQ | RWQQGNVFSCSVMHEALH | EC |
| | VYTLPPCRDELTKNQVSLW | NRFTQKSLSLSPG | (SEQ ID |
| | CLVKGFYPSDIAVEWESNG | (SEQ ID NO: 713) | NO: 714) |
| | QPENNYKTTPPVLDSDGSFF | | |
| | LYSKLTVDKSRWQQGNVFS | | |
| | CSVMHEALHNHYTQKSLSL | | |
| | SPG | | |
| | (SEQ ID NO: 712) | | |
| DGL407 | QVQLVESGGGVVQPGRSLR | QVQLVESGGGLVRPGGSLR | DIQMTQSPSSLSA |
| | LSCAASGFTFSSYGMHWVR | LSCAASGFTFSSNGMHWFR | SVGDRVTITCQAS |
| | QAPGKGLEWVAVIWNDGS | QAPGKGLEWVSSIGSGGTSI | QDISNYLNWYQQ |
| | NKYYADSVKGRFTISRDNS | YYADSVKGRFTISRDNAKN | KPGKAPKLLIYD |
| | KNTLYLQMNSLRAEDTAV | TLYLQMNSLKSDDTAVYY | ASNLETGVPSRFS |
| | YYCARGELFFDYWGQGTL | CSKEVTGATRGQGTQVTVE | GSGSGTDFTFTISS |
| | VTVSSASTKGPSVFPLAPSS | PKSSDKTHTCPPCPAPEAAG | LQPEDIATYYCQ |
| | KSTSGGTAALGCLVKDYFP | APSVFLFPPKPKDTLMISRT | QYDNLFTFGGGT |
| | EPVTVSWNSGALTSGVHTF | PEVTCVVVDVSHEDPEVKF | KVEIKRTVAAPSV |
| | PAVLQSSGLYSLSSVVTVPS | NWYVDGVEVHNAKTKPRE | FIFPPSDEQLKSGT |
| | SSLGTQTYICNVNHKPSNTK | EQYNSTYRVVSVLTVLHQD | ASVVCLLNNFYP |
| | VDKKVEPKSCDKTHTCPPC | WLNGKEYKCKVSNKALPA | REAKVQWKVDN |
| | PAPEAAGAPSVFLFPPKPKD | PIEKTISKAKGQPREPQVCT | ALQSGNSQESVT |
| | TLMISRTPEVTCVVVDVSH | LPPSRDELTKNQVSLSCAV | EQDSKDSTYSLSS |
| | EDPEVKFNWYVDGVEVHN | KGFYPSDIAVEWESNGQPE | TLTLSKADYEKH |
| | AKTKPREEQYNSTYRVVSV | NNYKTTPPVLDSDGSFFLVS | KVYACEVTHQGL |
| | LTVLHQDWLNGKEYKCKV | KLTVDKSRWQQGNVFSCS | SSPVTKSFNRGEC |
| | SNKALPAPIEKTISKAKGQP | VMHEALHNRFTQKSLSLSP | (SEQ ID |
| | REPQVYTLPPCRDELTKNQ | G | NO: 717) |
| | VSLWCLVKGFYPSDIAVEW | (SEQ ID NO: 716) | |
| | ESNGQPENNYKTTPPVLDS | | |
| | DGSFFLYSKLTVDKSRWQQ | | |
| | GNVFSCSVMHEALHNHYT | | |
| | QKSLSLSPG | | |
| | (SEQ ID NO: 715) | | |
| DGL408 | QVQLVESGGGVVQPGRSLR | QVQLVESGGGLVQPGGSLR | DIQMTQSPSSLSA |
| | LSCAASGFTFSSYGMHWVR | LSCAASGFTFSNYAMSWVR | SVGDRVTITCQAS |
| | QAPGKGLEWVAVIWNDGS | QAPGKGLEWVSAINSDGTS | QDISNYLNWYQQ |
| | NKYYADSVKGRFTISRDNS | TSYADSVKGQFTISRDNAK | KPGKAPKLLIYD |
| | KNTLYLQMNSLRAEDTAV | NTLYLQMNSLKPEDTAVYY | ASNLETGVPSRFS |
| | YYCARGELFFDYWGQGTL | CTKWEGDTDSDYKFKDYW | GSGSGTDFTFTISS |
| | VTVSSASTKGPSVFPLAPSS | GQGTQVTVEPKSSDKTHTC | LQPEDIATYYCQ |
| | KSTSGGTAALGCLVKDYFP | PPCPAPEAAGAPSVFLFPPK | QYDNLFTFGGGT |
| | EPVTVSWNSGALTSGVHTF | PKDTLMISRTPEVTCVVVD | KVEIKRTVAAPSV |
| | PAVLQSSGLYSLSSVVTVPS | VSHEDPEVKFNWYVDGVE | FIFPPSDEQLKSGT |
| | SSLGTQTYICNVNHKPSNTK | VHNAKTKPREEQYNSTYRV | ASVVCLLNNFYP |
| | VDKKVEPKSCDKTHTCPPC | VSVLTVLHQDWLNGKEYK | REAKVQWKVDN |
| | PAPEAAGAPSVFLFPPKPKD | CKVSNKALPAPIEKTISKAK | ALQSGNSQESVT |
| | TLMISRTPEVTCVVVDVSH | GQPREPQVCTLPPSRDELTK | EQDSKDSTYSLSS |
| | EDPEVKFNWYVDGVEVHN | NQVSLSCAVKGFYPSDIAV | TLTLSKADYEKH |
| | AKTKPREEQYNSTYRVVSV | EWESNGQPENNYKTTPPVL | KVYACEVTHQGL |
| | LTVLHQDWLNGKEYKCKV | DSDGSFFLVSKLTVDKSRW | SSPVTKSFNRGEC |
| | SNKALPAPIEKTISKAKGQP | QQGNVFSCSVMHEALHNRF | (SEQ ID |
| | REPQVYTLPPCRDELTKNQ | TQKSLSLSPG | NO: 720) |
| | VSLWCLVKGFYPSDIAVEW | (SEQ ID NO: 719) | |
| | ESNGQPENNYKTTPPVLDS | | |
| | DGSFFLYSKLTVDKSRWQQ | | |
| | GNVFSCSVMHEALHNHYT | | |
| | QKSLSLSPG | | |
| | (SEQ ID NO: 718) | | |
| DGL411 | EVQLVESGGGLVQPGGSLR | QVQLVESGGGLVQAGGSLR | EIVLTQSPGTLSLS |
| | LSCAASGFTFSSYWMSWVR | LSCAASGRTFSRYRMGWFR | PGERATLSCRASQ |
| | QAPGKGLEWVANIKQDGSE | QAPGKEREFVAAITKSGAT | SINSNYLAWYQQ |
| | KYYVDSVKGRFTISRDNAK | TYDADSVKGRFTISRDNAK | KPGQAPRLLIYGA |
| | NSLYLQMNSLRAEDTAVY | NTLYLQMNSLKPEDTAVYY | SSRATGIPDRFSG |
| | YCAREELTGLYWYFDLWG | CAASYHSNWWPKNADEYA | SGSGTDFTLTISR |
| | RGTLVTVSSASTKGPSVFPL | YWGQGTQVTVEPKSSDKT | LEPEDFAVYYCQ |
| | APSSKSTSGGTAALGCLVK | HTCPPCPAPEAAGAPSVFLF | QYDSFGGGTKVE |
| | DYFPEPVTVSWNSGALTSG | PPKPKDTLMISRTPEVTCVV | IKRTVAAPSVFIFP |
| | VHTFPAVLQSSGLYSLSSVV | VDVSHEDPEVKFNWYVDG | PSDEQLKSGTASV |
| | TVPSSSLGTQTYICNVNHKP | VEVHNAKTKPREEQYNSTY | VCLLNNFYPREA |
| | SNTKVDKKVEPKSCDKTHT | RVVSVLTVLHQDWLNGKE | KVQWKVDNALQ |
| | CPPCPAPEAAGAPSVFLFPP | YKCKVSNKALPAPIEKTISK | SGNSQESVTEQDS |
| | KPKDTLMISRTPEVTCVVV | AKGQPREPQVCTLPPSRDEL | KDSTYSLSSTLTL |
| | DVSHEDPEVKFNWYVDGV | TKNQVSLSCAVKGFYPSDI | SKADYEKHKVYA |
| | EVHNAKTKPREEQYNSTYR | AVEWESNGQPENNYKTTPP | CEVTHQGLSSPVT |
| | VVSVLTVLHQDWLNGKEY | VLDSDGSFFLVSKLTVDKS | KSFNRGEC |
| | KCKVSNKALPAPIEKTISKA | RWQQGNVFSCSVMHEALH | (SEQ ID |
| | KGQPREPQVYTLPPCRDEL | NRFTQKSLSLSPG | NO: 723) |
| | TKNQVSLWCLVKGFYPSDI | (SEQ ID NO: 722) | |
| | AVEWESNGQPENNYKTTPP | | |
| | VLDSDGSFFLYSKLTVDKS | | |
| | RWQQGNVFSCSVMHEALH | | |
| | NHYTQKSLSLSPG | | |
| | (SEQ ID NO: 721) | | |
| DGL412 | QVQLVQSGAEVKKPGASA | QVQLVESGGGLVRPGGSLR | DIQMTQSPSSLSA |
| | KVSCKASGYTFTDYYLHW | LSCAASGFTFSSNGMHWFR | SVGDRVTITCRAS |
| | VRQAPGQGLEWMGWINPN | QAPGKGLEWVSSIGSGGTSI | QGIGNYLAWFQQ |
| | NGGTHYAQKFQGRVTMTR | YYADSVKGRFTISRDNAKN | RPGKAPKSLIYAA |
| | DTSISTAYMELSRLRSDDTA | TLYLQMNSLKSDDTAVYY | SSLQSGVPSKFSG |
| | VYYCAREMGGTTAFDYWG | CSKEVTGATRGQGTQVTVE | SGSGTDFTLTISSL |
| | QGTLVTVSSASTKGPSVFPL | PKSSDKTHTCPPCPAPEAAG | QPADFAIYYCQQ |
| | APSSKSTSGGTAALGCLVK | APSVFLFPPKPKDTLMISRT | YNIYPYTFGPGTR |
| | DYFPEPVTVSWNSGALTSG | PEVTCVVVDVSHEDPEVKF | LEIKRTVAAPSVF |
| | VHTFPAVLQSSGLYSLSSVV | NWYVDGVEVHNAKTKPRE | IFPPSDEQLKSGT |
| | TVPSSSLGTQTYICNVNHKP | EQYNSTYRVVSVLTVLHQD | ASVVCLLNNFYP |
| | SNTKVDKKVEPKSCDKTHT | WLNGKEYKCKVSNKALPA | REAKVQWKVDN |
| | CPPCPAPEAAGAPSVFLFPP | PIEKTISKAKGQPREPQVCT | ALQSGNSQESVT |
| | KPKDTLMISRTPEVTCVVV | LPPSRDELTKNQVSLSCAV | EQDSKDSTYSLSS |
| | DVSHEDPEVKFNWYVDGV | KGFYPSDIAVEWESNGQPE | TLTLSKADYEKH |
| | EVHNAKTKPREEQYNSTYR | NNYKTTPPVLDSDGSFFLVS | KVYACEVTHQGL |
| | VVSVLTVLHQDWLNGKEY | KLTVDKSRWQQGNVFSCS | SSPVTKSFNRGEC |
| | KCKVSNKALPAPIEKTISKA | VMHEALHNRFTQKSLSLSP | (SEQ ID |
| | KGQPREPQVYTLPPCRDEL | G | NO: 726) |
| | TKNQVSLWCLVKGFYPSDI | (SEQ ID NO: 725) | |
| | AVEWESNGQPENNYKTTPP | | |
| | VLDSDGSFFLYSKLTVDKS | | |
| | RWQQGNVFSCSVMHEALH | | |
| | NHYTQKSLSLSPG | | |
| | (SEQ ID NO: 724) | | |
| DGL413 | QVQLVQSGAEVKKPGASA | QVQLVESGGGLVQAGGSLR | DIQMTQSPSSLSA |
| | KVSCKASGYTFTDYYLHW | LSCAASGRTFSRYRMGWFR | SVGDRVTITCRAS |
| | VRQAPGQGLEWMGWINPN | QAPGKEREFVAAITKSGAT | QGIGNYLAWFQQ |
| | NGGTHYAQKFQGRVTMTR | TYDADSVKGRFTISRDNAK | RPGKAPKSLIYAA |
| | DTSISTAYMELSRLRSDDTA | NTLYLQMNSLKPEDTAVYY | SSLQSGVPSKFSG |
| | VYYCAREMGGTTAFDYWG | CAASYHSNWWPKNADEYA | SGSGTDFTLTISSL |
| | QGTLVTVSSASTKGPSVFPL | YWGQGTQVTVEPKSSDKT | QPADFAIYYCQQ |
| | APSSKSTSGGTAALGCLVK | HTCPPCPAPEAAGAPSVFLF | YNIYPYTFGPGTR |
| | DYFPEPVTVSWNSGALTSG | PPKPKDTLMISRTPEVTCVV | LEIKRTVAAPSVF |
| | VHTFPAVLQSSGLYSLSSVV | VDVSHEDPEVKFNWYVDG | IFPPSDEQLKSGT |
| | TVPSSSLGTQTYICNVNHKP | VEVHNAKTKPREEQYNSTY | ASVVCLLNNFYP |
| | SNTKVDKKVEPKSCDKTHT | RVVSVLTVLHQDWLNGKE | REAKVQWKVDN |
| | CPPCPAPEAAGAPSVFLFPP | YKCKVSNKALPAPIEKTISK | ALQSGNSQESVT |
| | KPKDTLMISRTPEVTCVVV | AKGQPREPQVCTLPPSRDEL | EQDSKDSTYSLSS |
| | DVSHEDPEVKFNWYVDGV | TKNQVSLSCAVKGFYPSDI | TLTLSKADYEKH |
| | EVHNAKTKPREEQYNSTYR | AVEWESNGQPENNYKTTPP | KVYACEVTHQGL |
| | VVSVLTVLHQDWLNGKEY | VLDSDGSFFLVSKLTVDKS | SSPVTKSFNRGEC |
| | KCKVSNKALPAPIEKTISKA | RWQQGNVFSCSVMHEALH | (SEQ ID |
| | KGQPREPQVYTLPPCRDEL | NRFTQKSLSLSPG | NO: 729) |
| | TKNQVSLWCLVKGFYPSDI | (SEQ ID NO: 728) | |
| | AVEWESNGQPENNYKTTPP | | |
| | VLDSDGSFFLYSKLTVDKS | | |
| | RWQQGNVFSCSVMHEALH | | |
| | NHYTQKSLSLSPG | | |
| | (SEQ ID NO: 727) | | |
| DGL415 | QVQLVQSGAEVKKPGASV | QVQLVESGGGLVQAGGSLR | DIQMTQSPSSLSA |
| | KVSCKASGYTFTDYYIHWV | LSCAASGRTFSRYRMGWFR | SVGNRVTITCRAS |
| | RQAPGQGLEWMGWINPDS | QAPGKEREFVAAITKSGAT | QGINNYLAWFQQ |
| | GGTHYAQKFQGRVTMTRD | TYDADSVKGRFTISRDNAK | KPGKAPKSLIYAA |
| | TSISTVYMDLSRLRSDDTAV | NTLYLQMNSLKPEDTAVYY | SSLQSGVPSKFSG |
| | YYCAREGEGLASFDSWGQ | CAASYHSNWWPKNADEYA | SGSGTDFTLTISSL |
| | GTLVTVSSASTKGPSVFPLA | YWGQGTQVTVEPKSSDKT | QPEDFATYYCQQ |
| | PSSKSTSGGTAALGCLVKD | HTCPPCPAPEAAGAPSVFLF | YNNYPFTFGPGT |
| | YFPEPVTVSWNSGALTSGV | PPKPKDTLMISRTPEVTCVV | KVDIKRTVAAPS |
| | HTFPAVLQSSGLYSLSSVVT | VDVSHEDPEVKFNWYVDG | VFIFPPSDEQLKS |
| | VPSSSLGTQTYICNVNHKPS | VEVHNAKTKPREEQYNSTY | GTASVVCLLNNF |
| | NTKVDKKVEPKSCDKTHTC | RVVSVLTVLHQDWLNGKE | YPREAKVQWKV |
| | PPCPAPEAAGAPSVFLFPPK | YKCKVSNKALPAPIEKTISK | DNALQSGNSQES |
| | PKDTLMISRTPEVTCVVVD | AKGQPREPQVCTLPPSRDEL | VTEQDSKDSTYS |
| | VSHEDPEVKFNWYVDGVE | TKNQVSLSCAVKGFYPSDI | LSSTLTLSKADYE |
| | VHNAKTKPREEQYNSTYRV | AVEWESNGQPENNYKTTPP | KHKVYACEVTHQ |
| | VSVLTVLHQDWLNGKEYK | VLDSDGSFFLVSKLTVDKS | GLSSPVTKSFNRG |
| | CKVSNKALPAPIEKTISKAK | RWQQGNVFSCSVMHEALH | EC |
| | GQPREPQVYTLPPCRDELT | NRFTQKSLSLSPG | (SEQ ID |
| | KNQVSLWCLVKGFYPSDIA | (SEQ ID NO: 731) | NO: 732) |
| | VEWESNGQPENNYKTTPPV | | |
| | LDSDGSFFLYSKLTVDKSR | | |
| | WQQGNVFSCSVMHEALHN | | |
| | HYTQKSLSLSPG | | |
| | (SEQ ID NO: 730) | | |
| DGL421 | QITLKESGPTLVKPTQTLTL | QVQLQESGPGLVKPSQTLS | QTVVTQEPSFSVS |
| | TCTFFGFSLNTGGVGVGWI | LTCTVSGGSITTNYYYWSW | PGGTVTLTCGLTS |
| | RQPPGKALEWLTLIYWNDD | IRQPPGKGLEWMGAIAYSG | GSVSTSYYPSWY |
| | KRYSPSLKSRLTITKDTSKN | STYYSPSLKSRTSISRDTSKN | QQTPGQAPRTLIY |
| | QVVLTMTNMDPVDTATYY | QFTLQLSSVTPEDTAVYYC | STYTRSSGVPDRF |
| | CAHTLRWYHGFDIWGQGT | ASGINFYSNYPTLNEYDYW | SGSILGNKAALTI |
| | MVTVSSASTKGPSVFPLAPS | GQGTQVTVEPKSSDKTHTC | TGAQADDESDYY |
| | SKSTSGGTAALGCLVKDYF | PPCPAPEAAGAPSVFLFPPK | CVLYMGSGIWVF |
| | PEPVTVSWNSGALTSGVHT | PKDTLMISRTPEVTCVVVD | GGGTKLTVLGQP |
| | FPAVLQSSGLYSLSSVVTVP | VSHEDPEVKFNWYVDGVE | KAAPSVTLFPPSS |
| | SSSLGTQTYICNVNHKPSNT | VHNAKTKPREEQYNSTYRV | EELQANKATLVC |
| | KVDKKVEPKSCDKTHTCPP | VSVLTVLHQDWLNGKEYK | LISDFYPGAVTVA |
| | CPAPEAAGAPSVFLFPPKPK | CKVSNKALPAPIEKTISKAK | WKADSSPVKAGV |
| | DTLMISRTPEVTCVVVDVS | GQPREPQVCTLPPSRDELTK | ETTTPSKQSNNK |
| | HEDPEVKFNWYVDGVEVH | NQVSLSCAVKGFYPSDIAV | YAASSYLSLTPEQ |
| | NAKTKPREEQYNSTYRVVS | EWESNGQPENNYKTTPPVL | WKSHRSYSCQVT |
| | VLTVLHQDWLNGKEYKCK | DSDGSFFLVSKLTVDKSRW | HEGSTVEKTVAP |
| | VSNKALPAPIEKTISKAKGQ | QQGNVFSCSVMHEALHNRF | TECS |
| | PREPQVYTLPPCRDELTKN | TQKSLSLSPG | (SEQ ID |
| | QVSLWCLVKGFYPSDIAVE | (SEQ ID NO: 734) | NO: 735) |
| | WESNGQPENNYKTTPPVLD | | |
| | SDGSFFLYSKLTVDKSRWQ | | |
| | QGNVFSCSVMHEALHNHY | | |
| | TQKSLSLSPG | | |
| | (SEQ ID NO: 733) | | |
| DGL424 | QVQLVESGGGLVQAGGSLR | QVTLRESGPALVKPTQTLTL | QSVLTQPPSVSGA |
| | LSCAASGGPFSGYVMGWFR | TCTFSGFSLSTSGMCVSWIR | PGQRVTISCTGSS |
| | QVSGKEREFVAGITGISSTY | QPPGKALEWLALIYWDDD | SNIGAGYDVHWY |
| | FADSVKGRFTISRDNAKNM | KYYSTSLKTRLTISKDTSKN | QQLPGTAPKLLIF |
| | VYLQMNSLKPEDTAVYYC | QVVLTMTNMDPVDTATYY | GYSNRPSGVPVR |
| | AAVNSFGVIPTSPNGMDYW | CARMRRGIEAFDIWGQGTM | FSGSKSGTSASLA |
| | GKGTLVTVEPKSSDKTHTC | VTVFSASTKGPSVFPLAPSS | ITGLLAEDEADFY |
| | PPCPAPEAAGAPSVFLFPPK | KSTSGGTAALGCLVKDYFP | CQSFDISLTGWVF |
| | PKDTLMISRTPEVTCVVVD | EPVTVSWNSGALTSGVHTF | GGGTKLTVLGQP |
| | VSHEDPEVKFNWYVDGVE | PAVLQSSGLYSLSSVVTVPS | KAAPSVTLFPPSS |
| | VHNAKTKPREEQYNSTYRV | SSLGTQTYICNVNHKPSNTK | EELQANKATLVC |
| | VSVLTVLHQDWLNGKEYK | VDKKVEPKSCDKTHTCPPC | LISDFYPGAVTVA |
| | CKVSNKALPAPIEKTISKAK | PAPEAAGAPSVFLFPPKPKD | WKADSSPVKAGV |
| | GQPREPQVYTLPPCRDELT | TLMISRTPEVTCVVVDVSH | ETTTPSKQSNNK |
| | KNQVSLWCLVKGFYPSDIA | EDPEVKFNWYVDGVEVHN | YAASSYLSLTPEQ |
| | VEWESNGQPENNYKTTPPV | AKTKPREEQYNSTYRVVSV | WKSHRSYSCQVT |
| | LDSDGSFFLYSKLTVDKSR | LTVLHQDWLNGKEYKCKV | HEGSTVEKTVAP |
| | WQQGNVFSCSVMHEALHN | SNKALPAPIEKTISKAKGQP | TECS |
| | HYTQKSLSLSPG | REPQVCTLPPSRDELTKNQ | (SEQ ID |
| | (SEQ ID NO: 736) | VSLSCAVKGFYPSDIAVEW | NO: 738) |
| | | ESNGQPENNYKTTPPVLDS | |
| | | DGSFFLVSKLTVDKSRWQQ | |
| | | GNVFSCSVMHEALHNRFTQ | |
| | | KSLSLSPG | |
| | | (SEQ ID NO: 737) | |
| DGL425 | QVQLVESGGGLVQAGGSLR | EVQLVQSGTEVKKPGESLKI | DVVMTQSPLSLP |
| | LSCAASGGPFSGYVMGWFR | SCKGSGYSFTSYWIGWVRQ | VTLGQPASISCRS |
| | QVSGKEREFVAGITGISSTY | MPGKGLEWMGNIYPGDSD | SQSLVYSDGNTY |
| | FADSVKGRFTISRDNAKNM | TRYSPSFQGHVTISADKSIST | LNWFQQRPGQSP |
| | VYLQMNSLKPEDTAVYYC | AYLQWSSLKASDTAMYYC | RRLIYKVSNRDSG |
| | AAVNSFGVIPTSPNGMDYW | ARRGGGYWGQGTLVTVSS | VPDRFSGSGSGTD |
| | GKGTLVTVEPKSSDKTHTC | ASTKGPSVFPLAPSSKSTSG | FTLKISRVEADDV |
| | PPCPAPEAAGAPSVFLFPPK | GTAALGCLVKDYFPEPVTV | GVYYCMQGTHW |
| | PKDTLMISRTPEVTCVVVD | SWNSGALTSGVHTFPAVLQ | PITFGQGTRLEIK |
| | VSHEDPEVKFNWYVDGVE | SSGLYSLSSVVTVPSSSLGT | RTVAAPSVFIFPPS |
| | VHNAKTKPREEQYNSTYRV | QTYICNVNHKPSNTKVDKK | DEQLKSGTASVV |
| | VSVLTVLHQDWLNGKEYK | VEPKSCDKTHTCPPCPAPEA | CLLNNFYPREAK |
| | CKVSNKALPAPIEKTISKAK | AGAPSVFLFPPKPKDTLMIS | VQWKVDNALQS |
| | GQPREPQVYTLPPCRDELT | RTPEVTCVVVDVSHEDPEV | GNSQESVTEQDS |
| | KNQVSLWCLVKGFYPSDIA | KFNWYVDGVEVHNAKTKP | KDSTYSLSSTLTL |
| | VEWESNGQPENNYKTTPPV | REEQYNSTYRVVSVLTVLH | SKADYEKHKVYA |
| | LDSDGSFFLYSKLTVDKSR | QDWLNGKEYKCKVSNKAL | CEVTHQGLSSPVT |
| | WQQGNVFSCSVMHEALHN | PAPIEKTISKAKGQPREPQV | KSFNRGEC |
| | HYTQKSLSLSPG | CTLPPSRDELTKNQVSLSCA | (SEQ ID |
| | (SEQ ID NO: 739) | VKGFYPSDIAVEWESNGQP | NO: 741) |
| | | ENNYKTTPPVLDSDGSFFLV | |
| | | SKLTVDKSRWQQGNVFSCS | |
| | | VMHEALHNRFTQKSLSLSP | |
| | | G | |
| | | (SEQ ID NO: 740) | |
| DGL427 | QVQLVESGGGLVQAGGSLR | EVLLVESGGGLVKPGGSLR | DIQMTQSPSSVSA |
| | LSCAAPGRYAVGWFRQAP | LSCAASGFTFSSYSMNWVR | SVGDRVTITCRAS |
| | GKEYEFVAAITVSGGSKYY | QAPGKGLEWVSSISSSSDYI | QGISIWLAWYQQ |
| | EDSVKGRFAISRDNAKNTV | HYADSVRGRFTISRDNAKN | KPGKAPKPLIYAA |
| | YLQMNSLKAEDTAVYYCA | SLYLQMNSLRAEDTAVYYC | SSLQSGVPSRFSG |
| | ARTPKFGSGWYNRRGEYD | ARSELGFDYWGQGTLVTVS | SGSGTDFTLTISSL |
| | YWGQGTQVTVEPKSSDKT | SASTKGPSVFPLAPSSKSTS | QPEDFATYYCQQ |
| | HTCPPCPAPEAAGAPSVFLF | GGTAALGCLVKDYFPEPVT | ANSFPLTFGGGTK |
| | PPKPKDTLMISRTPEVTCVV | VSWNSGALTSGVHTFPAVL | VEIKRTVAAPSVF |
| | VDVSHEDPEVKFNWYVDG | QSSGLYSLSSVVTVPSSSLG | IFPPSDEQLKSGT |
| | VEVHNAKTKPREEQYNSTY | TQTYICNVNHKPSNTKVDK | ASVVCLLNNFYP |
| | RVVSVLTVLHQDWLNGKE | KVEPKSCDKTHTCPPCPAPE | REAKVQWKVDN |
| | YKCKVSNKALPAPIEKTISK | AAGAPSVFLFPPKPKDTLMI | ALQSGNSQESVT |
| | AKGQPREPQVYTLPPCRDE | SRTPEVTCVVVDVSHEDPE | EQDSKDSTYSLSS |
| | LTKNQVSLWCLVKGFYPSD | VKFNWYVDGVEVHNAKTK | TLTLSKADYEKH |
| | IAVEWESNGQPENNYKTTP | PREEQYNSTYRVVSVLTVL | KVYACEVTHQGL |
| | PVLDSDGSFFLYSKLTVDKS | HQDWLNGKEYKCKVSNKA | SSPVTKSFNRGEC |
| | RWQQGNVFSCSVMHEALH | LPAPIEKTISKAKGQPREPQ | (SEQ ID |
| | NHYTQKSLSLSPG | VCTLPPSRDELTKNQVSLSC | NO: 744) |
| | (SEQ ID NO: 742) | AVKGFYPSDIAVEWESNGQ | |
| | | PENNYKTTPPVLDSDGSFFL | |
| | | VSKLTVDKSRWQQGNVFS | |
| | | CSVMHEALHNRFTQKSLSL | |
| | | SPG | |
| | | (SEQ ID NO: 743) | |
| DGL428 | QVQLVESGGGLAQAGGSLR | QVQLQESGPGLVKPSETLSL | SYELTQPPSVSVS |
| | LSCAASGRTFSNDDMAWFR | TCTVSGGSISSYYWSWIRQP | PGQTASITCSGDK |
| | QAPGKEREFVAHIDRNAAS | AGKGLEWIGRIYTSGSTNY | LGDKYACWYQQ |
| | TNYADSVKGRFTISRDNAK | NPSLKSRVTMSVDTSKNQF | KPGQSPVLVIYQD |
| | NTVYLQMTRLKPEDTAVY | SLKLSSVTAADTAEYYCAR | SKRPSGIPERFSGS |
| | YCAADPDHFGTTWYQQYT | AGSSGSSYYYYGMDVWGQ | NSGNTATLTISGT |
| | YWGPGTQVTVEPKSSDKTH | GTTVTVSSASTKGPSVFPLA | QAMDEADYYCQ |
| | TCPPCPAPEAAGAPSVFLFP | PSSKSTSGGTAALGCLVKD | AWDSSTVVFGGG |
| | PKPKDTLMISRTPEVTCVVV | YFPEPVTVSWNSGALTSGV | TKLTVLGQPKAA |
| | DVSHEDPEVKFNWYVDGV | HTFPAVLQSSGLYSLSSVVT | PSVTLFPPSSEELQ |
| | EVHNAKTKPREEQYNSTYR | VPSSSLGTQTYICNVNHKPS | ANKATLVCLISDF |
| | VVSVLTVLHQDWLNGKEY | NTKVDKKVEPKSCDKTHTC | YPGAVTVAWKA |
| | KCKVSNKALPAPIEKTISKA | PPCPAPEAAGAPSVFLFPPK | DSSPVKAGVETT |
| | KGQPREPQVYTLPPCRDEL | PKDTLMISRTPEVTCVVVD | TPSKQSNNKYAA |
| | TKNQVSLWCLVKGFYPSDI | VSHEDPEVKFNWYVDGVE | SSYLSLTPEQWKS |
| | AVEWESNGQPENNYKTTPP | VHNAKTKPREEQYNSTYRV | HRSYSCQVTHEG |
| | VLDSDGSFFLYSKLTVDKS | VSVLTVLHQDWLNGKEYK | STVEKTVAPTECS |
| | RWQQGNVFSCSVMHEALH | CKVSNKALPAPIEKTISKAK | (SEQ ID |
| | NHYTQKSLSLSPG | GQPREPQVCTLPPSRDELTK | NO: 747) |
| | (SEQ ID NO: 745) | NQVSLSCAVKGFYPSDIAV | |
| | | EWESNGQPENNYKTTPPVL | |
| | | DSDGSFFLVSKLTVDKSRW | |
| | | QQGNVFSCSVMHEALHNRF | |
| | | TQKSLSLSPG | |
| | | (SEQ ID NO: 746) | |
| DGL430 | QVQLVESGGGLAQAGGSLR | QVHLQESGPGLVKPSETLSL | SYELTQPPSVSVS |
| | LSCAASGRTFSNDDMAWFR | TCTVSGGSISYYYWNWIRQ | PGQTASITCSGRE |
| | QAPGKEREFVAHIDRNAAS | PPGKGLEWIASIYNSGNINY | LGNKYAYWYQQ |
| | TNYADSVKGRFTISRDNAK | NPSLKSRVTISVDTSKNQFS | KPGQSPVLVIYED |
| | NTVYLQMTRLKPEDTAVY | LKLTSVTAADTAVYYCARG | YKRPSGIPERFSG |
| | YCAADPDHFGTTWYQQYT | GYSYGYFDNWGQGILVTVS | SNSGNTATLTISG |
| | YWGPGTQVTVEPKSSDKTH | SASTKGPSVFPLAPSSKSTS | TQAMDEADYYC |
| | TCPPCPAPEAAGAPSVFLFP | GGTAALGCLVKDYFPEPVT | QAWDSSTVIFGG |
| | PKPKDTLMISRTPEVTCVVV | VSWNSGALTSGVHTFPAVL | GTKLTVLGQPKA |
| | DVSHEDPEVKFNWYVDGV | QSSGLYSLSSVVTVPSSSLG | APSVTLFPPSSEEL |
| | EVHNAKTKPREEQYNSTYR | TQTYICNVNHKPSNTKVDK | QANKATLVCLIS |
| | VVSVLTVLHQDWLNGKEY | KVEPKSCDKTHTCPPCPAPE | DFYPGAVTVAW |
| | KCKVSNKALPAPIEKTISKA | AAGAPSVFLFPPKPKDTLMI | KADSSPVKAGVE |
| | KGQPREPQVYTLPPCRDEL | SRTPEVTCVVVDVSHEDPE | TTTPSKQSNNKY |
| | TKNQVSLWCLVKGFYPSDI | VKFNWYVDGVEVHNAKTK | AASSYLSLTPEQ |
| | AVEWESNGQPENNYKTTPP | PREEQYNSTYRVVSVLTVL | WKSHRSYSCQVT |
| | VLDSDGSFFLYSKLTVDKS | HQDWLNGKEYKCKVSNKA | HEGSTVEKTVAP |
| | RWQQGNVFSCSVMHEALH | LPAPIEKTISKAKGQPREPQ | TECS |
| | NHYTQKSLSLSPG | VCTLPPSRDELTKNQVSLSC | (SEQ ID |
| | (SEQ ID NO: 748) | AVKGFYPSDIAVEWESNGQ | NO: 750) |
| | | PENNYKTTPPVLDSDGSFFL | |
| | | VSKLTVDKSRWQQGNVFS | |
| | | CSVMHEALHNRFTQKSLSL | |
| | | SPG | |
| | | (SEQ ID NO: 749) | |
| DGL436 | EVQLVESGGGLVQPGGSLR | QVQLQESGPGLVKPSETLSL | SYELTQPPSVSVS |
| | LSCAASGFTLDYYAIGWFR | TCTVSGGSISSYYWSWIRQP | PGQTASITCSGDK |
| | QAPGKEREGVSCISSSDRST | AGKGLEWIGRIYTSGSTNY | LGDKYACWYQQ |
| | WYADSVKGRFTISRDNAKN | NPSLKSRVTMSVDTSKNQF | KPGQSPVLVIYQD |
| | TVYLQMHSLKPEDTAVYY | SLKLSSVTAADTAEYYCAR | SKRPSGIPERFSGS |
| | CATPCYSDYDPEGYEYDY | AGSSGSSYYYYGMDVWGQ | NSGNTATLTISGT |
| | WGQGTQVTVEPKSSDKTHT | GTTVTVSSASTKGPSVFPLA | QAMDEADYYCQ |
| | CPPCPAPEAAGAPSVFLFPP | PSSKSTSGGTAALGCLVKD | AWDSSTVVFGGG |
| | KPKDTLMISRTPEVTCVVV | YFPEPVTVSWNSGALTSGV | TKLTVLGQPKAA |
| | DVSHEDPEVKFNWYVDGV | HTFPAVLQSSGLYSLSSVVT | PSVTLFPPSSEELQ |
| | EVHNAKTKPREEQYNSTYR | VPSSSLGTQTYICNVNHKPS | ANKATLVCLISDF |
| | VVSVLTVLHQDWLNGKEY | NTKVDKKVEPKSCDKTHTC | YPGAVTVAWKA |
| | KCKVSNKALPAPIEKTISKA | PPCPAPEAAGAPSVFLFPPK | DSSPVKAGVETT |
| | KGQPREPQVYTLPPCRDEL | PKDTLMISRTPEVTCVVVD | TPSKQSNNKYAA |
| | TKNQVSLWCLVKGFYPSDI | VSHEDPEVKFNWYVDGVE | SSYLSLTPEQWKS |
| | AVEWESNGQPENNYKTTPP | VHNAKTKPREEQYNSTYRV | HRSYSCQVTHEG |
| | VLDSDGSFFLYSKLTVDKS | VSVLTVLHQDWLNGKEYK | STVEKTVAPTECS |
| | RWQQGNVFSCSVMHEALH | CKVSNKALPAPIEKTISKAK | (SEQ ID |
| | NHYTQKSLSLSPG | GQPREPQVCTLPPSRDELTK | NO: 753) |
| | (SEQ ID NO: 751) | NQVSLSCAVKGFYPSDIAV | |
| | | EWESNGQPENNYKTTPPVL | |
| | | DSDGSFFLVSKLTVDKSRW | |
| | | QQGNVFSCSVMHEALHNRF | |
| | | TQKSLSLSPG | |
| | | (SEQ ID NO: 752) | |
| DGL439 | QVQLVESGGGLVQAGGSLR | QVQLQESGPGLVKPSETLSL | SYELTQPPSVSVS |
| | LSCAASGRTFGTYAMGWF | TCTVSGGSISSYYWSWIRQP | PGQTASITCSGDK |
| | RQAPGKERDFVARISNSGA | AGKGLEWIGRIYTSGSTNY | LGDKYACWYQQ |
| | FTHYADSVKGRFTISRDNA | NPSLKSRVTMSVDTSKNQF | KPGQSPVLVIYQD |
| | KNTVYLHMNSLENGDTAA | SLKLSSVTAADTAEYYCAR | SKRPSGIPERFSGS |
| | YYCVATRGTNSAYYTRST | AGSSGSSYYYYGMDVWGQ | NSGNTATLTISGT |
| | MYEYWGQGTQVTVEPKSS | GTTVTVSSASTKGPSVFPLA | QAMDEADYYCQ |
| | DKTHTCPPCPAPEAAGAPS | PSSKSTSGGTAALGCLVKD | AWDSSTVVFGGG |
| | VFLFPPKPKDTLMISRTPEV | YFPEPVTVSWNSGALTSGV | TKLTVLGQPKAA |
| | TCVVVDVSHEDPEVKFNW | HTFPAVLQSSGLYSLSSVVT | PSVTLFPPSSEELQ |
| | YVDGVEVHNAKTKPREEQ | VPSSSLGTQTYICNVNHKPS | ANKATLVCLISDF |
| | YNSTYRVVSVLTVLHQDW | NTKVDKKVEPKSCDKTHTC | YPGAVTVAWKA |
| | LNGKEYKCKVSNKALPAPI | PPCPAPEAAGAPSVFLFPPK | DSSPVKAGVETT |
| | EKTISKAKGQPREPQVYTLP | PKDTLMISRTPEVTCVVVD | TPSKQSNNKYAA |
| | PCRDELTKNQVSLWCLVKG | VSHEDPEVKFNWYVDGVE | SSYLSLTPEQWKS |
| | FYPSDIAVEWESNGQPENN | VHNAKTKPREEQYNSTYRV | HRSYSCQVTHEG |
| | YKTTPPVLDSDGSFFLYSKL | VSVLTVLHQDWLNGKEYK | STVEKTVAPTECS |
| | TVDKSRWQQGNVFSCSVM | CKVSNKALPAPIEKTISKAK | (SEQ ID |
| | HEALHNHYTQKSLSLSPG | GQPREPQVCTLPPSRDELTK | NO: 756) |
| | (SEQ ID NO: 754) | NQVSLSCAVKGFYPSDIAV | |
| | | EWESNGQPENNYKTTPPVL | |
| | | DSDGSFFLVSKLTVDKSRW | |
| | | QQGNVFSCSVMHEALHNRF | |
| | | TQKSLSLSPG | |
| | | (SEQ ID NO: 755) | |
| DGL440 | QVQLVESGGGLVQAGGSLR | QVQLVQPGAEVKKPGASV | SYVLTQPPSVSVA |
| | LSCAASGRTFGTYAMGWF | KVSCKASGYTFTGYYMHW | PGQTARITCGGD |
| | RQAPGKERDFVARISNSGA | VRQAPGQGLEWMGWINPK | NIGSKSVHWYQQ |
| | FTHYADSVKGRFTISRDNA | SGGTNYAQKFQGRVTMTR | KPGQAPVPVVYD |
| | KNTVYLHMNSLENGDTAA | DTSISTAYMELTRLRSDDTA | DSDRPSGIPERFS |
| | YYCVATRGTNSAYYTRST | VYYCARDQGYGDYVLKH | GSNSGNTATLTIS |
| | MYEYWGQGTQVTVEPKSS | WGQGTLVTVSSASTKGPSV | RVEAGDEADYYC |
| | DKTHTCPPCPAPEAAGAPS | FPLAPSSKSTSGGTAALGCL | QVWDSSSDHVVF |
| | VFLFPPKPKDTLMISRTPEV | VKDYFPEPVTVSWNSGALT | GGGTKLTVLGQP |
| | TCVVVDVSHEDPEVKFNW | SGVHTFPAVLQSSGLYSLSS | KAAPSVTLFPPSS |
| | YVDGVEVHNAKTKPREEQ | VVTVPSSSLGTQTYICNVNH | EELQANKATLVC |
| | YNSTYRVVSVLTVLHQDW | KPSNTKVDKKVEPKSCDKT | LISDFYPGAVTVA |
| | LNGKEYKCKVSNKALPAPI | HTCPPCPAPEAAGAPSVFLF | WKADSSPVKAGV |
| | EKTISKAKGQPREPQVYTLP | PPKPKDTLMISRTPEVTCVV | ETTTPSKQSNNK |
| | PCRDELTKNQVSLWCLVKG | VDVSHEDPEVKFNWYVDG | YAASSYLSLTPEQ |
| | FYPSDIAVEWESNGQPENN | VEVHNAKTKPREEQYNSTY | WKSHRSYSCQVT |
| | YKTTPPVLDSDGSFFLYSKL | RVVSVLTVLHQDWLNGKE | HEGSTVEKTVAP |
| | TVDKSRWQQGNVFSCSVM | YKCKVSNKALPAPIEKTISK | TECS |
| | HEALHNHYTQKSLSLSPG | AKGQPREPQVCTLPPSRDEL | (SEQ ID |
| | (SEQ ID NO: 757) | TKNQVSLSCAVKGFYPSDI | NO: 759) |
| | | AVEWESNGQPENNYKTTPP | |
| | | VLDSDGSFFLVSKLTVDKS | |
| | | RWQQGNVFSCSVMHEALH | |
| | | NRFTQKSLSLSPG | |
| | | (SEQ ID NO: 758) | |
| DGL441 | QVQLVESGGGLVQAGGSLR | EVQLVESGGGLVQPGGSLR | QSALTQPPSVSGS |
| | LSCAASGRTFGTYAMGWF | LSCAASGFTFSTYDMHWVR | PGQSVTISCTGTS |
| | RQAPGKERDFVARISNSGA | QATGKGLEWVSLIDTAGDT | SDVGSYNRVSWY |
| | FTHYADSVKGRFTISRDNA | YYPDSVKGRFTISRENAKNS | QQPPGTAPKLMI |
| | KNTVYLHMNSLENGDTAA | LYLQMNSLRAGDTAVYYC | YEVSNRPSGVPD |
| | YYCVATRGTNSAYYTRST | AREGWGYFDYWGQGALVT | RFSGSKSGNTASL |
| | MYEYWGQGTQVTVEPKSS | VSSASTKGPSVFPLAPSSKS | TISGLQAEDEADY |
| | DKTHTCPPCPAPEAAGAPS | TSGGTAALGCLVKDYFPEP | YCSLYTGSSTVVF |
| | VFLFPPKPKDTLMISRTPEV | VTVSWNSGALTSGVHTFPA | GGGTKVTVLGQP |
| | TCVVVDVSHEDPEVKFNW | VLQSSGLYSLSSVVTVPSSS | KAAPSVTLFPPSS |
| | YVDGVEVHNAKTKPREEQ | LGTQTYICNVNHKPSNTKV | EELQANKATLVC |
| | YNSTYRVVSVLTVLHQDW | DKKVEPKSCDKTHTCPPCP | LISDFYPGAVTVA |
| | LNGKEYKCKVSNKALPAPI | APEAAGAPSVFLFPPKPKDT | WKADSSPVKAGV |
| | EKTISKAKGQPREPQVYTLP | LMISRTPEVTCVVVDVSHE | ETTTPSKQSNNK |
| | PCRDELTKNQVSLWCLVKG | DPEVKFNWYVDGVEVHNA | YAASSYLSLTPEQ |
| | FYPSDIAVEWESNGQPENN | KTKPREEQYNSTYRVVSVL | WKSHRSYSCQVT |
| | YKTTPPVLDSDGSFFLYSKL | TVLHQDWLNGKEYKCKVS | HEGSTVEKTVAP |
| | TVDKSRWQQGNVFSCSVM | NKALPAPIEKTISKAKGQPR | TECS |
| | HEALHNHYTQKSLSLSPG | EPQVCTLPPSRDELTKNQVS | (SEQ ID |
| | (SEQ ID NO: 760) | LSCAVKGFYPSDIAVEWES | NO: 762) |
| | | NGQPENNYKTTPPVLDSDG | |
| | | SFFLVSKLTVDKSRWQQGN | |
| | | VFSCSVMHEALHNRFTQKS | |
| | | LSLSPG | |
| | | (SEQ ID NO: 761) | |
| DGL442 | QVQLVESGGGLVQAGGSLR | QVHLQESGPGLVKPSETLSL | SYELTQPPSVSVS |
| | LSCAASGRTFGTYAMGWF | TCTVSGGSISYYYWNWIRQ | PGQTASITCSGRE |
| | RQAPGKERDFVARISNSGA | PPGKGLEWIASIYNSGNINY | LGNKYAYWYQQ |
| | FTHYADSVKGRFTISRDNA | NPSLKSRVTISVDTSKNQFS | KPGQSPVLVIYED |
| | KNTVYLHMNSLENGDTAA | LKLTSVTAADTAVYYCARG | YKRPSGIPERFSG |
| | YYCVATRGTNSAYYTRST | GYSYGYFDNWGQGILVTVS | SNSGNTATLTISG |
| | MYEYWGQGTQVTVEPKSS | SASTKGPSVFPLAPSSKSTS | TQAMDEADYYC |
| | DKTHTCPPCPAPEAAGAPS | GGTAALGCLVKDYFPEPVT | QAWDSSTVIFGG |
| | VFLFPPKPKDTLMISRTPEV | VSWNSGALTSGVHTFPAVL | GTKLTVLGQPKA |
| | TCVVVDVSHEDPEVKFNW | QSSGLYSLSSVVTVPSSSLG | APSVTLFPPSSEEL |
| | YVDGVEVHNAKTKPREEQ | TQTYICNVNHKPSNTKVDK | QANKATLVCLIS |
| | YNSTYRVVSVLTVLHQDW | KVEPKSCDKTHTCPPCPAPE | DFYPGAVTVAW |
| | LNGKEYKCKVSNKALPAPI | AAGAPSVFLFPPKPKDTLMI | KADSSPVKAGVE |
| | EKTISKAKGQPREPQVYTLP | SRTPEVTCVVVDVSHEDPE | TTTPSKQSNNKY |
| | PCRDELTKNQVSLWCLVKG | VKFNWYVDGVEVHNAKTK | AASSYLSLTPEQ |
| | FYPSDIAVEWESNGQPENN | PREEQYNSTYRVVSVLTVL | WKSHRSYSCQVT |
| | YKTTPPVLDSDGSFFLYSKL | HQDWLNGKEYKCKVSNKA | HEGSTVEKTVAP |
| | TVDKSRWQQGNVFSCSVM | LPAPIEKTISKAKGQPREPQ | TECS |
| | HEALHNHYTQKSLSLSPG | VCTLPPSRDELTKNQVSLSC | (SEQ ID |
| | (SEQ ID NO: 763) | AVKGFYPSDIAVEWESNGQ | NO: 765) |
| | | PENNYKTTPPVLDSDGSFFL | |
| | | VSKLTVDKSRWQQGNVFS | |
| | | CSVMHEALHNRFTQKSLSL | |
| | | SPG | |
| | | (SEQ ID NO: 764) | |
| DGL443 | QVQLVESGGGLVQAGGSLR | QVTLRESGPALVKPTQTLTL | QSVLTQPPSVSGA |
| | LSCAASGRTFGTYAMGWF | TCTFSGFSLSTSGMCVSWIR | PGQRVTISCTGSS |
| | RQAPGKERDFVARISNSGA | QPPGKALEWLALIYWDDD | SNIGAGYDVHWY |
| | FTHYADSVKGRFTISRDNA | KYYSTSLKTRLTISKDTSKN | QQLPGTAPKLLIF |
| | KNTVYLHMNSLENGDTAA | QVVLTMTNMDPVDTATYY | GYSNRPSGVPVR |
| | YYCVATRGTNSAYYTRST | CARMRRGIEAFDIWGQGTM | FSGSKSGTSASLA |
| | MYEYWGQGTQVTVEPKSS | VTVFSASTKGPSVFPLAPSS | ITGLLAEDEADFY |
| | DKTHTCPPCPAPEAAGAPS | KSTSGGTAALGCLVKDYFP | CQSFDISLTGWVF |
| | VFLFPPKPKDTLMISRTPEV | EPVTVSWNSGALTSGVHTF | GGGTKLTVLGQP |
| | TCVVVDVSHEDPEVKFNW | PAVLQSSGLYSLSSVVTVPS | KAAPSVTLFPPSS |
| | YVDGVEVHNAKTKPREEQ | SSLGTQTYICNVNHKPSNTK | EELQANKATLVC |
| | YNSTYRVVSVLTVLHQDW | VDKKVEPKSCDKTHTCPPC | LISDFYPGAVTVA |
| | LNGKEYKCKVSNKALPAPI | PAPEAAGAPSVFLFPPKPKD | WKADSSPVKAGV |
| | EKTISKAKGQPREPQVYTLP | TLMISRTPEVTCVVVDVSH | ETTTPSKQSNNK |
| | PCRDELTKNQVSLWCLVKG | EDPEVKFNWYVDGVEVHN | YAASSYLSLTPEQ |
| | FYPSDIAVEWESNGQPENN | AKTKPREEQYNSTYRVVSV | WKSHRSYSCQVT |
| | YKTTPPVLDSDGSFFLYSKL | LTVLHQDWLNGKEYKCKV | HEGSTVEKTVAP |
| | TVDKSRWQQGNVFSCSVM | SNKALPAPIEKTISKAKGQP | TECS |
| | HEALHNHYTQKSLSLSPG | REPQVCTLPPSRDELTKNQ | (SEQ ID |
| | (SEQ ID NO: 766) | VSLSCAVKGFYPSDIAVEW | NO: 768) |
| | | ESNGQPENNYKTTPPVLDS | |
| | | DGSFFLVSKLTVDKSRWQQ | |
| | | GNVFSCSVMHEALHNRFTQ | |
| | | KSLSLSPG | |
| | | (SEQ ID NO: 767) | |
| DGL444 | QVQLVESGGGLVQAGGSLR | QVQLVQSGAEVKKPGASV | QSVLTQPPSASGT |
| | LSCAASGRTFGTYAMGWF | KVSCKASGYTFIGYYIHWV | PGQRVTISCSGGS |
| | RQAPGKERDFVARISNSGA | RQAPGQGLEWMGWINPHS | SNIGTNSVNWYQ |
| | FTHYADSVKGRFTISRDNA | GGTNYAQKFQGRVTMTRD | QLPGTAPKLLIYY |
| | KNTVYLHMNSLENGDTAA | TSISTAYMELSRLRSDDTAI | NNQRPSGVPDRF |
| | YYCVATRGTNSAYYTRST | YYCTKEANNWGYAFDIWG | SGSKSGTSASLAI |
| | MYEYWGQGTQVTVEPKSS | QGTMVTVSSASTKGPSVFP | SGLQSEDEADYY |
| | DKTHTCPPCPAPEAAGAPS | LAPSSKSTSGGTAALGCLV | CAAWDDSLNGW |
| | VFLFPPKPKDTLMISRTPEV | KDYFPEPVTVSWNSGALTS | VFGGGTKLTVLG |
| | TCVVVDVSHEDPEVKFNW | GVHTFPAVLQSSGLYSLSSV | QPKAAPSVTLFPP |
| | YVDGVEVHNAKTKPREEQ | VTVPSSSLGTQTYICNVNHK | SSEELQANKATL |
| | YNSTYRVVSVLTVLHQDW | PSNTKVDKKVEPKSCDKTH | VCLISDFYPGAVT |
| | LNGKEYKCKVSNKALPAPI | TCPPCPAPEAAGAPSVFLFP | VAWKADSSPVKA |
| | EKTISKAKGQPREPQVYTLP | PKPKDTLMISRTPEVTCVVV | GVETTTPSKQSN |
| | PCRDELTKNQVSLWCLVKG | DVSHEDPEVKFNWYVDGV | NKYAASSYLSLTP |
| | FYPSDIAVEWESNGQPENN | EVHNAKTKPREEQYNSTYR | EQWKSHRSYSCQ |
| | YKTTPPVLDSDGSFFLYSKL | VVSVLTVLHQDWLNGKEY | VTHEGSTVEKTV |
| | TVDKSRWQQGNVFSCSVM | KCKVSNKALPAPIEKTISKA | APTECS |
| | HEALHNHYTQKSLSLSPG | KGQPREPQVCTLPPSRDELT | (SEQ ID |
| | (SEQ ID NO: 769) | KNQVSLSCAVKGFYPSDIA | NO: 771) |
| | | VEWESNGQPENNYKTTPPV | |
| | | LDSDGSFFLVSKLTVDKSR | |
| | | WQQGNVFSCSVMHEALHN | |
| | | RFTQKSLSLSPG | |
| | | (SEQ ID NO: 770) | |
| DGL445 | QVQLQESGGGLVQAGGSLR | QVQLVQPGAEVKKPGASV | SYVLTQPPSVSVA |
| | LSCAASGTIFSIDVMAYYRQ | KVSCKASGYTFTGYYMHW | PGQTARITCGGD |
| | APGKQRELVAAISSGGSLN | VRQAPGQGLEWMGWINPK | NIGSKSVHWYQQ |
| | YRDSVKGRFTISRDNAKNA | SGGTNYAQKFQGRVTMTR | KPGQAPVPVVYD |
| | VYLQMNSLKPDDTAVYYC | DTSISTAYMELTRLRSDDTA | DSDRPSGIPERFS |
| | YANIRTSPVTTRPMGNYWG | VYYCARDQGYGDYVLKH | GSNSGNTATLTIS |
| | QGTQVTVEPKSSDKTHTCP | WGQGTLVTVSSASTKGPSV | RVEAGDEADYYC |
| | PCPAPEAAGAPSVFLFPPKP | FPLAPSSKSTSGGTAALGCL | QVWDSSSDHVVF |
| | KDTLMISRTPEVTCVVVDV | VKDYFPEPVTVSWNSGALT | GGGTKLTVLGQP |
| | SHEDPEVKFNWYVDGVEV | SGVHTFPAVLQSSGLYSLSS | KAAPSVTLFPPSS |
| | HNAKTKPREEQYNSTYRVV | VVTVPSSSLGTQTYICNVNH | EELQANKATLVC |
| | SVLTVLHQDWLNGKEYKC | KPSNTKVDKKVEPKSCDKT | LISDFYPGAVTVA |
| | KVSNKALPAPIEKTISKAKG | HTCPPCPAPEAAGAPSVFLF | WKADSSPVKAGV |
| | QPREPQVYTLPPCRDELTK | PPKPKDTLMISRTPEVTCVV | ETTTPSKQSNNK |
| | NQVSLWCLVKGFYPSDIAV | VDVSHEDPEVKFNWYVDG | YAASSYLSLTPEQ |
| | EWESNGQPENNYKTTPPVL | VEVHNAKTKPREEQYNSTY | WKSHRSYSCQVT |
| | DSDGSFFLYSKLTVDKSRW | RVVSVLTVLHQDWLNGKE | HEGSTVEKTVAP |
| | QQGNVFSCSVMHEALHNH | YKCKVSNKALPAPIEKTISK | TECS |
| | YTQKSLSLSPG | AKGQPREPQVCTLPPSRDEL | (SEQ ID |
| | (SEQ ID NO: 772) | TKNQVSLSCAVKGFYPSDI | NO: 774) |
| | | AVEWESNGQPENNYKTTPP | |
| | | VLDSDGSFFLVSKLTVDKS | |
| | | RWQQGNVFSCSVMHEALH | |
| | | NRFTQKSLSLSPG | |
| | | (SEQ ID NO: 773) | |
| DGL495 | QVQLVESGGGLVQAGGSLR | QVQLVQSGAEVKKPGASV | QSVLTQPPSASGT |
| | LSCAASGGPFSGYVMGWFR | KVSCKASGYTFIGYYIHWV | PGQRVTISCSGGS |
| | QVSGKEREFVAGITGISSTY | RQAPGQGLEWMGWINPHS | SNIGTNSVNWYQ |
| | FADSVKGRFTISRDNAKNM | GGTNYAQKFQGRVTMTRD | QLPGTAPKLLIYY |
| | VYLQMNSLKPEDTAVYYC | TSISTAYMELSRLRSDDTAI | NNQRPSGVPDRF |
| | AAVNSFGVIPTSPNGMDYW | YYCTKEANNWGYAFDIWG | SGSKSGTSASLAI |
| | GKGTLVTVEPKSSDKTHTC | QGTMVTVSSASTKGPSVFP | SGLQSEDEADYY |
| | PPCPAPEAAGAPSVFLFPPK | LAPSSKSTSGGTAALGCLV | CAAWDDSLNGW |
| | PKDTLMISRTPEVTCVVVD | KDYFPEPVTVSWNSGALTS | VFGGGTKLTVLG |
| | VSHEDPEVKFNWYVDGVE | GVHTFPAVLQSSGLYSLSSV | QPKAAPSVTLFPP |
| | VHNAKTKPREEQYNSTYRV | VTVPSSSLGTQTYICNVNHK | SSEELQANKATL |
| | VSVLTVLHQDWLNGKEYK | PSNTKVDKKVEPKSCDKTH | VCLISDFYPGAVT |
| | CKVSNKALPAPIEKTISKAK | TCPPCPAPEAAGAPSVFLFP | VAWKADSSPVKA |
| | GQPREPQVYTLPPCRDELT | PKPKDTLMISRTPEVTCVVV | GVETTTPSKQSN |
| | KNQVSLWCLVKGFYPSDIA | DVSHEDPEVKFNWYVDGV | NKYAASSYLSLTP |
| | VEWESNGQPENNYKTTPPV | EVHNAKTKPREEQYNSTYR | EQWKSHRSYSCQ |
| | LDSDGSFFLYSKLTVDKSR | VVSVLTVLHQDWLNGKEY | VTHEGSTVEKTV |
| | WQQGNVFSCSVMHEALHN | KCKVSNKALPAPIEKTISKA | APTECS |
| | HYTQKSLSLSPG | KGQPREPQVCTLPPSRDELT | (SEQ ID |
| | (SEQ ID NO: 775) | KNQVSLSCAVKGFYPSDIA | NO: 777) |
| | | VEWESNGQPENNYKTTPPV | |
| | | LDSDGSFFLVSKLTVDKSR | |
| | | WQQGNVFSCSVMHEALHN | |
| | | RFTQKSLSLSPG | |
| | | (SEQ ID NO: 776) | |
| DGL426 | EVQLVESGGGLVQAGGSLR | QVQLVESGGGLVQPGGSLR | |
| | LSCAASGLTFRNYAMAWF | LSCAASGFTFSNYAMSWVR | |
| | RQAPGKEREFVAGITWGGG | QAPGKGLEWVSAINSDGTS | |
| | LTHYVDSVKGRFTISRDNG | TSYADSVKGQFTISRDNAK | |
| | KNTVYLQMNSLKSEDTAV | NTLYLQMNSLKPEDTAVYY | |
| | YYCAGKTQGSTWYHLTPD | CTKWEGDTDSDYKFKDYW | |
| | GYDYWGQGTQVTVEPKSS | GQGTQVTVEPKSSDKTHTC | |
| | DKTHTCPPCPAPEAAGAPS | PPCPAPEAAGAPSVFLFPPK | |
| | VFLFPPKPKDTLMISRTPEV | PKDTLMISRTPEVTCVVVD | |
| | TCVVVDVSHEDPEVKFNW | VSHEDPEVKFNWYVDGVE | |
| | YVDGVEVHNAKTKPREEQ | VHNAKTKPREEQYNSTYRV | |
| | YNSTYRVVSVLTVLHQDW | VSVLTVLHQDWLNGKEYK | |
| | LNGKEYKCKVSNKALPAPI | CKVSNKALPAPIEKTISKAK | |
| | EKTISKAKGQPREPQVYTLP | GQPREPQVCTLPPSRDELTK | |
| | PCRDELTKNQVSLWCLVKG | NQVSLSCAVKGFYPSDIAV | |
| | FYPSDIAVEWESNGQPENN | EWESNGQPENNYKTTPPVL | |
| | YKTTPPVLDSDGSFFLYSKL | DSDGSFFLVSKLTVDKSRW | |
| | TVDKSRWQQGNVFSCSVM | QQGNVFSCSVMHEALHNRF | |
| | HEALHNHYTQKSLSLSPG | TQKSLSLSPG | |
| | (SEQ ID NO: 778) | (SEQ ID NO: 779) | |
| DGL448 | QVQLVESGGGLVQAGGSLR | EVQLVESGGGLVQPGGSLR | QSALTQPPSVSGS |
| | LSCAASRSISSFNEMAWYR | LSCAASGFTFSTYDMHWVR | PGQSVTISCTGTS |
| | QAPGEQRELVATIVSTVGFT | QATGKGLEWVSLIDTAGDT | SDVGSYNRVSWY |
| | NYADSVKGRFTISRDNAKN | YYPDSVKGRFTISRENAKNS | QQPPGTAPKLMI |
| | TVYLQMNSLKAEDTAVYY | LYLQMNSLRAGDTAVYYC | YEVSNRPSGVPD |
| | CNARRISTDYWGQGTQVTV | AREGWGYFDYWGQGALVT | RFSGSKSGNTASL |
| | EPKSSDKTHTCPPCPAPEAA | VSSASTKGPSVFPLAPSSKS | TISGLQAEDEADY |
| | GAPSVFLFPPKPKDTLMISR | TSGGTAALGCLVKDYFPEP | YCSLYTGSSTVVF |
| | TPEVTCVVVDVSHEDPEVK | VTVSWNSGALTSGVHTFPA | GGGTKVTVLGQP |
| | FNWYVDGVEVHNAKTKPR | VLQSSGLYSLSSVVTVPSSS | KAAPSVTLFPPSS |
| | EEQYNSTYRVVSVLTVLHQ | LGTQTYICNVNHKPSNTKV | EELQANKATLVC |
| | DWLNGKEYKCKVSNKALP | DKKVEPKSCDKTHTCPPCP | LISDFYPGAVTVA |
| | APIEKTISKAKGQPREPQVY | APEAAGAPSVFLFPPKPKDT | WKADSSPVKAGV |
| | TLPPCRDELTKNQVSLWCL | LMISRTPEVTCVVVDVSHE | ETTTPSKQSNNK |
| | VKGFYPSDIAVEWESNGQP | DPEVKFNWYVDGVEVHNA | YAASSYLSLTPEQ |
| | ENNYKTTPPVLDSDGSFFLY | KTKPREEQYNSTYRVVSVL | WKSHRSYSCQVT |
| | SKLTVDKSRWQQGNVFSCS | TVLHQDWLNGKEYKCKVS | HEGSTVEKTVAP |
| | VMHEALHNHYTQKSLSLSP | NKALPAPIEKTISKAKGQPR | TECS |
| | G | EPQVCTLPPSRDELTKNQVS | (SEQ ID |
| | (SEQ ID NO: 780) | LSCAVKGFYPSDIAVEWES | NO: 908) |
| | | NGQPENNYKTTPPVLDSDG | |
| | | SFFLVSKLTVDKSRWQQGN | |
| | | VFSCSVMHEALHNRFTQKS | |
| | | LSLSPG | |
| | | (SEQ ID NO: 781) | |
| DGL453 | QVQLQESGGALVQPGGSLR | QVQLVESGGGLVQPGGSLR | |
| | LSCVVSGFTSDYFAIGWFR | LSCAASGFTFSNYAMSWIR | |
| | QAPGKEREGVSCIGSSDGST | QGPERGFEWVSRINSGGGT | |
| | IYSDSVKGRFTISRDNAKNT | TSYADSVKGRFTISRDNAK | |
| | VYLQMNSLKPEDTGVYYC | NTLYLQMNSLKPEDTAVYY | |
| | ALDRGRRTIIGYACYATMK | CAKHSHPDYSSNYYRPGQG | |
| | YWGKGTPVTVEPKSSDKTH | TQVTVEPKSSDKTHTCPPCP | |
| | TCPPCPAPEAAGAPSVFLFP | APEAAGAPSVFLFPPKPKDT | |
| | PKPKDTLMISRTPEVTCVVV | LMISRTPEVTCVVVDVSHE | |
| | DVSHEDPEVKFNWYVDGV | DPEVKFNWYVDGVEVHNA | |
| | EVHNAKTKPREEQYNSTYR | KTKPREEQYNSTYRVVSVL | |
| | VVSVLTVLHQDWLNGKEY | TVLHQDWLNGKEYKCKVS | |
| | KCKVSNKALPAPIEKTISKA | NKALPAPIEKTISKAKGQPR | |
| | KGQPREPQVYTLPPCRDEL | EPQVCTLPPSRDELTKNQVS | |
| | TKNQVSLWCLVKGFYPSDI | LSCAVKGFYPSDIAVEWES | |
| | AVEWESNGQPENNYKTTPP | NGQPENNYKTTPPVLDSDG | |
| | VLDSDGSFFLYSKLTVDKS | SFFLVSKLTVDKSRWQQGN | |
| | RWQQGNVFSCSVMHEALH | VFSCSVMHEALHNRFTQKS | |
| | NHYTQKSLSLSPG | LSLSPG | |
| | (SEQ ID NO: 782) | (SEQ ID NO: 783) | |
| DGL454 | QVQLQESGGALVQPGGSLR | QVQLVESGGGLVQPGGSLR | |
| | LSCVVSGFTSDYFAIGWFR | LSCAASGFTFEPYDMSWFR | |
| | QAPGKEREGVSCIGSSDGST | QAPGKGPEWVSAIDKGGG | |
| | IYSDSVKGRFTISRDNAKNT | AGATYYADSVKGRFTISRD | |
| | VYLQMNSLKPEDTGVYYC | NAKKTLYLQMKSLKPEDTA | |
| | ALDRGRRTIIGYACYATMK | VYYCAITAGPYSDSTPRVA | |
| | YWGKGTPVTVEPKSSDKTH | YRGEGTQVTVEPKSSDKTH | |
| | TCPPCPAPEAAGAPSVFLFP | TCPPCPAPEAAGAPSVFLFP | |
| | PKPKDTLMISRTPEVTCVVV | PKPKDTLMISRTPEVTCVVV | |
| | DVSHEDPEVKFNWYVDGV | DVSHEDPEVKFNWYVDGV | |
| | EVHNAKTKPREEQYNSTYR | EVHNAKTKPREEQYNSTYR | |
| | VVSVLTVLHQDWLNGKEY | VVSVLTVLHQDWLNGKEY | |
| | KCKVSNKALPAPIEKTISKA | KCKVSNKALPAPIEKTISKA | |
| | KGQPREPQVYTLPPCRDEL | KGQPREPQVCTLPPSRDELT | |
| | TKNQVSLWCLVKGFYPSDI | KNQVSLSCAVKGFYPSDIA | |
| | AVEWESNGQPENNYKTTPP | VEWESNGQPENNYKTTPPV | |
| | VLDSDGSFFLYSKLTVDKS | LDSDGSFFLVSKLTVDKSR | |
| | RWQQGNVFSCSVMHEALH | WQQGNVFSCSVMHEALHN | |
| | NHYTQKSLSLSPG | RFTQKSLSLSPG | |
| | (SEQ ID NO: 784) | (SEQ ID NO: 785) | |
| DGL461 | EVQLVESGGGLVQPGGSLR | QVQLVESGGGLVQPGGSLR | |
| | LSCAASGFTFQNYAMSWLR | LSCAASGFTFEPYDMSWFR | |
| | KAPGEGLEWVSVINSGGGS | QAPGKGPEWVSAIDKGGG | |
| | TLYADSVKGRFTISRDNAK | AGATYYADSVKGRFTISRD | |
| | NTLYLQMHSLKSEDTAVYF | NAKKTLYLQMKSLKPEDTA | |
| | CAKRRDDSTFGSLLYTHRG | VYYCAITAGPYSDSTPRVA | |
| | QGTQVTVEPKSSDKTHTCP | YRGEGTQVTVEPKSSDKTH | |
| | PCPAPEAAGAPSVFLFPPKP | TCPPCPAPEAAGAPSVFLFP | |
| | KDTLMISRTPEVTCVVVDV | PKPKDTLMISRTPEVTCVVV | |
| | SHEDPEVKFNWYVDGVEV | DVSHEDPEVKFNWYVDGV | |
| | HNAKTKPREEQYNSTYRVV | EVHNAKTKPREEQYNSTYR | |
| | SVLTVLHQDWLNGKEYKC | VVSVLTVLHQDWLNGKEY | |
| | KVSNKALPAPIEKTISKAKG | KCKVSNKALPAPIEKTISKA | |
| | QPREPQVYTLPPCRDELTK | KGQPREPQVCTLPPSRDELT | |
| | NQVSLWCLVKGFYPSDIAV | KNQVSLSCAVKGFYPSDIA | |
| | EWESNGQPENNYKTTPPVL | VEWESNGQPENNYKTTPPV | |
| | DSDGSFFLYSKLTVDKSRW | LDSDGSFFLVSKLTVDKSR | |
| | QQGNVFSCSVMHEALHNH | WQQGNVFSCSVMHEALHN | |
| | YTQKSLSLSPG | RFTQKSLSLSPG | |
| | (SEQ ID NO: 786) | (SEQ ID NO: 787) | |
| DGL462 | EVQLVESGGGLVQPGGSLR | QVQLVESGGGLVQAGGSLR | |
| | LSCAASGFTFQNYAMSWLR | LSCAASGRIFSAYRMGWFR | |
| | KAPGEGLEWVSVINSGGGS | QAPGKEREFVAGITWRGQT | |
| | TLYADSVKGRFTISRDNAK | TYYAASVKGRFTISKDNAK | |
| | NTLYLQMHSLKSEDTAVYF | KTVYLQMNSLKPEETAVYY | |
| | CAKRRDDSTFGSLLYTHRG | CAVETNGLGRWDRPSEYD | |
| | QGTQVTVEPKSSDKTHTCP | YWGQGTQVTVEPKSSDKT | |
| | PCPAPEAAGAPSVFLFPPKP | HTCPPCPAPEAAGAPSVFLF | |
| | KDTLMISRTPEVTCVVVDV | PPKPKDTLMISRTPEVTCVV | |
| | SHEDPEVKFNWYVDGVEV | VDVSHEDPEVKFNWYVDG | |
| | HNAKTKPREEQYNSTYRVV | VEVHNAKTKPREEQYNSTY | |
| | SVLTVLHQDWLNGKEYKC | RVVSVLTVLHQDWLNGKE | |
| | KVSNKALPAPIEKTISKAKG | YKCKVSNKALPAPIEKTISK | |
| | QPREPQVYTLPPCRDELTK | AKGQPREPQVCTLPPSRDEL | |
| | NQVSLWCLVKGFYPSDIAV | TKNQVSLSCAVKGFYPSDI | |
| | EWESNGQPENNYKTTPPVL | AVEWESNGQPENNYKTTPP | |
| | DSDGSFFLYSKLTVDKSRW | VLDSDGSFFLVSKLTVDKS | |
| | QQGNVFSCSVMHEALHNH | RWQQGNVFSCSVMHEALH | |
| | YTQKSLSLSPG | NRFTQKSLSLSPG | |
| | (SEQ ID NO: 788) | (SEQ ID NO: 789) | |
| DGL464 | QVQLVESGGGLVQAGGSLR | QVQLVESGGGLVQPGGSLR | |
| | LSCAASGRAFSRYSMGWFR | LSCAASGFTFSNYAMSWVR | |
| | QAPGKEREFVAANYWSNG | QAPGKGLEWVSAINSDGTS | |
| | WTDYADTVKGRFSISRDNA | TSYADSVKGQFTISRDNAK | |
| | KNTVYLQMNSLKPEDTAV | NTLYLQMNSLKPEDTAVYY | |
| | YYCAGRSSSIPSADVVGYD | CTKWEGDTDSDYKFKDYW | |
| | YWGQGTQVTVEPKSSDKT | GQGTQVTVEPKSSDKTHTC | |
| | HTCPPCPAPEAAGAPSVFLF | PPCPAPEAAGAPSVFLFPPK | |
| | PPKPKDTLMISRTPEVTCVV | PKDTLMISRTPEVTCVVVD | |
| | VDVSHEDPEVKFNWYVDG | VSHEDPEVKFNWYVDGVE | |
| | VEVHNAKTKPREEQYNSTY | VHNAKTKPREEQYNSTYRV | |
| | RVVSVLTVLHQDWLNGKE | VSVLTVLHQDWLNGKEYK | |
| | YKCKVSNKALPAPIEKTISK | CKVSNKALPAPIEKTISKAK | |
| | AKGQPREPQVYTLPPCRDE | GQPREPQVCTLPPSRDELTK | |
| | LTKNQVSLWCLVKGFYPSD | NQVSLSCAVKGFYPSDIAV | |
| | IAVEWESNGQPENNYKTTP | EWESNGQPENNYKTTPPVL | |
| | PVLDSDGSFFLYSKLTVDKS | DSDGSFFLVSKLTVDKSRW | |
| | RWQQGNVFSCSVMHEALH | QQGNVFSCSVMHEALHNRF | |
| | NHYTQKSLSLSPG | TQKSLSLSPG | |
| | (SEQ ID NO: 790) | (SEQ ID NO: 791) | |
| DGL465 | QVQLVESGGGLVQAGGSLR | QVQLVESGGGLVQPGGSLR | |
| | LSCAASGRAFSRYSMGWFR | LSCAASGFTFEPYDMSWFR | |
| | QAPGKEREFVAANYWSNG | QAPGKGPEWVSAIDKGGG | |
| | WTDYADTVKGRFSISRDNA | AGATYYADSVKGRFTISRD | |
| | KNTVYLQMNSLKPEDTAV | NAKKTLYLQMKSLKPEDTA | |
| | YYCAGRSSSIPSADVVGYD | VYYCAITAGPYSDSTPRVA | |
| | YWGQGTQVTVEPKSSDKT | YRGEGTQVTVEPKSSDKTH | |
| | HTCPPCPAPEAAGAPSVFLF | TCPPCPAPEAAGAPSVFLFP | |
| | PPKPKDTLMISRTPEVTCVV | PKPKDTLMISRTPEVTCVVV | |
| | VDVSHEDPEVKFNWYVDG | DVSHEDPEVKFNWYVDGV | |
| | VEVHNAKTKPREEQYNSTY | EVHNAKTKPREEQYNSTYR | |
| | RVVSVLTVLHQDWLNGKE | VVSVLTVLHQDWLNGKEY | |
| | YKCKVSNKALPAPIEKTISK | KCKVSNKALPAPIEKTISKA | |
| | AKGQPREPQVYTLPPCRDE | KGQPREPQVCTLPPSRDELT | |
| | LTKNQVSLWCLVKGFYPSD | KNQVSLSCAVKGFYPSDIA | |
| | IAVEWESNGQPENNYKTTP | VEWESNGQPENNYKTTPPV | |
| | PVLDSDGSFFLYSKLTVDKS | LDSDGSFFLVSKLTVDKSR | |
| | RWQQGNVFSCSVMHEALH | WQQGNVFSCSVMHEALHN | |
| | NHYTQKSLSLSPG | RFTQKSLSLSPG | |
| | (SEQ ID NO: 792) | (SEQ ID NO: 793) | |
| DGL478 | QVQLVESGGGLVQAGGSLR | QVQLVESGGGLVQAGGSLR | |
| | LSCAAPGRYAVGWFRQAP | LSCAASGRIFSAYRMGWFR | |
| | GKEYEFVAAITVSGGSKYY | QAPGKEREFVAGITWRGQT | |
| | EDSVKGRFAISRDNAKNTV | TYYAASVKGRFTISKDNAK | |
| | YLQMNSLKAEDTAVYYCA | KTVYLQMNSLKPEETAVYY | |
| | ARTPKFGSGWYNRRGEYD | CAVETNGLGRWDRPSEYD | |
| | YWGQGTQVTVEPKSSDKT | YWGQGTQVTVEPKSSDKT | |
| | HTCPPCPAPEAAGAPSVFLF | HTCPPCPAPEAAGAPSVFLF | |
| | PPKPKDTLMISRTPEVTCVV | PPKPKDTLMISRTPEVTCVV | |
| | VDVSHEDPEVKFNWYVDG | VDVSHEDPEVKFNWYVDG | |
| | VEVHNAKTKPREEQYNSTY | VEVHNAKTKPREEQYNSTY | |
| | RVVSVLTVLHQDWLNGKE | RVVSVLTVLHQDWLNGKE | |
| | YKCKVSNKALPAPIEKTISK | YKCKVSNKALPAPIEKTISK | |
| | AKGQPREPQVYTLPPCRDE | AKGQPREPQVCTLPPSRDEL | |
| | LTKNQVSLWCLVKGFYPSD | TKNQVSLSCAVKGFYPSDI | |
| | IAVEWESNGQPENNYKTTP | AVEWESNGQPENNYKTTPP | |
| | PVLDSDGSFFLYSKLTVDKS | VLDSDGSFFLVSKLTVDKS | |
| | RWQQGNVFSCSVMHEALH | RWQQGNVFSCSVMHEALH | |
| | NHYTQKSLSLSPG | NRFTQKSLSLSPG | |
| | (SEQ ID NO: 794) | (SEQ ID NO: 795) | |
| DGL479 | QVQLVESGGGLVQAGGSLR | QVQLVESGGGLVQAGGSLR | |
| | LSCAAPGRYAVGWFRQAP | LSCAASGRTFSSYAMGWFR | |
| | GKEYEFVAAITVSGGSKYY | QAPGKEREFVSAISWSGLST | |
| | EDSVKGRFAISRDNAKNTV | YYVESVQGRFTISRDNAKN | |
| | YLQMNSLKAEDTAVYYCA | TVYLQMNSLKPEDTAVYY | |
| | ARTPKFGSGWYNRRGEYD | CAASRIYTNYVARSYEYDD | |
| | YWGQGTQVTVEPKSSDKT | WGQGTQVTVEPKSSDKTHT | |
| | HTCPPCPAPEAAGAPSVFLF | CPPCPAPEAAGAPSVFLFPP | |
| | PPKPKDTLMISRTPEVTCVV | KPKDTLMISRTPEVTCVVV | |
| | VDVSHEDPEVKFNWYVDG | DVSHEDPEVKFNWYVDGV | |
| | VEVHNAKTKPREEQYNSTY | EVHNAKTKPREEQYNSTYR | |
| | RVVSVLTVLHQDWLNGKE | VVSVLTVLHQDWLNGKEY | |
| | YKCKVSNKALPAPIEKTISK | KCKVSNKALPAPIEKTISKA | |
| | AKGQPREPQVYTLPPCRDE | KGQPREPQVCTLPPSRDELT | |
| | LTKNQVSLWCLVKGFYPSD | KNQVSLSCAVKGFYPSDIA | |
| | IAVEWESNGQPENNYKTTP | VEWESNGQPENNYKTTPPV | |
| | PVLDSDGSFFLYSKLTVDKS | LDSDGSFFLVSKLTVDKSR | |
| | RWQQGNVFSCSVMHEALH | WQQGNVFSCSVMHEALHN | |
| | NHYTQKSLSLSPG | RFTQKSLSLSPG | |
| | (SEQ ID NO: 796) | (SEQ ID NO: 797) | |
| DGL481 | QVQLVESGGGLVQAGGSLR | QVQLVESGGGLVQPGGSLR | |
| | LSCAASARIFNSYAVAWFR | LSCAASGFTFSNYAMSWVR | |
| | QTPGKEREFVASISWSGGST | QAPGKGLEWVSAINSDGTS | |
| | NYADSVKGRFTISRDNAKN | TSYADSVKGQFTISRDNAK | |
| | TVFLQMNSLKPADTAVYYC | NTLYLQMNSLKPEDTAVYY | |
| | AADRNYYPSVLGKYTYWG | CTKWEGDTDSDYKFKDYW | |
| | QGTQVTVEPKSSDKTHTCP | GQGTQVTVEPKSSDKTHTC | |
| | PCPAPEAAGAPSVFLFPPKP | PPCPAPEAAGAPSVFLFPPK | |
| | KDTLMISRTPEVTCVVVDV | PKDTLMISRTPEVTCVVVD | |
| | SHEDPEVKFNWYVDGVEV | VSHEDPEVKFNWYVDGVE | |
| | HNAKTKPREEQYNSTYRVV | VHNAKTKPREEQYNSTYRV | |
| | SVLTVLHQDWLNGKEYKC | VSVLTVLHQDWLNGKEYK | |
| | KVSNKALPAPIEKTISKAKG | CKVSNKALPAPIEKTISKAK | |
| | QPREPQVYTLPPCRDELTK | GQPREPQVCTLPPSRDELTK | |
| | NQVSLWCLVKGFYPSDIAV | NQVSLSCAVKGFYPSDIAV | |
| | EWESNGQPENNYKTTPPVL | EWESNGQPENNYKTTPPVL | |
| | DSDGSFFLYSKLTVDKSRW | DSDGSFFLVSKLTVDKSRW | |
| | QQGNVFSCSVMHEALHNH | QQGNVFSCSVMHEALHNRF | |
| | YTQKSLSLSPG | TQKSLSLSPG | |
| | (SEQ ID NO: 798) | (SEQ ID NO: 799) | |
| DGL482 | QVQLVESGGGLVQAGGSLR | QVQLVESGGGLVQPGGSLR | |
| | LSCAASARIFNSYAVAWFR | LSCAASGFTFSSKAMHWFR | |
| | QTPGKEREFVASISWSGGST | QAPGKGLEWVSSINSGGAIT | |
| | NYADSVKGRFTISRDNAKN | YYKDSVKGRFTVSRDNAK | |
| | TVFLQMNSLKPADTAVYYC | NILYLEMNSLKPEDTALYY | |
| | AADRNYYPSVLGKYTYWG | CATDTDGRTRGQGTQVTVE | |
| | QGTQVTVEPKSSDKTHTCP | PKSSDKTHTCPPCPAPEAAG | |
| | PCPAPEAAGAPSVFLFPPKP | APSVFLFPPKPKDTLMISRT | |
| | KDTLMISRTPEVTCVVVDV | PEVTCVVVDVSHEDPEVKF | |
| | SHEDPEVKFNWYVDGVEV | NWYVDGVEVHNAKTKPRE | |
| | HNAKTKPREEQYNSTYRVV | EQYNSTYRVVSVLTVLHQD | |
| | SVLTVLHQDWLNGKEYKC | WLNGKEYKCKVSNKALPA | |
| | KVSNKALPAPIEKTISKAKG | PIEKTISKAKGQPREPQVCT | |
| | QPREPQVYTLPPCRDELTK | LPPSRDELTKNQVSLSCAV | |
| | NQVSLWCLVKGFYPSDIAV | KGFYPSDIAVEWESNGQPE | |
| | EWESNGQPENNYKTTPPVL | NNYKTTPPVLDSDGSFFLVS | |
| | DSDGSFFLYSKLTVDKSRW | KLTVDKSRWQQGNVFSCS | |
| | QQGNVFSCSVMHEALHNH | VMHEALHNRFTQKSLSLSP | |
| | YTQKSLSLSPG | G | |
| | (SEQ ID NO: 800) | (SEQ ID NO: 801) | |
| DGL484 | QVQLVESGGGLVQAGGSLR | QVQLVESGGGLVQPGGSLR | |
| | LSCAASARIFNSYAVAWFR | LSCAASGFTFEPYDMSWFR | |
| | QTPGKEREFVASISWSGGST | QAPGKGPEWVSAIDKGGG | |
| | NYADSVKGRFTISRDNAKN | AGATYYADSVKGRFTISRD | |
| | TVFLQMNSLKPADTAVYYC | NAKKTLYLQMKSLKPEDTA | |
| | AADRNYYPSVLGKYTYWG | VYYCAITAGPYSDSTPRVA | |
| | QGTQVTVEPKSSDKTHTCP | YRGEGTQVTVEPKSSDKTH | |
| | PCPAPEAAGAPSVFLFPPKP | TCPPCPAPEAAGAPSVFLFP | |
| | KDTLMISRTPEVTCVVVDV | PKPKDTLMISRTPEVTCVVV | |
| | SHEDPEVKFNWYVDGVEV | DVSHEDPEVKFNWYVDGV | |
| | HNAKTKPREEQYNSTYRVV | EVHNAKTKPREEQYNSTYR | |
| | SVLTVLHQDWLNGKEYKC | VVSVLTVLHQDWLNGKEY | |
| | KVSNKALPAPIEKTISKAKG | KCKVSNKALPAPIEKTISKA | |
| | QPREPQVYTLPPCRDELTK | KGQPREPQVCTLPPSRDELT | |
| | NQVSLWCLVKGFYPSDIAV | KNQVSLSCAVKGFYPSDIA | |
| | EWESNGQPENNYKTTPPVL | VEWESNGQPENNYKTTPPV | |
| | DSDGSFFLYSKLTVDKSRW | LDSDGSFFLVSKLTVDKSR | |
| | QQGNVFSCSVMHEALHNH | WQQGNVFSCSVMHEALHN | |
| | YTQKSLSLSPG | RFTQKSLSLSPG | |
| | (SEQ ID NO: 802) | (SEQ ID NO: 803) | |
| DGL487 | QVQLVESGGGLVQAGGSLR | QVQLQESGPGLVKPSQTLS | |
| | LSCAASARIFNSYAVAWFR | LTCTVSGGSITTNYYYWSW | |
| | QTPGKEREFVASISWSGGST | IRQPPGKGLEWMGAIAYSG | |
| | NYADSVKGRFTISRDNAKN | STYYSPSLKSRTSISRDTSKN | |
| | TVFLQMNSLKPADTAVYYC | QFTLQLSSVTPEDTAVYYC | |
| | AADRNYYPSVLGKYTYWG | ASGINFYSNYPTLNEYDYW | |
| | QGTQVTVEPKSSDKTHTCP | GQGTQVTVEPKSSDKTHTC | |
| | PCPAPEAAGAPSVFLFPPKP | PPCPAPEAAGAPSVFLFPPK | |
| | KDTLMISRTPEVTCVVVDV | PKDTLMISRTPEVTCVVVD | |
| | SHEDPEVKFNWYVDGVEV | VSHEDPEVKFNWYVDGVE | |
| | HNAKTKPREEQYNSTYRVV | VHNAKTKPREEQYNSTYRV | |
| | SVLTVLHQDWLNGKEYKC | VSVLTVLHQDWLNGKEYK | |
| | KVSNKALPAPIEKTISKAKG | CKVSNKALPAPIEKTISKAK | |
| | QPREPQVYTLPPCRDELTK | GQPREPQVCTLPPSRDELTK | |
| | NQVSLWCLVKGFYPSDIAV | NQVSLSCAVKGFYPSDIAV | |
| | EWESNGQPENNYKTTPPVL | EWESNGQPENNYKTTPPVL | |
| | DSDGSFFLYSKLTVDKSRW | DSDGSFFLVSKLTVDKSRW | |
| | QQGNVFSCSVMHEALHNH | QQGNVFSCSVMHEALHNRF | |
| | YTQKSLSLSPG | TQKSLSLSPG | |
| | (SEQ ID NO: 804) | (SEQ ID NO: 805) | |
| DGL490 | QVQLVESGGGLVQAGGSLR | QVQLVESGGGLVQAGGSLR | |
| | LSCAASARIFNSYAVAWFR | LSCAASGRTFSSYAMGWFR | |
| | QTPGKEREFVASISWSGGST | QAPGKEREFVSAISWSGLST | |
| | NYADSVKGRFTISRDNAKN | YYVESVQGRFTISRDNAKN | |
| | TVFLQMNSLKPADTAVYYC | TVYLQMNSLKPEDTAVYY | |
| | AADRNYYPSVLGKYTYWG | CAASRIYTNYVARSYEYDD | |
| | QGTQVTVEPKSSDKTHTCP | WGQGTQVTVEPKSSDKTHT | |
| | PCPAPEAAGAPSVFLFPPKP | CPPCPAPEAAGAPSVFLFPP | |
| | KDTLMISRTPEVTCVVVDV | KPKDTLMISRTPEVTCVVV | |
| | SHEDPEVKFNWYVDGVEV | DVSHEDPEVKFNWYVDGV | |
| | HNAKTKPREEQYNSTYRVV | EVHNAKTKPREEQYNSTYR | |
| | SVLTVLHQDWLNGKEYKC | VVSVLTVLHQDWLNGKEY | |
| | KVSNKALPAPIEKTISKAKG | KCKVSNKALPAPIEKTISKA | |
| | QPREPQVYTLPPCRDELTK | KGQPREPQVCTLPPSRDELT | |
| | NQVSLWCLVKGFYPSDIAV | KNQVSLSCAVKGFYPSDIA | |
| | EWESNGQPENNYKTTPPVL | VEWESNGQPENNYKTTPPV | |
| | DSDGSFFLYSKLTVDKSRW | LDSDGSFFLVSKLTVDKSR | |
| | QQGNVFSCSVMHEALHNH | WQQGNVFSCSVMHEALHN | |
| | YTQKSLSLSPG | RFTQKSLSLSPG | |
| | (SEQ ID NO: 806) | (SEQ ID NO: 807) | |
| DGL496 | EVQLVESGGGLVQAGGSLR | QVQLVESGGGLVQPGGSLR | |
| | LSCAASGLTFRNYAMAWF | LSCAASGFTFSSKAMHWFR | |
| | RQAPGKEREFVAGITWGGG | QAPGKGLEWVSSINSGGAIT | |
| | LTHYVDSVKGRFTISRDNG | YYKDSVKGRFTVSRDNAK | |
| | KNTVYLQMNSLKSEDTAV | NILYLEMNSLKPEDTALYY | |
| | YYCAGKTQGSTWYHLTPD | CATDTDGRTRGQGTQVTVE | |
| | GYDYWGQGTQVTVEPKSS | PKSSDKTHTCPPCPAPEAAG | |
| | DKTHTCPPCPAPEAAGAPS | APSVFLFPPKPKDTLMISRT | |
| | VFLFPPKPKDTLMISRTPEV | PEVTCVVVDVSHEDPEVKF | |
| | TCVVVDVSHEDPEVKFNW | NWYVDGVEVHNAKTKPRE | |
| | YVDGVEVHNAKTKPREEQ | EQYNSTYRVVSVLTVLHQD | |
| | YNSTYRVVSVLTVLHQDW | WLNGKEYKCKVSNKALPA | |
| | LNGKEYKCKVSNKALPAPI | PIEKTISKAKGQPREPQVCT | |
| | EKTISKAKGQPREPQVYTLP | LPPSRDELTKNQVSLSCAV | |
| | PCRDELTKNQVSLWCLVKG | KGFYPSDIAVEWESNGQPE | |
| | FYPSDIAVEWESNGQPENN | NNYKTTPPVLDSDGSFFLVS | |
| | YKTTPPVLDSDGSFFLYSKL | KLTVDKSRWQQGNVFSCS | |
| | TVDKSRWQQGNVFSCSVM | VMHEALHNRFTQKSLSLSP | |
| | HEALHNHYTQKSLSLSPG | G | |
| | (SEQ ID NO: 808) | (SEQ ID NO: 809) | |
| DGL497 | EVQLVESGGGLVQAGGSLR | QVQLVESGGGLVQPGGSLR | |
| | LSCAASGLTFRNYAMAWF | LSCAASGFTFSNYAMSWIR | |
| | RQAPGKEREFVAGITWGGG | QGPERGFEWVSRINSGGGT | |
| | LTHYVDSVKGRFTISRDNG | TSYADSVKGRFTISRDNAK | |
| | KNTVYLQMNSLKSEDTAV | NTLYLQMNSLKPEDTAVYY | |
| | YYCAGKTQGSTWYHLTPD | CAKHSHPDYSSNYYRPGQG | |
| | GYDYWGQGTQVTVEPKSS | TQVTVEPKSSDKTHTCPPCP | |
| | DKTHTCPPCPAPEAAGAPS | APEAAGAPSVFLFPPKPKDT | |
| | VFLFPPKPKDTLMISRTPEV | LMISRTPEVTCVVVDVSHE | |
| | TCVVVDVSHEDPEVKFNW | DPEVKFNWYVDGVEVHNA | |
| | YVDGVEVHNAKTKPREEQ | KTKPREEQYNSTYRVVSVL | |
| | YNSTYRVVSVLTVLHQDW | TVLHQDWLNGKEYKCKVS | |
| | LNGKEYKCKVSNKALPAPI | NKALPAPIEKTISKAKGQPR | |
| | EKTISKAKGQPREPQVYTLP | EPQVCTLPPSRDELTKNQVS | |
| | PCRDELTKNQVSLWCLVKG | LSCAVKGFYPSDIAVEWES | |
| | FYPSDIAVEWESNGQPENN | NGQPENNYKTTPPVLDSDG | |
| | YKTTPPVLDSDGSFFLYSKL | SFFLVSKLTVDKSRWQQGN | |
| | TVDKSRWQQGNVFSCSVM | VFSCSVMHEALHNRFTQKS | |
| | HEALHNHYTQKSLSLSPG | LSLSPG | |
| | (SEQ ID NO: 810) | (SEQ ID NO: 811) | |
| DGL499 | EVQLVESGGGLVQAGGSLR | QVQLVESGGGLVQPGGSLR | |
| | LSCAASGLTFRNYAMAWF | LSCAASGFTFEPYDMSWFR | |
| | RQAPGKEREFVAGITWGGG | QAPGKGPEWVSAIDKGGG | |
| | LTHYVDSVKGRFTISRDNG | AGATYYADSVKGRFTISRD | |
| | KNTVYLQMNSLKSEDTAV | NAKKTLYLQMKSLKPEDTA | |
| | YYCAGKTQGSTWYHLTPD | VYYCAITAGPYSDSTPRVA | |
| | GYDYWGQGTQVTVEPKSS | YRGEGTQVTVEPKSSDKTH | |
| | DKTHTCPPCPAPEAAGAPS | TCPPCPAPEAAGAPSVFLFP | |
| | VFLFPPKPKDTLMISRTPEV | PKPKDTLMISRTPEVTCVVV | |
| | TCVVVDVSHEDPEVKFNW | DVSHEDPEVKFNWYVDGV | |
| | YVDGVEVHNAKTKPREEQ | EVHNAKTKPREEQYNSTYR | |
| | YNSTYRVVSVLTVLHQDW | VVSVLTVLHQDWLNGKEY | |
| | LNGKEYKCKVSNKALPAPI | KCKVSNKALPAPIEKTISKA | |
| | EKTISKAKGQPREPQVYTLP | KGQPREPQVCTLPPSRDELT | |
| | PCRDELTKNQVSLWCLVKG | KNQVSLSCAVKGFYPSDIA | |
| | FYPSDIAVEWESNGQPENN | VEWESNGQPENNYKTTPPV | |
| | YKTTPPVLDSDGSFFLYSKL | LDSDGSFFLVSKLTVDKSR | |
| | TVDKSRWQQGNVFSCSVM | WQQGNVFSCSVMHEALHN | |
| | HEALHNHYTQKSLSLSPG | RFTQKSLSLSPG | |
| | (SEQ ID NO: 812) | (SEQ ID NO: 813) | |
| DGL503 | EVQLVESGGGLVQAGGSLR | QVQLVESGGGLVQAGGSLR | |
| | LSCAASGLTFRNYAMAWF | LSCAASGRIFSAYRMGWFR | |
| | RQAPGKEREFVAGITWGGG | QAPGKEREFVAGITWRGQT | |
| | LTHYVDSVKGRFTISRDNG | TYYAASVKGRFTISKDNAK | |
| | KNTVYLQMNSLKSEDTAV | KTVYLQMNSLKPEETAVYY | |
| | YYCAGKTQGSTWYHLTPD | CAVETNGLGRWDRPSEYD | |
| | GYDYWGQGTQVTVEPKSS | YWGQGTQVTVEPKSSDKT | |
| | DKTHTCPPCPAPEAAGAPS | HTCPPCPAPEAAGAPSVFLF | |
| | VFLFPPKPKDTLMISRTPEV | PPKPKDTLMISRTPEVTCVV | |
| | TCVVVDVSHEDPEVKFNW | VDVSHEDPEVKFNWYVDG | |
| | YVDGVEVHNAKTKPREEQ | VEVHNAKTKPREEQYNSTY | |
| | YNSTYRVVSVLTVLHQDW | RVVSVLTVLHQDWLNGKE | |
| | LNGKEYKCKVSNKALPAPI | YKCKVSNKALPAPIEKTISK | |
| | EKTISKAKGQPREPQVYTLP | AKGQPREPQVCTLPPSRDEL | |
| | PCRDELTKNQVSLWCLVKG | TKNQVSLSCAVKGFYPSDI | |
| | FYPSDIAVEWESNGQPENN | AVEWESNGQPENNYKTTPP | |
| | YKTTPPVLDSDGSFFLYSKL | VLDSDGSFFLVSKLTVDKS | |
| | TVDKSRWQQGNVFSCSVM | RWQQGNVFSCSVMHEALH | |
| | HEALHNHYTQKSLSLSPG | NRFTQKSLSLSPG | |
| | (SEQ ID NO: 814) | (SEQ ID NO: 815) | |
| DGL504 | EVQLVESGGGLVQAGGSLR | QVQLVESGGGLVQAGGSLR | |
| | LSCAASGLTFRNYAMAWF | LSCAASGRTFSSYAMGWFR | |
| | RQAPGKEREFVAGITWGGG | QAPGKEREFVSAISWSGLST | |
| | LTHYVDSVKGRFTISRDNG | YYVESVQGRFTISRDNAKN | |
| | KNTVYLQMNSLKSEDTAV | TVYLQMNSLKPEDTAVYY | |
| | YYCAGKTQGSTWYHLTPD | CAASRIYTNYVARSYEYDD | |
| | GYDYWGQGTQVTVEPKSS | WGQGTQVTVEPKSSDKTHT | |
| | DKTHTCPPCPAPEAAGAPS | CPPCPAPEAAGAPSVFLFPP | |
| | VFLFPPKPKDTLMISRTPEV | KPKDTLMISRTPEVTCVVV | |
| | TCVVVDVSHEDPEVKFNW | DVSHEDPEVKFNWYVDGV | |
| | YVDGVEVHNAKTKPREEQ | EVHNAKTKPREEQYNSTYR | |
| | YNSTYRVVSVLTVLHQDW | VVSVLTVLHQDWLNGKEY | |
| | LNGKEYKCKVSNKALPAPI | KCKVSNKALPAPIEKTISKA | |
| | EKTISKAKGQPREPQVYTLP | KGQPREPQVCTLPPSRDELT | |
| | PCRDELTKNQVSLWCLVKG | KNQVSLSCAVKGFYPSDIA | |
| | FYPSDIAVEWESNGQPENN | VEWESNGQPENNYKTTPPV | |
| | YKTTPPVLDSDGSFFLYSKL | LDSDGSFFLVSKLTVDKSR | |
| | TVDKSRWQQGNVFSCSVM | WQQGNVFSCSVMHEALHN | |
| | HEALHNHYTQKSLSLSPG | RFTQKSLSLSPG | |
| | (SEQ ID NO: 816) | (SEQ ID NO: 817) | |
| DGL505 | EVQLVESGGGLVQAGGSLR | QVQLVESGGGLVQLGDSLR | |
| | LSCAASGLTFRNYAMAWF | LSCAASGRGFSSWTMAWF | |
| | RQAPGKEREFVAGITWGGG | RQALGKRREFVAAITATGA | |
| | LTHYVDSVKGRFTISRDNG | RTSSAESEGRFTISRDNAKN | |
| | KNTVYLQMNSLKSEDTAV | MVFLQMNSLKAEDTAVYY | |
| | YYCAGKTQGSTWYHLTPD | CAATGIASWDPGMADKYT | |
| | GYDYWGQGTQVTVEPKSS | YWGQGTQVTVEPKSSDKT | |
| | DKTHTCPPCPAPEAAGAPS | HTCPPCPAPEAAGAPSVFLF | |
| | VFLFPPKPKDTLMISRTPEV | PPKPKDTLMISRTPEVTCVV | |
| | TCVVVDVSHEDPEVKFNW | VDVSHEDPEVKFNWYVDG | |
| | YVDGVEVHNAKTKPREEQ | VEVHNAKTKPREEQYNSTY | |
| | YNSTYRVVSVLTVLHQDW | RVVSVLTVLHQDWLNGKE | |
| | LNGKEYKCKVSNKALPAPI | YKCKVSNKALPAPIEKTISK | |
| | EKTISKAKGQPREPQVYTLP | AKGQPREPQVCTLPPSRDEL | |
| | PCRDELTKNQVSLWCLVKG | TKNQVSLSCAVKGFYPSDI | |
| | FYPSDIAEWESNGQPENN | AVEWESNGQPENNYKTTPP | |
| | YKTTPPVLDSDGSFFLYSKL | VLDSDGSFFLVSKLTVDKS | |
| | TVDKSRWQQGNVFSCSVM | RWQQGNVFSCSVMHEALH | |
| | HEALHNHYTQKSLSLSPG | NRFTQKSLSLSPG | |
| | (SEQ ID NO: 818) | (SEQ ID NO: 819) | |
| DGL506 | EVQLVESGGGLVQAGGSLR | QVQLVESGGGLVQAGGSL | |
| | LSCAASGLTFRNYAMAWF | KLSCAASGGTFSSYAMGWF | |
| | RQAPGKEREFVAGITWGGG | RQAPGKEREFVAAIMRSGG | |
| | LTHYVDSVKGRFTISRDNG | TTVYVNSVKGRFTVSRDNA | |
| | KNTVYLQMNSLKSEDTAV | KNTVYLQMNSLKPEDAAV | |
| | YYCAGKTQGSTWYHLTPD | YYCAAGRPVSRTSYYTYTY | |
| | GYDYWGQGTQVTVEPKSS | PHDYDYWGQGTQVSVEPK | |
| | DKTHTCPPCPAPEAAGAPS | SSDKTHTCPPCPAPEAAGAP | |
| | VFLFPPKPKDTLMISRTPEV | SVFLFPPKPKDTLMISRTPE | |
| | TCVVVDVSHEDPEVKFNW | VTCVVVDVSHEDPEVKFN | |
| | YVDGVEVHNAKTKPREEQ | WYVDGVEVHNAKTKPREE | |
| | YNSTYRVVSVLTVLHQDW | QYNSTYRVVSVLTVLHQD | |
| | LNGKEYKCKVSNKALPAPI | WLNGKEYKCKVSNKALPA | |
| | EKTISKAKGQPREPQVYTLP | PIEKTISKAKGQPREPQVCT | |
| | PCRDELTKNQVSLWCLVKG | LPPSRDELTKNQVSLSCAV | |
| | FYPSDIAVEWESNGQPENN | KGFYPSDIAVEWESNGQPE | |
| | YKTTPPVLDSDGSFFLYSKL | NNYKTTPPVLDSDGSFFLVS | |
| | TVDKSRWQQGNVFSCSVM | KLTVDKSRWQQGNVFSCS | |
| | HEALHNHYTQKSLSLSPG | VMHEALHNRFTQKSLSLSP | |
| | (SEQ ID NO: 820) | G | |
| | | (SEQ ID NO: 821) | |
| DGL508 | EVQLVESGGGLVQAGGSLR | QVQLVESGGGLVQAGGSLR | |
| | LSCAASGLTFRNYAMAWF | LSCAASGRTFSRYIMGWFR | |
| | RQAPGKEREFVAGITWGGG | QATGKEREFVAAISWSGLS | |
| | LTHYVDSVKGRFTISRDNG | THYADSVQGRFTISRDNAE | |
| | KNTVYLQMNSLKSEDTAV | NTVYLQMNSLKPEDTAVY | |
| | YYCAGKTQGSTWYHLTPD | YCAADRHWETRLLPREYD | |
| | GYDYWGQGTQVTVEPKSS | YWGQGTQVTVEPKSSDKT | |
| | DKTHTCPPCPAPEAAGAPS | HTCPPCPAPEAAGAPSVFLF | |
| | VFLFPPKPKDTLMISRTPEV | PPKPKDTLMISRTPEVTCVV | |
| | TCVVVDVSHEDPEVKFNW | VDVSHEDPEVKFNWYVDG | |
| | YVDGVEVHNAKTKPREEQ | VEVHNAKTKPREEQYNSTY | |
| | YNSTYRVVSVLTVLHQDW | RVVSVLTVLHQDWLNGKE | |
| | LNGKEYKCKVSNKALPAPI | YKCKVSNKALPAPIEKTISK | |
| | EKTISKAKGQPREPQVYTLP | AKGQPREPQVCTLPPSRDEL | |
| | PCRDELTKNQVSLWCLVKG | TKNQVSLSCAVKGFYPSDI | |
| | FYPSDIAVEWESNGQPENN | AVEWESNGQPENNYKTTPP | |
| | YKTTPPVLDSDGSFFLYSKL | VLDSDGSFFLVSKLTVDKS | |
| | TVDKSRWQQGNVFSCSVM | RWQQGNVFSCSVMHEALH | |
| | HEALHNHYTQKSLSLSPG | NRFTQKSLSLSPG | |
| | (SEQ ID NO: 822) | (SEQ ID NO: 823) | |
| DGL514 | QVQLVESGGGLAQAGGSLR | QVQLVESGGGLVQPGGSLR | |
| | LSCAASGRTFSNDDMAWFR | LSCAASGFTFSNYAMSWIR | |
| | QAPGKEREFVAHIDRNAAS | QGPERGFEWVSRINSGGGT | |
| | TNYADSVKGRFTISRDNAK | TSYADSVKGRFTISRDNAK | |
| | NTVYLQMTRLKPEDTAVY | NTLYLQMNSLKPEDTAVYY | |
| | YCAADPDHFGTTWYQQYT | CAKHSHPDYSSNYYRPGQG | |
| | YWGPGTQVTVEPKSSDKTH | TQVTVEPKSSDKTHTCPPCP | |
| | TCPPCPAPEAAGAPSVFLFP | APEAAGAPSVFLFPPKPKDT | |
| | PKPKDTLMISRTPEVTCVVV | LMISRTPEVTCVVVDVSHE | |
| | DVSHEDPEVKFNWYVDGV | DPEVKFNWYVDGVEVHNA | |
| | EVHNAKTKPREEQYNSTYR | KTKPREEQYNSTYRVVSVL | |
| | VVSVLTVLHQDWLNGKEY | TVLHQDWLNGKEYKCKVS | |
| | KCKVSNKALPAPIEKTISKA | NKALPAPIEKTISKAKGQPR | |
| | KGQPREPQVYTLPPCRDEL | EPQVCTLPPSRDELTKNQVS | |
| | TKNQVSLWCLVKGFYPSDI | LSCAVKGFYPSDIAVEWES | |
| | AVEWESNGQPENNYKTTPP | NGQPENNYKTTPPVLDSDG | |
| | VLDSDGSFFLYSKLTVDKS | SFFLVSKLTVDKSRWQQGN | |
| | RWQQGNVFSCSVMHEALH | VFSCSVMHEALHNRFTQKS | |
| | NHYTQKSLSLSPG | LSLSPG | |
| | (SEQ ID NO: 824) | (SEQ ID NO: 825) | |
| DGL515 | QVQLVESGGGLAQAGGSLR | QVQLVESGGGLVQAGGSLR | |
| | LSCAASGRTFSNDDMAWFR | VSCAASGRIPSLNTMAWYR | |
| | QAPGKEREFVAHIDRNAAS | QAPGKQREFVAFITNGGTT | |
| | TNYADSVKGRFTISRDNAK | NYVDSVEGRFSISRDNTKN | |
| | NTVYLQMTRLKPEDTAVY | AVDLQMNSLKPEDTGVYY | |
| | YCAADPDHFGTTWYQQYT | CNVQISQYPYNYWGQGTQ | |
| | YWGPGTQVTVEPKSSDKTH | VTVEPKSSDKTHTCPPCPAP | |
| | TCPPCPAPEAAGAPSVFLFP | EAAGAPSVFLFPPKPKDTL | |
| | PKPKDTLMISRTPEVTCVVV | MISRTPEVTCVVVDVSHED | |
| | DVSHEDPEVKFNWYVDGV | PEVKFNWYVDGVEVHNAK | |
| | EVHNAKTKPREEQYNSTYR | TKPREEQYNSTYRVVSVLT | |
| | VVSVLTVLHQDWLNGKEY | VLHQDWLNGKEYKCKVSN | |
| | KCKVSNKALPAPIEKTISKA | KALPAPIEKTISKAKGQPRE | |
| | KGQPREPQVYTLPPCRDEL | PQVCTLPPSRDELTKNQVSL | |
| | TKNQVSLWCLVKGFYPSDI | SCAVKGFYPSDIAVEWESN | |
| | AVEWESNGQPENNYKTTPP | GQPENNYKTTPPVLDSDGS | |
| | VLDSDGSFFLYSKLTVDKS | FFLVSKLTVDKSRWQQGNV | |
| | RWQQGNVFSCSVMHEALH | FSCSVMHEALHNRFTQKSL | |
| | NHYTQKSLSLSPG | SLSPG | |
| | (SEQ ID NO: 826) | (SEQ ID NO: 827) | |
| DGL516 | QVQLVESGGGLAQAGGSLR | QVQLVESGGGLVQPGGSLR | |
| | LSCAASGRTFSNDDMAWFR | LSCAASGFTFEPYDMSWFR | |
| | QAPGKEREFVAHIDRNAAS | QAPGKGPEWVSAIDKGGG | |
| | TNYADSVKGRFTISRDNAK | AGATYYADSVKGRFTISRD | |
| | NTVYLQMTRLKPEDTAVY | NAKKTLYLQMKSLKPEDTA | |
| | YCAADPDHFGTTWYQQYT | VYYCAITAGPYSDSTPRVA | |
| | YWGPGTQVTVEPKSSDKTH | YRGEGTQVTVEPKSSDKTH | |
| | TCPPCPAPEAAGAPSVFLFP | TCPPCPAPEAAGAPSVFLFP | |
| | PKPKDTLMISRTPEVTCVVV | PKPKDTLMISRTPEVTCVVV | |
| | DVSHEDPEVKFNWYVDGV | DVSHEDPEVKFNWYVDGV | |
| | EVHNAKTKPREEQYNSTYR | EVHNAKTKPREEQYNSTYR | |
| | VVSVLTVLHQDWLNGKEY | VVSVLTVLHQDWLNGKEY | |
| | KCKVSNKALPAPIEKTISKA | KCKVSNKALPAPIEKTISKA | |
| | KGQPREPQVYTLPPCRDEL | KGQPREPQVCTLPPSRDELT | |
| | TKNQVSLWCLVKGFYPSDI | KNQVSLSCAVKGFYPSDIA | |
| | AVEWESNGQPENNYKTTPP | VEWESNGQPENNYKTTPPV | |
| | VLDSDGSFFLYSKLTVDKS | LDSDGSFFLVSKLTVDKSR | |
| | RWQQGNVFSCSVMHEALH | WQQGNVFSCSVMHEALHN | |
| | NHYTQKSLSLSPG | RFTQKSLSLSPG | |
| | (SEQ ID NO: 828) | (SEQ ID NO: 829) | |
| DGL518 | EVQLVESGGGLVQPGGSLR | QVQLVESGGGLVRPGGSLR | |
| | LFCAASRSIFNINIMGWYRQ | LSCAASGFTFSSNGMHWFR | |
| | APGKQREWLASITSGGSTF | QAPGKGLEWVSSIGSGGTSI | |
| | YADSVKDRFTISRDSVKNT | YYADSVKGRFTISRDNAKN | |
| | VYLQMNSLKPEDTAAYYC | TLYLQMNSLKSDDTAVYY | |
| | NRYRSVPLNTVYPWGQGTL | CSKEVTGATRGQGTQVTVE | |
| | VTVEPKSSDKTHTCPPCPAP | PKSSDKTHTCPPCPAPEAAG | |
| | EAAGAPSVFLFPPKPKDTL | APSVFLFPPKPKDTLMISRT | |
| | MISRTPEVTCVVVDVSHED | PEVTCVVVDVSHEDPEVKF | |
| | PEVKFNWYVDGVEVHNAK | NWYVDGVEVHNAKTKPRE | |
| | TKPREEQYNSTYRVVSVLT | EQYNSTYRVVSVLTVLHQD | |
| | VLHQDWLNGKEYKCKVSN | WLNGKEYKCKVSNKALPA | |
| | KALPAPIEKTISKAKGQPRE | PIEKTISKAKGQPREPQVCT | |
| | PQVYTLPPCRDELTKNQVS | LPPSRDELTKNQVSLSCAV | |
| | LWCLVKGFYPSDIAVEWES | KGFYPSDIAVEWESNGQPE | |
| | NGQPENNYKTTPPVLDSDG | NNYKTTPPVLDSDGSFFLVS | |
| | SFFLYSKLTVDKSRWQQGN | KLTVDKSRWQQGNVFSCS | |
| | VFSCSVMHEALHNHYTQKS | VMHEALHNRFTQKSLSLSP | |
| | LSLSPG | G | |
| | (SEQ ID NO: 830) | (SEQ ID NO: 831) | |
| DGL524 | EVQLVESGGGLVQPGGSLR | QVQLVESGGGLVQAGGSLR | |
| | LFCAASRSIFNINIMGWYRQ | LSCAASGRIFSAYRMGWFR | |
| | APGKQREWLASITSGGSTF | QAPGKEREFVAGITWRGQT | |
| | YADSVKDRFTISRDSVKNT | TYYAASVKGRFTISKDNAK | |
| | VYLQMNSLKPEDTAAYYC | KTVYLQMNSLKPEETAVYY | |
| | NRYRSVPLNTVYPWGQGTL | CAVETNGLGRWDRPSEYD | |
| | VTVEPKSSDKTHTCPPCPAP | YWGQGTQVTVEPKSSDKT | |
| | EAAGAPSVFLFPPKPKDTL | HTCPPCPAPEAAGAPSVFLF | |
| | MISRTPEVTCVVVDVSHED | PPKPKDTLMISRTPEVTCVV | |
| | PEVKFNWYVDGVEVHNAK | VDVSHEDPEVKFNWYVDG | |
| | TKPREEQYNSTYRVVSVLT | VEVHNAKTKPREEQYNSTY | |
| | VLHQDWLNGKEYKCKVSN | RVVSVLTVLHQDWLNGKE | |
| | KALPAPIEKTISKAKGQPRE | YKCKVSNKALPAPIEKTISK | |
| | PQVYTLPPCRDELTKNQVS | AKGQPREPQVCTLPPSRDEL | |
| | LWCLVKGFYPSDIAVEWES | TKNQVSLSCAVKGFYPSDI | |
| | NGQPENNYKTTPPVLDSDG | AVEWESNGQPENNYKTTPP | |
| | SFFLYSKLTVDKSRWQQGN | VLDSDGSFFLVSKLTVDKS | |
| | VFSCSVMHEALHNHYTQKS | RWQQGNVFSCSVMHEALH | |
| | LSLSPG | NRFTQKSLSLSPG | |
| | (SEQ ID NO: 832) | (SEQ ID NO: 833) | |
| DGL525 | EVQLVESGGGLVQPGGSLR | QVQLVESGGGLVQAGGSLR | |
| | LFCAASRSIFNINIMGWYRQ | LSCAASGRTFSSYAMGWFR | |
| | APGKQREWLASITSGGSTF | QAPGKEREFVSAISWSGLST | |
| | YADSVKDRFTISRDSVKNT | YYVESVQGRFTISRDNAKN | |
| | VYLQMNSLKPEDTAAYYC | TVYLQMNSLKPEDTAVYY | |
| | NRYRSVPLNTVYPWGQGTL | CAASRIYTNYVARSYEYDD | |
| | VTVEPKSSDKTHTCPPCPAP | WGQGTQVTVEPKSSDKTHT | |
| | EAAGAPSVFLFPPKPKDTL | CPPCPAPEAAGAPSVFLFPP | |
| | MISRTPEVTCVVVDVSHED | KPKDTLMISRTPEVTCVVV | |
| | PEVKFNWYVDGVEVHNAK | DVSHEDPEVKFNWYVDGV | |
| | TKPREEQYNSTYRVVSVLT | EVHNAKTKPREEQYNSTYR | |
| | VLHQDWLNGKEYKCKVSN | VVSVLTVLHQDWLNGKEY | |
| | KALPAPIEKTISKAKGQPRE | KCKVSNKALPAPIEKTISKA | |
| | PQVYTLPPCRDELTKNQVS | KGQPREPQVCTLPPSRDELT | |
| | LWCLVKGFYPSDIAVEWES | KNQVSLSCAVKGFYPSDIA | |
| | NGQPENNYKTTPPVLDSDG | VEWESNGQPENNYKTTPPV | |
| | SFFLYSKLTVDKSRWQQGN | LDSDGSFFLVSKLTVDKSR | |
| | VFSCSVMHEALHNHYTQKS | WQQGNVFSCSVMHEALHN | |
| | LSLSPG | RFTQKSLSLSPG | |
| | (SEQ ID NO: 835) | (SEQ ID NO: 836) | |
| DGL527 | EVQLVESGGGLVQPGGSLT | QVQLVESGGGLVRPGGSLR | |
| | LSCTASGFRFSDRSMGWYR | LSCAASGFTFSSNGMHWFR | |
| | QAPGKQRELVAAITVGGST | QAPGKGLEWVSSIGSGGTSI | |
| | NYADSVKGRFTISRDNAKN | YYADSVKGRFTISRDNAKN | |
| | TVYLQMNSLKPEDTALYYC | TLYLQMNSLKSDDTAVYY | |
| | NRRYPPDDYWGQGTQVTV | CSKEVTGATRGQGTQVTVE | |
| | EPKSSDKTHTCPPCPAPEAA | PKSSDKTHTCPPCPAPEAAG | |
| | GAPSVFLFPPKPKDTLMISR | APSVFLFPPKPKDTLMISRT | |
| | TPEVTCVVVDVSHEDPEVK | PEVTCVVVDVSHEDPEVKF | |
| | FNWYVDGVEVHNAKTKPR | NWYVDGVEVHNAKTKPRE | |
| | EEQYNSTYRVVSVLTVLHQ | EQYNSTYRVVSVLTVLHQD | |
| | DWLNGKEYKCKVSNKALP | WLNGKEYKCKVSNKALPA | |
| | APIEKTISKAKGQPREPQVY | PIEKTISKAKGQPREPQVCT | |
| | TLPPCRDELTKNQVSLWCL | LPPSRDELTKNQVSLSCAV | |
| | VKGFYPSDIAEWESNGQP | KGFYPSDIAVEWESNGQPE | |
| | ENNYKTTPPVLDSDGSFFLY | NNYKTTPPVLDSDGSFFLVS | |
| | SKLTVDKSRWQQGNVFSCS | KLTVDKSRWQQGNVFSCS | |
| | VMHEALHNHYTQKSLSLSP | VMHEALHNRFTQKSLSLSP | |
| | G | G | |
| | (SEQ ID NO: 837) | (SEQ ID NO: 838) | |
| DGL533 | EVQLVESGGGLVQPGGSLT | QVQLVESGGGLVQAGGSLR | |
| | LSCTASGFRFSDRSMGWYR | LSCAASGRTFSSYAMGWFR | |
| | QAPGKQRELVAAITVGGST | QAPGKEREFVSAISWSGLST | |
| | NYADSVKGRFTISRDNAKN | YYVESVQGRFTISRDNAKN | |
| | TVYLQMNSLKPEDTALYYC | TVYLQMNSLKPEDTAVYY | |
| | NRRYPPDDYWGQGTQVTV | CAASRIYTNYVARSYEYDD | |
| | EPKSSDKTHTCPPCPAPEAA | WGQGTQVTVEPKSSDKTHT | |
| | GAPSVFLFPPKPKDTLMISR | CPPCPAPEAAGAPSVFLFPP | |
| | TPEVTCVVVDVSHEDPEVK | KPKDTLMISRTPEVTCVVV | |
| | FNWYVDGVEVHNAKTKPR | DVSHEDPEVKFNWYVDGV | |
| | EEQYNSTYRVVSVLTVLHQ | EVHNAKTKPREEQYNSTYR | |
| | DWLNGKEYKCKVSNKALP | VVSVLTVLHQDWLNGKEY | |
| | APIEKTISKAKGQPREPQVY | KCKVSNKALPAPIEKTISKA | |
| | TLPPCRDELTKNQVSLWCL | KGQPREPQVCTLPPSRDELT | |
| | VKGFYPSDIAVEWESNGQP | KNQVSLSCAVKGFYPSDIA | |
| | ENNYKTTPPVLDSDGSFFLY | VEWESNGQPENNYKTTPPV | |
| | SKLTVDKSRWQQGNVFSCS | LDSDGSFFLVSKLTVDKSR | |
| | VMHEALHNHYTQKSLSLSP | WQQGNVFSCSVMHEALHN | |
| | G | RFTQKSLSLSPG | |
| | (SEQ ID NO: 839) | (SEQ ID NO: 840) | |
| DGL534 | QVQLVESGGGLVQAGGSLR | QVQLVESGGGLVQPGGSLR | |
| | LSCLASGRTSNYFMGWFRQ | LSCAASGFTFSNYAMSWVR | |
| | APGKEREFVAAISRSGGGT | QAPGKGLEWVSAINSDGTS | |
| | WYADSVKGRFAISRDNAK | TSYADSVKGQFTISRDNAK | |
| | NTVNLQMNSLKREDTAVY | NTLYLQMNSLKPEDTAVYY | |
| | YCAADRRTGTSVYTVDEY | CTKWEGDTDSDYKFKDYW | |
| | DYWGQGTQVTVEPKSSDK | GQGTQVTVEPKSSDKTHTC | |
| | THTCPPCPAPEAAGAPSVFL | PPCPAPEAAGAPSVFLFPPK | |
| | FPPKPKDTLMISRTPEVTCV | PKDTLMISRTPEVTCVVVD | |
| | VVDVSHEDPEVKFNWYVD | VSHEDPEVKFNWYVDGVE | |
| | GVEVHNAKTKPREEQYNST | VHNAKTKPREEQYNSTYRV | |
| | YRVVSVLTVLHQDWLNGK | VSVLTVLHQDWLNGKEYK | |
| | EYKCKVSNKALPAPIEKTIS | CKVSNKALPAPIEKTISKAK | |
| | KAKGQPREPQVYTLPPCRD | GQPREPQVCTLPPSRDELTK | |
| | ELTKNQVSLWCLVKGFYPS | NQVSLSCAVKGFYPSDIAV | |
| | DIAVEWESNGQPENNYKTT | EWESNGQPENNYKTTPPVL | |
| | PPVLDSDGSFFLYSKLTVDK | DSDGSFFLVSKLTVDKSRW | |
| | SRWQQGNVFSCSVMHEAL | QQGNVFSCSVMHEALHNRF | |
| | HNHYTQKSLSLSPG | TQKSLSLSPG | |
| | (SEQ ID NO: 841) | (SEQ ID NO: 842) | |
| DGL537 | QVQLVESGGGLVQAGGSLR | QVQLVESGGGLVQAGGSLR | |
| | LSCLASGRTSNYFMGWFRQ | LSCAASGRIFSAYRMGWFR | |
| | APGKEREFVAAISRSGGGT | QAPGKEREFVAGITWRGQT | |
| | WYADSVKGRFAISRDNAK | TYYAASVKGRFTISKDNAK | |
| | NTVNLQMNSLKREDTAVY | KTVYLQMNSLKPEETAVYY | |
| | YCAADRRTGTSVYTVDEY | CAVETNGLGRWDRPSEYD | |
| | DYWGQGTQVTVEPKSSDK | YWGQGTQVTVEPKSSDKT | |
| | THTCPPCPAPEAAGAPSVFL | HTCPPCPAPEAAGAPSVFLF | |
| | FPPKPKDTLMISRTPEVTCV | PPKPKDTLMISRTPEVTCVV | |
| | VVDVSHEDPEVKFNWYVD | VDVSHEDPEVKFNWYVDG | |
| | GVEVHNAKTKPREEQYNST | VEVHNAKTKPREEQYNSTY | |
| | YRVVSVLTVLHQDWLNGK | RVVSVLTVLHQDWLNGKE | |
| | EYKCKVSNKALPAPIEKTIS | YKCKVSNKALPAPIEKTISK | |
| | KAKGQPREPQVYTLPPCRD | AKGQPREPQVCTLPPSRDEL | |
| | ELTKNQVSLWCLVKGFYPS | TKNQVSLSCAVKGFYPSDI | |
| | DIAVEWESNGQPENNYKTT | AVEWESNGQPENNYKTTPP | |
| | PPVLDSDGSFFLYSKLTVDK | VLDSDGSFFLVSKLTVDKS | |
| | SRWQQGNVFSCSVMHEAL | RWQQGNVFSCSVMHEALH | |
| | HNHYTQKSLSLSPG | NRFTQKSLSLSPG | |
| | (SEQ ID NO: 843) | (SEQ ID NO: 844) | |
| DGL541 | QVQLVESGGGLVQAGGSLR | QVQLVESGGGLVQAGGSLR | |
| | LSCAASGRTFRTYAMGWFR | LSCAASGRTFSSYAMGWFR | |
| | QAPGKDREVVGIAAWKVA | QAPGKEREFVSAISWSGLST | |
| | STYYVDYGDSVRGRFTISR | YYVESVQGRFTISRDNAKN | |
| | DDAKSTVYLQMNNLMPED | TVYLQMNSLKPEDTAVYY | |
| | TAVYYCAAGAFRTDWRSY | CAASRIYTNYVARSYEYDD | |
| | AYWGQGTQVTVEPKSSDK | WGQGTQVTVEPKSSDKTHT | |
| | THTCPPCPAPEAAGAPSVFL | CPPCPAPEAAGAPSVFLFPP | |
| | FPPKPKDTLMISRTPEVTCV | KPKDTLMISRTPEVTCVVV | |
| | VVDVSHEDPEVKFNWYVD | DVSHEDPEVKFNWYVDGV | |
| | GVEVHNAKTKPREEQYNST | EVHNAKTKPREEQYNSTYR | |
| | YRVVSVLTVLHQDWLNGK | VVSVLTVLHQDWLNGKEY | |
| | EYKCKVSNKALPAPIEKTIS | KCKVSNKALPAPIEKTISKA | |
| | KAKGQPREPQVYTLPPCRD | KGQPREPQVCTLPPSRDELT | |
| | ELTKNQVSLWCLVKGFYPS | KNQVSLSCAVKGFYPSDIA | |
| | DIAVEWESNGQPENNYKTT | VEWESNGQPENNYKTTPPV | |
| | PPVLDSDGSFFLYSKLTVDK | LDSDGSFFLVSKLTVDKSR | |
| | SRWQQGNVFSCSVMHEAL | WQQGNVFSCSVMHEALHN | |
| | HNHYTQKSLSLSPG | RFTQKSLSLSPG | |
| | (SEQ ID NO: 845) | (SEQ ID NO: 846) | |
| DGL545 | QVQLVESGGGAVQAGGSL | QVQLVESGGGLVQAGGSLR | |
| | RLSCAASGLTSRHYVMAW | LSCAASGRTFSSYAMGWFR | |
| | FRQAPGIEREFVASIWWGG | QAPGKEREFVSAISWSGLST | |
| | STYYADSVKDRFTISRDNA | YYVESVQGRFTISRDNAKN | |
| | ENTVYLQMNSLKPEDTAVY | TVYLQMNSLKPEDTAVYY | |
| | RCAVGTKIGTMGPRYDYW | CAASRIYTNYVARSYEYDD | |
| | GQGTQVTVEPKSSDKTHTC | WGQGTQVTVEPKSSDKTHT | |
| | PPCPAPEAAGAPSVFLFPPK | CPPCPAPEAAGAPSVFLFPP | |
| | PKDTLMISRTPEVTCVVVD | KPKDTLMISRTPEVTCVVV | |
| | VSHEDPEVKFNWYVDGVE | DVSHEDPEVKFNWYVDGV | |
| | VHNAKTKPREEQYNSTYRV | EVHNAKTKPREEQYNSTYR | |
| | VSVLTVLHQDWLNGKEYK | VVSVLTVLHQDWLNGKEY | |
| | CKVSNKALPAPIEKTISKAK | KCKVSNKALPAPIEKTISKA | |
| | GQPREPQVYTLPPCRDELT | KGQPREPQVCTLPPSRDELT | |
| | KNQVSLWCLVKGFYPSDIA | KNQVSLSCAVKGFYPSDIA | |
| | VEWESNGQPENNYKTTPPV | VEWESNGQPENNYKTTPPV | |
| | LDSDGSFFLYSKLTVDKSR | LDSDGSFFLVSKLTVDKSR | |
| | WQQGNVFSCSVMHEALHN | WQQGNVFSCSVMHEALHN | |
| | HYTQKSLSLSPG | RFTQKSLSLSPG | |
| | (SEQ ID NO: 847) | (SEQ ID NO: 848) | |
| DGL547 | EVQLVESGGGLVQPGGSLR | QLQLVESGGGLVQAGGSLR | |
| | LSCAASGFTLDYYAIGWFR | LSCAASGLTFSGYTVGWFR | |
| | QAPGKEREGVSCISSSDRST | QAPGKEREFVAAINWSSAY | |
| | WYADSVKGRFTISRDNAKN | TYSEFVKGRFTISRDNAKNT | |
| | TVYLQMHSLKPEDTAVYY | VYLQMSSLKPEDTAVYYCA | |
| | CATPCYSDYDPEGYEYDY | AQRSRTSWYASTAEAYDY | |
| | WGQGTQVTVEPKSSDKTHT | WGQGTQVTVEPKSSDKTHT | |
| | CPPCPAPEAAGAPSVFLFPP | CPPCPAPEAAGAPSVFLFPP | |
| | KPKDTLMISRTPEVTCVVV | KPKDTLMISRTPEVTCVVV | |
| | DVSHEDPEVKFNWYVDGV | DVSHEDPEVKFNWYVDGV | |
| | EVHNAKTKPREEQYNSTYR | EVHNAKTKPREEQYNSTYR | |
| | VVSVLTVLHQDWLNGKEY | VVSVLTVLHQDWLNGKEY | |
| | KCKVSNKALPAPIEKTISKA | KCKVSNKALPAPIEKTISKA | |
| | KGQPREPQVYTLPPCRDEL | KGQPREPQVCTLPPSRDELT | |
| | TKNQVSLWCLVKGFYPSDI | KNQVSLSCAVKGFYPSDIA | |
| | AVEWESNGQPENNYKTTPP | VEWESNGQPENNYKTTPPV | |
| | VLDSDGSFFLYSKLTVDKS | LDSDGSFFLVSKLTVDKSR | |
| | RWQQGNVFSCSVMHEALH | WQQGNVFSCSVMHEALHN | |
| | NHYTQKSLSLSPG | RFTQKSLSLSPG | |
| | (SEQ ID NO: 849) | (SEQ ID NO: 850) | |
| DGL551 | QVQLVESGGGLVQAGGSLR | QVQLVESGGGLVQAGGSLR | |
| | LSCAASGRTFGTYAMGWF | LSCAASGRTFSRYRMGWFR | |
| | RQAPGKERDFVARISNSGA | QAPGKEREFVAAITKSGAT | |
| | FTHYADSVKGRFTISRDNA | TYDADSVKGRFTISRDNAK | |
| | KNTVYLHMNSLENGDTAA | NTLYLQMNSLKPEDTAVYY | |
| | YYCVATRGTNSAYYTRST | CAASYHSNWWPKNADEYA | |
| | MYEYWGQGTQVTVEPKSS | YWGQGTQVTVEPKSSDKT | |
| | DKTHTCPPCPAPEAAGAPS | HTCPPCPAPEAAGAPSVFLF | |
| | VFLFPPKPKDTLMISRTPEV | PPKPKDTLMISRTPEVTCVV | |
| | TCVVVDVSHEDPEVKFNW | VDVSHEDPEVKFNWYVDG | |
| | YVDGVEVHNAKTKPREEQ | VEVHNAKTKPREEQYNSTY | |
| | YNSTYRVVSVLTVLHQDW | RVVSVLTVLHQDWLNGKE | |
| | LNGKEYKCKVSNKALPAPI | YKCKVSNKALPAPIEKTISK | |
| | EKTISKAKGQPREPQVYTLP | AKGQPREPQVCTLPPSRDEL | |
| | PCRDELTKNQVSLWCLVKG | TKNQVSLSCAVKGFYPSDI | |
| | FYPSDIAVEWESNGQPENN | AVEWESNGQPENNYKTTPP | |
| | YKTTPPVLDSDGSFFLYSKL | VLDSDGSFFLVSKLTVDKS | |
| | TVDKSRWQQGNVFSCSVM | RWQQGNVFSCSVMHEALH | |
| | HEALHNHYTQKSLSLSPG | NRFTQKSLSLSPG | |
| | (SEQ ID NO: 851) | (SEQ ID NO: 852) | |
| DGL552 | QVQLVESGGGLVQAGGSLR | QVQLVESGGGLVQAGGSLR | |
| | LSCAASGRTFGTYAMGWF | LSCAASGRIFSAYRMGWFR | |
| | RQAPGKERDFVARISNSGA | QAPGKEREFVAGITWRGQT | |
| | FTHYADSVKGRFTISRDNA | TYYAASVKGRFTISKDNAK | |
| | KNTVYLHMNSLENGDTAA | KTVYLQMNSLKPEETAVYY | |
| | YYCVATRGTNSAYYTRST | CAVETNGLGRWDRPSEYD | |
| | MYEYWGQGTQVTVEPKSS | YWGQGTQVTVEPKSSDKT | |
| | DKTHTCPPCPAPEAAGAPS | HTCPPCPAPEAAGAPSVFLF | |
| | VFLFPPKPKDTLMISRTPEV | PPKPKDTLMISRTPEVTCVV | |
| | TCVVVDVSHEDPEVKFNW | VDVSHEDPEVKFNWYVDG | |
| | YVDGVEVHNAKTKPREEQ | VEVHNAKTKPREEQYNSTY | |
| | YNSTYRVVSVLTVLHQDW | RVVSVLTVLHQDWLNGKE | |
| | LNGKEYKCKVSNKALPAPI | YKCKVSNKALPAPIEKTISK | |
| | EKTISKAKGQPREPQVYTLP | AKGQPREPQVCTLPPSRDEL | |
| | PCRDELTKNQVSLWCLVKG | TKNQVSLSCAVKGFYPSDI | |
| | FYPSDIAVEWESNGQPENN | AVEWESNGQPENNYKTTPP | |
| | YKTTPPVLDSDGSFFLYSKL | VLDSDGSFFLVSKLTVDKS | |
| | TVDKSRWQQGNVFSCSVM | RWQQGNVFSCSVMHEALH | |
| | HEALHNHYTQKSLSLSPG | NRFTQKSLSLSPG | |
| | (SEQ ID NO: 853) | (SEQ ID NO: 854) | |
| DGL568 | QVQLVESGGGLVQAGGSLR | QVQLVESGGGLVRPGGSLR | |
| | LSCAASRSISSFNEMAWYR | LSCAASGFTFSSNGMHWFR | |
| | QAPGEQRELVATIVSTVGFT | QAPGKGLEWVSSIGSGGTSI | |
| | NYADSVKGRFTISRDNAKN | YYADSVKGRFTISRDNAKN | |
| | TVYLQMNSLKAEDTAVYY | TLYLQMNSLKSDDTAVYY | |
| | CNARRISTDYWGQGTQVTV | CSKEVTGATRGQGTQVTVE | |
| | EPKSSDKTHTCPPCPAPEAA | PKSSDKTHTCPPCPAPEAAG | |
| | GAPSVFLFPPKPKDTLMISR | APSVFLFPPKPKDTLMISRT | |
| | TPEVTCVVVDVSHEDPEVK | PEVTCVVVDVSHEDPEVKF | |
| | FNWYVDGVEVHNAKTKPR | NWYVDGVEVHNAKTKPRE | |
| | EEQYNSTYRVVSVLTVLHQ | EQYNSTYRVVSVLTVLHQD | |
| | DWLNGKEYKCKVSNKALP | WLNGKEYKCKVSNKALPA | |
| | APIEKTISKAKGQPREPQVY | PIEKTISKAKGQPREPQVCT | |
| | TLPPCRDELTKNQVSLWCL | LPPSRDELTKNQVSLSCAV | |
| | VKGFYPSDIAVEWESNGQP | KGFYPSDIAVEWESNGQPE | |
| | ENNYKTTPPVLDSDGSFFLY | NNYKTTPPVLDSDGSFFLVS | |
| | SKLTVDKSRWQQGNVFSCS | KLTVDKSRWQQGNVFSCS | |
| | VMHEALHNHYTQKSLSLSP | VMHEALHNRFTQKSLSLSP | |
| | G | G | |
| | (SEQ ID NO: 855) | (SEQ ID NO: 856) | |
| DGL569 | QVQLVESGGGLVQAGGSLR | QVQLVESGGGLVQPGGSLR | |
| | LSCAASRSISSFNEMAWYR | LSCAASGFTFSNYAMSWVR | |
| | QAPGEQRELVATIVSTVGFT | QAPGKGLEWVSAINSDGTS | |
| | NYADSVKGRFTISRDNAKN | TSYADSVKGQFTISRDNAK | |
| | TVYLQMNSLKAEDTAVYY | NTLYLQMNSLKPEDTAVYY | |
| | CNARRISTDYWGQGTQVTV | CTKWEGDTDSDYKFKDYW | |
| | EPKSSDKTHTCPPCPAPEAA | GQGTQVTVEPKSSDKTHTC | |
| | GAPSVFLFPPKPKDTLMISR | PPCPAPEAAGAPSVFLFPPK | |
| | TPEVTCVVVDVSHEDPEVK | PKDTLMISRTPEVTCVVVD | |
| | FNWYVDGVEVHNAKTKPR | VSHEDPEVKFNWYVDGVE | |
| | EEQYNSTYRVVSVLTVLHQ | VHNAKTKPREEQYNSTYRV | |
| | DWLNGKEYKCKVSNKALP | VSVLTVLHQDWLNGKEYK | |
| | APIEKTISKAKGQPREPQVY | CKVSNKALPAPIEKTISKAK | |
| | TLPPCRDELTKNQVSLWCL | GQPREPQVCTLPPSRDELTK | |
| | VKGFYPSDIAVEWESNGQP | NQVSLSCAVKGFYPSDIAV | |
| | ENNYKTTPPVLDSDGSFFLY | EWESNGQPENNYKTTPPVL | |
| | SKLTVDKSRWQQGNVFSCS | DSDGSFFLVSKLTVDKSRW | |
| | VMHEALHNHYTQKSLSLSP | QQGNVFSCSVMHEALHNRF | |
| | G | TQKSLSLSPG | |
| | (SEQ ID NO: 857) | (SEQ ID NO: 858) | |
| DGL570 | QVQLVESGGGLVQAGGSLR | QVQLVESGGGLVQPGGSLR | |
| | LSCAASRSISSFNEMAWYR | LSCAASGFTFSSKAMHWFR | |
| | QAPGEQRELVATIVSTVGFT | QAPGKGLEWVSSINSGGAIT | |
| | NYADSVKGRFTISRDNAKN | YYKDSVKGRFTVSRDNAK | |
| | TVYLQMNSLKAEDTAVYY | NILYLEMNSLKPEDTALYY | |
| | CNARRISTDYWGQGTQVTV | CATDTDGRTRGQGTQVTVE | |
| | EPKSSDKTHTCPPCPAPEAA | PKSSDKTHTCPPCPAPEAAG | |
| | GAPSVFLFPPKPKDTLMISR | APSVFLFPPKPKDTLMISRT | |
| | TPEVTCVVVDVSHEDPEVK | PEVTCVVVDVSHEDPEVKF | |
| | FNWYVDGVEVHNAKTKPR | NWYVDGVEVHNAKTKPRE | |
| | EEQYNSTYRVVSVLTVLHQ | EQYNSTYRVVSVLTVLHQD | |
| | DWLNGKEYKCKVSNKALP | WLNGKEYKCKVSNKALPA | |
| | APIEKTISKAKGQPREPQVY | PIEKTISKAKGQPREPQVCT | |
| | TLPPCRDELTKNQVSLWCL | LPPSRDELTKNQVSLSCAV | |
| | VKGFYPSDIAVEWESNGQP | KGFYPSDIAVEWESNGQPE | |
| | ENNYKTTPPVLDSDGSFFLY | NNYKTTPPVLDSDGSFFLVS | |
| | SKLTVDKSRWQQGNVFSCS | KLTVDKSRWQQGNVFSCS | |
| | VMHEALHNHYTQKSLSLSP | VMHEALHNRFTQKSLSLSP | |
| | G | G | |
| | (SEQ ID NO: 859) | (SEQ ID NO: 860) | |
| DGL571 | QVQLVESGGGLVQAGGSLR | QVQLVESGGGLVQPGGSLR | |
| | LSCAASRSISSFNEMAWYR | LSCAASGFTFSNYAMSWIR | |
| | QAPGEQRELVATIVSTVGFT | QGPERGFEWVSRINSGGGT | |
| | NYADSVKGRFTISRDNAKN | TSYADSVKGRFTISRDNAK | |
| | TVYLQMNSLKAEDTAVYY | NTLYLQMNSLKPEDTAVYY | |
| | CNARRISTDYWGQGTQVTV | CAKHSHPDYSSNYYRPGQG | |
| | EPKSSDKTHTCPPCPAPEAA | TQVTVEPKSSDKTHTCPPCP | |
| | GAPSVFLFPPKPKDTLMISR | APEAAGAPSVFLFPPKPKDT | |
| | TPEVTCVVVDVSHEDPEVK | LMISRTPEVTCVVVDVSHE | |
| | FNWYVDGVEVHNAKTKPR | DPEVKFNWYVDGVEVHNA | |
| | EEQYNSTYRVVSVLTVLHQ | KTKPREEQYNSTYRVVSVL | |
| | DWLNGKEYKCKVSNKALP | TVLHQDWLNGKEYKCKVS | |
| | APIEKTISKAKGQPREPQVY | NKALPAPIEKTISKAKGQPR | |
| | TLPPCRDELTKNQVSLWCL | EPQVCTLPPSRDELTKNQVS | |
| | VKGFYPSDIAVEWESNGQP | LSCAVKGFYPSDIAVEWES | |
| | ENNYKTTPPVLDSDGSFFLY | NGQPENNYKTTPPVLDSDG | |
| | SKLTVDKSRWQQGNVFSCS | SFFLVSKLTVDKSRWQQGN | |
| | VMHEALHNHYTQKSLSLSP | VFSCSVMHEALHNRFTQKS | |
| | G | LSLSPG | |
| | (SEQ ID NO: 861) | (SEQ ID NO: 862) | |
| DGL572 | QVQLVESGGGLVQAGGSLR | QVQLVESGGGLVQPGGSLR | |
| | LSCAASRSISSFNEMAWYR | LSCAASGFTFEPYDMSWFR | |
| | QAPGEQRELVATIVSTVGFT | QAPGKGPEWVSAIDKGGG | |
| | NYADSVKGRFTISRDNAKN | AGATYYADSVKGRFTISRD | |
| | TVYLQMNSLKAEDTAVYY | NAKKTLYLQMKSLKPEDTA | |
| | CNARRISTDYWGQGTQVTV | VYYCAITAGPYSDSTPRVA | |
| | EPKSSDKTHTCPPCPAPEAA | YRGEGTQVTVEPKSSDKTH | |
| | GAPSVFLFPPKPKDTLMISR | TCPPCPAPEAAGAPSVFLFP | |
| | TPEVTCVVVDVSHEDPEVK | PKPKDTLMISRTPEVTCVVV | |
| | FNWYVDGVEVHNAKTKPR | DVSHEDPEVKFNWYVDGV | |
| | EEQYNSTYRVVSVLTVLHQ | EVHNAKTKPREEQYNSTYR | |
| | DWLNGKEYKCKVSNKALP | VVSVLTVLHQDWLNGKEY | |
| | APIEKTISKAKGQPREPQVY | KCKVSNKALPAPIEKTISKA | |
| | TLPPCRDELTKNQVSLWCL | KGQPREPQVCTLPPSRDELT | |
| | VKGFYPSDIAVEWESNGQP | KNQVSLSCAVKGFYPSDIA | |
| | ENNYKTTPPVLDSDGSFFLY | VEWESNGQPENNYKTTPPV | |
| | SKLTVDKSRWQQGNVFSCS | LDSDGSFFLVSKLTVDKSR | |
| | VMHEALHNHYTQKSLSLSP | WQQGNVFSCSVMHEALHN | |
| | G | RFTQKSLSLSPG | |
| | (SEQ ID NO: 863) | (SEQ ID NO: 864) | |
| DGL574 | QVQLVESGGGLVQAGGSLR | QVQLVESGGGLVQAGGSLR | |
| | LSCAASRSISSFNEMAWYR | LSCAASGRTFSRYRMGWFR | |
| | QAPGEQRELVATIVSTVGFT | QAPGKEREFVAAITKSGAT | |
| | NYADSVKGRFTISRDNAKN | TYDADSVKGRFTISRDNAK | |
| | TVYLQMNSLKAEDTAVYY | NTLYLQMNSLKPEDTAVYY | |
| | CNARRISTDYWGQGTQVTV | CAASYHSNWWPKNADEYA | |
| | EPKSSDKTHTCPPCPAPEAA | YWGQGTQVTVEPKSSDKT | |
| | GAPSVFLFPPKPKDTLMISR | HTCPPCPAPEAAGAPSVFLF | |
| | TPEVTCVVVDVSHEDPEVK | PPKPKDTLMISRTPEVTCVV | |
| | FNWYVDGVEVHNAKTKPR | VDVSHEDPEVKFNWYVDG | |
| | EEQYNSTYRVVSVLTVLHQ | VEVHNAKTKPREEQYNSTY | |
| | DWLNGKEYKCKVSNKALP | RVVSVLTVLHQDWLNGKE | |
| | APIEKTISKAKGQPREPQVY | YKCKVSNKALPAPIEKTISK | |
| | TLPPCRDELTKNQVSLWCL | AKGQPREPQVCTLPPSRDEL | |
| | VKGFYPSDIAVEWESNGQP | TKNQVSLSCAVKGFYPSDI | |
| | ENNYKTTPPVLDSDGSFFLY | AVEWESNGQPENNYKTTPP | |
| | SKLTVDKSRWQQGNVFSCS | VLDSDGSFFLVSKLTVDKS | |
| | VMHEALHNHYTQKSLSLSP | RWQQGNVFSCSVMHEALH | |
| | G | NRFTQKSLSLSPG | |
| | (SEQ ID NO: 865) | (SEQ ID NO: 866) | |
| DGL575 | QVQLVESGGGLVQAGGSLR | QVQLQESGPGLVKPSQTLS | |
| | LSCAASRSISSFNEMAWYR | LTCTVSGGSITTNYYYWSW | |
| | QAPGEQRELVATIVSTVGFT | IRQPPGKGLEWMGAIAYSG | |
| | NYADSVKGRFTISRDNAKN | STYYSPSLKSRTSISRDTSKN | |
| | TVYLQMNSLKAEDTAVYY | QFTLQLSSVTPEDTAVYYC | |
| | CNARRISTDYWGQGTQVTV | ASGINFYSNYPTLNEYDYW | |
| | EPKSSDKTHTCPPCPAPEAA | GQGTQVTVEPKSSDKTHTC | |
| | GAPSVFLFPPKPKDTLMISR | PPCPAPEAAGAPSVFLFPPK | |
| | TPEVTCVVVDVSHEDPEVK | PKDTLMISRTPEVTCVVVD | |
| | FNWYVDGVEVHNAKTKPR | VSHEDPEVKFNWYVDGVE | |
| | EEQYNSTYRVVSVLTVLHQ | VHNAKTKPREEQYNSTYRV | |
| | DWLNGKEYKCKVSNKALP | VSVLTVLHQDWLNGKEYK | |
| | APIEKTISKAKGQPREPQVY | CKVSNKALPAPIEKTISKAK | |
| | TLPPCRDELTKNQVSLWCL | GQPREPQVCTLPPSRDELTK | |
| | VKGFYPSDIAVEWESNGQP | NQVSLSCAVKGFYPSDIAV | |
| | ENNYKTTPPVLDSDGSFFLY | EWESNGQPENNYKTTPPVL | |
| | SKLTVDKSRWQQGNVFSCS | DSDGSFFLVSKLTVDKSRW | |
| | VMHEALHNHYTQKSLSLSP | QQGNVFSCSVMHEALHNRF | |
| | G | TQKSLSLSPG | |
| | (SEQ ID NO: 867) | (SEQ ID NO: 868) | |
| DGL579 | QVQLVESGGGLVQAGGSLR | QVQLVESGGGLVQAGGSLR | |
| | LSCAASRSISSFNEMAWYR | LSCAASGRTFSSYAMGWFR | |
| | QAPGEQRELVATIVSTVGFT | QAPGKEREFVSAISWSGLST | |
| | NYADSVKGRFTISRDNAKN | YYVESVQGRFTISRDNAKN | |
| | TVYLQMNSLKAEDTAVYY | TVYLQMNSLKPEDTAVYY | |
| | CNARRISTDYWGQGTQVTV | CAASRIYTNYVARSYEYDD | |
| | EPKSSDKTHTCPPCPAPEAA | WGQGTQVTVEPKSSDKTHT | |
| | GAPSVFLFPPKPKDTLMISR | CPPCPAPEAAGAPSVFLFPP | |
| | TPEVTCVVVDVSHEDPEVK | KPKDTLMISRTPEVTCVVV | |
| | FNWYVDGVEVHNAKTKPR | DVSHEDPEVKFNWYVDGV | |
| | EEQYNSTYRVVSVLTVLHQ | EVHNAKTKPREEQYNSTYR | |
| | DWLNGKEYKCKVSNKALP | VVSVLTVLHQDWLNGKEY | |
| | APIEKTISKAKGQPREPQVY | KCKVSNKALPAPIEKTISKA | |
| | TLPPCRDELTKNQVSLWCL | KGQPREPQVCTLPPSRDELT | |
| | VKGFYPSDIAVEWESNGQP | KNQVSLSCAVKGFYPSDIA | |
| | ENNYKTTPPVLDSDGSFFLY | VEWESNGQPENNYKTTPPV | |
| | SKLTVDKSRWQQGNVFSCS | LDSDGSFFLVSKLTVDKSR | |
| | VMHEALHNHYTQKSLSLSP | WQQGNVFSCSVMHEALHN | |
| | G | RFTQKSLSLSPG | |
| | (SEQ ID NO: 869) | (SEQ ID NO: 870) | |
| DGL584 | QVQLQESGGGLVQAGGSLR | QVQLVESGGGLVQPGGSLR | |
| | LSCAASGSIASINAMGWYR | LSCAASGFTFSSKAMHWFR | |
| | QAPGKQRDFVARIQLSGST | QAPGKGLEWVSSINSGGAIT | |
| | NYAHSVRGRFTISRDNAKN | YYKDSVKGRFTVSRDNAK | |
| | TVYLQMNNLKPEDTAVYV | NILYLEMNSLKPEDTALYY | |
| | CAASNTGSLYGLRNYWGQ | CATDTDGRTRGQGTQVTVE | |
| | GTQVTVEPKSSDKTHTCPP | PKSSDKTHTCPPCPAPEAAG | |
| | CPAPEAAGAPSVFLFPPKPK | APSVFLFPPKPKDTLMISRT | |
| | DTLMISRTPEVTCVVVDVS | PEVTCVVVDVSHEDPEVKF | |
| | HEDPEVKFNWYVDGVEVH | NWYVDGVEVHNAKTKPRE | |
| | NAKTKPREEQYNSTYRVVS | EQYNSTYRVVSVLTVLHQD | |
| | VLTVLHQDWLNGKEYKCK | WLNGKEYKCKVSNKALPA | |
| | VSNKALPAPIEKTISKAKGQ | PIEKTISKAKGQPREPQVCT | |
| | PREPQVYTLPPCRDELTKN | LPPSRDELTKNQVSLSCAV | |
| | QVSLWCLVKGFYPSDIAVE | KGFYPSDIAVEWESNGQPE | |
| | WESNGQPENNYKTTPPVLD | NNYKTTPPVLDSDGSFFLVS | |
| | SDGSFFLYSKLTVDKSRWQ | KLTVDKSRWQQGNVFSCS | |
| | QGNVFSCSVMHEALHNHY | VMHEALHNRFTQKSLSLSP | |
| | TQKSLSLSPG | G | |
| | (SEQ ID NO: 871) | (SEQ ID NO: 872) | |
| DGL585 | QVQLVESGGGLVQAGGSLR | QVQLVESGGGLVRPGGSLR | |
| | LSCAASGRTFSSYVMGWFR | LSCAASGFTFSSNGMHWFR | |
| | QAPGQEREFVAAIMRNGIM | QAPGKGLEWVSSIGSGGTSI | |
| | TYYADSVKGRFTISRDNAK | YYADSVKGRFTISRDNAKN | |
| | NTVYLQMNTLKPDDTAVY | TLYLQMNSLKSDDTAVYY | |
| | YCAAASRVGVSISQSSSYTS | CSKEVTGATRGQGTQVTVE | |
| | WGQGTQVTVEPKSSDKTHT | PKSSDKTHTCPPCPAPEAAG | |
| | CPPCPAPEAAGAPSVFLFPP | APSVFLFPPKPKDTLMISRT | |
| | KPKDTLMISRTPEVTCVVV | PEVTCVVVDVSHEDPEVKF | |
| | DVSHEDPEVKFNWYVDGV | NWYVDGVEVHNAKTKPRE | |
| | EVHNAKTKPREEQYNSTYR | EQYNSTYRVVSVLTVLHQD | |
| | VVSVLTVLHQDWLNGKEY | WLNGKEYKCKVSNKALPA | |
| | KCKVSNKALPAPIEKTISKA | PIEKTISKAKGQPREPQVCT | |
| | KGQPREPQVYTLPPCRDEL | LPPSRDELTKNQVSLSCAV | |
| | TKNQVSLWCLVKGFYPSDI | KGFYPSDIAVEWESNGQPE | |
| | AVEWESNGQPENNYKTTPP | NNYKTTPPVLDSDGSFFLVS | |
| | VLDSDGSFFLYSKLTVDKS | KLTVDKSRWQQGNVFSCS | |
| | RWQQGNVFSCSVMHEALH | VMHEALHNRFTQKSLSLSP | |
| | NHYTQKSLSLSPG | G | |
| | (SEQ ID NO: 873) | (SEQ ID NO: 874) | |
| DGL586 | QVQLVESGGGLVQAGGSLR | QVQLVESGGGLVQPGGSLR | |
| | LSCAASGRTFSSYVMGWFR | LSCAASGFTFSNYAMSWVR | |
| | QAPGQEREFVAAIMRNGIM | QAPGKGLEWVSAINSDGTS | |
| | TYYADSVKGRFTISRDNAK | TSYADSVKGQFTISRDNAK | |
| | NTVYLQMNTLKPDDTAVY | NTLYLQMNSLKPEDTAVYY | |
| | YCAAASRVGVSISQSSSYTS | CTKWEGDTDSDYKFKDYW | |
| | WGQGTQVTVEPKSSDKTHT | GQGTQVTVEPKSSDKTHTC | |
| | CPPCPAPEAAGAPSVFLFPP | PPCPAPEAAGAPSVFLFPPK | |
| | KPKDTLMISRTPEVTCVVV | PKDTLMISRTPEVTCVVVD | |
| | DVSHEDPEVKFNWYVDGV | VSHEDPEVKFNWYVDGVE | |
| | EVHNAKTKPREEQYNSTYR | VHNAKTKPREEQYNSTYRV | |
| | VVSVLTVLHQDWLNGKEY | VSVLTVLHQDWLNGKEYK | |
| | KCKVSNKALPAPIEKTISKA | CKVSNKALPAPIEKTISKAK | |
| | KGQPREPQVYTLPPCRDEL | GQPREPQVCTLPPSRDELTK | |
| | TKNQVSLWCLVKGFYPSDI | NQVSLSCAVKGFYPSDIAV | |
| | AVEWESNGQPENNYKTTPP | EWESNGQPENNYKTTPPVL | |
| | VLDSDGSFFLYSKLTVDKS | DSDGSFFLVSKLTVDKSRW | |
| | RWQQGNVFSCSVMHEALH | QQGNVFSCSVMHEALHNRF | |
| | NHYTQKSLSLSPG | TQKSLSLSPG | |
| | (SEQ ID NO: 875) | (SEQ ID NO: 876) | |
| DGL587 | QVQLVESGGGLVQAGGSLR | QVQLVESGGGLVQPGGSLR | |
| | LSCAASGRTFSSYVMGWFR | LSCAASGFTFSSKAMHWFR | |
| | QAPGQEREFVAAIMRNGIM | QAPGKGLEWVSSINSGGAIT | |
| | TYYADSVKGRFTISRDNAK | YYKDSVKGRFTVSRDNAK | |
| | NTVYLQMNTLKPDDTAVY | NILYLEMNSLKPEDTALYY | |
| | YCAAASRVGVSISQSSSYTS | CATDTDGRTRGQGTQVTVE | |
| | WGQGTQVTVEPKSSDKTHT | PKSSDKTHTCPPCPAPEAAG | |
| | CPPCPAPEAAGAPSVFLFPP | APSVFLFPPKPKDTLMISRT | |
| | KPKDTLMISRTPEVTCVVV | PEVTCVVVDVSHEDPEVKF | |
| | DVSHEDPEVKFNWYVDGV | NWYVDGVEVHNAKTKPRE | |
| | EVHNAKTKPREEQYNSTYR | EQYNSTYRVVSVLTVLHQD | |
| | VVSVLTVLHQDWLNGKEY | WLNGKEYKCKVSNKALPA | |
| | KCKVSNKALPAPIEKTISKA | PIEKTISKAKGQPREPQVCT | |
| | KGQPREPQVYTLPPCRDEL | LPPSRDELTKNQVSLSCAV | |
| | TKNQVSLWCLVKGFYPSDI | KGFYPSDIAVEWESNGQPE | |
| | AVEWESNGQPENNYKTTPP | NNYKTTPPVLDSDGSFFLVS | |
| | VLDSDGSFFLYSKLTVDKS | KLTVDKSRWQQGNVFSCS | |
| | RWQQGNVFSCSVMHEALH | VMHEALHNRFTQKSLSLSP | |
| | NHYTQKSLSLSPG | G | |
| | (SEQ ID NO: 877) | (SEQ ID NO: 878) | |
| DGL594 | EVQLVESGGGLVQAGGSLR | QVQLVESGGGLVQPGGSLR | |
| | LSCAASGSKFSFNAMGWYR | LSCAASGFTFSSKAMHWFR | |
| | QTPGKQRELVAIIINGDMIN | QAPGKGLEWVSSINSGGAIT | |
| | YADSVKGRFTISRDNAKNT | YYKDSVKGRFTVSRDNAK | |
| | MYLQMNSLKPEDTAVYYC | NILYLEMNSLKPEDTALYY | |
| | KMTRYVYDYWGQGTQVT | CATDTDGRTRGQGTQVTVE | |
| | VEPKSSDKTHTCPPCPAPEA | PKSSDKTHTCPPCPAPEAAG | |
| | AGAPSVFLFPPKPKDTLMIS | APSVFLFPPKPKDTLMISRT | |
| | RTPEVTCVVVDVSHEDPEV | PEVTCVVVDVSHEDPEVKF | |
| | KFNWYVDGVEVHNAKTKP | NWYVDGVEVHNAKTKPRE | |
| | REEQYNSTYRVVSVLTVLH | EQYNSTYRVVSVLTVLHQD | |
| | QDWLNGKEYKCKVSNKAL | WLNGKEYKCKVSNKALPA | |
| | PAPIEKTISKAKGQPREPQV | PIEKTISKAKGQPREPQVCT | |
| | YTLPPCRDELTKNQVSLWC | LPPSRDELTKNQVSLSCAV | |
| | LVKGFYPSDIAVEWESNGQ | KGFYPSDIAVEWESNGQPE | |
| | PENNYKTTPPVLDSDGSFFL | NNYKTTPPVLDSDGSFFLVS | |
| | YSKLTVDKSRWQQGNVFS | KLTVDKSRWQQGNVFSCS | |
| | CSVMHEALHNHYTQKSLSL | VMHEALHNRFTQKSLSLSP | |
| | SPG | G | |
| | (SEQ ID NO: 879) | (SEQ ID NO: 880) | |
| DGL599 | EVQLVESGGGLVQAGGSLR | QVQLQESGPGLVKPSQTLS | |
| | LSCAASGSKFSFNAMGWYR | LTCTVSGGSITTNYYYWSW | |
| | QTPGKQRELVAIIINGDMIN | IRQPPGKGLEWMGAIAYSG | |
| | YADSVKGRFTISRDNAKNT | STYYSPSLKSRTSISRDTSKN | |
| | MYLQMNSLKPEDTAVYYC | QFTLQLSSVTPEDTAVYYC | |
| | KMTRYVYDYWGQGTQVT | ASGINFYSNYPTLNEYDYW | |
| | VEPKSSDKTHTCPPCPAPEA | GQGTQVTVEPKSSDKTHTC | |
| | AGAPSVFLFPPKPKDTLMIS | PPCPAPEAAGAPSVFLFPPK | |
| | RTPEVTCVVVDVSHEDPEV | PKDTLMISRTPEVTCVVVD | |
| | KFNWYVDGVEVHNAKTKP | VSHEDPEVKFNWYVDGVE | |
| | REEQYNSTYRVVSVLTVLH | VHNAKTKPREEQYNSTYRV | |
| | QDWLNGKEYKCKVSNKAL | VSVLTVLHQDWLNGKEYK | |
| | PAPIEKTISKAKGQPREPQV | CKVSNKALPAPIEKTISKAK | |
| | YTLPPCRDELTKNQVSLWC | GQPREPQVCTLPPSRDELTK | |
| | LVKGFYPSDIAVEWESNGQ | NQVSLSCAVKGFYPSDIAV | |
| | PENNYKTTPPVLDSDGSFFL | EWESNGQPENNYKTTPPVL | |
| | YSKLTVDKSRWQQGNVFS | DSDGSFFLVSKLTVDKSRW | |
| | CSVMHEALHNHYTQKSLSL | QQGNVFSCSVMHEALHNRF | |
| | SPG | TQKSLSLSPG | |
| | (SEQ ID NO: 881) | (SEQ ID NO: 882) | |
| DGL601 | QVQLVQSGAEVKKPGASV | QVQLVESGGGLVQPGGSLR | SSELTQDPAVSVA |
| | RVSCKASGYTFTSYYIHWV | LSCAASGFTFSNYAMSWVR | LGQTVRITCQGDS |
| | RQAPGQGLEWMGIINPSGG | QAPGKGLEWVSAINSDGTS | LSSYYAGWYQQ |
| | STSYAQKFQGRVTMTRDTS | TSYADSVKGQFTISRDNAK | KPGQAPVLVIYG |
| | TTTVYMELSSLRSEDTAVY | NTLYLQMNSLKPEDTAVYY | KNNRPSGIPDRES |
| | YCAKSNWAYAFDIWGQGT | CTKWEGDTDSDYKFKDYW | GSSSEDTASLTIT |
| | MVTVSSASTKGPSVFPLAPS | GQGTQVTVEPKSSDKTHTC | GAQAEDEADYYC |
| | SKSTSGGTAALGCLVKDYF | PPCPAPEAAGAPSVFLFPPK | NSRDSSDNHLVF |
| | PEPVTVSWNSGALTSGVHT | PKDTLMISRTPEVTCVVVD | GGGTKLTVLGQP |
| | FPAVLQSSGLYSLSSVVTVP | VSHEDPEVKFNWYVDGVE | KAAPSVTLFPPSS |
| | SSSLGTQTYICNVNHKPSNT | VHNAKTKPREEQYNSTYRV | EELQANKATLVC |
| | KVDKKVEPKSCDKTHTCPP | VSVLTVLHQDWLNGKEYK | LISDFYPGAVTVA |
| | CPAPEAAGAPSVFLFPPKPK | CKVSNKALPAPIEKTISKAK | WKADSSPVKAGV |
| | DTLMISRTPEVTCVVVDVS | GQPREPQVCTLPPSRDELTK | ETTTPSKQSNNK |
| | HEDPEVKFNWYVDGVEVH | NQVSLSCAVKGFYPSDIAV | YAASSYLSLTPEQ |
| | NAKTKPREEQYNSTYRVVS | EWESNGQPENNYKTTPPVL | WKSHRSYSCQVT |
| | VLTVLHQDWLNGKEYKCK | DSDGSFFLVSKLTVDKSRW | HEGSTVEKTVAP |
| | VSNKALPAPIEKTISKAKGQ | QQGNVFSCSVMHEALHNRF | TECS |
| | PREPQVYTLPPCRDELTKN | TQKSLSLSPG | (SEQ ID |
| | QVSLWCLVKGFYPSDIAVE | (SEQ ID NO: 883) | NO: 884) |
| | WESNGQPENNYKTTPPVLD | | |
| | SDGSFFLYSKLTVDKSRWQ | | |
| | QGNVFSCSVMHEALHNHY | | |
| | TQKSLSLSPG | | |
| | (SEQ ID NO: 909) | | |
| DGL603 | EVQLVESGGGLVQPGGSLR | QVQLVESGGGLVQPGGSLR | |
| | LSCAASGFTFQNYAMSWLR | LSCAASGFTFSNYAMSWVR | |
| | KAPGEGLEWVSVINSGGGS | QAPGKGLEWVSAINSDGTS | |
| | TLYADSVKGRFTISRDNAK | TSYADSVKGQFTISRDNAK | |
| | NTLYLQMHSLKSEDTAVYF | NTLYLQMNSLKPEDTAVYY | |
| | CAKRRDDSTFGSLLYTHRG | CTKWEGDTDSDYKFKDYW | |
| | QGTQVTVEPKSSDKTHTCP | GQGTQVTVEPKSSDKTHTC | |
| | PCPAPEAAGAPSVFLFPPKP | PPCPAPEAAGAPSVFLFPPK | |
| | KDTLMISRTPEVTCVVVDV | PKDTLMISRTPEVTCVVVD | |
| | SHEDPEVKFNWYVDGVEV | VSHEDPEVKFNWYVDGVE | |
| | HNAKTKPREEQYNSTYRVV | VHNAKTKPREEQYNSTYRV | |
| | SVLTVLHQDWLNGKEYKC | VSVLTVLHQDWLNGKEYK | |
| | KVSNKALPAPIEKTISKAKG | CKVSNKALPAPIEKTISKAK | |
| | QPREPQVYTLPPCRDELTK | GQPREPQVCTLPPSRDELTK | |
| | NQVSLWCLVKGFYPSDIAV | NQVSLSCAVKGFYPSDIAV | |
| | EWESNGQPENNYKTTPPVL | EWESNGQPENNYKTTPPVL | |
| | DSDGSFFLYSKLTVDKSRW | DSDGSFFLVSKLTVDKSRW | |
| | QQGNVFSCSVMHEALHNH | QQGNVFSCSVMHEALHNRF | |
| | YTQKSLSLSPG | TQKSLSLSPG | |
| | (SEQ ID NO: 885) | (SEQ ID NO: 886) | |
| DGL604 | QVQLVESGGGLVQAGGSLR | QLQLVESGGGLVQAGGSLR | |
| | LSCAASRSISSFNEMAWYR | LSCAASGRTFGIHGMGWFR | |
| | QAPGEQRELVATIVSTVGFT | QAPGKEREFAAAIRWSDGT | |
| | NYADSVKGRFTISRDNAKN | TIHADSVKGRLTISRDNAKN | |
| | TVYLQMNSLKAEDTAVYY | TVYLQMNSLKPEDTAVYY | |
| | CNARRISTDYWGQGTQVTV | CAATSNPQFGRNWYTVAA | |
| | EPKSSDKTHTCPPCPAPEAA | GVEYGYWGQGTQVTVEPK | |
| | GAPSVFLFPPKPKDTLMISR | SSDKTHTCPPCPAPEAAGAP | |
| | TPEVTCVVVDVSHEDPEVK | SVFLFPPKPKDTLMISRTPE | |
| | FNWYVDGVEVHNAKTKPR | VTCVVVDVSHEDPEVKEN | |
| | EEQYNSTYRVVSVLTVLHQ | WYVDGVEVHNAKTKPREE | |
| | DWLNGKEYKCKVSNKALP | QYNSTYRVVSVLTVLHQD | |
| | APIEKTISKAKGQPREPQVY | WLNGKEYKCKVSNKALPA | |
| | TLPPCRDELTKNQVSLWCL | PIEKTISKAKGQPREPQVCT | |
| | VKGFYPSDIAVEWESNGQP | LPPSRDELTKNQVSLSCAV | |
| | ENNYKTTPPVLDSDGSFFLY | KGFYPSDIAVEWESNGQPE | |
| | SKLTVDKSRWQQGNVFSCS | NNYKTTPPVLDSDGSFFLVS | |
| | VMHEALHNHYTQKSLSLSP | KLTVDKSRWQQGNVFSCS | |
| | G | VMHEALHNRFTQKSLSLSP | |
| | (SEQ ID NO: 887) | G | |
| | | (SEQ ID NO: 888) | |
| DGL605 | QLQLVESGGGLVQPGGSLR | QVQLVESGGGLVQPGGSLR | |
| | LSCAASGFTFSSYGMSWVR | LSCAASGFTFEPYDMSWFR | |
| | QAPGKGLEWVSYISAGGGT | QAPGKGPEWVSAIDKGGG | |
| | TTYVDFVKGRFTISRDNAK | AGATYYADSVKGRFTISRD | |
| | NMLYLQMNSLKPEDTAVY | NAKKTLYLQMKSLKPEDTA | |
| | YCVHAADGSRGQGTQVTV | VYYCAITAGPYSDSTPRVA | |
| | EPKSSDKTHTCPPCPAPEAA | YRGEGTQVTVEPKSSDKTH | |
| | GAPSVFLFPPKPKDTLMISR | TCPPCPAPEAAGAPSVFLFP | |
| | TPEVTCVVVDVSHEDPEVK | PKPKDTLMISRTPEVTCVVV | |
| | FNWYVDGVEVHNAKTKPR | DVSHEDPEVKFNWYVDGV | |
| | EEQYNSTYRVVSVLTVLHQ | EVHNAKTKPREEQYNSTYR | |
| | DWLNGKEYKCKVSNKALP | VVSVLTVLHQDWLNGKEY | |
| | APIEKTISKAKGQPREPQVY | KCKVSNKALPAPIEKTISKA | |
| | TLPPCRDELTKNQVSLWCL | KGQPREPQVCTLPPSRDELT | |
| | VKGFYPSDIAVEWESNGQP | KNQVSLSCAVKGFYPSDIA | |
| | ENNYKTTPPVLDSDGSFFLY | VEWESNGQPENNYKTTPPV | |
| | SKLTVDKSRWQQGNVFSCS | LDSDGSFFLVSKLTVDKSR | |
| | VMHEALHNHYTQKSLSLSP | WQQGNVFSCSVMHEALHN | |
| | G | RFTQKSLSLSPG | |
| | (SEQ ID NO: 890) | (SEQ ID NO: 891) | |
| DGL606 | QLQLVESGGGLVQPGGSLR | QVQLEESGGGLVQAGGSLR | |
| | LSCAASGFTFSSYGMSWVR | LSCAASGRTFSSYVVGWFR | |
| | QAPGKGLEWVSYISAGGGT | QAPGKEREFVGRISWSGAR | |
| | TTYVDFVKGRFTISRDNAK | TYYADSVEGRFTISRDNAK | |
| | NMLYLQMNSLKPEDTAVY | NTVYLQMNSLKPEDTAVY | |
| | YCVHAADGSRGQGTQVTV | YCAADLKWEVLLSEHYEY | |
| | EPKSSDKTHTCPPCPAPEAA | WGQGTQVTVEPKSSDKTHT | |
| | GAPSVFLFPPKPKDTLMISR | CPPCPAPEAAGAPSVFLFPP | |
| | TPEVTCVVVDVSHEDPEVK | KPKDTLMISRTPEVTCVVV | |
| | FNWYVDGVEVHNAKTKPR | DVSHEDPEVKFNWYVDGV | |
| | EEQYNSTYRVVSVLTVLHQ | EVHNAKTKPREEQYNSTYR | |
| | DWLNGKEYKCKVSNKALP | VVSVLTVLHQDWLNGKEY | |
| | APIEKTISKAKGQPREPQVY | KCKVSNKALPAPIEKTISKA | |
| | TLPPCRDELTKNQVSLWCL | KGQPREPQVCTLPPSRDELT | |
| | VKGFYPSDIAVEWESNGQP | KNQVSLSCAVKGFYPSDIA | |
| | ENNYKTTPPVLDSDGSFFLY | VEWESNGQPENNYKTTPPV | |
| | SKLTVDKSRWQQGNVFSCS | LDSDGSFFLVSKLTVDKSR | |
| | VMHEALHNHYTQKSLSLSP | WQQGNVFSCSVMHEALHN | |
| | G | RFTQKSLSLSPG | |
| | (SEQ ID NO: 892) | (SEQ ID NO: 893) | |
| DGL607 | QVQLVESGGGLMQAGGSL | QVQLVESGGGLVQAGGSL | |
| | RLSCALSRGIFSAYAMGWF | KLSCAASGGTFSSYAMGWF | |
| | RQVQGKEREFVSGISRSGST | RQAPGKEREFVAVIMRSGD | |
| | TDYVDTAKGRFTISRDNAR | TTAYVDSVKGRFTVSRDNA | |
| | DTVYLQMNSLKPEDTAVY | KNTVYLQMNSLKPEDAAV | |
| | YCAAVTGTFPSSTYHNANA | YFCAAGRPVTRTYYHTYTY | |
| | YHHWGQGTQVTVEPKSSD | PHDYDYWGQGTQVTVEPK | |
| | KTHTCPPCPAPEAAGAPSVF | SSDKTHTCPPCPAPEAAGAP | |
| | LFPPKPKDTLMISRTPEVTC | SVFLFPPKPKDTLMISRTPE | |
| | VVVDVSHEDPEVKFNWYV | VTCVVVDVSHEDPEVKEN | |
| | DGVEVHNAKTKPREEQYNS | WYVDGVEVHNAKTKPREE | |
| | TYRVVSVLTVLHQDWLNG | QYNSTYRVVSVLTVLHQD | |
| | KEYKCKVSNKALPAPIEKTI | WLNGKEYKCKVSNKALPA | |
| | SKAKGQPREPQVYTLPPCR | PIEKTISKAKGQPREPQVCT | |
| | DELTKNQVSLWCLVKGFYP | LPPSRDELTKNQVSLSCAV | |
| | SDIAVEWESNGQPENNYKT | KGFYPSDIAVEWESNGQPE | |
| | TPPVLDSDGSFFLYSKLTVD | NNYKTTPPVLDSDGSFFLVS | |
| | KSRWQQGNVFSCSVMHEA | KLTVDKSRWQQGNVFSCS | |
| | LHNHYTQKSLSLSPG | VMHEALHNRFTQKSLSLSP | |
| | (SEQ ID NO: 894) | G | |
| | | (SEQ ID NO: 895) | |
| DGL617 | EVQLVESGGGLVQPGGSLR | QLQLVESGGGLVQAGGSLR | |
| | LSCAASGFTLDYYAIGWFR | LSCAASGLTFSGYTVGWFR | |
| | QAPGKEREGVSCISSSDRST | QAPGKEREFVAAIQWSSAY | |
| | WYADSVKGRFTISRDNAKN | TYSEFVKGRFTISRDNAKNT | |
| | TVYLQMHSLKPEDTAVYY | VYLQMSSLKPEDTAVYYCA | |
| | CATPCYSDYDPEGYEYDY | AQRSRTSWYASTAEAYDY | |
| | WGQGTQVTVEPKSSDKTHT | WGQGTQVTVEPKSSDKTHT | |
| | CPPCPAPEAAGAPSVFLFPP | CPPCPAPEAAGAPSVFLFPP | |
| | KPKDTLMISRTPEVTCVVV | KPKDTLMISRTPEVTCVVV | |
| | DVSHEDPEVKFNWYVDGV | DVSHEDPEVKFNWYVDGV | |
| | EVHNAKTKPREEQYNSTYR | EVHNAKTKPREEQYNSTYR | |
| | VVSVLTVLHQDWLNGKEY | VVSVLTVLHQDWLNGKEY | |
| | KCKVSNKALPAPIEKTISKA | KCKVSNKALPAPIEKTISKA | |
| | KGQPREPQVYTLPPCRDEL | KGQPREPQVCTLPPSRDELT | |
| | TKNQVSLWCLVKGFYPSDI | KNQVSLSCAVKGFYPSDIA | |
| | AVEWESNGQPENNYKTTPP | VEWESNGQPENNYKTTPPV | |
| | VLDSDGSFFLYSKLTVDKS | LDSDGSFFLVSKLTVDKSR | |
| | RWQQGNVFSCSVMHEALH | WQQGNVFSCSVMHEALHN | |
| | NHYTQKSLSLSPG | RFTQKSLSLSPG | |
| | (SEQ ID NO: 896) | (SEQ ID NO: 897) | |
| DGL557 | QVQLQESGGGLVQAGGSLR | QVQLVESGGGLVRPGGSLR | |
| | LSCAASGTIFSIDVMAYYRQ | LSCAASGFTFSSNGMHWFR | |
| | APGKQRELVAAISSGGSLN | QAPGKGLEWVSSIGSGGTSI | |
| | YRDSVKGRFTISRDNAKNA | YYADSVKGRFTISRDNAKN | |
| | VYLQMNSLKPDDTAVYYC | TLYLQMNSLKSDDTAVYY | |
| | YANIRTSPVTTRPMGNYWG | CSKEVTGATRGQGTQVTVE | |
| | QGTQVTVEPKSSDKTHTCP | PKSSDKTHTCPPCPAPEAAG | |
| | PCPAPEAAGAPSVFLFPPKP | APSVFLFPPKPKDTLMISRT | |
| | KDTLMISRTPEVTCVVVDV | PEVTCVVVDVSHEDPEVKF | |
| | SHEDPEVKFNWYVDGVEV | NWYVDGVEVHNAKTKPRE | |
| | HNAKTKPREEQYNSTYRVV | EQYNSTYRVVSVLTVLHQD | |
| | SVLTVLHQDWLNGKEYKC | WLNGKEYKCKVSNKALPA | |
| | KVSNKALPAPIEKTISKAKG | PIEKTISKAKGQPREPQVCT | |
| | QPREPQVYTLPPCRDELTK | LPPSRDELTKNQVSLSCAV | |
| | NQVSLWCLVKGFYPSDIAV | KGFYPSDIAVEWESNGQPE | |
| | EWESNGQPENNYKTTPPVL | NNYKTTPPVLDSDGSFFLVS | |
| | DSDGSFFLYSKLTVDKSRW | KLTVDKSRWQQGNVFSCS | |
| | QQGNVFSCSVMHEALHNH | VMHEALHNRFTQKSLSLSP | |
| | YTQKSLSLSPG | G | |
| | (SEQ ID NO: 898) | (SEQ ID NO: 899) | |
| DGL559 | QVQLQESGGGLVQAGGSLR | QVQLVESGGGLVQPGGSLR | |
| | LSCAASGTIFSIDVMAYYRQ | LSCAASGFTFSSKAMHWFR | |
| | APGKQRELVAAISSGGSLN | QAPGKGLEWVSSINSGGAIT | |
| | YRDSVKGRFTISRDNAKNA | YYKDSVKGRFTVSRDNAK | |
| | VYLQMNSLKPDDTAVYYC | NILYLEMNSLKPEDTALYY | |
| | YANIRTSPVTTRPMGNYWG | CATDTDGRTRGQGTQVTVE | |
| | QGTQVTVEPKSSDKTHTCP | PKSSDKTHTCPPCPAPEAAG | |
| | PCPAPEAAGAPSVFLFPPKP | APSVFLFPPKPKDTLMISRT | |
| | KDTLMISRTPEVTCVVVDV | PEVTCVVVDVSHEDPEVKF | |
| | SHEDPEVKFNWYVDGVEV | NWYVDGVEVHNAKTKPRE | |
| | HNAKTKPREEQYNSTYRVV | EQYNSTYRVVSVLTVLHQD | |
| | SVLTVLHQDWLNGKEYKC | WLNGKEYKCKVSNKALPA | |
| | KVSNKALPAPIEKTISKAKG | PIEKTISKAKGQPREPQVCT | |
| | QPREPQVYTLPPCRDELTK | LPPSRDELTKNQVSLSCAV | |
| | NQVSLWCLVKGFYPSDIAV | KGFYPSDIAVEWESNGQPE | |
| | EWESNGQPENNYKTTPPVL | NNYKTTPPVLDSDGSFFLVS | |
| | DSDGSFFLYSKLTVDKSRW | KLTVDKSRWQQGNVFSCS | |
| | QQGNVFSCSVMHEALHNH | VMHEALHNRFTQKSLSLSP | |
| | YTQKSLSLSPG | G | |
| | (SEQ ID NO: 900) | (SEQ ID NO: 901) | |
| DGL562 | QVQLQESGGGLVQAGGSLR | QVQLVESGGGLVQAGGSLR | |
| | LSCAASGTIFSIDVMAYYRQ | LSCAASGRTFSRYRMGWFR | |
| | APGKQRELVAAISSGGSLN | QAPGKEREFVAAITKSGAT | |
| | YRDSVKGRFTISRDNAKNA | TYDADSVKGRFTISRDNAK | |
| | VYLQMNSLKPDDTAVYYC | NTLYLQMNSLKPEDTAVYY | |
| | YANIRTSPVTTRPMGNYWG | CAASYHSNWWPKNADEYA | |
| | QGTQVTVEPKSSDKTHTCP | YWGQGTQVTVEPKSSDKT | |
| | PCPAPEAAGAPSVFLFPPKP | HTCPPCPAPEAAGAPSVFLF | |
| | KDTLMISRTPEVTCVVVDV | PPKPKDTLMISRTPEVTCVV | |
| | SHEDPEVKFNWYVDGVEV | VDVSHEDPEVKFNWYVDG | |
| | HNAKTKPREEQYNSTYRVV | VEVHNAKTKPREEQYNSTY | |
| | SVLTVLHQDWLNGKEYKC | RVVSVLTVLHQDWLNGKE | |
| | KVSNKALPAPIEKTISKAKG | YKCKVSNKALPAPIEKTISK | |
| | QPREPQVYTLPPCRDELTK | AKGQPREPQVCTLPPSRDEL | |
| | NQVSLWCLVKGFYPSDIAV | TKNQVSLSCAVKGFYPSDI | |
| | EWESNGQPENNYKTTPPVL | AVEWESNGQPENNYKTTPP | |
| | DSDGSFFLYSKLTVDKSRW | VLDSDGSFFLVSKLTVDKS | |
| | QQGNVFSCSVMHEALHNH | RWQQGNVFSCSVMHEALH | |
| | YTQKSLSLSPG | NRFTQKSLSLSPG | |
| | (SEQ ID NO: 902) | (SEQ ID NO: 903) | |
| DGL577 | QVQLVESGGGLVQAGGSLR | QVQLEESGGGLVQAGGSLR | |
| | LSCAASRSISSFNEMAWYR | LSCAASGRTFSSYVVGWFR | |
| | QAPGEQRELVATIVSTVGFT | QAPGKEREFVGRISWSGAR | |
| | NYADSVKGRFTISRDNAKN | TYYADSVEGRFTISRDNAK | |
| | TVYLQMNSLKAEDTAVYY | NTVYLQMNSLKPEDTAVY | |
| | CNARRISTDYWGQGTQVTV | YCAADLKWEVLLSEHYEY | |
| | EPKSSDKTHTCPPCPAPEAA | WGQGTQVTVEPKSSDKTHT | |
| | GAPSVFLFPPKPKDTLMISR | CPPCPAPEAAGAPSVFLFPP | |
| | TPEVTCVVVDVSHEDPEVK | KPKDTLMISRTPEVTCVVV | |
| | FNWYVDGVEVHNAKTKPR | DVSHEDPEVKFNWYVDGV | |
| | EEQYNSTYRVVSVLTVLHQ | EVHNAKTKPREEQYNSTYR | |
| | DWLNGKEYKCKVSNKALP | VVSVLTVLHQDWLNGKEY | |
| | APIEKTISKAKGQPREPQVY | KCKVSNKALPAPIEKTISKA | |
| | TLPPCRDELTKNQVSLWCL | KGQPREPQVCTLPPSRDELT | |
| | VKGFYPSDIAVEWESNGQP | KNQVSLSCAVKGFYPSDIA | |
| | ENNYKTTPPVLDSDGSFFLY | VEWESNGQPENNYKTTPPV | |
| | SKLTVDKSRWQQGNVFSCS | LDSDGSFFLVSKLTVDKSR | |
| | VMHEALHNHYTQKSLSLSP | WQQGNVFSCSVMHEALHN | |
| | G | RFTQKSLSLSP | |
| | (SEQ ID NO: 904) | (SEQ ID NO: 905) | |
| DGL616 | EVQLVESGGGLVQAGGSLR | QLQLVESGGGLVQAGGSLR | |
| | LSCAASGLTFRNYAMAWF | LSCAASGLTFSGYTVGWFR | |
| | RQAPGKEREFVAGITWGGG | QAPGKEREFVAAIQWSSAY | |
| | LTHYVDSVKGRFTISRDNG | TYSEFVKGRFTISRDNAKNT | |
| | KNTVYLQMNSLKSEDTAV | VYLQMSSLKPEDTAVYYCA | |
| | YYCAGKTQGSTWYHLTPD | AQRSRTSWYASTAEAYDY | |
| | GYDYWGQGTQVTVEPKSS | WGQGTQVTVEPKSSDKTHT | |
| | DKTHTCPPCPAPEAAGAPS | CPPCPAPEAAGAPSVFLFPP | |
| | VFLFPPKPKDTLMISRTPEV | KPKDTLMISRTPEVTCVVV | |
| | TCVVVDVSHEDPEVKFNW | DVSHEDPEVKFNWYVDGV | |
| | YVDGVEVHNAKTKPREEQ | EVHNAKTKPREEQYNSTYR | |
| | YNSTYRVVSVLTVLHQDW | VVSVLTVLHQDWLNGKEY | |
| | LNGKEYKCKVSNKALPAPI | KCKVSNKALPAPIEKTISKA | |
| | EKTISKAKGQPREPQVYTLP | KGQPREPQVCTLPPSRDELT | |
| | PCRDELTKNQVSLWCLVKG | KNQVSLSCAVKGFYPSDIA | |
| | FYPSDIAVEWESNGQPENN | VEWESNGQPENNYKTTPPV | |
| | YKTTPPVLDSDGSFFLYSKL | LDSDGSFFLVSKLTVDKSR | |
| | TVDKSRWQQGNVFSCSVM | WQQGNVFSCSVMHEALHN | |
| | HEALHNHYTQKSLSLSPG | RFTQKSLSLSPG | |
| | (SEQ ID NO: 906) | (SEQ ID NO: 907) | |

In some embodiments, the multi-specific binding protein is a bivalent binding protein (e.g., comprises two antigen binding domains). In some embodiments, the multi-specific binding protein comprises more than two antigen binding domains. In some embodiments, the multi-specific binding protein is a trivalent binding protein (e.g., comprises three antigen binding domains). In some embodiments, the multi-specific binding protein is a tetravalent binding protein (e.g., comprises four antigen binding domains). In some embodiments, the at least first and second binding domains are two different polypeptides. In some embodiments, the at least first and second binding domains are part of the same polypeptide.

In certain exemplary embodiments, the multi-specific binding proteins of the disclosure are agonistic to the IL-12R signaling pathway, i.e., they are not antagonistic to the IL-12R pathway. In some embodiments, agonism may be measured using an IL-12 potency assay (e.g., HEK-Blue™ IL-12 potency assay (InVivogen)). In this assay, HEK-Blue™ IL-12 cells are generated by stably transfecting HEK293-derived cells with the genes encoding IL-12Rβ1 and IL-12Rβ2.

The HEK-Blue™ IL-12 cells also express a STAT4-inducible secreted embryonic alkaline phosphatase (SEAP) reporter gene. The binding of bispecific antibodies to the heterodimeric IL-12 receptor on the surface of these cells triggers a signaling cascade leading to the activation of STAT4 and the subsequent production of SEAP which can be quantified.

As used herein, the term “inducing proximity” between IL-12Rβ1 and IL-12Rβ2 refers to bringing IL-12Rβ 1 and IL-12Rβ2 together such that the IL-12Rβ 1/IL-12Rβ2 signaling cascade is stimulated. In certain embodiments, the proximity induced by the multispecific binding proteins of the disclosure is the same or similar to the proximity induced when IL-12 brings IL-12Rβ1 and IL-12Rβ2 together. Stimulation of the IL-12Rβ 1/IL-12Rβ2 signaling cascade may be detected through the above recited HEK-Blue™ IL-12 potency assay.

Alternatively, stimulation of the IL-12Rβ1/IL-12Rβ2 signaling cascade may be detected through any of the downstream results of said signaling cascade, including, but not limited to, detection of phosphorylated STAT4, expression of IFN-γ, or expression of any one or more of CXCR3, IL-12Rβ 1, CCL3, and CCL4.

### Ligand Blocking Activity

In certain aspects, the multi-specific binding proteins of the disclosure are capable of binding specifically to IL-12R β1 and IL-12R β2 and induce IL-12 receptor signaling in the presence of IL-12. The ability to induce IL-12 receptor signaling in the presence of IL-12 has therapeutic advantages, in that the multi-specific binding protein may retain activity despite IL-12 in the subject.

Thus, in certain embodiments, the multi-specific binding proteins of the disclosure are capable of inducing IL-12 receptor signaling in the presence of IL-12. In certain embodiments, the induction of IL-12 receptor signaling is detected via a surface plasmon resonance (SPR) assay.

In certain embodiments, the SPR assay comprises the following steps: 1) contacting the first binding moiety and/or the second binding moiety with an extracellular domain (ECD) of one or both of IL-12R β1 and IL-12R β2 and isolated IL-12; and 2) detecting binding of the first binding moiety and/or the second binding moiety with the ECD of one or both of IL-12R β1 and IL-12R β2, wherein detection of binding indicates that the multi-specific binding protein is capable of inducing IL-12 receptor signaling in the presence of IL-12.

In certain embodiments, the multi-specific binding proteins of the disclosure are capable of binding specifically to human IL-12Rβ1 subunit and human IL-12Rβ2 subunit in the presence of IL-12. In certain embodiments, binding specifically to human IL-12Rβ1 subunit and human IL-12Rβ2 subunit is detected via a surface plasmon resonance (SPR) assay.

In certain embodiments, the SPR assay comprises the following steps: 1) contacting the first binding moiety and/or the second binding moiety with an extracellular domain (ECD) of one or both of IL-12R β1 and IL-12R β2 and isolated IL-12; and 2) detecting binding of the first binding moiety and/or the second binding moiety with the ECD of one or both of IL-12R β1 and IL-12R β2, wherein detection of binding indicates that the multi-specific binding protein is capable of binding specifically to human IL-12Rβ1 subunit and human IL-12Rβ2 subunit in the presence of IL-12.

The ligand blocking activity of the multi-specific binding proteins against IL-12Rβ 1 or IL-12Rβ2 can be characterized using procedures well known in the art including but not limited to the following: competition ELISA assay, ligand binding assay with radio-labeled ligand (e.g. saturation binding, Scatchard plot, dose-dependent competition binding assay), ligand binding assay with fluorescently labeled ligand (e.g., fluorescence polarization (FP), fluorescence resonance energy transfer (FRET)), surface plasmon resonance (SPR) ligand competition assay with instrumentation commercially available (e.g., Biocore 8K from Cytiva Life Sciences, Carterra LSA from Carterra Bio), liquid phase ligand binding assay (e.g., real-time polymerase chain reaction (RT-qPCR), and immunoprecipitation), and solid phase ligand binding assays (e.g., multiwell plate assays, on-cell ligand binding assays, on-bead ligand binding assays, on-column ligand binding assays, and filter assays).

### Multispecific Binding Protein Formats

The multi-specific binding proteins of the disclosure may employ at least one modified hinge region. The modified hinge region serves as a linker to connect different domains of the multi-specific binding protein. In certain embodiments, the modified hinge region links a first binding moiety to a second binding moiety. In certain embodiments, the modified hinge region links a first variable heavy chain domain (VH1) to a second variable heavy chain domain (VH2), and/or the modified hinge region links a first variable light chain domain (VL1) linked to a second variable light chain domain (VL2). In another embodiment, the modified hinge region links a first scFv to a second scFv. In certain embodiments, the modified hinge region comprises; i) an upper hinge region of up to 7 amino acids in length or is absent; and ii) a lower hinge region. In certain embodiments, the modified hinge region comprises or consists of an amino acid sequence of PLAP(SEQ ID NO: 911) or PAPNLLGGP(SEQ ID NO:915).

The multi-specific binding proteins have greater agonist activity compared to a multi-specific binding protein that lacks at least one modified hinge region. Agonist activity may be measured using a specific receptor potency assay (e.g., HEK-Blue™ IL-12 cells from InvivoGen that stably express human IL-12 receptor genes and the genes of the IL-12 signaling pathway including a STAT4-inducible SEAP reporter gene, or Pathhunter U2OS dimerization assay (DiscoverX) Potency assays (e.g., Pathhunter) involve a cell line (e.g., U2OS) that expresses the target receptors of interest). The binding of the multi-specific binding proteins to the receptors triggers a signaling cascade leading to the expression of a reporter gene which can be quantified.

In certain embodiments, the multispecific binding protein comprises a dual variable domain format. “Dual variable domain” (“DVD”) binding proteins of the disclosure comprise two or more antigen binding sites and are tetravalent or multivalent binding proteins. The DVDs of the disclosure are multispecific, i.e., capable of binding IL-12Rβ1 and IL-12Rβ2. A DVD binding protein comprising two heavy chain DVD polypeptides and two light chain DVD polypeptides is referred to as a “DVD immunoglobulin” or “DVD-Ig”. Each half of a DVD-Ig comprises a heavy chain DVD polypeptide and a light chain DVD polypeptide, and two or more antigen binding sites. Each binding site comprises a heavy chain variable domain and a light chain variable domain with a total of six CDRs involved in antigen binding per antigen binding site.

A description of the design, expression, and characterization of DVD-Ig molecules is provided in PCT Publication No. WO 2007/024715; U.S. Pat. No. 7,612,181; and Wu et al., Nature Biotechnol., 25: 1290-1297 (2007). An example of such DVD-Ig molecules comprises a heavy chain that comprises the structural formula VD1-(X1)n-VD2-C-(X2)n, wherein VD1 is a first heavy chain variable domain, VD2 is a second heavy chain variable domain, C is a heavy chain constant domain, X1 is a linker with the proviso that it is not CH1, X2 is an Fc region, and n is 0 or 1; and a light chain that comprises the structural formula VD1-(X1)n-VD2-C-(X2)n, wherein VD1 is a first light chain variable domain, VD2 is a second light chain variable domain, C is a light chain constant domain, X1 is a linker with the proviso that it is not CH1, and X2 does not comprise an Fc region; and n is 0 or 1. Such a DVD-Ig may comprise two such heavy chains and two such light chains, wherein each chain comprises variable domains linked in tandem without an intervening constant region between variable regions, wherein a heavy chain and a light chain associate to form tandem functional antigen binding sites, and a pair of heavy and light chains may associate with another pair of heavy and light chains to form a tetrameric binding protein with four functional antigen binding sites. In another example, a DVD-Ig molecule may comprise heavy and light chains that each comprise three variable domains (VD1, VD2, VD3) linked in tandem without an intervening constant region between variable domains, wherein a pair of heavy and light chains may associate to form three antigen binding sites, and wherein a pair of heavy and light chains may associate with another pair of heavy and light chains to form a tetrameric binding protein with six antigen binding sites.

In an embodiment, the disclosure provides a binding protein comprising first and second polypeptide chains, wherein said first polypeptide chain comprises a first VD1-(X1)n-VD2-C-(X2)n, wherein: VD1 is a first heavy chain variable domain; VD2 is a second heavy chain variable domain; C is a heavy chain constant domain; X1 is a linker with the proviso that it is not CH1; X2 is an Fc region; and n is independently 0 or 1; and wherein said second polypeptide chain comprises a second VD1-(X1)n-VD2-C-(X2)n, wherein: VD1 is a first light chain variable domain; VD2 is a second light chain variable domain; C is a light chain constant domain; X1 is a linker with the proviso that it is not CH1; X2 does not comprise an Fc region; and n is independently 0 or 1.

With respect to constructing DVD-Ig or other binding protein molecules, a “linker” is used to denote a single amino acid or a polypeptide (“linker polypeptide”) comprising two or more amino acid residues joined by peptide bonds and used to link one or more antigen binding portions. Such linker polypeptides are well known in the art (see, e.g., Holliger et al., Proc. Natl. Acad. Sci. USA, 90: 6444-6448 (1993); Poljak, R. J., Structure, 2: 1121-1123 (1994)). Flexible linkers may be employed, which are generally composed of small, non-polar (e.g. Gly) or polar (e.g. Ser or Thr) amino acids. Exemplary flexible linkers include, but are not limited to, GGGGSG (SEQ ID NO: 916), GGSGG (SEQ ID NO: 917), GGGGSGGGGS (SEQ ID NO: 918), GGSGGGGSG (SEQ ID NO: 919), GGSGGGGSGS (SEQ ID NO: 920), GGSGGGGSGGGGS (SEQ ID NO: 921), GGGGSGGGGSGGGG (SEQ ID NO: 922), GGGGSGGGGSGGGGS (SEQ ID NO: 923), and RADAAAAGGGGSGGGGSGGGGSGGGGS (SEQ ID NO: 924).

Alternatively, rigid linkers may be employed to join one or more antigen binding proteins. Said rigid linkers may allow for the maintenance of fixed distances between linked antigen binding proteins, thereby promoting the activity of each individual protein. Rigid linkers may employ one of more proline amino acids to confer the rigidity. Exemplary rigid linkers include, but are not limited to, ASTKGP (SEQ ID NO: 925), ASTKGPSVFPLAP (SEQ ID NO: 926), TVAAP (SEQ ID NO: 927), RTVAAP (SEQ ID NO: 928), TVAAPSVFIFPP (SEQ ID NO: 929), RTVAAPSVFIFPP (SEQ ID NO: 930), AKTTPKLEEGEFSEAR (SEQ ID NO: 931), AKTTPKLEEGEFSEARV (SEQ ID NO: 932), AKTTPKLGG (SEQ ID NO: 933), SAKTTPKLGG (SEQ ID NO: 934), SAKTTP (SEQ ID NO: 935), RADAAP (SEQ ID NO: 936), RADAAPTVS (SEQ ID NO: 937), RADAAAAGGPGS (SEQ ID NO: 938), SAKTTPKLEEGEFSEARV (SEQ ID NO: 939), ADAAP (SEQ ID NO: 940), ADAAPTVSIFPP (SEQ ID NO: 941), QPKAAP (SEQ ID NO: 942), QPKAAPSVTLFPP (SEQ ID NO: 943), AKTTPP (SEQ ID NO: 944), AKTTPPSVTPLAP (SEQ ID NO: 945), AKTTAP (SEQ ID NO: 946), AKTTAPSVYPLAP (SEQ ID NO: 947), GENKVEYAPALMALS (SEQ ID NO: 948), GPAKELTPLKEAKVS (SEQ ID NO: 949), and GHEAAAVMQVQYPAS (SEQ ID NO: 950).

In certain embodiments, the linker comprises a modified hinge region as described herein.

In certain embodiments, the linker comprises or consists of PLAP(SEQ ID NO: 911, PAPNLLGGP(SEQ ID NO:915), PLAPDKTHT(SEQ ID NO: 910), EKSYGPP(SEQ ID NO: 913), or DKTHT(SEQ ID NO: 912).

In certain embodiments, the multispecific binding protein comprises a first and a second polypeptide chain, wherein:
said first polypeptide chain comprises VH1-(HX1)n-VH2-C-(HX2)n, wherein:
VH1 is a first heavy chain variable domain; VH2 is a second heavy chain variable domain; C is a heavy chain constant domain; HX1 is a linker; HX2 is an Fc region; and n is independently 0 or 1; and said second polypeptide chain comprises VL1-(LX1)n-VL2-C-(LX2)n, wherein:
VL1 is a first light chain variable domain; VL2 is a second light chain variable domain; C is a light chain constant domain; LX1 is a linker; LX2 does not comprise an Fc region; and n is independently 0 or 1.

In certain embodiments, VH1 binds specifically to human IL-12Rβ1 and VH2 binds specifically to human IL-12Rβ2.

In certain embodiments, VL1 binds specifically to human IL-12Rβ1 and VL2 binds specifically to human IL-12Rβ2.

In certain embodiments, VH1 binds specifically to human IL-12Rβ2 and VH2 binds specifically to human IL-12RP 1.

In certain embodiments, VL1 binds specifically to human IL-12Rβ2 and VL2 binds specifically to human IL-12RP 1.

In certain embodiments, linker HX1 comprises an amino acid sequence of PLAP(SEQ ID NO: 911 or PAPNLLGGP(SEQ ID NO:915).

In certain embodiments, linker LX1 comprises an amino acid sequence of PLAP(SEQ ID NO: 911 or PAPNLLGGP(SEQ ID NO:915).

In certain embodiments, linker HX1 comprises an amino acid sequence of PLAP(SEQ ID NO: 911 and linker LX1 comprises an amino acid sequence of PLAP(SEQ ID NO: 911 or PAPNLLGGP(SEQ ID NO:915).

In certain embodiments, the multispecific binding protein comprises two polypeptide chains of VH1-(HX1)n-VH2-C-(HX2)n and two polypeptide chains of VL1-(LX1)n-VL2-C-(LX2)n.

In certain embodiments, for (HX1)n, n is 1 and for (HX2)n, n is 1.

In certain embodiments, for (LX1)n, n is 1 and for (LX2)n, n is 0.

In certain embodiments, the multispecific binding protein comprises a first and a second polypeptide chain, wherein:
said first polypeptide chain comprises VH1-(HX1)n-VH2-C-Fc, wherein:
VH1 is a first heavy chain variable domain; VH2 is a second heavy chain variable domain; C is a heavy chain constant domain; HX1 is a linker; Fc is an Fc region; and n is independently 0 or 1; and
said second polypeptide chain comprises VL1-(LX1)n-VL2-C, wherein:
VL1 is a first light chain variable domain; VL2 is a second light chain variable domain; C is a light chain constant domain; LX1 is a linker; and n is independently 0 or 1.

### Non-DVD-Ig Formats

In another aspect of the disclosure, the multispecific binding protein comprises from N-terminus to C-terminus:
ai) a first polypeptide chain comprising a first antigen binding domain, a first linker (e.g., a modified hinge region), and a first constant region; and
bi) a second polypeptide chain comprising a second antigen binding domain, a second linker (e.g., a modified hinge region), and a second constant region;
aii) a first polypeptide chain comprising a second antigen binding domain, a first antigen binding domain, a first linker (e.g., a modified hinge region), and a first constant region; and
bii) a second polypeptide chain comprising a second linker (e.g., a modified hinge region) or the absence of a linker, and a second constant region;
aiii) a first polypeptide chain comprising a first linker (e.g., a modified hinge region) or the absence of a linker, and a first constant region; and
biii) a second polypeptide chain comprising a second antigen binding domain, a first antigen binding domain, a second linker (e.g., a modified hinge region), and a second constant region; or
aiv) a first polypeptide chain comprising a first antigen binding domain, an optional first linker (e.g., a modified hinge region), a second antigen binding domain, an optional second linker (e.g., a modified hinge region), and a first constant region; and
biv) a second polypeptide chain comprising a third antigen binding domain, an optional third linker (e.g., a modified hinge region), a fourth antigen binding domain, an optional fourth linker (e.g., a modified hinge region), and a second constant region.

In certain embodiments, the first antigen binding domain comprises an scFv, VHH, Fab, F(ab′)2, or a single domain antibody.

In certain embodiments, the second antigen binding domain comprises an scFv, VHH, Fab, F(ab′)2, or a single domain antibody.

In certain embodiments, the third antigen binding domain comprises an scFv, VHH, Fab, F(ab′)2, or a single domain antibody.

In certain embodiments, the fourth antigen binding domain comprises an scFv, VHH, Fab, F(ab′)2, or a single domain antibody.

In certain embodiments, any one or more of the first antigen binding domain, second antigen binding domain, third antigen binding domain, and fourth antigen binding domain comprise an scFv, VHH, Fab, F(ab′)2, or a single domain antibody.

In certain embodiments, the first antigen binding domain, second antigen binding domain, third antigen binding domain, and fourth antigen binding domain each comprise an scFv.

In some embodiments, the upper hinge region comprises an amino acid sequence derived from an upper hinge region of a human IgG antibody. In some embodiments, the IgG antibody is selected from IgG1, IgG2, IgG3, and IgG4. In some embodiments, the IgG antibody is IgG1.

The Fc polypeptides employed in the multi-specific binding proteins as disclosed herein generally comprise a CH2 domain and a CH3 domain, wherein the C-terminus of the CH2 domain is linked (directly or indirectly) to the N-terminus of the CH3 domain. Any naturally occurring or variant CH2 and/or CH3 domain can be used. For example, in certain embodiments, the CH2 and/or CH3 domain is a naturally occurring CH2 or CH3 domain from an IgG1, IgG2, IgG3, IgG4, IgA1, or IgA2 antibody heavy chain, e.g., a human IgG1, IgG2, IgG3, IgG4, IgA1, or IgA2 antibody heavy chain. The CH2 and CH3 domains can be from the same or different antibody heavy chains. In certain embodiments, the Fc polypeptide comprises a CH2 and CH3 domain-containing portion from a single antibody heavy chain. In certain embodiments, the CH2 and/or CH3 domain is a variant of a naturally occurring CH2 or CH3 domain, respectively. In certain embodiments, the CH2 and/or CH3 domain is a variant comprising one or more amino acid insertions, deletion, substitutions, or modifications relative to a naturally occurring CH2 or CH3 domain, respectively. In certain embodiments, the CH2 and/or CH3 domain is a chimera of one or more CH2 or CH3 domains, respectively. In certain embodiments, the CH2 domain comprises amino acid positions 231-340 of a naturally occurring hinge region (e.g., human IgG1), according to the EU index. In certain embodiments, the CH3 domain comprises amino acid positions 341-447 of a naturally occurring hinge region (e.g., human IgG1), according to the EU index.

In certain embodiments, the Fc polypeptides further comprise a hinge region, wherein the C-terminus of hinge region is linked (directly or indirectly) to the N-terminus of the CH2 domain. For example, in certain embodiments, the hinge region is a naturally occurring hinge region from an IgG1, IgG2, IgG3, IgG4, IgA1, or IgA2 antibody heavy chain, e.g., a human IgG1, IgG2, IgG3, IgG4, IgA1, or IgA2 antibody heavy chain. The hinge region can be from the same or different antibody heavy chain than the CH2 and/or CH3 domains. In certain embodiments, the hinge region is a variant comprising one or more amino acid insertions, deletion, substitutions, or modifications relative to a naturally occurring hinge region. In certain embodiments, the hinge region is a chimera of one or more hinge regions. In certain embodiments, the hinge region comprises amino acid positions 226-229 of a naturally occurring hinge region (e.g., human IgG1), according to the EU index. In certain embodiments, the hinge region comprises amino acid positions 216-230 of a naturally occurring hinge region (e.g., human IgG1), according to the EU index. In certain embodiments, the hinge region comprises amino acid positions 216-230 of a naturally occurring hinge region (e.g., human IgG1), according to the EU index. In certain embodiments, the hinge region is a variant IgG4 hinge region comprising a serine (S) at amino acid position 228, according to the EU index.

In some embodiments, the multi-specific binding protein further comprises one or more modified hinge regions. In some embodiments, the one or more modified hinges comprises an upper hinge region of up to 7 amino acids in length or is absent; and a middle hinge region and a lower hinge region, wherein the lower hinge region is linked to the N-terminus of a heavy chain constant region. In some embodiments, the upper hinge region of the first and the second modified hinge regions are the same sequence. In some embodiments, the upper hinge region of the first and the second modified hinge regions are different sequences.

In certain embodiments, the Fc polypeptides further comprise a CH1 domain, wherein the C-terminus of CH1 domain is linked (directly or indirectly) to the N-terminus of the hinge region. For example, in certain embodiments, the CH1 domain is a naturally occurring CH1 domain from an IgG1, IgG2, IgG3, IgG4, IgA1, or IgA2 antibody heavy chain, e.g., a human IgG1, IgG2, IgG3, IgG4, IgA1, or IgA2 antibody heavy chain. The CH1 domain can be from the same or different antibody heavy chain than the hinge region, CH2 domain and/or CH3 domain. In certain embodiments, the CH1 domain is a variant comprising one or more amino acid insertions, deletions, substitutions, or modifications relative to a naturally occurring CH1 domain. In certain embodiments, the CH1 domain is a chimera of one or more CH1 domain. In certain embodiments, the CH1 domain comprises amino acid positions 118-215 of a naturally occurring hinge region (e.g., human IgG1), according to the EU index.

In certain embodiment, the Fc polypeptide lacks a CH1 domain or comprises mutations in a CH1 domain or heavy chain variable domain that prevent association of the heavy chain with an antibody light chain. In certain embodiments, the antibody heavy chain lacks a portion of a hinge region.

### Heterodimerization Motifs

In certain exemplary embodiments, the first and the second Fc domains are further engineered to enhance heterodimerization of the IL-12Rβ1 and IL-12Rβ2 binding domains and minimize the effects of incorrect chain pairing (i.e., pairing of IL-12Rβ1 binding domains or identical IL-12Rβ2 domains).

Any art-recognized approach that addresses the problem of incorrect chain pairing can be employed to improve desired multi-specific antibody production. For instance, US2010/0254989 A1 describes the construction of bispecific cMet-ErbB1 antibodies, where the VH and VL of the individual antibodies are fused genetically via a GlySer linker. For bispecific antibodies including an Fc domain, mutations may be introduced into the Fc to promote the correct heterodimerization of the Fc portion. Several such approaches are reviewed in Klein et al. (mAbs (2012) 4:6, 1-11), the contents of which are incorporated herein by reference in their entirety.

In certain embodiments, the IL-12Rβ 1 and IL-12Rβ2 binding specificities of the multi-specific antibody are heterodimerized through knobs-into-holes (KiH) pairing of Fc domains. This dimerization technique utilizes “protuberances” or “knobs” with “cavities” or “holes” engineered into the interface of CH3 domains. Where a suitably positioned and dimensioned knob or hole exists at the interface of either the first or second CH3 domain, it is only necessary to engineer a corresponding hole or knob, respectively, at the adjacent interface, thus promoting and strengthening Fc domain pairing in the CH3/CH3 domain interface. The IgG Fc domain that is fused to the VHH is provided with a knob, and the IgG Fc domain of the conventional antibody is provided with a hole designed to accommodate the knob, or vice-versa. A “knob” refers to an at least one amino acid side chain, typically a larger side chain, that protrudes from the interface of the CH3 portion of a first Fc domain. The protrusion creates a “knob” which is complementary to and received by a “hole” in the CH3 portion of a second Fc domain. The “hole” is an at least one amino acid side chain, typically a smaller side chain, which recedes from the interface of the CH3 portion of the second Fc domain. This technology is described, for example, in U.S. Pat. Nos. 5,821,333; 5,731,168 and 8,216,805; Ridgway et al. Protein Engineering (1996) 9:617-621); and Carter P. J. Immunol. Methods (2001) 248: 7-15, which are herein incorporated by reference.

Exemplary amino acid residues that may act as the knob include arginine (R), phenylalanine (F), tyrosine (Y) or tryptophan (W). An existing amino acid residue in the CH3 domain may be replaced or substituted with a knob amino acid residue. Preferred amino acids to substitute may include any amino acids with a small side chain, such as alanine (A), asparagine (N), aspartic acid (D), glycine (G), serine (S), threonine (T), or valine (V).

Exemplary amino acid residues that may act as the hole include alanine (A), serine (S), threonine (T), or valine (V). An existing amino acid residue in the CH3 domain may be replaced or substituted with a hole amino acid residue. Preferred amino acids to substitute may include any amino acids with a large side chain, such as arginine (R), phenylalanine (F), tyrosine (Y) or tryptophan (W).

The CH3 domain is preferably derived from a human IgG1 antibody. Exemplary amino acid substitutions to the CH3 domain include Y349C, S354C, T366S, T366Y, T366W, F405A, F405W, Y407T, Y407A, Y407V, T394S, or combinations thereof. A preferred exemplary combination is S354C, T366Y or T366W for the knob mutation on a first CH3 domain and Y349C, T366S, L368A, Y407T or Y407V for the hole mutation on a second CH3 domain.

In certain embodiments, the two Fc domains of the antigen binding construct are heterodimerized through Fab arm exchange (FAE). A human IgG1 possessing a P228S hinge mutation may contain an F405L or K409R CH3 domain mutation. Mixing of the two antibodies with a reducing agent leads to FAE. This technology is described in U.S. Pat. No. 9,212,230 and Labrijn A. F. PNAS (2013) 110(13):5145-5150, which are incorporated herein by reference.

In other embodiments, the two Fc domains of the antigen binding construct are heterodimerized through electrostatic steering effects. This dimerization technique utilizes electrostatic steering to promote and strengthen Fc domain pairing in the CH3/CH3 domain interface. The charge complementarity between two CH3 domains is altered to favor heterodimerization (opposite charge paring) over homodimerization (same charge pairing). In this method, the electrostatic repulsive forces prevent homodimerization. Certain exemplary amino acid residue substitutions which confer electrostatic steering effects include K409D, K392D, and/or K370D in a first CH3 domain and D399K, E356K, and/or E357K in a second CH3 domain. This technology is described in US Patent Publication No. 2014/0154254 A1 and Gunasekaran K. JBC (2010) 285(25):19637-19646, which are incorporated herein by reference.

In other embodiments, the charge complementarity is formed by a first Fc domain comprising a N297K and/or a T299K mutation, and a second Fc domain comprising a N297D and/or a T299D mutation.

In an aspect of the invention, the two Fc domains of the antigen binding construct are heterodimerized through hydrophobic interaction effects. This dimerization technique utilizes hydrophobic interactions instead of electrostatic ones to promote and strengthen Fc domain pairing in the CH3/CH3 domain interface. Exemplary amino acid residue substitution may include K409W, K360E, Q347E, Y349S, and/or S354C in a first CH3 domain and D399V, F405T, Q347R, E357W, and/or Y349C in a second CH3 domain. Preferred pairs of amino acid residue substitutions between a first CH3 domain and a second CH3 domain include K409W:D399V, K409W:F405T, K360E:Q347R, Y349S:E357W, and S354C:Y349C. This technology is described in US Patent Publication No. 2015/0307628 A1.

In an aspect of the invention, heterodimerization can be mediated through the use of leucine zipper fusions. Leucine zipper domains fused to the C terminus of each CH3 domain of the antibody chains force heterodimerization. This technology is described in Wranik B. JBC (2012) 287(52):43331-43339.

In an aspect of the invention, heterodimerization can be mediated through the use of a Strand Exchange Engineered Domain (SEED) body. CH3 domains derived from an IgG and IgA format force heterodimerization. This technology is described in Muda M. PEDS (2011) 24(5): 447-454.

In other embodiments, the heterodimerization motif may comprise non-native, disulfide bonds formed by engineered cysteine residues. In certain embodiments, the first set of disulfide may comprise a Y349C mutation in the first Fc domain and a S354C mutation in the second Fc domain. In other embodiment, an engineered disulfide bond may be introduced by fusion a C-terminal extension peptide with an engineered cysteine residue to the C-terminus of each of the two Fe domains. In certain embodiments, the first Fc domain may comprise the substitution of the carboxyl-terminal as “PGK” with “GEC”, and the second Fc domain may comprise the substitution of the carboxyl terminal amino acids “PGK” with “KSCDKT (SEQ ID NO:952)”.

In yet another approach, the multi-specific antibodies may employ the CrossMab principle (as reviewed in Klein et al.), which involves domain swapping between heavy and light chains so as to promote the formation of the correct pairings. Yet another approach involves engineering the interfaces between the paired VH-VL domains or paired CH1-CL domains of the heavy and light chains so as to increase the affinity between the heavy chain and its cognate light chain (Lewis et al. Nature Biotechnology (2014) 32: 191-198).

An alternative approach to the production of multi-specific antibody preparations having the correct antigen specificity has been the development of methods that enrich for antibodies having the correct heavy chain-light chain pairings. For example, Spiess et al. (Nature Biotechnology (2013) 31: 753-758) describe a method for the production of a MET-EGFR bispecific antibody from a co-culture of bacteria expressing two distinct half-antibodies.

Methods have also been described wherein the constant region of at least one of the heavy chains of a bispecific antibody is mutated so as to alter its binding affinity for an affinity agent, for example Protein A. This allows correctly paired heavy chain heterodimers to be isolated based on a purification technique that exploits the differential binding of the two heavy chains to an affinity agent (see US2010/0331527, WO2013/136186).

International patent application no. PCT/EP2012/071866 (WO2013/064701) addresses the problem of incorrect chain pairing using a method for multi-specific antibody isolation based on the use of anti-idiotypic binding agents, in particular anti-idiotypic antibodies. The anti-idiotype binding agents are employed in a two-step selection method in which a first agent is used to capture antibodies having a VH-VL domain pairing specific for a first antigen and a second agent is subsequently used to capture antibodies also having a second VH-VL domain pairing specific for a second antigen.

In yet another embodiment, the multi-specific antibody employs a first binding specificity having a conventional Fab binding region and a second binding specificity comprising a single domain antibody (VHH) binding region. The heterodimerization method employed forces the binding of the heavy chain region of the Fab and the full, heavy chain only, of the VHH. Because the VHH chain does not associate with light chains, the light chain region of the Fab portion will only associate with its corresponding heavy chain.

In certain other embodiments, the multi-specific binding protein described herein further comprises a common light chain. The term “common light chain” as used herein refers to a light chain which is capable of pairing with a first heavy chain of an antibody which binds to a first antigen in order to form a binding site specifically binding to said first antigen and which is also capable of pairing with a second heavy chain of an antibody which binds to a second antigen in order to form a binding site specifically binding to said second antigen. A common light chain is a polypeptide comprising in N-terminal to C-terminal direction an antibody light chain variable domain (VL), and an antibody light chain constant domain (CL), which is herein also abbreviated as “VL-CL”. Multi-specific binding proteins with a common light chain require heterodimerization of the distinct heavy chains. In certain embodiments, the heterodimerization methods listed above may be used with a common light chain. In certain exemplary embodiments, the heterodimerization motif may comprise non-native, disulfide bonds formed by engineered cysteine residues. Adding disulfide bonds, both between the heavy and light chain of an antibody has been shown to improve stability. Additionally, disulfide bonds have also been used as a solution to improve light-chain pairing within bispecific antibodies (Geddie M. L. et al, mAbs (2022) 14(1)).

Unless otherwise stated, all antibody constant region numbering employed herein corresponds to the EU numbering scheme, as described in Edelman et al. (Proc. Natl. Acad. Sci. 63(1): 78-85. 1969).

Additional methods of heterodimerization of heavy and/or light chains and the generation and purification of asymmetric antibodies are known in the art. See, for example, Klein C. mAbs (2012) 4(6): 653-663, and U.S. Pat. No. 9,499,634, each of which is incorporated herein by reference.

### Effector Function Mutations

As discussed above, multi-specific binding proteins of the disclosure can be provided in various isotypes and with different constant regions. The Fc region of the multi-specific binding primarily determines its effector function in terms of Fc binding, antibody-dependent cell-mediated cytotoxicity (ADCC) activity, complement dependent cytotoxicity (CDC) activity, and antibody-dependent cell phagocytosis (ADCP) activity. These “cellular effector functions”, as distinct from effector T cell function, involve the recruitment of cells bearing Fc receptors to the site of the target cells, resulting in killing of the antibody-bound cell.

An antibody according to the present invention may be one that exhibits reduced effector function. In certain embodiments, the one or more mutations reduces one or more of antibody dependent cellular cytotoxicity (ADCC), antibody dependent cellular phagocytosis (ADCP), or complement dependent cytotoxicity (CDC). In certain embodiments, an antibody according to the present invention may lack ADCC, ADCP and/or CDC activity. In either case, an antibody according to the present invention may comprise, or may optionally lack, an Fc region that binds to one or more types of Fc receptor. Use of different antibody formats, and the presence or absence of FcR binding and cellular effector functions, allow the antibody to be tailored for use in particular therapeutic purposes as discussed elsewhere herein.

In certain embodiments, the first and the second Fc domain comprise one or more mutations that reduces Fc effector function. In certain embodiments, the first Fc domain and the second Fc domain each comprise a L234A and L235A mutation. These IgG1 mutations are also known as the “LALA” mutations and are described in further detail in Xu et al. (Cell Immunol. 2000; 200:16-26). In certain embodiments the first Fc domain and the second Fc domain each comprise a L234A, L235A, G237A, and/or P329G mutation. The Fc domain amino acid positions referred to herein are based on EU antibody numbering. Alternatively, an antibody may have a constant region which is effector null. An antibody may have a heavy chain constant region that does not bind Fc receptors, for example the constant region may comprise a L235E mutation. Another optional mutation for a heavy chain constant region is S228P, which increases stability. A heavy chain constant region may be an IgG4 comprising both the L235E mutation and the S228P mutation. This “IgG4-PE” heavy chain constant region is effector null. A disabled IgG1 heavy chain constant region is also effector null. A disabled IgG1 heavy chain constant region may contain alanine at position 234, 235 and/or 237 (EU index numbering), e.g., it may be an IgG1 sequence comprising the L234A, L235A and/or G237A mutations (“LALAGA”).

Human IgG1 constant regions containing specific mutations or altered glycosylation on residue Asn297 (e.g., N297Q, N297D, and N297K, EU index numbering) have been shown to reduce binding to Fc receptors.

In other embodiments, it may be desirable to enhance the binding of the Fc region of a multi-specific antibody to human Fc gamma receptor IIIA (FcγRIIIA) relative to that of the Fc region of a corresponding naturally occurring antibody. In certain embodiments, a constant region may be engineered for enhanced ADCC and/or CDC and/or ADCP. The potency of Fc-mediated effects may be enhanced by engineering the Fc domain by various established techniques. Such methods increase the affinity for certain Fc-receptors, thus creating potential diverse profiles of activation enhancement. This can be achieved by modification of one or several amino acid residues. Example mutations are one or more of the residues selected from 239, 332 and 330 for human IgG1 constant regions (or the equivalent positions in other IgG isotypes). An antibody may thus comprise a human IgG1 constant region having one or more mutations independently selected from S239D, 1332E and A330L (EU index numbering).

Increased affinity for Fc receptors can also be achieved by altering the natural glycosylation profile of the Fc domain by, for example, generating under fucosylated or de-fucosylated variants. Non-fucosylated antibodies harbor a tri-mannosyl core structure of complex-type N-glycans of Fc without fucose residue. These glycoengineered antibodies that lack core fucose residue from the Fc N-glycans may exhibit stronger ADCC than fucosylated equivalents due to enhancement of FcγRIIIA binding capacity. For example, to increase ADCC, residues in the hinge region can be altered to increase binding to FcγRIIIA. Thus, an antibody may comprise a human IgG heavy chain constant region that is a variant of a wild-type human IgG heavy chain constant region. In certain embodiments, the variant human IgG heavy chain constant region binds to human Fcγ receptors selected from the group consisting of FcγRIIB and FcγRIIA with higher affinity than the wild type human IgG heavy chain constant region binds to the human FcγRIIIA. The antibody may comprise a human IgG heavy chain constant region that is a variant of a wild type human IgG heavy chain constant region, wherein the variant human IgG heavy chain constant region binds to human FcγRIIB with higher affinity than the wild type human IgG heavy chain constant region binds to human FcγRIIB. The variant human IgG heavy chain constant region can be a variant human IgG1, a variant human IgG2, or a variant human IgG4 heavy chain constant region. In one embodiment, the variant human IgG heavy chain constant region comprises one or more amino acid mutations selected from G236D, P238D, S239D, S267E, L328F, and L328E (EU index numbering system). In another embodiment, the variant human IgG heavy chain constant region comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F (EU index numbering system). In some embodiments, the variant human IgG heavy chain constant region comprises the set of amino acid mutations consisting of G236A, S239D, and 1332E.

The enhancement of CDC may be achieved by amino acid changes that increase affinity for C1q, the first component of the classic complement activation cascade. Another approach is to create a chimeric Fc domain created from human IgG1 and human IgG3 segments that exploit the higher affinity of IgG3 for C1q. Antibodies of the present invention may comprise mutated amino acids at residues 329, 331 and/or 322 to alter the C1q binding and/or reduced or abolished CDC activity. In another embodiment, the antibodies or antibody fragments disclosed herein may contain Fc regions with modifications at residues 231 and 239, whereby the amino acids are replaced to alter the ability of the antibody to fix complement. In one embodiment, the antibody or fragment has a constant region comprising one or more mutations selected from E345K, E430G, R344D and D356R, in particular a double mutation comprising R344D and D356R (EU index numbering system).

The functional properties of the multi-specific binding proteins may be further tuned by combining amino acid substitutions that alter Fc binding affinity with amino acid substitutions that affect binding to FcRn. Binding proteins with amino acid substitutions that affect binding to FcRn (also referred to herein as “FcRn variants”) may in certain situations also increase serum half-life in vivo as compared to an unmodified binding protein. As will be appreciated, any combination of Fc and FcRn variants may be used to tune clearance of the antigen-antibody complex. Suitable FcRn variants that may be combined with any of the Fc variants described herein that include without limitation N434A, N434S, M428L, V308F, V259I, M428L/N434S, V259I/V308F, Y436I/M428L, Y436I/N434S, Y436V/N434S, Y436V/M428L, M252Y, M252Y/S254T/T256E, and V259I/V308F/M428L.

### Half-Life Extending Mutations

In some embodiments, it is desirable to substitute certain amino acids in the Fc domain to extend the half-life of the multi-specific binding protein as disclosed herein. In some embodiments, the half-life of the multi-specific binding protein is improved relative to the half-life of a parent multi-specific binding protein (e.g., a multi-specific binding protein without an amino acid substitution that extends half-life).

In some embodiments, the substitution is at amino acid position 428, according to EU numbering. In some embodiments, the substitution is at amino acid position 434, according to EU numbering. In some embodiments, the substitution is at amino acid position 428 and amino acid position 434, according to EU numbering. In some embodiments, the amino acid substitution at position 428 is a leucine (L). In some embodiments, the substitution at amino acid position 434 is a serine (S).

In some embodiments, the substitution is at amino acid position 433, according to EU numbering. In some embodiments, the substitution is at amino acid position 434, according to EU numbering. In some embodiments, the substitution is at amino acid position 433 and amino acid position 434, according to EU numbering. In some embodiments, the amino acid substitution at position 433 is a lysine (K). In some embodiments, the amino acid substitution at position 434 is asparagine (N).

### Differential Binding

As defined in this disclosure, the term affinity refers to the strength of the interaction between an antigen binding moiety and the epitope to which it binds. As readily understood by those skilled in the art, an antigen binding moiety affinity may be reported as a dissociation constant (K_{D}) in molarity (M). Many antigen binding moieties have K_{D} values in the range of 10-6 to 10-9 M. High affinity antibodies have K_{D} values of 10-9 M (1 nanomolar, nM) and lower. For example, a high affinity antigen binding moiety may have K_{D} value in the range of about 1 nM to about 0.01 nM. A high affinity antibody may have K_{D} value of about 1 nM, about 0.9 nM, about 0.8 nM, about 0.7 nM, about 0.6 nM, about 0.5 nM, about 0.4 nM, about 0.3 nM, about 0.2 nM, or about 0.1 nM. Very high affinity antibodies have K_{D} values of 10⁻¹² M (1 picomolar, pM) and lower.

Low to medium affinity antibodies have K_{D} values of greater than about 10-9 M (1 nanomolar, nM). For example, a low to medium affinity antibody may have K_{D} value in the range of about 1 nM to about 100 nM. A low affinity antibody may have K_{D} value in the range of about 10 nM to about 100 nM. A low affinity antibody may have K_{D} value in the range of about 10 nM to about 80 nM. A low affinity antibody may have K_{D} value of about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 75 nM, about 80 nM, about 85 nM, about 90 nM, about 95 nM, about 100 nM, or greater than 100 nM.

In some embodiments, the antigen binding moiety with high affinity is the binding moiety that specifically binds to the IL-12Rβ2 receptor subunit. In some embodiments, the binding moiety that specifically binds to the IL-12Rβ2 receptor subunit has higher affinity to the IL-12Rβ2 receptor subunit compared to the affinity the binding moiety that specifically binds to the IL-12Rβ1 receptor subunit exhibits toward the IL-12Rβ1 receptor subunit. In some embodiments, the antigen binding moiety with low affinity is the binding moiety that specifically binds to the IL-12Rβ1 receptor subunit. In some embodiments, the higher affinity to the IL-12Rβ2 receptor subunit allows for selective binding to NK and/or T cells.

In some embodiments, the binding affinity of the binding moiety for the IL-12Rβ2 subunit is at least 2-fold greater than the binding affinity of the first binding moiety for the IL-12Rβ 1 subunit. In some embodiments, the binding affinity of the binding moiety for the IL-12Rβ2 subunit is at least 5-fold greater than the binding affinity of the first binding moiety for the IL-12Rβ 1 subunit. In some embodiments, the binding affinity of the binding moiety for the IL-12Rβ2 subunit is at least 10-fold greater than the binding affinity of the first binding moiety for the IL-12Rβ 1 subunit. In some embodiments, the binding affinity of the binding moiety for the IL-12Rβ2 subunit is at least 15-fold greater than the binding affinity of the first binding moiety for the IL-12Rβ 1 subunit. In some embodiments, the binding affinity of the binding moiety for the IL-12Rβ2 subunit is at least 20-fold greater than the binding affinity of the first binding moiety for the IL-12Rβ 1 subunit. In some embodiments, the binding affinity of the binding moiety for the IL-12Rβ2 subunit is at least 25-fold greater than the binding affinity of the first binding moiety for the IL-12Rβ 1 subunit. In some embodiments, the binding affinity of the binding moiety for the IL-12Rβ2 subunit is at least 30-fold greater than the binding affinity of the first binding moiety for the IL-12Rβ 1 subunit. In some embodiments, the binding affinity of the binding moiety for the IL-12Rβ2 subunit is at least 35-fold greater than the binding affinity of the first binding moiety for the IL-12Rβ 1 subunit. In some embodiments, the binding affinity of the binding moiety for the IL-12Rβ2 subunit is at least 40-fold greater than the binding affinity of the first binding moiety for the IL-12Rβ 1 subunit. In some embodiments, the binding affinity of the binding moiety for the IL-12Rβ2 subunit is at least 45-fold greater than the binding affinity of the first binding moiety for the IL-12Rβ 1 subunit. In some embodiments, the binding affinity of the binding moiety for the IL-12Rβ2 subunit is at least 50-fold greater than the binding affinity of the first binding moiety for the IL-12Rβ 1 subunit. In some embodiments, the binding affinity of the binding moiety for the IL-12Rβ2 subunit is at least 60-fold greater than the binding affinity of the first binding moiety for the IL-12Rβ 1 subunit. In some embodiments, the binding affinity of the binding moiety for the IL-12Rβ2 subunit is at least 70-fold greater than the binding affinity of the first binding moiety for the IL-12Rβ 1 subunit. In some embodiments, the binding affinity of the binding moiety for the IL-12Rβ2 subunit is at least 90-fold greater than the binding affinity of the first binding moiety for the IL-12Rβ 1 subunit. In some embodiments, the binding affinity of the binding moiety for the IL-12Rβ2 subunit is at least 100-fold greater than the binding affinity of the first binding moiety for the IL-12Rβ 1 subunit. In some embodiments, the binding affinity of the binding moiety for the IL-12Rβ2 subunit is at least 150-fold greater than the binding affinity of the first binding moiety for the IL-12Rβ 1 subunit. In some embodiments, the binding affinity of the binding moiety for the IL-12Rβ2 subunit is at least 200-fold greater than the binding affinity of the first binding moiety for the IL-12Rβ 1 subunit. In some embodiments, the binding affinity of the binding moiety for the IL-12Rβ2 subunit is at least 250-fold greater than the binding affinity of the first binding moiety for the IL-12Rβ 1 subunit. In some embodiments, the binding affinity of the binding moiety for the IL-12Rβ2 subunit is at least 300-fold greater than the binding affinity of the first binding moiety for the IL-12Rβ 1 subunit. In some embodiments, the binding affinity of the binding moiety for the IL-12Rβ2 subunit is at least 350-fold greater than the binding affinity of the first binding moiety for the IL-12Rβ 1 subunit. In some embodiments, the binding affinity of the binding moiety for the IL-12Rβ2 subunit is at least 400-fold greater than the binding affinity of the first binding moiety for the IL-12Rβ 1 subunit. In some embodiments, the binding affinity of the binding moiety for the IL-12Rβ2 subunit is at least 450-fold greater than the binding affinity of the first binding moiety for the IL-12Rβ 1 subunit. In some embodiments, the binding affinity of the binding moiety for the IL-12Rβ2 subunit is at least 500-fold greater than the binding affinity of the first binding moiety for the IL-12Rβ 1 subunit. In some embodiments, the binding affinity of the binding moiety for the IL-12Rβ2 subunit is at least 600-fold greater than the binding affinity of the first binding moiety for the IL-12Rβ 1 subunit. In some embodiments, the binding affinity of the binding moiety for the IL-12Rβ2 subunit is at least 700-fold greater than the binding affinity of the first binding moiety for the IL-12Rβ 1 subunit. In some embodiments, the binding affinity of the binding moiety for the IL-12Rβ2 subunit is at least 800-fold greater than the binding affinity of the first binding moiety for the IL-12Rβ 1 subunit. In some embodiments, the binding affinity of the binding moiety for the IL-12Rβ2 subunit is at least 900-fold greater than the binding affinity of the first binding moiety for the IL-12Rβ 1 subunit. In some embodiments, the binding affinity of the binding moiety for the IL-12Rβ2 subunit is at least 1000-fold greater than the binding affinity of the first binding moiety for the IL-12Rβ 1 subunit. In some embodiments, the binding affinity of the binding moiety for the IL-12Rβ2 subunit is at least 1500-fold greater than the binding affinity of the first binding moiety for the IL-12Rβ 1 subunit. In some embodiments, the binding affinity of the binding moiety for the IL-12Rβ2 subunit is at least 2000-fold greater than the binding affinity of the first binding moiety for the IL-12Rβ 1 subunit. In some embodiments, the binding affinity of the binding moiety for the IL-12Rβ2 subunit is at least 5000-fold greater than the binding affinity of the first binding moiety for the IL-12Rβ 1 subunit. In some embodiments, the binding affinity of the binding moiety for the IL-12Rβ2 subunit is at least 10000-fold greater than the binding affinity of the first binding moiety for the IL-12Rβ 1 subunit.

### Expression of Antigen-Binding Proteins

In one aspect, polynucleotides encoding the binding proteins (e.g., antigen-binding proteins and antigen-binding fragments thereof) disclosed herein are provided. Methods of making binding proteins comprising expressing these polynucleotides are also provided.

Polynucleotides encoding the binding proteins disclosed herein are typically inserted in an expression vector for introduction into host cells that may be used to produce the desired quantity of the binding proteins. Accordingly, in certain aspects, the disclosure provides expression vectors comprising polynucleotides disclosed herein and host cells comprising these vectors and polynucleotides.

The term “vector” or “expression vector” is used herein to mean vectors used in accordance with the present disclosure as a vehicle for introducing into and expressing a desired gene in a cell. As known to those skilled in the art, such vectors may readily be selected from the group consisting of plasmids, phages, viruses and retroviruses. In general, vectors compatible with the disclosure will comprise a selection marker, appropriate restriction sites to facilitate cloning of the desired gene and the ability to enter and/or replicate in eukaryotic or prokaryotic cells.

Numerous expression vector systems may be employed for the purposes of this disclosure. For example, one class of vector utilizes DNA elements which are derived from animal viruses such as bovine papilloma virus, polyoma virus, adenovirus, vaccinia virus, baculovirus, retroviruses (RSV, MMTV or MOMLV), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by co-transformation. Additional elements may also be needed for optimal synthesis of mRNA. These elements may include signal sequences, splice signals, as well as transcriptional promoters, enhancers, and termination signals. In some embodiments, the cloned variable region genes are inserted into an expression vector along with the heavy and light chain constant region genes (e.g., human constant region genes) synthesized as discussed above.

In other embodiments, the binding proteins may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980, which is incorporated by reference herein in its entirety for all purposes. Those skilled in the art will appreciate that such expression systems may be used to effectively produce the full range of polypeptides disclosed in the instant application.

More generally, once a vector or DNA sequence encoding a binding protein, e.g. an antibody or fragment thereof, has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Plasmid introduction into the host can be by electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

As used herein, the term “transformation” shall be used in a broad sense to refer to the introduction of DNA into a recipient host cell that changes the genotype.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, from supernatant of lysed cells culture, or from the cell culture containing both the medium and the suspended cells.

In one embodiment, a host cell line used for antibody expression is of mammalian origin. Those skilled in the art can determine particular host cell lines which are best suited for the desired gene product to be expressed therein. Exemplary host cell lines include, but are not limited to, GS-CHO and CHO-K1 (Chinese Hamster Ovary lines), DG44 and DUXB11 (Chinese Hamster Ovary lines, DHFR minus), HELA (human cervical carcinoma), CV-1 (monkey kidney line), COS (a derivative of CV-1 with SV40 T antigen), R1610 (Chinese hamster fibroblast) BALBC/3T3 (mouse fibroblast), HEK (human kidney line), SP2/O (mouse myeloma), BFA-1c1BPT (bovine endothelial cells), RAJI (human lymphocyte), 293 (human kidney). In one embodiment, the cell line provides for altered glycosylation, e.g., afucosylation, of the antibody expressed therefrom (e.g., PER.C6® (Crucell) or FUT8-knock-out CHO cell lines (POTELLIGENT® cells) (Biowa, Princeton, N.J.)). In one embodiment, NS0 cells may be used. CHO cells are particularly useful. Host cell lines are typically available from commercial services, e.g., the American Tissue Culture Collection, or from authors of published literature.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

Genes encoding the binding proteins featured in the disclosure can also be expressed in non-mammalian cells such as bacteria or yeast or plant cells. In this regard, it will be appreciated that various unicellular non-mammalian microorganisms such as bacteria can also be transformed, i.e., those capable of being grown in cultures or fermentation. Bacteria, which are susceptible to transformation, include members of the Enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the binding proteins can become part of inclusion bodies. In some embodiments, the binding proteins are then isolated, purified and assembled into functional molecules. In some embodiments, the binding proteins of the disclosure are expressed in a bacterial host cell. In some embodiments, the bacterial host cell is transformed with an expression vector comprising a nucleic acid molecule encoding a binding protein of the disclosure.

In addition to prokaryotes, eukaryotic microbes may also be used. Saccharomyces cerevisiae, or common baker's yeast, is the most commonly used among eukaryotic microbes, although a number of other strains are commonly available. For expression in Saccharomyces, the plasmid YRp7, for example (Stinchcomb et al., Nature, 282:39 (1979); Kingsman et al., Gene, 7:141 (1979); Tschemper et al., Gene, 10:157 (1980)), is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trp1 lesion as a characteristic of the yeast host cell genome then provides an effective environment for detecting transformation by growth in the absence of tryptophan.

### Formulations/Pharmaceutical Compositions

In certain embodiments, a pharmaceutical composition comprising a pharmaceutically acceptable carrier and a therapeutically effective amount of an antigen-binding protein described herein is provided. Some embodiments include pharmaceutical compositions comprising a therapeutically effective amount of any one of the binding proteins as described herein, or a binding protein-drug conjugate, in admixture with a pharmaceutically or physiologically acceptable formulation agent selected for suitability with the mode of administration.

Acceptable formulation materials are typically non-toxic to recipients at the dosages and concentrations employed.

In some embodiments, the pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides, e.g., sodium or potassium chloride, or mannitol sorbitol), delivery vehicles, diluents, excipients and/or pharmaceutical adjuvants (see, e.g., REMINGTON'S PHARMACEUTICAL SCIENCES (18th Ed., A. R. Gennaro, ed., Mack Publishing Company 1990), and subsequent editions of the same, incorporated herein by reference for any purpose).

In some embodiments the optimal pharmaceutical composition will be determined by a skilled artisan depending upon, for example, the intended route of administration, delivery format, and desired dosage. Such compositions can influence the physical state, stability, rate of in vivo release, and rate of in vivo clearance of the binding protein.

In some embodiments the primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, histidine buffer of above pH 5.5-6.5, or acetate buffer of about pH 4.0-5.5, which can further include sorbitol or a suitable substitute. In one embodiment of the disclosure, binding protein compositions can be prepared for storage by mixing the selected composition having the desired degree of purity with optional formulation agents in the form of a lyophilized cake or an aqueous solution. Further, the binding protein can be formulated as a lyophilizate using appropriate excipients such as sucrose.

In some embodiments, the pharmaceutical compositions of the disclosure can be selected for parenteral delivery or subcutaneous delivery. Alternatively, the compositions can be selected for inhalation or for delivery through the digestive tract, such as orally. The preparation of such pharmaceutically acceptable compositions is within the skill of the art.

In some embodiments, the formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use can be in the form of a pyrogen-free, parenterally acceptable, aqueous solution comprising the desired binding protein in a pharmaceutically acceptable vehicle. A particularly suitable vehicle for parenteral injection is sterile distilled water in which a binding protein is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which can then be delivered via a depot injection. Hyaluronic acid can also be used, and this can have the effect of promoting sustained duration in the circulation. Other suitable means for the introduction of the desired molecule include implantable drug delivery devices.

In one embodiment, a pharmaceutical composition can be formulated for inhalation. For example, a binding protein can be formulated as a dry powder for inhalation. Binding protein inhalation solutions can also be formulated with a propellant for aerosol delivery. In yet another embodiment, solutions can be nebulized.

It is also contemplated that certain formulations can be administered orally. In one embodiment of the disclosure, multi-specific binding proteins that are administered in this fashion can be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents can be included to facilitate absorption of the binding protein. Diluents, flavorings, low melting point waxes, vegetable oils, lubricants, suspending agents, tablet disintegrating agents, and binders can also be employed.

Another pharmaceutical composition can involve an effective quantity of multi-specific binding proteins in a mixture with non-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions of the disclosure will be evident to those skilled in the art, including formulations involving binding proteins in sustained- or controlled-delivery formulations. Techniques for formulating a variety of other sustained- or controlled-delivery means, such as liposome carriers, bio-erodible microparticles or porous beads and depot injections, are also known to those skilled in the art. Additional examples of sustained-release preparations include semipermeable polymer matrices in the form of shaped articles, e.g., films, or microcapsules. Sustained release matrices can include polyesters, hydrogels, polylactides, copolymers of L-glutamic acid and gamma ethyl-L-glutamate, poly(2-hydroxyethyl-methacrylate), ethylene vinyl acetate, or poly-D(−)-3-hydroxybutyric acid. Sustained-release compositions can also include liposomes, which can be prepared by any of several methods known in the art.

In some embodiments, pharmaceutical compositions are to be used for in vivo administration typically must be sterile. This can be accomplished by filtration through sterile filtration membranes. Where the composition is lyophilized, sterilization using this method can be conducted either prior to, or following, lyophilization and reconstitution. The composition for parenteral administration can be stored in lyophilized form or in a solution. In addition, parenteral compositions generally are placed into a container having a sterile access port, for example, an intravenous solution bag or vial having a stopper that can be pierced by a hypodermic injection needle.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

The disclosure also encompasses kits for producing a single dose administration unit. The kits can each contain both a first container having a dried multi-specific binding protein and a second container having an aqueous formulation. Also included within the scope of this disclosure are kits containing single and multi-chambered pre-filled syringes (e.g., liquid syringes and lyosyringes).

The effective amount of a binding protein pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding protein is being used, the route of administration, and the size (body weight, body surface, or organ size) and condition (the age and general health) of the patient. Accordingly, the clinician can titer the dosage and modify the route of administration to obtain the optimal therapeutic effect.

Dosing frequency will depend upon the pharmacokinetic parameters of the binding protein in the formulation being used. Typically, a clinician will administer the composition until a dosage is reached that achieves the desired effect. The composition can therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages can be ascertained through use of appropriate dose-response data.

The route of administration of the pharmaceutical composition is in accord with known methods, e.g., orally; through injection by intravenous, intraperitoneal, intracerebral (intraparenchymal), intracerebroventricular, intramuscular, intraocular, intraarterial, intraportal, or intralesional routes; by sustained release systems; or by implantation devices. Where desired, the compositions can be administered by bolus injection or continuously by infusion, or by implantation device.

In some embodiments, the composition can also be administered locally via implantation of a membrane, sponge, or other appropriate material onto which the desired molecule has been absorbed or encapsulated. Where an implantation device is used, the device can be implanted into any suitable tissue or organ, and delivery of the desired molecule can be via diffusion, timed-release bolus, or continuous administration.
Multi-specific binding proteins disclosed herein can be formulated as an aerosol for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma and are herein incorporated by reference in their entireties). These formulations for administration to the respiratory tract can be in the form of an aerosol or solution for a nebulizer, or as a microfine powder for insufflations, alone or in combination with an inert carrier such as lactose. In such a case, the particles of the formulation will, in one embodiment, have diameters of less than 50 microns, in one embodiment less than 10 microns.
A multi-specific binding protein disclosed herein can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the heterodimeric protein alone or in combination with other pharmaceutically acceptable excipients can also be administered.
Transdermal patches, including iontophoretic and electrophoretic devices, are well known to those of skill in the art, and can be used to administer a heterodimeric protein. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957, all of which are herein incorporated by reference in their entireties.
In certain embodiments, a pharmaceutical composition comprising a multi-specific binding protein described herein is a lyophilized powder, which can be reconstituted for administration as solutions, emulsions and other mixtures. It may also be reconstituted and formulated as solids or gels. The lyophilized powder is prepared by dissolving heterodimeric protein described herein, or a pharmaceutically acceptable derivative thereof, in a suitable solvent. In certain embodiments, the lyophilized powder is sterile. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature. Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined. Multi-specific binding proteins provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874, all of which are herein incorporated by reference in their entireties. In a specific embodiment, a heterodimeric protein described herein is targeted to a tumor.

### Methods of Treatment/Use

Another aspect of the disclosure is a multi-specific antibody and/or an antigen-binding protein as described herein for use as a medicament.

In a particular embodiment, a method of treating a disorder through the activation of IL-18R is provided, the method comprising administering to a subject in need thereof an effective amount of an antigen-binding protein described herein.

The binding proteins can be employed in any known assay method, such as competitive binding assays, direct and indirect sandwich assays, and immunoprecipitation assays for the detection and quantitation of one or more target antigens. The binding proteins will bind the one or more target antigens with an affinity that is appropriate for the assay method being employed.

For diagnostic applications, in some embodiments, binding proteins can be labeled with a detectable moiety. The detectable moiety can be any one that is capable of producing, either directly or indirectly, a detectable signal. For example, the detectable moiety can be a radioisotope, such as 3H, 14C, 32P, 35S, 1251, 99Tc, 111In, or 67Ga; a fluorescent or chemiluminescent compound, such as fluorescein isothiocyanate, rhodamine, or luciferin; or an enzyme, such as alkaline phosphatase, β-galactosidase, or horseradish peroxidase.

The binding proteins are also useful for in vivo imaging. A binding protein labeled with a detectable moiety can be administered to an animal, e.g., into the bloodstream, and the presence and location of the labeled antibody in the host assayed. The binding protein can be labeled with any moiety that is detectable in an animal, whether by nuclear magnetic resonance, radiology, or other detection means known in the art.

The disclosure also relates to a kit comprising a binding protein and other reagents useful for detecting target antigen levels in biological samples. Such reagents can include a detectable label, blocking serum, positive and negative control samples, and detection reagents. In some embodiments, the kit comprises a composition comprising any binding protein, polynucleotide, vector, vector system, and/or host cell described herein. In some embodiments, the kit comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing a condition and may have a sterile access port (for example the container may be an intravenous solution bag or a vial having a stopper that can be pierced by a hypodermic injection needle). In some embodiments, the label or package insert indicates that the composition is used for preventing, diagnosing, and/or treating the condition of choice. Alternatively, or additionally, the article of manufacture or kit may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In some embodiments, the present disclosure relates to a method of preventing and/or treating a disease or disorder (e.g., cancer). In some embodiments, the method comprises administering to a patient a therapeutically effective amount of at least one of the binding proteins, or pharmaceutical compositions related thereto, described herein. In some embodiments, the patient is a human.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present disclosure has been described with reference to the specific embodiments thereof, it should be understood by those skilled in the art that various changes may be made and equivalents may be substituted without departing from the true spirit and scope of the disclosure. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present disclosure. All such modifications are intended to be within the scope of the claims appended hereto. Having now described certain embodiments in detail, the same will be more clearly understood by reference to the following examples, which are included for purposes of illustration only and are not intended to be limiting.

### EXAMPLES

The following examples are put forth so as to provide those of ordinary skill in the art with a complete disclosure and description of how to make and use the methods and compositions featured in the invention and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Generation of Anti-IL-12R Antibodies from an Immunized VHH Library

Two llamas per target were injected five times with purified recombinant protein (human IL-12Rβ1-Fc, Acro Biosystems or human 12Rβ2-Fc, R&D Systems) weekly. Following the final protein immunization, blood samples were collected and PBMCs were isolated. RNA was then extracted and stored. The RNA purity and integrity was analyzed by micro-capillary electrophoresis using the 2100 Bioanalyzer (Agilent). RNA was converted to cDNA using reverse transcriptase and random primers. The VHHs were amplified using multiple primers and cloned into a phagemid vector. Phage were prepared and a library from each llama was generated and analyzed. Each library had a size of greater than 2.3E+08 and contained more than 95% VHH insert.

Recombinant proteins (human IL-12Rβ 1-his, Acro Biosystems, cyno IL-12Rβ 1-his, Sino Biological, human IL-12Rβ2-huFc-his, R&D system, cyno IL-12Rβ2-moFc, Sino Biological) was biotinylated using standard protocols and used to probe the libraries. The biotinylated antigen was incubated with the phage at various concentrations over multiple rounds of panning. E. coli were infected with output phage after each selection round to use for subsequent selections or characterization of individual clones. Periplasmic extract (PE) samples were tested for binding to ELISA and then sequenced.

To measure the activity of the PE samples in the ELISA assay, plates were coated with Neutravidin following by blocking with 1% Casein in PBS. Biotinylated human IL-12Rβ1-his or biotinylated human IL-12Rβ2-huFc-his were used to capture. PE Samples were diluted 1:5 in ELISA Buffer (0.1% casein in 1×PBS). Detection was done using an anti-c-myc antibody (Roche) followed by an anti-mouse IgG-HRP antibody (JIR). Plates were then incubated with TMB substrate (Invitrogen) followed by the addition of H2SO4 (Fisher) to stop the reaction. Plates were read at 450 nm using a microplate spectrophotometer.

To measure the activity of the PE samples in a FACS assay, DNA containing the open reading frame with a C-terminal tag (FLAG for IL-12RB1, HA for IL-12RB2) were transiently transfected using lipofectamine into HEK-293T cells. Expression was validated using commercially available antibodies (anti-huIL-12Rβ 1(R&D systems, anti-huIL-12Rβ2 (R&D systems) or anti-huIL-12Rβ2 (Biolegend). After transfection, cells were recovered and resuspended to a final concentration of 1.0E+06 cells/ml in FACS buffer and aliquoted in a 96-well plate. PE samples were added, and binding was detected using a primary antibody against the c-myc tag (Roche) and the secondary antibody against mouse IgG conjugated to APC (Invitrogen). All steps were completed in FACS buffer (0.5% FBS, 0.5 mM EDTA in 1×PBS). Analysis was performed using an iQUE3. Data is reported as fold over background (FOB), where the background are cells without any PE samples.

TABLE 12
ELISA and FACS data for VHHs against IL-12Rβ1

FACS data

| | | FOB | FOB |
|---|---|---|---|
| Clone | Bio-huIL-12Rβ1-His | human | cyno |
| nr. | OD450 nm | IL-12RB1 | IL-12RB1 |
| VHH25B1 | 0.479 | 160 | 121 |
| VHH32B1 | 1.280 | 78 | 129 |
| VHH41B1 | 0.413 | 48 | 19 |
| VHH50B1 | 0.151 | 132 | 106 |
| VHH54B1 | 0.822 | 43 | 19 |
| VHH58B1 | 3.033 | 82 | 88 |
| VHH67B1 | 2.928 | 88 | 82 |
| VHH72B1 | 1.980 | 117 | 120 |
| VHH86B1 | 0.422 | 104 | 64 |
| VHH97B1 | 0.409 | 133 | 99 |
| VHH104B1 | 2.972 | 152 | 161 |
| VHH108B1 | 2.735 | 260 | 184 |
| VHH115B1 | 1.901 | 101 | 62 |
| VHH136B1 | 2.841 | 80 | 88 |
| VHH137B1 | 3.314 | 116 | 152 |
| VHH172B1 | 3.142 | 75.09 | 50 |
| VHH154B1 | 2.846 | 24 | 21 |
| VHH174B1 | 1.192 | 80 | 68 |
| VHH187B1 | 0.125 | 86 | 42 |
| VHH195B1 | 2.931 | 78 | 58 |
| VHH202B1 | 3.304 | 86 | 55 |
| VHH233B1 | 0.349 | 146 | 81 |
| VHH238B1 | 3.332 | 50 | 44 |
| VHH245B1 | 3.124 | 78 | 36 |
| VHH258B1 | 3.297 | 183 | 91 |
| VHH263B1 | 0.144 | 105 | 89 |
| VHH276B1 | 3.202 | 31 | 24 |
| VHH321B1 | 1.064 | 119 | 93 |

TABLE 13
ELISA and FACS data for VHHs against IL-12Rβ2

| | Bio-huIL-12Rβ2- | FOB: | FOB: |
|---|---|---|---|
| Clone | huFc-His | human | cyno |
| nr. | OD450 nm | IL-12Rβ2 | IL-12Rβ2 |
| VHH13B2 | 1.713 | 1.77 | 2.65 |
| VHH19B2 | 1.303 | 1.63 | 2.75 |
| VHH64B2 | 2.220 | 1.68 | 2.69 |
| VHH67B2 | 0.581 | 1.73 | 2.32 |
| VHH68B2 | 2.135 | 2.25 | 3.47 |
| VHH85B2 | 2.988 | 1.64 | 2.31 |
| VHH93B2 | 0.151 | 1.98 | 1.35 |
| VHH94B2 | 0.190 | 3.19 | 3.84 |
| VHH185B2 | 2.100 | 2.71 | 4.18 |
| VHH190B2 | 0.174 | 2.25 | 3.78 |
| VHH194B2 | 0.602 | 1.68 | 3.06 |
| VHH211B2 | 0.227 | 1.64 | 0.99 |
| VHH216B2 | 0.586 | 0.99 | 0.98 |
| VHH219B2 | 0.237 | 1.54 | 0.99 |
| VHH222B2 | 0.961 | 3.17 | 1.28 |
| VHH229B2 | 0.138 | 1.42 | 1.00 |
| VHH230B2 | 0.308 | 2.05 | 1.66 |
| VHH241B2 | 0.139 | 1.63 | 2.37 |
| VHH277B2 | 0.137 | 1.21 | 1.55 |
| VHH278B2 | 0.794 | 2.46 | 3.97 |
| VHH289B2 | 3.195 | 1.91 | 2.88 |
| VHH294B2 | 2.196 | 1.98 | 3.19 |
| VHH307B2 | 0.626 | 1.95 | 3.16 |
| VHH322B2 | 1.672 | 1.99 | 3.21 |
| VHH356B2 | 2.860 | 1.43 | 2.00 |
| VHH361B2 | 1.351 | 2.16 | 4.01 |
| VHH375B2 | 3.201 | 1.88 | 2.86 |

### Example 2. Generation of Antibodies from an Immunized Human Transgenic Mouse

For each target receptor, eight mice immunized using protein and/or DNA until a robust titer was observed. After confirmation of titer, two fusions were performed on one mouse, seeding five 384 well plates per mouse. Hybridomas were screened using FACS with 293T cells overexpressing either the human or cyno versions of IL-12RB1 or IL-12RB2, as well as parental 293T cells as a control. For IL-12Rβ1, 376 hybridomas showed confirmed binding and were grown as saturated supernatants. For IL-12Rβ2, 321 hybridomas showed binding to human and cyno IL-12RB2 and were grown as saturated supernatants. The FACS experiment was repeated for the saturated supernatant. Data in Table 14 and Table 15 shows the binding of the hybridomas of HEK293T cell lines overexpressing the receptor of interest compared to the parent HEK293T cell line. In addition, saturated hybridomas were screened for binding to THP-1 cells (ATCC), a cell line that shows high endogenous expression of the IL-12 receptor. The binding of the hybridomas to the THP-1 cells was compared to buffer only wells (Table 14 and Table 15).

TABLE 14
FACS data for hybridomas against IL-12Rβ1

| | FACS: 293T | FACS: 293T | |
|---|---|---|---|
| | human | cyno | FACS: |
| | IL-12RB1 | IL-12RB1 | THP-1 |
| Hybridoma | (Fold over | (Fold over | (Fold over |
| ID | parental 293T) | parental 293T) | buffer) |
| 85801_01D14 | 116 | 52 | 24 |
| 85801_01L02 | 117 | 32 | 41 |
| 85801_01M07 | 91 | 28 | 7 |
| 85801_01002 | 107 | 13 | 38 |
| 85801_02G10 | 78 | 40 | 5 |
| 85801_02K24 | 130 | 53 | 33 |
| 85801_02N03 | 221 | 85 | 41 |
| 85801_02011 | 114 | 56 | 35 |
| 85801_03P23 | 69 | 23 | 39 |
| 85801_04D10 | 76 | 20 | 42 |
| 85801_04D14 | 202 | 44 | 17 |
| 85801_04G19 | 262 | 116 | 40 |
| 85801_04G23 | 73 | 27 | 46 |
| 85801_04M04 | 620 | 195 | 12 |
| 85801_04M10 | 336 | 122 | 36 |
| 85801_04M12 | 192 | 71 | 45 |
| 85801_04N20 | 317 | 116 | 41 |
| 85801_05F05 | 311 | 118 | 56 |
| 85801_05F12 | 80 | 34 | 44 |
| 85801_05G01 | 170 | 73 | 7 |
| 85801_06L19 | 73 | 25 | 9 |
| 85801_07A01 | 57 | 21 | 37 |
| 85801_10F19 | 41 | 16 | 8 |
| 85801_10K15 | 57 | 21 | 7 |
| 85801_10M19 | 58 | 21 | 35 |

TABLE 15
FACS data for hybridomas against IL-12Rβ2

| | FACS: 293T | FACS: 293T | |
|---|---|---|---|
| | human | cyno | FACS: |
| | IL-12RB2 | IL-12RB2 | THP1 |
| Hybridoma | (Fold over | (Fold over | (Fold over |
| ID | parental 293T) | parental 293T) | buffer) |
| 85802_10D03 | 3.4 | 6 | 24 |
| 85802_08G21 | 3.8 | 10 | 26 |
| 85802_06H06 | 2.9 | 10 | 34 |
| 85802_06F06 | 3.3 | 14 | 35 |
| 85802_06A17 | 2.8 | 11 | 28 |
| 85802_10K12 | 3.3 | 13 | 27 |
| 85802_02K15 | 3.3 | 6 | 53 |
| 85802_10M17 | 3.8 | 12 | 21 |
| 85802_08F20 | 3.4 | 8 | 21 |
| 85802_09B22 | 3.4 | 11 | 19 |

### Example 3. Identification and Generation of Heteromeric Antibodies

To establish the screening triage, an epitopically diverse set of VHHs and/or Fabs was selected by the DIAGONAL platform for the assembly into heteromeric molecules. Binding moieties were fused to an Fc containing mutations to promote heterodimerization ((Y349C, S354C, T366S, L368A, Y470V, T366W), as well as mutations (L234A, L235A, and G237A) to reduce effector function. Constructs also contained H435R and Y436F to ablate protein A binding on the “hole” Fe side to facilitate high throughput purification. 1254 heteromeric molecules were assembled and tested; 87 of these were chosen for further characterization (see Table 16).

Antibodies were transiently transfected into HEK293 cells. Cells were harvested six days post transfection and purified using a HiTrap MabSelect SuRe column. Antibodies that had less than 88% monomer were further purified by preparative SEC. Purity of the final product was assessed using a LabChip® and analytical gel filtration with a Superdex™ 200 10/30 column.

TABLE 16
IL-12 receptor heteromeric antibodies. Each binding domain is indicated
by the clone number or hybridoma ID of the Tables above.

| Antibody ID | IL-12Rβ1 Binder | IL-12Rβ2 Binder |
|---|---|---|
| DGL393 | 85801_01L02A | 185B2 |
| DGL395 | 85801_02K24A | 230B2 |
| DGL397 | 85801_02N03A | 222B2 |
| DGL398 | 85801_02N03A | 230B2 |
| DGL400 | 85801_04D14A | 185B2 |
| DGL405 | 85801_04G19A | 222B2 |
| DGL407 | 85801_04G23A | 13B2 |
| DGL408 | 85801_04G23A | 19B2 |
| DGL411 | 85801_04N20A | 185B2 |
| DGL412 | 85801_05G01A | 13B2 |
| DGL413 | 85801_05G01A | 185B2 |
| DGL415 | 85801_06L19A | 185B2 |
| DGL421 | 85801_10K15A | 190B2 |
| DGL424 | 54B1 | 85802_10K12A |
| DGL425 | 54B1 | 85802_02K15A |
| DGL427 | 58B1 | 85802_10M17A |
| DGL428 | 97B1 | 85802_10D03A |
| DGL430 | 97B1 | 85802_06H06A |
| DGL436 | 187B1 | 85802_10D03A |
| DGL439 | 195B1 | 85802_10D03A |
| DGL440 | 195B1 | 85802_08F06A |
| DGL441 | 195B1 | 85802_08G21A |
| DGL442 | 195B1 | 85802_06H06A |
| DGL443 | 195B1 | 85802_10K12A |
| DGL444 | 195B1 | 85802_08F20A |
| DGL445 | 202B1 | 85802_08F06A |
| DGL426 | 54B1 | 85802_08F20A |
| DGL495 | 72B1 | 19B2 |
| DGL448 | 233B1 | 85802_08621A |
| DGL453 | 25B1 | 67B2 |
| DGL454 | 25B1 | 93B2 |
| DGL461 | 32B1 | 93B2 |
| DGL462 | 32B1 | 222B2 |
| DGL464 | 50B1 | 19B2 |
| DGL465 | 50B1 | 93B2 |
| DGL478 | 58B1 | 222B2 |
| DGL479 | 58B1 | 230B2 |
| DGL481 | 67B1 | 19B2 |
| DGL482 | 67B1 | 64B2 |
| DGL484 | 67B1 | 93B2 |
| DGL487 | 67B1 | 190B2 |
| DGL490 | 67B1 | 230B2 |
| DGL496 | 72B1 | 64B2 |
| DGL497 | 72B1 | 67B2 |
| DGL499 | 72B1 | 93B2 |
| DGL503 | 72B1 | 222B2 |
| DGL504 | 72B1 | 230B2 |
| DGL505 | 72B1 | 241B2v2 |
| DGL506 | 72B1 | 289B2 |
| DGL508 | 72B1 | 307B2 |
| DGL514 | 97B1 | 67B2 |
| DGL515 | 97B1 | 85B2 |
| DGL516 | 97B1 | 93B2 |
| DGL518 | 104B1 | 13B2 |
| DGL524 | 104B1 | 222B2 |
| DGL525 | 104B1 | 230B2 |
| DGL527 | 108B1 | 13B2 |
| DGL533 | 108B1 | 230B2 |
| DGL534 | 115B1 | 19B2 |
| DGL537 | 115B1 | 222B2 |
| DGL541 | 137B1 | 230B2 |
| DGL545 | 174B1 | 230B2 |
| DGL547 | 187B1 | 294B2v2 |
| DGL551 | 195B1 | 185B2 |
| DGL552 | 195B1 | 222B2 |
| DGL557 | 202B1 | 13B2 |
| DGL559 | 202B1 | 64B2 |
| DGL562 | 202B1 | 185B2 |
| DGL568 | 233B1 | 13B2 |
| DGL569 | 233B1 | 19B2 |
| DGL570 | 233B1 | 64B2 |
| DGL571 | 233B1 | 67B2 |
| DGL572 | 233B1 | 93B2 |
| DGL574 | 233B1 | 185B2 |
| DGL575 | 233B1 | 190B2 |
| DGL577 | 233B1 | 219B2 |
| DGL579 | 233B1 | 230B2 |
| DGL584 | 245B1v2 | 64B2 |
| DGL585 | 258B1 | 13B2 |
| DGL586 | 258B1 | 19B2 |
| DGL587 | 258B1 | 64B2 |
| DGL594 | 263B1 | 64B2 |
| DGL599 | 263B1 | 190B2 |
| DGL601 | 85801_04M04A | 19B2 |
| DGL603 | 32B1 | 19B2 |
| DGL604 | 233B1 | 322B2 |
| DGL605 | 238B1 | 93B2 |
| DGL606 | 238B1 | 219B2 |
| DGL607 | 86B1 | 278B2 |
| DGL616 | 72B1 | 294B2v2 |
| DGL617 | 187B1 | 294B2v2 |

### Example 4. Screen for Agonist Activity

HEK-Blue™ IL-12 cells were purchased from InvivoGen. HEK-Blue™ IL-12 cells were generated by stably introducing the human genes for the IL-12 receptor and the genes of the IL-12 signaling pathway including a STAT4-inducible SEAP reporter gene. The binding of IL-12 to the IL-12R on the surface of HEK-Blue™ IL-12 cells triggers a signaling cascade leading to the activation of STAT-4 with the subsequent production of SEAP. Reporter cells were revived and cultured according to the supplier's recommendations. Cells were rinsed with PBS and added to 96-well plates at a density of about 50,000 cells/well. Controls or the heteromeric antibodies were added to the wells at a volume of 20 μL/well at the final assay condition listed in Table X. The plate was incubated at 37° C. in a CO₂ incubator for 20-24 hours. QUANTI-Blue™ Solution (InvivoGen) used to detect SEAP activity was prepared using manufacturer's instructions and 180 μL were added to a new plate. 20 μL of the induced HEK-Blue IL-12 supernatant was added to each well and the plate was incubated at 37° C. for 3 hours and read on a spectrophotometer at 630 nm.

To determine EC₅₀, antibodies were diluted to 100 nM, then seven 4-fold dilutions were also measured. EC₅₀ values were calculated in PRISM by using the log(agonist) vs. response (three parameters) fit.

The agonist activity of the heteromeric antibodies is shown in Table 17. below.

TABLE 17
Agonistic activity of heteromeric IL-12 receptor antibodies.

| Antibody ID | EC₅₀ | Max Absorbance |
|---|---|---|
| DGL424 | 2.6E−10 | 1.49 |
| DGL570 | >100 nM | 1.44 |
| DGL412 | >100 nM | 1.28 |
| DGL426 | 2.7E−10 | 1.24 |
| DGL569 | 5.2E−09 | 1.19 |
| DGL572 | 1.2E−08 | 1.05 |
| DGL516 | 6.5E−11 | 1.05 |
| DGL428 | 8.8E−10 | 1.03 |
| DGL495 | 6.4E−10 | 0.96 |
| DGL481 | 3.3E−10 | 0.93 |
| DGL606 | 8.8E−09 | 0.92 |
| DGL568 | >100 nM | 0.91 |
| DGL421 | 1.1E−09 | 0.90 |
| DGL514 | 3.5E−11 | 0.85 |
| DGL601 | 6.6E−11 | 0.79 |
| DGL605 | 4.6E−10 | 0.74 |
| DGL534 | 4.5E−10 | 0.69 |
| DGL465 | >100 nM | 0.69 |
| DGL482 | 2.9E−09 | 0.68 |
| DGL515 | >100 nM | 0.64 |
| DGL571 | >100 nM | 0.64 |
| DGL487 | 3.1E−09 | 0.64 |
| DGL607 | 7.0E−09 | 0.61 |
| DGL444 | 1.5E−10 | 0.61 |
| DGL586 | 7.7E−11 | 0.60 |
| DGL551 | 4.7E−09 | 0.59 |
| DGL545 | >100 nM | 0.59 |
| DGL524 | 4.4E−10 | 0.58 |
| DGL584 | >100 nM | 0.58 |
| DGL499 | 8.1E−09 | 0.56 |
| DGL537 | 5.9E−10 | 0.55 |
| DGL484 | 9.7E−09 | 0.54 |
| DGL527 | >100 nM | 0.54 |
| DGL462 | 1.2E−09 | 0.52 |
| DGL599 | >100 nM | 0.48 |
| DGL464 | >100 nM | 0.47 |
| DGL454 | >100 nM | 0.47 |
| DGL441 | 4.0E−11 | 0.45 |
| DGL575 | >100 nM | 0.43 |
| DGL445 | >100 nM | 0.42 |
| DGL533 | >100 nM | 0.42 |
| DGL504 | >100 nM | 0.41 |
| DGL574 | >100 nM | 0.41 |
| DGL478 | 4.0E−09 | 0.40 |
| DGL552 | >100 nM | 0.40 |
| DGL448 | 1.0E−09 | 0.39 |
| DGL541 | N.C. | 0.37 |
| DGL585 | 3.2E−09 | 0.37 |
| DGL436 | >100 nM | 0.36 |
| DGL579 | N.C. | 0.36 |
| DGL503 | N.C. | 0.35 |
| DGL594 | N.C. | 0.33 |
| DGL497 | N.C. | 0.32 |
| DGL587 | N.C. | 0.32 |
| DGL603 | N.C. | 0.31 |
| DGL506 | N.C. | 0.31 |
| DGL461 | N.C. | 0.31 |
| DGL413 | N.C. | 0.29 |
| DGL479 | N.C. | 0.29 |
| DGL397 | 1.6E−11 | 0.28 |
| DGL508 | N.C. | 0.28 |
| DGL518 | N.C. | 0.27 |
| DGL496 | N.C. | 0.27 |
| DGL395 | 2.3E−10 | 0.26 |
| DGL405 | N.C. | 0.26 |
| DGL505 | N.C. | 0.26 |
| DGL490 | N.C. | 0.25 |
| DGL453 | N.C. | 0.25 |
| DGL427 | N.C. | 0.25 |
| DGL443 | N.C. | 0.24 |
| DGL507 | N.C. | 0.24 |
| DGL393 | >100 nM | 0.23 |
| DGL415 | N.C. | 0.23 |
| DGL604 | N.C. | 0.23 |
| DGL411 | N.C. | 0.23 |
| DGL440 | N.C. | 0.22 |
| DGL442 | N.C. | 0.22 |
| DGL525 | N.C. | 0.21 |
| DGL430 | N.C. | 0.21 |
| DGL400 | N.C. | 0.21 |
| DGL439 | N.C. | 0.21 |
| DGL602 | N.C. | 0.19 |
| DGL425 | N.C. | 0.19 |
| DGL547 | N.C. | 0.18 |
| DGL407 | N.C. | 0.17 |
| DGL398 | N.C. | 0.17 |
| DGL408 | N.C. | 0.17 |

### Example 5. Characterization of Binding to Each IL-12 Receptor Subunit

The disassociation constant (K_{D}) was measured for a subset of the generated bispecific antibodies as described above. To measure the KD, purified bispecifics were loaded onto an AHC biosensor tip (Sartorius) to a threshold of 0.8 in a Octet BLI system. Analytes (human IL-12Rβ1, Acro and human IL-12Rβ2, Acro) were measured at three concentrations (100 nM, 33 nM, 11 nM) and a buffer control. Binding association was measured for 300 seconds, followed by a 600 second dissociation time. K_{D} values were obtained for each arm—the IL-12Rβ1 binding domain and the IL-12Rβ2 binding domain. The results can be found in Table 18.

TABLE 18
K_{D} measurements for the IL-12Rβ1 and the IL-12Rβ2
binding domains of bispecific agonistic antibodies.

| Antibody ID | K_{D} (M) IL-12Rβ1 | K_{D} (M) IL-12Rβ2 |
|---|---|---|
| DGL514 | 9.86E−09 | 5.48E−09 |
| DGL534 | 5.95E−08 | 7.06E−10 |
| DGL551 | 1.06E−08 | 8.29E−09 |
| DGL444 | 8.96E−09 | 3.86E−10 |
| DGL568 | 1.78E−09 | 2.34E−09 |
| DGL569 | 1.70E−09 | 7.23E−10 |
| DGL570 | 1.38E−09 | 7.66E−09 |
| DGL572 | 1.48E−09 | 2.41E−09 |
| DGL606 | 2.68E−09 | 6.03E−09 |
| DGL605 | 2.25E−09 | 1.87E−09 |
| DGL586 | 7.26E−09 | 7.68E−10 |
| DGL426 | 1.59E−08 | 3.21E−10 |
| DGL424 | 1.72E−08 | 7.26E−10 |
| DGL487 | 5.49E−09 | 1.61E−08 |
| DGL481 | 7.17E−09 | 7.72E−10 |
| DGL482 | 6.83E−09 | 7.40E−09 |
| DGL495 | 1.07E−08 | 7.70E−10 |
| DGL601 | 1.63E−09 | 6.82E−10 |
| DGL412 | 1.13E−08 | 2.21E−09 |
| DGL421 | 6.06E−09 | 1.95E−08 |
| DGL607 | 2.74E−08 | 1.18E−08 |
| DGL428 | 1.66E−08 | 1.97E−09 |
| DGL515 | 2.08E−08 | 1.00E−12 |
| DGL516 | 2.73E−08 | 3.41E−09 |

For many of these antibodies, the IL-12Rβ2 binding domain of the bispecific antibodies bind tighter to the IL-12Rβ2 receptor subunit than the IL-12Rβ31 binding domain to the IL-12Rβ31 receptor subunit.

### Example 6. Epitope Binning of Selected Antibodies

Epitope binning was performed using Carterra's LSA SPR (surface plasmon resonance) platform in the classic binning assay format. A HC30M chip was conditioned with a base and salt solution (50 mM NaOH, 10 mM NaCl) followed by an acid solution (10 mM glycine, pH 2.0) using 25 mM MES, pH5.5, 0.01% tween-20 as the running buffer. The binning surface lawn was activated with freshly prepared 1:1:1 v/v/v mix of 0.4 M EDC (Pierce), 0.1 M sulfo-NHS (Pierce), and 0.1 M MES, pH 5.5 for 7 minutes. The binning antibodies were coupled at 20ug/mL in 10 mM sodium acetate at 3 different pHs (4.5, 4.75 and 5.0) by using all 4 print blocks with 15 minutes total coupling time. Unreactive esters on the chip surface were quenched using 1M ethanolamine pH 8.5 (Carterra) for 7 minutes and then the antibody coupled chip was washed with 25 mM MES, pH 5.5, 0.01% tween-20 buffer twice for 15 seconds each. The binning experiment was performed on the coupled chip by injecting 200 nM recombinant human IL-12R β1 (Acro Biosystem #ILB-H52H9) or IL-12Rβ2 (Acro Biosystem #TLB-H52H6) in 1×HBSTE, 0.5 mg/L BSA buffer for 5 minutes, and then 20 μg/mL antibody analyte in 1×HBSTE, 0.5 mg/L BSA for 5 minutes. After every binning cycle, the arrayed antibodies were regenerated using 10 mM glycine, pH 2.0 (2, 30-sec pulses). Carterra's epitope binning software was used to analyze the experimental data.

TABLE 19
Epitope Community of IL-12Rβ1

| DGL | IL-12RB1 binder | IL-12RB2 binder | Community Group |
|---|---|---|---|
| DGL515 | 97B1 | 85B2 | Group 1 |
| DGL594 | 263B1 | 64B2 | Group 1 |
| DGL534 | 115B1 | 19B2 | Group 2 |
| DGL585 | 258B1 | 13B2 | Group 2 |
| DGL586 | 258B1 | 19B2 | Group 2 |
| DGL587 | 258B1 | 64B2 | Group 2 |
| DGL603 | 32B1 | 19B2 | Group 2 |
| DGL616 | 72B1 | 294B2v2 | Group 2 |
| DGL557 | 202B1 | 13B2 | Group 3 |
| DGL559 | 202B1 | 64B2 | Group 3 |
| DGL562 | 202B1 | 185B2 | Group 3 |
| DGL570 | 233B1 | 64B2 | Group 4 |
| DGL577 | 233B1 | 219B2 | Group 4 |
| DGL579 | 233B1 | 230B2 | Group 4 |
| DGL584 | 245B1v2 | 64B2 | Group 5 |
| DGL617 | 187B1 | 294B2v2 | Group 6 |

TABLE 20
Epitope Community of IL-12Rβ2

| DGL | IL-12RB1 binder | IL-12RB2 binder | Group |
|---|---|---|---|
| DGL515 | 97B1 | 85B2 | Group 1 |
| DGL534 | 115B1 | 19B2 | Group 2 |
| DGL586 | 258B1 | 19B2 | Group 2 |
| DGL603 | 32B1 | 19B2 | Group 2 |
| DGL557 | 202B1 | 13B2 | Group 3 |
| DGL559 | 202B1 | 64B2 | Group 3 |
| DGL570 | 233B1 | 64B2 | Group 3 |
| DGL584 | 245B1v2 | 64B2 | Group 3 |
| DGL585 | 258B1 | 13B2 | Group 3 |
| DGL587 | 258B1 | 64B2 | Group 3 |
| DGL594 | 263B1 | 64B2 | Group 3 |
| DGL562 | 202B1 | 185B2 | Group 4 |
| DGL577 | 233B1 | 219B2 | Group 4 |
| DGL579 | 233B1 | 230B2 | Group 5 |
| DGL616 | 72B1 | 294B2v2 | Group 6 |
| DGL617 | 187B1 | 294B2v2 | Group 6 |

### Example 7. Characterization of Antibodies for Ligand Blocking

To characterize the ligand blocking activity of recombinant anti-IL12Rβ1 or anti-IL12Rβ2 antibodies, an SPR ligand competition assay is deployed using a Carterra LSA instrument (Carterra Bio). Anti-IL12Rβ1 or anti-IL 12Rβ2 is first immobilized via affinity capture with a custom anti-human Fc sensor chip (Goat anti-human IgG (H+L) from Southern Biotech (Cat. #2087-01) coupled to HC30M sensor chip (Carterra Bio)). Subsequently, recombinant extracellular domain (ECD) of IL-12Rβ1/IL-12Rβ2 is injected to the captured surface with or without molar excess of recombinant IL-12 ligand. In a scenario where the anti-IL12Rβ1/IL12Rβ2 antibody binds to a surface on the receptor protein that is non-overlapping with IL-12 ligand binding interface, a robust, positive response is observed, consistent with the antibody capturing the receptor-ligand complex. Conversely, in a scenario where the anti-IL12Rβ1/IL 2Rβ2 antibody binds to a surface on the receptor protein that is overlapping with IL-12 ligand binding surface, no response is observed, consistent with the IL-12 ligand preventing anti-IL12Rβ1/IL12Rβ2 antibody from capturing the receptor ECD protein. Using this assay, a panel of anti-IL-12Rβ1 or IL-12Rβ2 binding antibodies are classified as ligand blocking or non-blocking antibodies.

1. A multi-specific binding protein comprising at least a first binding moiety which binds specifically to a human interleukin-12 receptor β1 (IL-12Rβ1) subunit, and at least a second binding moiety which binds specifically to a human IL-12 receptor β2 (IL-12Rβ2) subunit, wherein the second binding moiety exhibits higher binding affinity (KD) for the human IL-12Rβ2 subunit than the first binding moiety exhibits for the human IL-12Rβ1 subunit, and is capable of inducing IL-12 receptor signaling by inducing proximity between the IL-12Rβ1 and IL-12Rβ2 subunits of human IL-12 receptor.

2. The multi-specific binding protein of claim 1, wherein the binding affinity of the second binding moiety for the IL-12Rβ2 subunit is:
at least 5-fold greater than the binding affinity of the first binding moiety for the IL-12Rβ1 subunit,
at least 10-fold greater than the binding affinity of the first binding moiety for the IL-12Rβ1 subunit;
at least 20-fold greater than the binding affinity of the first binding moiety for the IL-12Rβ1 subunit:
at least 40-fold greater than the binding affinity of the first binding moiety for the IL-12Rβ1 subunit;
at least 100-fold greater than the binding affinity of the first binding moiety for the IL-12Rβ1 subunit; or
at least 1000-fold greater than the binding affinity of the first binding moiety for the IL-12Rβ1 subunit.

3-7. (canceled)

8. The multi-specific binding protein of claim 1, wherein the binding affinity of the second binding moiety for the IL-12Rβ2 subunit promotes selective binding to activated T cells and/or NK cells.

9. The multi-specific binding protein of claim 1, wherein:
a. the first binding moiety comprises an IL-12Rβ1 VHH domain and the second binding moiety comprises an IL-12Rβ2 VHH domain;
b. the first binding moiety comprises an IL-12Rβ1 Fab domain and the second binding moiety comprises an IL-12Rβ2 Fab domain;
c. the first binding moiety comprises an IL-12Rβ1 VHH domain and the second binding moiety comprises an IL-12Rβ2 Fab domain;
d. the first binding moiety comprises an IL-12Rβ1 Fab domain and the second binding moiety comprises an IL-12Rβ2 VHH domain;
e. the first binding moiety comprises an IL-12Rβ1 Fab domain and the second binding moiety comprises an IL-12Rβ2 scFv domain;
f. the first binding moiety comprises an IL-12Rβ1 scFv domain and the second binding moiety comprises an IL-12Rβ2 Fab domain;
g. the first binding moiety comprises an IL-12Rβ1 scFv domain and the second binding moiety comprises an IL-12Rβ2 scFv domain;
h. the first binding moiety comprises an IL-12Rβ1 scFv domain and the second binding moiety comprises an IL-12Rβ2 VHH domain; or
i. the first binding moiety comprises an IL-12Rβ1 VHH domain and the second binding moiety comprises an IL-12Rβ2 scFv domain.

10. The multi-specific binding protein of claim 1, wherein the first binding moiety comprises a IL-12Rβ1 VHH domain, optionally wherein:
the IL-12Rβ1 VHH domain comprises a HCDR1 sequence, a HCDR2 sequence, and a HCDR3 sequence as found in Table 4; and/or
the IL-12Rβ1 VHH domain comprises a sequence that is at least about 90% identical, at least about 95% identical, at least about 96%, at least about 97%, at least about 98%, or at least 99% identical to the amino acid sequence of any one of the amino acid sequences in Table 5.

11-12. (canceled)

13. The multi-specific binding protein of claim 1, wherein the second binding moiety comprises an IL-12Rβ2 VHH domain, optionally wherein:
the IL-12Rβ2 VHH domain comprises a HCDR1 sequence, a HCDR2 sequence, and a HCDR3 sequence as found in Table 9; and/or
the IL-12Rβ2 VHH domain comprises a sequence that is at least about 90% identical, at least about 95% identical, at least about 96%, at least about 97%, at least about 98%, or at least about 99% identical t the amino acid sequence of any one of the amino acid sequences in Table 10.

14-15. (canceled)

16. The multi-specific binding protein of claim 1, wherein the IL-12Rβ1 VHH domain comprises the amino acid sequence of any one of the amino acid sequences in Table 5 and/or the IL-12Rβ2 VHH domain comprises the amino acid sequence of any one of the amino acid sequences in Table 10.

17. (canceled)

18. The multi-specific binding protein of claim 1, wherein the first binding moiety or second binding moiety is a Fab or an scFv, optionally wherein:
the Fab or scFv comprises a variable heavy chain region (VH) and a variable light chain region (VL), optionally wherein the VH comprises a HCDR1 sequence, a HCDR2 sequence, and a HCDR3 sequence as found in Table 1 and/or the VL comprises a LCDR1 sequence, a LCDR2 sequence, and a LCDR3 sequence as found in Table 2:
the VH of the Fab or scFv of the first binding moiety comprises a sequence that is at least 90% identical, at least about 95% identical, at least about 96% identical, at least about 97% identical, at least about 98%, or at least about 99% identical to an amino acid sequence of Table 3;
the VL of the Fab or scFv of the first binding moiety comprises a sequence that is at least 90% identical, at least about 95% identical, at least about 96% identical, at least about 97% identical, at least about 98% identical, or at least about 99% identical to an amino acid sequence of Table 3;
the VH of the Fab or scFv of the first binding moiety comprises the amino acid sequence of Table 3;
the VL of the Fab or scFv of the first binding moiety comprises the amino acid sequence of Table 3;
the VH of the Fab or scFv of the second binding moiety comprises a sequence that is at least 90% identical, at least about 95% identical, at least about 96% identical, at least about 97% identical, at least about 98% identical, or at least about 99% identical to an amino acid sequence of Table 8;
the VL of the Fab or scFv of the second binding moiety comprises a sequence that is at least 90% identical, at least about 95% identical, at least about 96% identical, at least about 97% identical, at least about 98% identical, or at least about 99% identical to an amino acid sequence of Table 8;
the VH of the Fab or scFv of the second binding moiety comprises the amino acid sequence of an amino acid sequence of Table 8; and/or
the VL of the Fab or scFv of the second binding moiety comprises the amino acid sequence of an amino acid sequence of Table 8.

19-29. (canceled)

30. The multi-specific binding protein of claim 1, capable of inducing IL-12 receptor signaling in the presence of IL-12, optionally wherein induction of IL-12 receptor signaling is detected via a surface plasmon resonance (SPR) assay, optionally wherein the SPR assay comprises the following steps:
1) contacting the first binding moiety and/or the second binding moiety with an extracellular domain (ECD) of one or both of IL-12R β1 and IL-12R β2 and isolated IL-12; and
2) detecting binding of the first binding moiety and/or the second binding moiety with the ECD of one or both of IL-12R β1 and IL-12R β2,
wherein detection of binding indicates that the multi-specific binding protein is capable of inducing IL-12 receptor signaling in the presence of IL-12.

31-32. (canceled)

33. The multi-specific binding protein of claim 1, capable of binding specifically to human IL-12Rβ1 subunit and human IL-12Rβ2 subunit in the presence of IL-12: optionally wherein binding specifically to human IL-12Rβ1 subunit and human IL-12Rβ2 subunit is detected via a surface plasmon resonance (SPR) assay, optionally wherein the SPR assay comprises the following steps:
1) contacting the first binding moiety and/or the second binding moiety with an extracellular domain (ECD) of one or both of IL-12R β1 and IL-12R β2 and isolated IL-12; and 2) detecting binding of the first binding moiety and/or the second binding moiety with the ECD of one or both of IL-12R β1 and IL-12R β2,
wherein detection of binding indicates that the multi-specific binding protein is capable of binding specifically to human IL-12Rβ1 subunit and human IL-12Rβ2 subunit in the presence of IL-12.

34-35. (canceled)

36. The multi-specific binding protein of claim 1, wherein:
the first binding moiety designated 97B1 and 263B1 of the multi-specific binding protein compete for binding to IL-12Rβ1;
the first binding moiety designated 115B1, 258B1, 32B1, and 72B1 of the multi-specific binding protein compete for binding to IL-12Rβ1
the first binding moiety designated 202B1 of the multi-specific binding protein competes for binding to IL-12Rβ1 with one or more anti-IL-12Rβ1 binding moieties disclosed herein:
the first binding moiety designated 233B1 of the multi-specific binding protein competes for binding to IL-12Rβ1 with one or more anti-IL-12Rβ1 binding moieties disclosed herein;
the first binding moiety designated 245B1v2 of the multi-specific binding protein competes for binding to IL-12Rβ1 with one or more anti-IL-12Rβ1 binding moieties disclosed herein;
the first binding moiety designated 187B1 of the multi-specific binding protein competes for binding to IL-12Rβ1 with one or more anti-IL-12Rβ1 binding moieties disclosed herein;
the second binding moiety designated 13B2 and 64B2 of the multi-specific binding protein compete for binding to IL-12Rβ2;
the second binding moiety designated 185B2 and 219B2 of the multi-specific binding protein compete for binding to IL-12Rβ2;
the second binding moiety designated 85B2 of the multi-specific binding protein competes for binding to IL-12Rβ2 with one or more anti-IL-12Rβ2 binding moieties disclosed herein;
the second binding moiety designated 19B2 of the mutely-specific binding protein competes for binding to IL-12Rβ2 with one or more anti-IL-12Rβ2 binding moieties disclosed herein; or
the second binding moiety designated 230B2 of the multi-specific binding protein competes for binding to IL-12Rβ2 with one or more anti-IL-12Rβ2 binding moieties disclosed herein.

37-46. (canceled)

47. The multi-specific binding protein of claim 1, further comprising all or part of an immunoglobulin Fc domain or variant thereof-optionally wherein the Fc domain or variant thereof comprises a first Fc heavy chain and a second Fc heavy chain.

48. (canceled)

49. The multi-specific binding protein of claim 47, further comprising a variant Fc domain with reduced effector function, optionally wherein at least one Fc heavy chain comprises:
a substitution at amino acid position 234, according to EU numbering, optionally wherein the substitution at amino acid position 234 is an alanine (A):
a substitution at amino acid position 235, according to EU numbering, optionally wherein the substitution at amino acid position 235 is an alanine (A):
a substitution at amino acid position 237, according to EU numbering, optionally wherein the substitution at amino acid position 237 is an alanine (A): or
one or more substitutions at amino acid positions 234, 235, or 237, according to EU numbering, optionally wherein the substitution at amino acid position 234 is an alanine (A), wherein the substitution at amino acid position 235 is an alanine, and wherein the substitution at amino acid position 237 is an alanine (A).

50-57. (canceled)

58. The multi-specific binding protein of claim 1, wherein the Fc domain comprises heterodimerization mutations to promote heterodimerization of the first binding moiety with the second binding moiety, optionally wherein the heterodimerization mutations are Knob-in-Hole (KIH) mutations, optionally wherein:
the first Fc heavy chain comprises an amino acid substitution at position at position 366, 368, or 407 which produced a hole, and the second Fc heavy chain comprises an amino acid substitution at position 366 which produce a knob, optionally wherein the first:
the heterodimerization mutations are charge stabilization mutations, optionally wherein the first Fc heavy chain comprises the amino acid substitution N297K, and the second Fc heavy chain comprises the amino acid substitution N297D or wherein the first Fc heavy chain comprises the amino acid substitution T299K, and the second Fc heavy chain comprises the amino acid substitution T299D: or
the heterodimerization mutations comprise an engineered disulfide bond, optionally wherein the engineered disulfide bond is formed by a first Fc heavy chain comprising the amino acid substitution Y349C, and a second Fc heavy chain comprising the amino acid substitution S354C or wherein the engineered disulfide bond is formed by a C-terminal extension peptide fused to the C-terminus of each of the first Fc heavy chain and the second Fc heavy chain, optionally wherein the first Fc heavy chain C-terminal extension comprises the amino acid sequence GEC, and the second Fc heavy chain C-terminal extension comprises the amino acid sequence SCDKT (SEQ ID NO: 951).

59-68. (canceled)

69. The multi-specific binding protein of claim 1, wherein at least one Fc domain comprises one or more mutations to promote increased half-life, optionally wherein at least one Fc heavy chain comprises:
one or more substitutions at amino acid positions 252, 254, or 256, according to EU numbering, optionally wherein the substitution at amino acid position 252 is a tyrosine (Y), wherein the substitution at amino acid position 254 is a threonine (T), and wherein the substitution at amino acid position 236 is a glutamic acid (E); or
one or more substitutions at amino acid positions 428 or 434, according to EU numbering, wherein the substitution at amino acid position 428 is a leucine (L), and wherein the substitution at amino acid position 434 is a serine (S).

70-73. (canceled)

74. The multi-specific binding protein of claim 1, wherein:
the first binding moiety which specifically binds to human IL-12Rβ1 comprises heavy chain domain comprising an amino acid sequence set forth in any one of the sequences of Table 11;
the first binding moiety which specifically binds to human IL-12Rβ1 comprises a light chain domain comprising an amino acid sequence set forth in any one of the sequences in Table 11;
the second binding moiety which specifically binds to human IL-12Rβ2 comprises a heavy chain domain comprising an amino acid sequence set forth in any one of the sequences of Table 11;
the second binding moiety which specifically binds to human IL-12Rβ2 comprises a light chain domain comprising an amino acid sequence set forth in any one of the sequences of Table 11; and/or
the multi-specific binding protein comprises the HC and LC of any of the antibodies of Table 11.

75-78. (canceled)

79. A pharmaceutical composition comprising the multi-specific binding protein of claim 1 and a pharmaceutically acceptable carrier.

80. An isolated nucleic acid molecule encoding the multi-specific binding protein of claim 1.

81. An expression vector comprising the nucleic acid molecule of claim 80.

82. A host cell comprising the expression vector of claim 81.

83. A method for treating a disease or disorder in a subject, comprising administering to a subject in need thereof the multi-specific binding protein according to an claim 1, optionally wherein the disease or disorder is a cancer.

84-85. (canceled)

86. A multi-specific binding protein comprising at least a first binding moiety which binds specifically to a human interleukin-12 receptor β1 (IL-12Rβ1) subunit, and at least a second binding moiety which binds specifically to a human IL-12 receptor β2 (IL-12Rβ2) subunit, wherein:
a) the first binding moiety comprises:
i) a HCDR1 amino acid sequence, a HCDR2 amino acid sequence, and a HCDR3 amino acid sequence disclosed in Tables 1, 3, 4, 5, or 11;
ii) a LCDR1 amino acid sequence, a LCDR1 amino acid sequence, and a LCDR3 amino acid sequence disclosed in Tables 2, 3, or 11; and
b) the second binding moiety comprises:
i) a HCDR1 amino acid sequence, a HCDR2 amino acid sequence, and a HCDR3 amino acid sequence disclosed in Tables 6, 8, 9, 10, or 11;
ii) a LCDR1 amino acid sequence, a LCDR1 amino acid sequence, and a LCDR3 amino acid sequence disclosed in Tables 7, 8, or 11.


SEQUENCE LISTING (952 sequences)

SEQ ID NO: 1 - protein, 5 aa, synthetic construct
NAWMN

SEQ ID NO: 2 - protein, 19 aa, synthetic construct
RIKSKTDGGTTDYAAPVKG

SEQ ID NO: 3 - protein, 8 aa, synthetic construct
DPGWEFDY

SEQ ID NO: 4 - protein, 5 aa, synthetic construct
NYYWS

SEQ ID NO: 5 - protein, 16 aa, synthetic construct
YIFYNGITDYNPSLKS

SEQ ID NO: 6 - protein, 13 aa, synthetic construct
ESGMGATLMAFDI

SEQ ID NO: 7 - protein, 7 aa, synthetic construct
NARMGVS

SEQ ID NO: 8 - protein, 16 aa, synthetic construct
HIFSNDEKSYSTSLKS

SEQ ID NO: 9 - protein, 12 aa, synthetic construct
IGSGTYYGWFDP

SEQ ID NO: 10 - protein, 5 aa, synthetic construct
DYYMH

SEQ ID NO: 11 - protein, 17 aa, synthetic construct
WINPDSGGTNYAQKFQG

SEQ ID NO: 12 - protein, 9 aa, synthetic construct
EGATSNFDY

SEQ ID NO: 13 - protein, 5 aa, synthetic construct
DDYMH

SEQ ID NO: 14 - protein, 17 aa, synthetic construct
WINPNSGGTNYAQKFQG

SEQ ID NO: 15 - protein, 12 aa, synthetic construct
GGLNWGWDWFDP

SEQ ID NO: 16 - protein, 5 aa, synthetic construct
DYYMH

SEQ ID NO: 17 - protein, 17 aa, synthetic construct
WINPDNGGANYAQRFLG

SEQ ID NO: 18 - protein, 11 aa, synthetic construct
EGSGSPYAFDL

SEQ ID NO: 19 - protein, 5 aa, synthetic construct
SYYIH

SEQ ID NO: 20 - protein, 17 aa, synthetic construct
IINPSGGITSYAQQFQG

SEQ ID NO: 21 - protein, 8 aa, synthetic construct
EGDWYFDL

SEQ ID NO: 22 - protein, 5 aa, synthetic construct
SYDWS

SEQ ID NO: 23 - protein, 16 aa, synthetic construct
RIYTSGSTIYNPSLKS

SEQ ID NO: 24 - protein, 12 aa, synthetic construct
DRGNYPHDAFDI

SEQ ID NO: 25 - protein, 5 aa, synthetic construct
DYYIH

SEQ ID NO: 26 - protein, 17 aa, synthetic construct
WINPNSGGTHYAQKFQG

SEQ ID NO: 27 - protein, 12 aa, synthetic construct
EGGWNYFSGMDV

SEQ ID NO: 28 - protein, 5 aa, synthetic construct
SYDMN

SEQ ID NO: 29 - protein, 17 aa, synthetic construct
YINSRSSTIYYADSVKG

SEQ ID NO: 30 - protein, 8 aa, synthetic construct
EIEDSMDV

SEQ ID NO: 31 - protein, 5 aa, synthetic construct
SYWMS

SEQ ID NO: 32 - protein, 17 aa, synthetic construct
NIKQDGSEKYYVDSVKG

SEQ ID NO: 33 - protein, 9 aa, synthetic construct
GSIAAPFDY

SEQ ID NO: 34 - protein, 5 aa, synthetic construct
SYAMS

SEQ ID NO: 35 - protein, 17 aa, synthetic construct
GISGSGGGIYYADSVKG

SEQ ID NO: 36 - protein, 10 aa, synthetic construct
RLIGYWYFDL

SEQ ID NO: 37 - protein, 5 aa, synthetic construct
DYYMH

SEQ ID NO: 38 - protein, 17 aa, synthetic construct
WINPNSGGTHYSQKFQG

SEQ ID NO: 39 - protein, 12 aa, synthetic construct
EGDYISSSAFDY

SEQ ID NO: 40 - protein, 5 aa, synthetic construct
GYYMH

SEQ ID NO: 41 - protein, 17 aa, synthetic construct
WINPNSSGTNYAQKFQG

SEQ ID NO: 42 - protein, 10 aa, synthetic construct
MKRWLQGIDF

SEQ ID NO: 43 - protein, 5 aa, synthetic construct
SYYWS

SEQ ID NO: 44 - protein, 16 aa, synthetic construct
RIYTSGSTIYNPSLKS

SEQ ID NO: 45 - protein, 11 aa, synthetic construct
SRSYPHDAFDI

SEQ ID NO: 46 - protein, 5 aa, synthetic construct
SYGMH

SEQ ID NO: 47 - protein, 17 aa, synthetic construct
VIWNDGSNKYYADSVKG

SEQ ID NO: 48 - protein, 7 aa, synthetic construct
GELFFDY

SEQ ID NO: 49 - protein, 5 aa, synthetic construct
SYYIH

SEQ ID NO: 50 - protein, 17 aa, synthetic construct
IINPSGGSTSYAQKFQG

SEQ ID NO: 51 - protein, 9 aa, synthetic construct
SNWAYAFDI

SEQ ID NO: 52 - protein, 5 aa, synthetic construct
SYYWS

SEQ ID NO: 53 - protein, 16 aa, synthetic construct
RFYTSGSTIYNPSLES

SEQ ID NO: 54 - protein, 11 aa, synthetic construct
ERDYPHDAFDI

SEQ ID NO: 55 - protein, 5 aa, synthetic construct
SHGMH

SEQ ID NO: 56 - protein, 17 aa, synthetic construct
IIWYAGNKKYYADSVKG

SEQ ID NO: 57 - protein, 10 aa, synthetic construct
ESLLNSASDI

SEQ ID NO: 58 - protein, 5 aa, synthetic construct
SYWMS

SEQ ID NO: 59 - protein, 17 aa, synthetic construct
NIKQDGSEKYYVDSVKG

SEQ ID NO: 60 - protein, 12 aa, synthetic construct
EELTGLYWYFDL

SEQ ID NO: 61 - protein, 5 aa, synthetic construct
NYYMH

SEQ ID NO: 62 - protein, 17 aa, synthetic construct
IITSSGGSTSYAQKFQG

SEQ ID NO: 63 - protein, 8 aa, synthetic construct
EDLYYFDY

SEQ ID NO: 64 - protein, 5 aa, synthetic construct
GYYWN

SEQ ID NO: 65 - protein, 16 aa, synthetic construct
EINHSGSTHYNPSLKS

SEQ ID NO: 66 - protein, 8 aa, synthetic construct
ALTGYFDY

SEQ ID NO: 67 - protein, 5 aa, synthetic construct
DYYMH

SEQ ID NO: 68 - protein, 17 aa, synthetic construct
WINPNSGGTHYAQKFQG

SEQ ID NO: 69 - protein, 11 aa, synthetic construct
EGETVAGYFDY

SEQ ID NO: 70 - protein, 5 aa, synthetic construct
DYYLH

SEQ ID NO: 71 - protein, 17 aa, synthetic construct
WINPNNGGTHYAQKFQG

SEQ ID NO: 72 - protein, 10 aa, synthetic construct
EMGGTTAFDY

SEQ ID NO: 73 - protein, 5 aa, synthetic construct
DYYIH

SEQ ID NO: 74 - protein, 17 aa, synthetic construct
WINPDSGGTHYAQKFQG

SEQ ID NO: 75 - protein, 10 aa, synthetic construct
EGEGLASFDS

SEQ ID NO: 76 - protein, 5 aa, synthetic construct
SYWMS

SEQ ID NO: 77 - protein, 17 aa, synthetic construct
NIKQDGSEKYYVDSVKG

SEQ ID NO: 78 - protein, 10 aa, synthetic construct
EGLQLGIFDY

SEQ ID NO: 79 - protein, 5 aa, synthetic construct
SYFWS

SEQ ID NO: 80 - protein, 16 aa, synthetic construct
YLDYRGSTSYNPSVKS

SEQ ID NO: 81 - protein, 8 aa, synthetic construct
DLEDAFDV

SEQ ID NO: 82 - protein, 5 aa, synthetic construct
DYYMH

SEQ ID NO: 83 - protein, 17 aa, synthetic construct
WINPNSGGTNYAQKFQG

SEQ ID NO: 84 - protein, 13 aa, synthetic construct
EVGDIAVAGVFDY

SEQ ID NO: 85 - protein, 5 aa, synthetic construct
SYSMN

SEQ ID NO: 86 - protein, 17 aa, synthetic construct
YFSSRSGTKYYADSVKG

SEQ ID NO: 87 - protein, 9 aa, synthetic construct
EPHWDAFDL

SEQ ID NO: 88 - protein, 5 aa, synthetic construct
SYYMH

SEQ ID NO: 89 - protein, 17 aa, synthetic construct
IINPSGGITSYAQKFQG

SEQ ID NO: 90 - protein, 9 aa, synthetic construct
DPDWGFFDY

SEQ ID NO: 91 - protein, 5 aa, synthetic construct
SDYWI

SEQ ID NO: 92 - protein, 16 aa, synthetic construct
RIYTSGSTNYNPSLKS

SEQ ID NO: 93 - protein, 8 aa, synthetic construct
EVTGEFDY

SEQ ID NO: 94 - protein, 5 aa, synthetic construct
DYYIH

SEQ ID NO: 95 - protein, 17 aa, synthetic construct
WINPDSGGTTYAQKFQG

SEQ ID NO: 96 - protein, 10 aa, synthetic construct
EGSGSSSFDY

SEQ ID NO: 97 - protein, 5 aa, synthetic construct
DYYMH

SEQ ID NO: 98 - protein, 17 aa, synthetic construct
WINPNSGGTHYAQKFQG

SEQ ID NO: 99 - protein, 10 aa, synthetic construct
EGATTVYFDY

SEQ ID NO: 100 - protein, 7 aa, synthetic construct
TGGVGVG

SEQ ID NO: 101 - protein, 16 aa, synthetic construct
LIYWNDDKRYSPSLKS

SEQ ID NO: 102 - protein, 10 aa, synthetic construct
TLRWYHGFDI

SEQ ID NO: 103 - protein, 5 aa, synthetic construct
SYDMH

SEQ ID NO: 104 - protein, 16 aa, synthetic construct
VIGTAGDTYYPGSVKG

SEQ ID NO: 105 - protein, 11 aa, synthetic construct
SRGETDWYFDL

SEQ ID NO: 106 - protein, 11 aa, synthetic construct
QASQDISNFLN

SEQ ID NO: 107 - protein, 7 aa, synthetic construct
DASDLET

SEQ ID NO: 108 - protein, 9 aa, synthetic construct
QQYDNLPLT

SEQ ID NO: 109 - protein, 17 aa, synthetic construct
KSSQSVLYSSNNKNYLA

SEQ ID NO: 110 - protein, 7 aa, synthetic construct
WASTRES

SEQ ID NO: 111 - protein, 9 aa, synthetic construct
QQYYSSPLT

SEQ ID NO: 112 - protein, 14 aa, synthetic construct
TGTSSDIGSYNLVS

SEQ ID NO: 113 - protein, 7 aa, synthetic construct
EGSKRPS

SEQ ID NO: 114 - protein, 10 aa, synthetic construct
CSYAGTSTFV

SEQ ID NO: 115 - protein, 11 aa, synthetic construct
RASQGISNYLA

SEQ ID NO: 116 - protein, 7 aa, synthetic construct
AASNLQG

SEQ ID NO: 117 - protein, 9 aa, synthetic construct
QQYRYYPFT

SEQ ID NO: 118 - protein, 12 aa, synthetic construct
RASQNINNNYLA

SEQ ID NO: 119 - protein, 7 aa, synthetic construct
GASSRAT

SEQ ID NO: 120 - protein, 8 aa, synthetic construct
QQYGSSLT

SEQ ID NO: 121 - protein, 11 aa, synthetic construct
RASQGISSYLA

SEQ ID NO: 122 - protein, 7 aa, synthetic construct
AASTLQS

SEQ ID NO: 123 - protein, 9 aa, synthetic construct
QQLNRYPFT

SEQ ID NO: 124 - protein, 12 aa, synthetic construct
RASQSVSSSYLA

SEQ ID NO: 125 - protein, 7 aa, synthetic construct
GASSRAT

SEQ ID NO: 126 - protein, 8 aa, synthetic construct
QQYGSSYT

SEQ ID NO: 127 - protein, 11 aa, synthetic construct
RASQGISSFLA

SEQ ID NO: 128 - protein, 7 aa, synthetic construct
AASTLQS

SEQ ID NO: 129 - protein, 9 aa, synthetic construct
QQLNGYPFT

SEQ ID NO: 130 - protein, 11 aa, synthetic construct
RASQGISSYLA

SEQ ID NO: 131 - protein, 7 aa, synthetic construct
AASTLHS

SEQ ID NO: 132 - protein, 9 aa, synthetic construct
QQLNRYPYT

SEQ ID NO: 133 - protein, 13 aa, synthetic construct
SGSSSNIGNNYVS

SEQ ID NO: 134 - protein, 7 aa, synthetic construct
DNNERPS

SEQ ID NO: 135 - protein, 11 aa, synthetic construct
GTWDSSLSAGV

SEQ ID NO: 136 - protein, 11 aa, synthetic construct
QASQDISNYLN

SEQ ID NO: 137 - protein, 7 aa, synthetic construct
DASNLET

SEQ ID NO: 138 - protein, 9 aa, synthetic construct
QQYDNLPIT

SEQ ID NO: 139 - protein, 11 aa, synthetic construct
RASQGISNYLA

SEQ ID NO: 140 - protein, 7 aa, synthetic construct
AASSLQS

SEQ ID NO: 141 - protein, 9 aa, synthetic construct
QQYNSYPPT

SEQ ID NO: 142 - protein, 11 aa, synthetic construct
RASQGISSYLA

SEQ ID NO: 143 - protein, 7 aa, synthetic construct
AASTLQS

SEQ ID NO: 144 - protein, 9 aa, synthetic construct
QQLHRYPYT

SEQ ID NO: 145 - protein, 17 aa, synthetic construct
RSSQSLLDSNNGNTYLD

SEQ ID NO: 146 - protein, 7 aa, synthetic construct
TLSYRAS

SEQ ID NO: 147 - protein, 9 aa, synthetic construct
MQRIDFPLT

SEQ ID NO: 148 - protein, 11 aa, synthetic construct
RASQGISSYLA

SEQ ID NO: 149 - protein, 7 aa, synthetic construct
AASTLQS

SEQ ID NO: 150 - protein, 9 aa, synthetic construct
QQLNGYPFT

SEQ ID NO: 151 - protein, 11 aa, synthetic construct
QASQDISNYLN

SEQ ID NO: 152 - protein, 7 aa, synthetic construct
DASNLET

SEQ ID NO: 153 - protein, 8 aa, synthetic construct
QQYDNLFT

SEQ ID NO: 154 - protein, 11 aa, synthetic construct
QGDSLSSYYAG

SEQ ID NO: 155 - protein, 7 aa, synthetic construct
GKNNRPS

SEQ ID NO: 156 - protein, 11 aa, synthetic construct
NSRDSSDNHLV

SEQ ID NO: 157 - protein, 11 aa, synthetic construct
RASQDISSFLA

SEQ ID NO: 158 - protein, 7 aa, synthetic construct
AASTLQS

SEQ ID NO: 159 - protein, 9 aa, synthetic construct
QQLNGYPLT

SEQ ID NO: 160 - protein, 13 aa, synthetic construct
SGSSSNIGNNYVS

SEQ ID NO: 161 - protein, 7 aa, synthetic construct
DNNKRPS

SEQ ID NO: 162 - protein, 11 aa, synthetic construct
GTWDSSLSAVV

SEQ ID NO: 163 - protein, 12 aa, synthetic construct
RASQSINSNYLA

SEQ ID NO: 164 - protein, 7 aa, synthetic construct
GASSRAT

SEQ ID NO: 165 - protein, 5 aa, synthetic construct
QQYDS

SEQ ID NO: 166 - protein, 14 aa, synthetic construct
TGTSSDVGSYNLVS

SEQ ID NO: 167 - protein, 7 aa, synthetic construct
EGSKRPS

SEQ ID NO: 168 - protein, 11 aa, synthetic construct
CSYAGSSTFYV

SEQ ID NO: 169 - protein, 13 aa, synthetic construct
SGSSSNIGSNYVY

SEQ ID NO: 170 - protein, 7 aa, synthetic construct
SNNQRPS

SEQ ID NO: 171 - protein, 12 aa, synthetic construct
AAWDDSLSGPVV

SEQ ID NO: 172 - protein, 11 aa, synthetic construct
RASQGISSYLA

SEQ ID NO: 173 - protein, 7 aa, synthetic construct
AASTLQN

SEQ ID NO: 174 - protein, 9 aa, synthetic construct
QQHNSYPYT

SEQ ID NO: 175 - protein, 11 aa, synthetic construct
RASQGIGNYLA

SEQ ID NO: 176 - protein, 7 aa, synthetic construct
AASSLQS

SEQ ID NO: 177 - protein, 9 aa, synthetic construct
QQYNIYPYT

SEQ ID NO: 178 - protein, 11 aa, synthetic construct
RASQGINNYLA

SEQ ID NO: 179 - protein, 7 aa, synthetic construct
AASSLQS

SEQ ID NO: 180 - protein, 9 aa, synthetic construct
QQYNNYPFT

SEQ ID NO: 181 - protein, 11 aa, synthetic construct
QASQDISNYLN

SEQ ID NO: 182 - protein, 7 aa, synthetic construct
DASNLET

SEQ ID NO: 183 - protein, 9 aa, synthetic construct
QQYDNLPFT

SEQ ID NO: 184 - protein, 13 aa, synthetic construct
SGSSSNIGNNYVS

SEQ ID NO: 185 - protein, 7 aa, synthetic construct
DNNERPS

SEQ ID NO: 186 - protein, 11 aa, synthetic construct
GTWDSNLSAGV

SEQ ID NO: 187 - protein, 11 aa, synthetic construct
RASQGISSYLA

SEQ ID NO: 188 - protein, 7 aa, synthetic construct
AASTLQS

SEQ ID NO: 189 - protein, 9 aa, synthetic construct
QQHSRYPYT

SEQ ID NO: 190 - protein, 13 aa, synthetic construct
SGSSSNIGSNTVN

SEQ ID NO: 191 - protein, 7 aa, synthetic construct
SNNQRPS

SEQ ID NO: 192 - protein, 11 aa, synthetic construct
AAWDDSLNGHV

SEQ ID NO: 193 - protein, 11 aa, synthetic construct
RASQSISSWLA

SEQ ID NO: 194 - protein, 7 aa, synthetic construct
KASSLES

SEQ ID NO: 195 - protein, 9 aa, synthetic construct
QQYTSYMYT

SEQ ID NO: 196 - protein, 11 aa, synthetic construct
QASQDISNYLN

SEQ ID NO: 197 - protein, 7 aa, synthetic construct
DASNLET

SEQ ID NO: 198 - protein, 8 aa, synthetic construct
QQYDNLYT

SEQ ID NO: 199 - protein, 11 aa, synthetic construct
RASQAISNYLA

SEQ ID NO: 200 - protein, 7 aa, synthetic construct
TASSLQS

SEQ ID NO: 201 - protein, 9 aa, synthetic construct
QQYRFYPYT

SEQ ID NO: 202 - protein, 11 aa, synthetic construct
RASQGINNYLA

SEQ ID NO: 203 - protein, 7 aa, synthetic construct
DASTLQS

SEQ ID NO: 204 - protein, 9 aa, synthetic construct
QQLNMYPYT

SEQ ID NO: 205 - protein, 14 aa, synthetic construct
GLTSGSVSTSYYPS

SEQ ID NO: 206 - protein, 7 aa, synthetic construct
STYTRSS

SEQ ID NO: 207 - protein, 10 aa, synthetic construct
VLYMGSGIWV

SEQ ID NO: 208 - protein, 11 aa, synthetic construct
RASQSVSRLLA

SEQ ID NO: 209 - protein, 7 aa, synthetic construct
DASNRAT

SEQ ID NO: 210 - protein, 8 aa, synthetic construct
QQRRNWLT

SEQ ID NO: 211 - protein, 119 aa, synthetic construct
EVQLVESGGGLVKPGGSLRLSCAASGFTFSNAWMNWVRQAPGKGLEWVGRIKSKTDGGTTDYAAPVKGRFTVSRDDPKNTLYLQMNSLKTEDTAVYYCTTDPGWEFDYWGQGILVTVSS

SEQ ID NO: 212 - protein, 107 aa, synthetic construct
DIQMTQSPSSLSASVGDRVTITCQASQDISNFLNWYQQKPGKAPNLLIYDASDLETGVPSRFSGSGSGTDFTFTISSLQPEDIATYYCQQYDNLPLTFGGGTKVEIK

SEQ ID NO: 213 - protein, 121 aa, synthetic construct
QVQLQESGPGLVKPSETLSLTCTVSGGSISNYYWSWIRQPPGKGLEWIGYIFYNGITDYNPSLKSRVTISVDTSKNQFSLKLSSVTAADTAMYFCVRESGMGATLMAFDIWGQGTMVTVSS

SEQ ID NO: 214 - protein, 113 aa, synthetic construct
DIVMTQSPDSLAVSLGERATINCKSSQSVLYSSNNKNYLAWYQQKPGQPPKMVIYWASTRESGVPDRFSGSGSGTDFTLTISSLQAEDVAVYYCQQYYSSPLTFGGGTKVEIK

SEQ ID NO: 215 - protein, 122 aa, synthetic construct
QVTLKESGPVLVKPTETLTLTCTVSGFSLTNARMGVSWIRQPPGKALEWLAHIFSNDEKSYSTSLKSRFTISKDTSKSQVVLTMTNLDPVDTATYYCARIGSGTYYGWFDPWGQGTLVTVSS

SEQ ID NO: 216 - protein, 110 aa, synthetic construct
QSALTQPASVSGSPGQSITISCTGTSSDIGSYNLVSWYQQHPGKAPKLLIYEGSKRPSGVSNRFSGSKSGTTASLTISGLQAEDEADYYCCSYAGTSTFVFGTGTKVTVL

SEQ ID NO: 217 - protein, 118 aa, synthetic construct
QVQLVHSGPEVKKPGASVKVSCKASGYTFTDYYMHWVRQAPGQGLEWMGWINPDSGGTNYAQKFQGRVTMTRDTSISTAYMELSRLRSDDTAVYHCAREGATSNFDYWGQGTLVTVSS

SEQ ID NO: 218 - protein, 107 aa, synthetic construct
DIQMTQSPSSLSASVGDRVTITCRASQGISNYLAWFQQKPGKAPTSLIYAASNLQGGVPSKFSGSGSGTDFTLTISSLQPEDFATYYCQQYRYYPFTFGPGTKVDIK

SEQ ID NO: 219 - protein, 121 aa, synthetic construct
QVQLVQSGAEVKKPGASVKVSCKASEYTFTDDYMHWVRQAPGQGLVWMGWINPNSGGTNYAQKFQGRVTMTRDTSISTAYMELSRLRSDDTAVYYCARGGLNWGWDWFDPWGQGTLVTVSS

SEQ ID NO: 220 - protein, 107 aa, synthetic construct
EIVLTQSPGTLSLSPGERATLSCRASQNINNNYLAWYQRKPGQAPRLLIYGASSRATGIPDRFSGSGSGTDFTLTISRLEPEDFAMYYCQQYGSSLTFGGGTKVEIK

SEQ ID NO: 221 - protein, 120 aa, synthetic construct
QVQLVQSGAEVRKPGASVKVSCKASGYTFTDYYMHWVRQAPGQGLEWMGWINPDNGGANYAQRFLGRVTMTRDTSISTAYMELSRLRSDDTAVYYCAREGSGSPYAFDLWGLGTVVTVSS

SEQ ID NO: 222 - protein, 107 aa, synthetic construct
DIQLTQSPSFLSASVGDRVTITCRASQGISSYLAWYQQKPGKAPKLLIYAASTLQSGVPSRFSGSGSGTEFTLTISSLQPEDFATYCCQQLNRYPFTFGQGTKLEIK

SEQ ID NO: 223 - protein, 117 aa, synthetic construct
QVQLVQSGAEVKKPGASVKVSCKTSGYTFTSYYIHWVRQAPGQGLEWMGIINPSGGITSYAQQFQGRVTMTRDTSTSTVYMELSSLRSEDTAVYYCAREGDWYFDLWGRGTLVTVSS

SEQ ID NO: 224 - protein, 107 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSYTFGQGTKLEIK

SEQ ID NO: 225 - protein, 120 aa, synthetic construct
QVQLQESGPGLVKPSETLSLTCTVSGGSISSYDWSWIRQSAGKGLEWIGRIYTSGSTIYNPSLKSRVTMSVDTSKNEISLKLSSVTAADTAFYYCAKDRGNYPHDAFDIWGQGTMITVSS

SEQ ID NO: 226 - protein, 107 aa, synthetic construct
DIQLTQSPSFLSASVGDRVTITCRASQGISSFLAWYQQKPGKAPKLLIYAASTLQSGVPSRFSGSGSGTEFTLTISSLQPEDFATYYCQQLNGYPFTFGPGTKVDIK

SEQ ID NO: 227 - protein, 121 aa, synthetic construct
QVHLVQSGAEVKKPGASVKVSCEASGYTFTDYYIHWLRQAPGQGLEWMGWINPNSGGTHYAQKFQGRVTMTRDTSISTAYMELSRLSSDDTAVYYCAREGGWNYFSGMDVWGQGTTVTVSS

SEQ ID NO: 228 - protein, 107 aa, synthetic construct
DIQLTQSPSFLSASVGDRVTITCRASQGISSYLAWYQQQPGKPPKLLIYAASTLHSGVPSRFSGSGSGTEFTLTISSLQPEDFATYYCQQLNRYPYTCGQGTKLEIK

SEQ ID NO: 229 - protein, 117 aa, synthetic construct
EVQLVESGGGLVQPGGSLRLSCAASGFTFSSYDMNWVRQAPGKGLEWISYINSRSSTIYYADSVKGRFTISRDNAKNSLYLQMNSLRDEDTALYYCAREIEDSMDVWGQGTTVTVSS

SEQ ID NO: 230 - protein, 110 aa, synthetic construct
QSVLTQPPSVSAAPGQKVTISCSGSSSNIGNNYVSWYQQLPGTAPKLLIYDNNERPSGIPDRFSGSKSGTSATLGITGLQPGDEADYYCGTWDSSLSAGVFGGGTKLTVL

SEQ ID NO: 231 - protein, 118 aa, synthetic construct
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARGSIAAPFDYWGQGTLVTVSS

SEQ ID NO: 232 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPITFGQGTRLEIK

SEQ ID NO: 233 - protein, 119 aa, synthetic construct
EVQLLESGGGLVQPGGSLRLSCAASGFTFSSYAMSWVRQAPGKGLEWVAGISGSGGGIYYADSVKGRFTISRDNSKNTLYLQLNSLRADDTAVYYCARRLIGYWYFDLWGRGTLVPVSS

SEQ ID NO: 234 - protein, 107 aa, synthetic construct
DIQMTQSPSSLSASVGDRVTITCRASQGISNYLAWFQQKPGKAPKSLIYAASSLQSGVPSKFSGSGSGTDFTLTISSLQPEDFATYYCQQYNSYPPTFGGGTKVEIK

SEQ ID NO: 235 - protein, 121 aa, synthetic construct
QVHLVQSGAEVKKPGASVKVSCKASGYTFIDYYMHWVRQAPGQGLEWMGWINPNSGGTHYSQKFQGRVTMTWDTSISTAYMELSRLTSDDTAVYYCAREGDYISSSAFDYWGQGTLVTVSS

SEQ ID NO: 236 - protein, 107 aa, synthetic construct
DIQLTQSPSFLSASVGDRVTITCRASQGISSYLAWYQQKPGKAPKVLIYAASTLQSGVPSRFSGSGSGTEFTLTISSLLPEDFATYFCQQLHRYPYTFGQGAKLEIK

SEQ ID NO: 237 - protein, 119 aa, synthetic construct
QVQLVQSGAEVMKPGASVKVSCKASGYTFTGYYMHWIRQAPGQGLEWMGWINPNSSGTNYAQKFQGRVTMTRDTSISTAYMELSRLRSDDTAVYYCARMKRWLQGIDFWGQGILVTVSS

SEQ ID NO: 238 - protein, 113 aa, synthetic construct
DIVMTQTPLSLPVTPGEPASISCRSSQSLLDSNNGNTYLDWYLQKPGQSPHLLIYTLSYRASGVPDRFSGSGSGTDFTLKISRVEAEDVGVYYCMQRIDFPLTFGGGTKVEIK

SEQ ID NO: 239 - protein, 119 aa, synthetic construct
QVQLQESGPGLVKPSETLSLTCTVSGGSISSYYWSWIRQSAGKELEWIGRIYTSGSTIYNPSLKSRVTMSVDPSKNQFSLKLSSVTAADTAVYYCANSRSYPHDAFDIWGQGTMVTVSS

SEQ ID NO: 240 - protein, 107 aa, synthetic construct
DIQLTQSPSFLSASVGYRVTITCRASQGISSYLAWYQQKPGKAPQLLIYAASTLQSGVPSRFSGRGSGTEFTLTISSLQPEDFATYYCQQLNGYPFTFGPGTKVNIK

SEQ ID NO: 241 - protein, 116 aa, synthetic construct
QVQLVESGGGVVQPGRSLRLSCAASGFTFSSYGMHWVRQAPGKGLEWVAVIWNDGSNKYYADSVKGRFTISRDNSKNTLYLQMNSLRAEDTAVYYCARGELFFDYWGQGTLVTVSS

SEQ ID NO: 242 - protein, 106 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLFTFGGGTKVEIK

SEQ ID NO: 243 - protein, 118 aa, synthetic construct
QVQLVQSGAEVKKPGASVRVSCKASGYTFTSYYIHWVRQAPGQGLEWMGIINPSGGSTSYAQKFQGRVTMTRDTSTTTVYMELSSLRSEDTAVYYCAKSNWAYAFDIWGQGTMVTVSS

SEQ ID NO: 244 - protein, 108 aa, synthetic construct
SSELTQDPAVSVALGQTVRITCQGDSLSSYYAGWYQQKPGQAPVLVIYGKNNRPSGIPDRFSGSSSEDTASLTITGAQAEDEADYYCNSRDSSDNHLVFGGGTKLTVL

SEQ ID NO: 245 - protein, 119 aa, synthetic construct
QVQLQESGPGLVKPSETLSLTCTVSGGSISSYYWSWIRQPAGTGLEWIGRFYTSGSTIYNPSLESRVTMSVDTSKNQFSLKLSSVTAADTAVYYCATERDYPHDAFDIWGQGTMVTVSS

SEQ ID NO: 246 - protein, 107 aa, synthetic construct
DIQLTQSPSFLSASVGDRVTIPCRASQDISSFLAWYQQKPGKAPNLLIYAASTLQSGVPSRFSGSGSGTEFTLTISSLQPEDFATYYCQQLNGYPLTFGGGTKVEIK

SEQ ID NO: 247 - protein, 119 aa, synthetic construct
QVQLVESGGGVVQPGRSLRLSCAASGFTFSSHGMHWVRQAPGKGLEWVAIIWYAGNKKYYADSVKGRFTISRDNSKNTLYLQMNSLRAEDTAVYYCARESLLNSASDIWGQGTMVTVSS

SEQ ID NO: 248 - protein, 110 aa, synthetic construct
QSVLTQPPSVSAAPGQTVTISCSGSSSNIGNNYVSWYQQLPGTAPKLLIYDNNKRPSGIPDRFSGSKSGTSATLGITGLQTGDEADYYCGTWDSSLSAVVFGGGTKLTVL

SEQ ID NO: 249 - protein, 121 aa, synthetic construct
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAREELTGLYWYFDLWGRGTLVTVSS

SEQ ID NO: 250 - protein, 104 aa, synthetic construct
EIVLTQSPGTLSLSPGERATLSCRASQSINSNYLAWYQQKPGQAPRLLIYGASSRATGIPDRFSGSGSGTDFTLTISRLEPEDFAVYYCQQYDSFGGGTKVEIK

SEQ ID NO: 251 - protein, 117 aa, synthetic construct
QVQLVQSGAEVKKPGASVKVSCKASGYTFTNYYMHWVRQAPGQGLEWMGIITSSGGSTSYAQKFQGRVTMTRDTSTSTVYMELSSLKSEDTAVYYCAREDLYYFDYWGQGTLVTVSS

SEQ ID NO: 252 - protein, 111 aa, synthetic construct
QSALTQPASVSGSPGQSITISCTGTSSDVGSYNLVSWYQQHPGKAPKLMIYEGSKRPSGVSNRFSGSKSGNTASLTISGLQAEDEADYYCCSYAGSSTFYVFGTGTKVTVL

SEQ ID NO: 253 - protein, 116 aa, synthetic construct
QVQLQQWGAGLLKPSETLSLTCAVYGGSFSGYYWNWIRQPPGKGLEWIGEINHSGSTHYNPSLKSRVTISVDTSKNQFSLKLSSVTAADTALYYCARALTGYFDYWGQGTLVTVSS

SEQ ID NO: 254 - protein, 111 aa, synthetic construct
QSVLTQPPSASGTPGQRVTISCSGSSSNIGSNYVYWYQQLPGTAPKLLIYSNNQRPSGVPDRFSGSKSGTSASLAISGLRSEDEADYYCAAWDDSLSGPVVFGGGTKLTVL

SEQ ID NO: 255 - protein, 120 aa, synthetic construct
QVQLVQSGAEVKKPGASVKVSCKASGYTFTDYYMHWVRQAPGQGLEWMGWINPNSGGTHYAQKFQGRVTMTKDTSISTAYMELSRLRSDDTAVYYCAREGETVAGYFDYWGQGILVTVSS

SEQ ID NO: 256 - protein, 107 aa, synthetic construct
DIQLTQSPSFLSASVGDRVTITSRASQGISSYLAWYQQKPGKAPKLLIYAASTLQNGVPSRFSGSGSGTEFTLTISSLQPEDFATYYCQQHNSYPYTFGQGTKLEIK

SEQ ID NO: 257 - protein, 119 aa, synthetic construct
QVQLVQSGAEVKKPGASAKVSCKASGYTFTDYYLHWVRQAPGQGLEWMGWINPNNGGTHYAQKFQGRVTMTRDTSISTAYMELSRLRSDDTAVYYCAREMGGTTAFDYWGQGTLVTVSS

SEQ ID NO: 258 - protein, 107 aa, synthetic construct
DIQMTQSPSSLSASVGDRVTITCRASQGIGNYLAWFQQRPGKAPKSLIYAASSLQSGVPSKFSGSGSGTDFTLTISSLQPADFAIYYCQQYNIYPYTFGPGTRLEIK

SEQ ID NO: 259 - protein, 119 aa, synthetic construct
QVQLVQSGAEVKKPGASVKVSCKASGYTFTDYYIHWVRQAPGQGLEWMGWINPDSGGTHYAQKFQGRVTMTRDTSISTVYMDLSRLRSDDTAVYYCAREGEGLASFDSWGQGTLVTVSS

SEQ ID NO: 260 - protein, 107 aa, synthetic construct
DIQMTQSPSSLSASVGNRVTITCRASQGINNYLAWFQQKPGKAPKSLIYAASSLQSGVPSKFSGSGSGTDFTLTISSLQPEDFATYYCQQYNNYPFTFGPGTKVDIK

SEQ ID NO: 261 - protein, 119 aa, synthetic construct
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAREGLQLGIFDYWGQGTLVTVSS

SEQ ID NO: 262 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPFTFGPGTKVDIK

SEQ ID NO: 263 - protein, 116 aa, synthetic construct
QVQLQESGPGLVKPSETLSLTCTVSGVSISSYFWSWIRQPPGKGLEWIGYLDYRGSTSYNPSVKSRVTISLDTSKNQFSLKLTSVTAADTAAYYCARDLEDAFDVWGQGTMVTVSS

SEQ ID NO: 264 - protein, 110 aa, synthetic construct
QSVLTQPPSVSAAPGQKVTISCSGSSSNIGNNYVSWYQQLPGTAPKLLIYDNNERPSGIPDRFSGSKSGTSATLGITGLQTGDEADYYCGTWDSNLSAGVFGGGTRLTVL

SEQ ID NO: 265 - protein, 122 aa, synthetic construct
QVQLVQSGAEVKKPGASVKVSCKASGYTFTDYYMHWVRQAPGQGLEWMGWINPNSGGTNYAQKFQGRVTMTRDTSISTAYMELSRLRSDDTAVYYCAREVGDIAVAGVFDYWGQGTLVTVSS

SEQ ID NO: 266 - protein, 107 aa, synthetic construct
DIQLTQSPSFLSASVGDRVTITCRASQGISSYLAWYQQKPGKAPKLLIYAASTLQSGVPSRFSGSGSGTEFTVTISSLQPEDFATYYCQQHSRYPYTFGQGTKVEIK

SEQ ID NO: 267 - protein, 118 aa, synthetic construct
EVQLVESGGGLVQPGGSLRLSCAASGFTFSSYSMNWVRQAPGKGLEWLSYFSSRSGTKYYADSVKGRFTISRDNAKNSLYLQMNSLRDEDTAVYYCAREPHWDAFDLWGQGTMVTVSS

SEQ ID NO: 268 - protein, 110 aa, synthetic construct
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGHVFGTGTKVTVL

SEQ ID NO: 269 - protein, 118 aa, synthetic construct
QVQLVQSGAEVKKPGASVKVSCKPSGYTFTSYYMHWVRQAPGQGLEWMGIINPSGGITSYAQKFQGRVTMTRDTSTSTVYMELSSLRSEDTAVYYCARDPDWGFFDYWGQGSLVTVSS

SEQ ID NO: 270 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYTSYMYTFGQGTKLEIK

SEQ ID NO: 271 - protein, 116 aa, synthetic construct
QVHLQESGPGLVKPSETLSLTCTVSGVSISSDYWIWIRQPAGKGLEWIGRIYTSGSTNYNPSLKSRVTMSVDTSKNQFSLKLSSVTAADTAVYYCAREVTGEFDYWGQGTLVTVSS

SEQ ID NO: 272 - protein, 106 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLYTFGQGTKLEIK

SEQ ID NO: 273 - protein, 119 aa, synthetic construct
QVQLVQTGAEVKKPGASVKVSCKASRYTFTDYYIHWVRQAPGQGLEWMGWINPDSGGTTYAQKFQGRVTMTRDTSINTAYMELSRLRSDDTAVYFCAREGSGSSSFDYWGQGTLVTVSS

SEQ ID NO: 274 - protein, 107 aa, synthetic construct
DIQMTQSPSSLSASVGDRVTITCRASQAISNYLAWFQQKPGKAPKSLIYTASSLQSGVPSKFSGSGSGTDFTLTISSLHPEDFATYYCQQYRFYPYTFGLGTKLEIK

SEQ ID NO: 275 - protein, 119 aa, synthetic construct
QVQLVQSGAEVRKPGASVKVSCKASGYTFTDYYMHWVRQAPGQGREWMGWINPNSGGTHYAQKFQGRVTMTWDTSISTAYMELSRLRSDDTAVYYCAREGATTVYFDYWGQGTLVTVSS

SEQ ID NO: 276 - protein, 107 aa, synthetic construct
DIQLTQSPSFLSASVGDRVTLTCRASQGINNYLAWYQEKPGKAPNPLIYDASTLQSGVPSRFSGSGSGTEFTLTISSLQPEDFATYYCQQLNMYPYTFGQGTKLEIK

SEQ ID NO: 277 - protein, 120 aa, synthetic construct
QITLKESGPTLVKPTQTLTLTCTFFGFSLNTGGVGVGWIRQPPGKALEWLTLIYWNDDKRYSPSLKSRLTITKDTSKNQVVLTMTNMDPVDTATYYCAHTLRWYHGFDIWGQGTMVTVSS

SEQ ID NO: 278 - protein, 110 aa, synthetic construct
QTVVTQEPSFSVSPGGTVTLTCGLTSGSVSTSYYPSWYQQTPGQAPRTLIYSTYTRSSGVPDRFSGSILGNKAALTITGAQADDESDYYCVLYMGSGIWVFGGGTKLTVL

SEQ ID NO: 279 - protein, 119 aa, synthetic construct
EVQLVESGGGLVQPGGSLRLSCAASGFTFSSYDMHWVRQATGKGLEWVSVIGTAGDTYYPGSVKGRFTISRENAKNSLYLQMNSLRAGDTAVYYCARSRGETDWYFDLWGRGTLVTVSS

SEQ ID NO: 280 - protein, 106 aa, synthetic construct
EIVLTQSPATLSLSPGERATLSCRASQSVSRLLAWYQQKPGQAPRLLIYDASNRATGIPARFSGSGSGTDFTLTISSLEPEDFAVYYCQQRRNWLTFGGGTKVEIK

SEQ ID NO: 281 - protein, 5 aa, synthetic construct
YFAIG

SEQ ID NO: 282 - protein, 17 aa, synthetic construct
CIGSSDGSTIYSDSVKG

SEQ ID NO: 283 - protein, 18 aa, synthetic construct
DRGRRTIIGYACYATMKY

SEQ ID NO: 284 - protein, 5 aa, synthetic construct
NYAMS

SEQ ID NO: 285 - protein, 17 aa, synthetic construct
VINSGGGSTLYADSVKG

SEQ ID NO: 286 - protein, 14 aa, synthetic construct
RRDDSTFGSLLYTH

SEQ ID NO: 287 - protein, 5 aa, synthetic construct
TYAMA

SEQ ID NO: 288 - protein, 17 aa, synthetic construct
HILWSGGSTVYPDSVRG

SEQ ID NO: 289 - protein, 17 aa, synthetic construct
KSGRQVYRIDSWDSFDY

SEQ ID NO: 290 - protein, 5 aa, synthetic construct
RYSMG

SEQ ID NO: 291 - protein, 17 aa, synthetic construct
ANYWSNGWTDYADTVKG

SEQ ID NO: 292 - protein, 15 aa, synthetic construct
RSSSIPSADVVGYDY

SEQ ID NO: 293 - protein, 5 aa, synthetic construct
GYVMG

SEQ ID NO: 294 - protein, 16 aa, synthetic construct
GITGISSTYFADSVKG

SEQ ID NO: 295 - protein, 16 aa, synthetic construct
VNSFGVIPTSPNGMDY

SEQ ID NO: 296 - protein, 4 aa, synthetic construct
YAVG

SEQ ID NO: 297 - protein, 19 aa, synthetic construct
VAAITVSGGSKYYEDSVKG

SEQ ID NO: 298 - protein, 18 aa, synthetic construct
RTPKFGSGWYNRRGEYDY

SEQ ID NO: 299 - protein, 5 aa, synthetic construct
SYAVA

SEQ ID NO: 300 - protein, 17 aa, synthetic construct
SISWSGGSTNYADSVKG

SEQ ID NO: 301 - protein, 14 aa, synthetic construct
DRNYYPSVLGKYTY

SEQ ID NO: 302 - protein, 5 aa, synthetic construct
NYAMA

SEQ ID NO: 303 - protein, 17 aa, synthetic construct
GITWGGGLTHYVDSVKG

SEQ ID NO: 304 - protein, 17 aa, synthetic construct
KTQGSTWYHLTPDGYDY

SEQ ID NO: 305 - protein, 5 aa, synthetic construct
AYAMG

SEQ ID NO: 306 - protein, 17 aa, synthetic construct
GISRSGSTTDYVDTAKG

SEQ ID NO: 307 - protein, 18 aa, synthetic construct
VTGTFPSSTYHNANAYHH

SEQ ID NO: 308 - protein, 5 aa, synthetic construct
NDDMA

SEQ ID NO: 309 - protein, 17 aa, synthetic construct
HIDRNAASTNYADSVKG

SEQ ID NO: 310 - protein, 15 aa, synthetic construct
DPDHFGTTWYQQYTY

SEQ ID NO: 311 - protein, 5 aa, synthetic construct
INIMG

SEQ ID NO: 312 - protein, 16 aa, synthetic construct
SITSGGSTFYADSVKD

SEQ ID NO: 313 - protein, 11 aa, synthetic construct
YRSVPLNTVYP

SEQ ID NO: 314 - protein, 5 aa, synthetic construct
DRSMG

SEQ ID NO: 315 - protein, 16 aa, synthetic construct
AITVGGSTNYADSVKG

SEQ ID NO: 316 - protein, 7 aa, synthetic construct
RYPPDDY

SEQ ID NO: 317 - protein, 4 aa, synthetic construct
YFMG

SEQ ID NO: 318 - protein, 17 aa, synthetic construct
AISRSGGGTWYADSVKG

SEQ ID NO: 319 - protein, 16 aa, synthetic construct
DRRTGTSVYTVDEYDY

SEQ ID NO: 320 - protein, 4 aa, synthetic construct
YAVG

SEQ ID NO: 321 - protein, 19 aa, synthetic construct
VAAITVSGGSKYYEDSVKG

SEQ ID NO: 322 - protein, 18 aa, synthetic construct
RTPKFGSGWYNRRGEYDY

SEQ ID NO: 323 - protein, 5 aa, synthetic construct
TYAMG

SEQ ID NO: 324 - protein, 20 aa, synthetic construct
IAAWKVASTYYVDYGDSVRG

SEQ ID NO: 325 - protein, 12 aa, synthetic construct
GAFRTDWRSYAY

SEQ ID NO: 326 - protein, 4 aa, synthetic construct
YAVG

SEQ ID NO: 327 - protein, 19 aa, synthetic construct
VAAITVSGGSKYYEDSVKG

SEQ ID NO: 328 - protein, 18 aa, synthetic construct
RTPKFGSGWYNRRGEYDY

SEQ ID NO: 329 - protein, 5 aa, synthetic construct
VNTRA

SEQ ID NO: 330 - protein, 16 aa, synthetic construct
QISSGGNINYADSVKG

SEQ ID NO: 331 - protein, 9 aa, synthetic construct
HRQHPYADV

SEQ ID NO: 332 - protein, 5 aa, synthetic construct
HYVMA

SEQ ID NO: 333 - protein, 16 aa, synthetic construct
SIWWGGSTYYADSVKD

SEQ ID NO: 334 - protein, 13 aa, synthetic construct
GTKIGTMGPRYDY

SEQ ID NO: 335 - protein, 5 aa, synthetic construct
YYAIG

SEQ ID NO: 336 - protein, 17 aa, synthetic construct
CISSSDRSTWYADSVKG

SEQ ID NO: 337 - protein, 15 aa, synthetic construct
PCYSDYDPEGYEYDY

SEQ ID NO: 338 - protein, 5 aa, synthetic construct
TYAMG

SEQ ID NO: 339 - protein, 17 aa, synthetic construct
RISNSGAFTHYADSVKG

SEQ ID NO: 340 - protein, 17 aa, synthetic construct
TRGTNSAYYTRSTMYEY

SEQ ID NO: 341 - protein, 5 aa, synthetic construct
IDVMA

SEQ ID NO: 342 - protein, 16 aa, synthetic construct
AISSGGSLNYRDSVKG

SEQ ID NO: 343 - protein, 15 aa, synthetic construct
NIRTSPVTTRPMGNY

SEQ ID NO: 344 - protein, 5 aa, synthetic construct
FNEMA

SEQ ID NO: 345 - protein, 17 aa, synthetic construct
TIVSTVGFTNYADSVKG

SEQ ID NO: 346 - protein, 7 aa, synthetic construct
RRISTDY

SEQ ID NO: 347 - protein, 5 aa, synthetic construct
SYGMS

SEQ ID NO: 348 - protein, 17 aa, synthetic construct
YISAGGGTTTYVDFVKG

SEQ ID NO: 349 - protein, 5 aa, synthetic construct
AADGS

SEQ ID NO: 350 - protein, 5 aa, synthetic construct
INAMG

SEQ ID NO: 351 - protein, 15 aa, synthetic construct
IQLSGQTSYAHSVRG

SEQ ID NO: 352 - protein, 12 aa, synthetic construct
SNTGSLYGLRNY

SEQ ID NO: 353 - protein, 5 aa, synthetic construct
SYVMG

SEQ ID NO: 354 - protein, 17 aa, synthetic construct
AIMRNGIMTYYADSVKG

SEQ ID NO: 355 - protein, 16 aa, synthetic construct
ASRVGVSISQSSSYTS

SEQ ID NO: 356 - protein, 5 aa, synthetic construct
FNAMG

SEQ ID NO: 357 - protein, 16 aa, synthetic construct
IIINGDMINYADSVKG

SEQ ID NO: 358 - protein, 7 aa, synthetic construct
TRYVYDY

SEQ ID NO: 359 - protein, 5 aa, synthetic construct
SYAMG

SEQ ID NO: 360 - protein, 21 aa, synthetic construct
AISGGTPNNIYYTNYKDAVRG

SEQ ID NO: 361 - protein, 13 aa, synthetic construct
TMGRYWNTGGYDY

SEQ ID NO: 362 - protein, 5 aa, synthetic construct
DYAMK

SEQ ID NO: 363 - protein, 17 aa, synthetic construct
VIDSEGPTIKYSDAVRG

SEQ ID NO: 364 - protein, 14 aa, synthetic construct
SNGGSAFSSVLYTD

SEQ ID NO: 365 - protein, 127 aa, synthetic construct
QVQLQESGGALVQPGGSLRLSCVVSGFTSDYFAIGWFRQAPGKEREGVSCIGSSDGSTIYSDSVKGRFTISRDNAKNTVYLQMNSLKPEDTGVYYCALDRGRRTIIGYACYATMKYWGKGTPVTVSS

SEQ ID NO: 366 - protein, 123 aa, synthetic construct
EVQLVESGGGLVQPGGSLRLSCAASGFTFQNYAMSWLRKAPGEGLEWVSVINSGGGSTLYADSVKGRFTISRDNAKNTLYLQMHSLKSEDTAVYFCAKRRDDSTFGSLLYTHRGQGTQVTVSS

SEQ ID NO: 367 - protein, 126 aa, synthetic construct
EVQLVESGGGLVQAGGSLRVACAASGRTFSTYAMAWFRQAPGKEREFVAHILWSGGSTVYPDSVRGRFTISRDNAKNTVYLQMNSLKPEDTAVYYCAQKSGRQVYRIDSWDSFDYWGQGTSVTVSS

SEQ ID NO: 368 - protein, 124 aa, synthetic construct
QVQLVESGGGLVQAGGSLRLSCAASGRAFSRYSMGWFRQAPGKEREFVAANYWSNGWTDYADTVKGRFSISRDNAKNTVYLQMNSLKPEDTAVYYCAGRSSSIPSADVVGYDYWGQGTQVTVSS

SEQ ID NO: 369 - protein, 124 aa, synthetic construct
QVQLVESGGGLVQAGGSLRLSCAASGGPFSGYVMGWFRQVSGKEREFVAGITGISSTYFADSVKGRFTISRDNAKNMVYLQMNSLKPEDTAVYYCAAVNSFGVIPTSPNGMDYWGKGTLVTVSS

SEQ ID NO: 370 - protein, 123 aa, synthetic construct
QVQLVESGGGLVQAGGSLRLSCAAPGRYAVGWFRQAPGKEYEFVAAITVSGGSKYYEDSVKGRFAISRDNAKNTVYLQMNSLKAEDTAVYYCAARTPKFGSGWYNRRGEYDYWGQGTQVTVSS

SEQ ID NO: 371 - protein, 123 aa, synthetic construct
QVQLVESGGGLVQAGGSLRLSCAASARIFNSYAVAWFRQTPGKEREFVASISWSGGSTNYADSVKGRFTISRDNAKNTVFLQMNSLKPADTAVYYCAADRNYYPSVLGKYTYWGQGTQVTVSS

SEQ ID NO: 372 - protein, 126 aa, synthetic construct
EVQLVESGGGLVQAGGSLRLSCAASGLTFRNYAMAWFRQAPGKEREFVAGITWGGGLTHYVDSVKGRFTISRDNGKNTVYLQMNSLKSEDTAVYYCAGKTQGSTWYHLTPDGYDYWGQGTQVTVSS

SEQ ID NO: 373 - protein, 127 aa, synthetic construct
QVQLVESGGGLMQAGGSLRLSCALSRGIFSAYAMGWFRQVQGKEREFVSGISRSGSTTDYVDTAKGRFTISRDNARDTVYLQMNSLKPEDTAVYYCAAVTGTFPSSTYHNANAYHHWGQGTQVTVSS

SEQ ID NO: 374 - protein, 124 aa, synthetic construct
QVQLVESGGGLAQAGGSLRLSCAASGRTFSNDDMAWFRQAPGKEREFVAHIDRNAASTNYADSVKGRFTISRDNAKNTVYLQMTRLKPEDTAVYYCAADPDHFGTTWYQQYTYWGPGTQVTVSS

SEQ ID NO: 375 - protein, 119 aa, synthetic construct
EVQLVESGGGLVQPGGSLRLFCAASRSIFNINIMGWYRQAPGKQREWLASITSGGSTFYADSVKDRFTISRDSVKNTVYLQMNSLKPEDTAAYYCNRYRSVPLNTVYPWGQGTLVTVSS

SEQ ID NO: 376 - protein, 115 aa, synthetic construct
EVQLVESGGGLVQPGGSLTLSCTASGFRFSDRSMGWYRQAPGKQRELVAAITVGGSTNYADSVKGRFTISRDNAKNTVYLQMNSLKPEDTALYYCNRRYPPDDYWGQGTQVTVSS

SEQ ID NO: 377 - protein, 124 aa, synthetic construct
QVQLVESGGGLVQAGGSLRLSCLASGRTSNYFMGWFRQAPGKEREFVAAISRSGGGTWYADSVKGRFAISRDNAKNTVNLQMNSLKREDTAVYYCAADRRTGTSVYTVDEYDYWGQGTQVTVSS

SEQ ID NO: 378 - protein, 123 aa, synthetic construct
QVQLQESGGGLVQAGGSLRLSCAAPGRYAVGWFRQAPGKEYEFVAAITVSGGSKYYEDSVKGRFTISRDNAKNTVYLEMNSLKAEDTAVYYCAARTPKFGSGWYNRRGEYDYWGQGTQVTVSS

SEQ ID NO: 379 - protein, 124 aa, synthetic construct
QVQLVESGGGLVQAGGSLRLSCAASGRTFRTYAMGWFRQAPGKDREVVGIAAWKVASTYYVDYGDSVRGRFTISRDDAKSTVYLQMNNLMPEDTAVYYCAAGAFRTDWRSYAYWGQGTQVTVSS

SEQ ID NO: 380 - protein, 123 aa, synthetic construct
QVQLVESGGGLVQAGGSLRLSCAAPGRYAVGWFRQAPGKEYEFVAAITVSGGSKYYEDSVKGRFTISRDNAKNTVYLEMNSLKAEDTAVYYCAARTPKFGSGWYNRRGEYDYWGQGTQVAVSS

SEQ ID NO: 381 - protein, 117 aa, synthetic construct
QVQLQESGGGLVQVGDSLRLSCVASGRIFGVNTRAWYRQAPGNQRELVAQISSGGNINYADSVKGRFTISMDKFTDTVYLQMNSLKPEDTAIYRCAAHRQHPYADVWGQGTQVSVSS

SEQ ID NO: 382 - protein, 121 aa, synthetic construct
QVQLVESGGGAVQAGGSLRLSCAASGLTSRHYVMAWFRQAPGIEREFVASIWWGGSTYYADSVKDRFTISRDNAENTVYLQMNSLKPEDTAVYRCAVGTKIGTMGPRYDYWGQGTQVTVSS

SEQ ID NO: 383 - protein, 124 aa, synthetic construct
EVQLVESGGGLVQPGGSLRLSCAASGFTLDYYAIGWFRQAPGKEREGVSCISSSDRSTWYADSVKGRFTISRDNAKNTVYLQMHSLKPEDTAVYYCATPCYSDYDPEGYEYDYWGQGTQVTVSS

SEQ ID NO: 384 - protein, 126 aa, synthetic construct
QVQLVESGGGLVQAGGSLRLSCAASGRTFGTYAMGWFRQAPGKERDFVARISNSGAFTHYADSVKGRFTISRDNAKNTVYLHMNSLENGDTAAYYCVATRGTNSAYYTRSTMYEYWGQGTQVTVSS

SEQ ID NO: 385 - protein, 123 aa, synthetic construct
QVQLQESGGGLVQAGGSLRLSCAASGTIFSIDVMAYYRQAPGKQRELVAAISSGGSLNYRDSVKGRFTISRDNAKNAVYLQMNSLKPDDTAVYYCYANIRTSPVTTRPMGNYWGQGTQVTVSS

SEQ ID NO: 386 - protein, 116 aa, synthetic construct
QVQLVESGGGLVQAGGSLRLSCAASRSISSFNEMAWYRQAPGEQRELVATIVSTVGFTNYADSVKGRFTISRDNAKNTVYLQMNSLKAEDTAVYYCNARRISTDYWGQGTQVTVSS

SEQ ID NO: 387 - protein, 114 aa, synthetic construct
QLQLVESGGGLVQPGGSLRLSCAASGFTFSSYGMSWVRQAPGKGLEWVSYISAGGGTTTYVDFVKGRFTISRDNAKNMLYLQMNSLKPEDTAVYYCVHAADGSRGQGTQVTVSS

SEQ ID NO: 388 - protein, 120 aa, synthetic construct
QVQLQESGGGLVQAGGSLRLSCAASGSIASINAMGWYRQAPGKQRDFVARIQLSGQTSYAHSVRGRFTISRDNAKNTVYLQMNNLKPEDTAVYVCAASNTGSLYGLRNYWGQGTQVTVSS

SEQ ID NO: 389 - protein, 125 aa, synthetic construct
QVQLVESGGGLVQAGGSLRLSCAASGRTFSSYVMGWFRQAPGQEREFVAAIMRNGIMTYYADSVKGRFTISRDNAKNTVYLQMNTLKPDDTAVYYCAAASRVGVSISQSSSYTSWGQGTQVTVSS

SEQ ID NO: 390 - protein, 115 aa, synthetic construct
EVQLVESGGGLVQAGGSLRLSCAASGSKFSFNAMGWYRQTPGKQRELVAIIINGDMINYADSVKGRFTISRDNAKNTMYLQMNSLKPEDTAVYYCKMTRYVYDYWGQGTQVTVSS

SEQ ID NO: 391 - protein, 126 aa, synthetic construct
QVQLVESGGGLVQAGASLRLSCAASGRTFSSYAMGWFRQAPGKEREFVAAISGGTPNNIYYTNYKDAVRGRFTISRDNAKNTVYLQMSSLKPEDTAVYYCGATMGRYWNTGGYDYWGQGTQVTVSS

SEQ ID NO: 392 - protein, 123 aa, synthetic construct
QVQLVESGGGLVQPGGSLRLSCIASGFTFSDYAMKWVRRPPGKGLEWVSVIDSEGPTIKYSDAVRGRFTISRDNAKNTLYLQMNSLKPEDTAVYYCAKSNGGSAFSSVLYTDRGQGTQVTVSS

SEQ ID NO: 393 - protein, 5 aa, synthetic construct
SYWIG

SEQ ID NO: 394 - protein, 17 aa, synthetic construct
NIYPGDSDTRYSPSFQG

SEQ ID NO: 395 - protein, 5 aa, synthetic construct
RGGGY

SEQ ID NO: 396 - protein, 5 aa, synthetic construct
SYWIG

SEQ ID NO: 397 - protein, 17 aa, synthetic construct
IIYPGDSDTRYSPSFQG

SEQ ID NO: 398 - protein, 10 aa, synthetic construct
HEGAYWYFDL

SEQ ID NO: 399 - protein, 5 aa, synthetic construct
SYYWS

SEQ ID NO: 400 - protein, 16 aa, synthetic construct
YIYDSGSTNYNPSLKS

SEQ ID NO: 401 - protein, 9 aa, synthetic construct
GSYFWYFDL

SEQ ID NO: 402 - protein, 5 aa, synthetic construct
YYYWN

SEQ ID NO: 403 - protein, 16 aa, synthetic construct
SIYNSGNINYNPSLKS

SEQ ID NO: 404 - protein, 10 aa, synthetic construct
GGYSYGYFDN

SEQ ID NO: 405 - protein, 5 aa, synthetic construct
SYAMS

SEQ ID NO: 406 - protein, 17 aa, synthetic construct
AISGSGGSTYYADSVKG

SEQ ID NO: 407 - protein, 7 aa, synthetic construct
DGGYFDY

SEQ ID NO: 408 - protein, 5 aa, synthetic construct
GYYMH

SEQ ID NO: 409 - protein, 17 aa, synthetic construct
WINPKSGGTNYAQKFQG

SEQ ID NO: 410 - protein, 11 aa, synthetic construct
DQGYGDYVLKH

SEQ ID NO: 411 - protein, 5 aa, synthetic construct
GYYIH

SEQ ID NO: 412 - protein, 17 aa, synthetic construct
WINPHSGGTNYAQKFQG

SEQ ID NO: 413 - protein, 11 aa, synthetic construct
EANNWGYAFDI

SEQ ID NO: 414 - protein, 5 aa, synthetic construct
NYYMH

SEQ ID NO: 415 - protein, 17 aa, synthetic construct
IINPSGGSTSYSQKFQG

SEQ ID NO: 416 - protein, 8 aa, synthetic construct
DLGTTTDY

SEQ ID NO: 417 - protein, 5 aa, synthetic construct
TYDMH

SEQ ID NO: 418 - protein, 16 aa, synthetic construct
LIDTAGDTYYPDSVKG

SEQ ID NO: 419 - protein, 8 aa, synthetic construct
EGWGYFDY

SEQ ID NO: 420 - protein, 5 aa, synthetic construct
DYYWS

SEQ ID NO: 421 - protein, 16 aa, synthetic construct
YIYYSGSTTYSPSLKS

SEQ ID NO: 422 - protein, 9 aa, synthetic construct
DNGDYGFDY

SEQ ID NO: 423 - protein, 5 aa, synthetic construct
DYHWS

SEQ ID NO: 424 - protein, 16 aa, synthetic construct
EINHSGSTIYKSSLKS

SEQ ID NO: 425 - protein, 9 aa, synthetic construct
VDWANAFDI

SEQ ID NO: 426 - protein, 5 aa, synthetic construct
GSVMH

SEQ ID NO: 427 - protein, 19 aa, synthetic construct
RIRSKANSYATAFPASVKG

SEQ ID NO: 428 - protein, 8 aa, synthetic construct
GSLDVFDF

SEQ ID NO: 429 - protein, 7 aa, synthetic construct
TSGMCVN

SEQ ID NO: 430 - protein, 16 aa, synthetic construct
LIDWDDDKYYSTSLKT

SEQ ID NO: 431 - protein, 12 aa, synthetic construct
GIAAAGSNWFDP

SEQ ID NO: 432 - protein, 5 aa, synthetic construct
GYWIG

SEQ ID NO: 433 - protein, 17 aa, synthetic construct
IIYPGDSDTRYSPSFQG

SEQ ID NO: 434 - protein, 10 aa, synthetic construct
HPRGWWYFDL

SEQ ID NO: 435 - protein, 5 aa, synthetic construct
DSSMH

SEQ ID NO: 436 - protein, 19 aa, synthetic construct
RIRDKANTYATAYAASVKG

SEQ ID NO: 437 - protein, 10 aa, synthetic construct
HEWDLDAFDI

SEQ ID NO: 438 - protein, 5 aa, synthetic construct
SYAMS

SEQ ID NO: 439 - protein, 17 aa, synthetic construct
AINDRGNSPYYADSVKG

SEQ ID NO: 440 - protein, 10 aa, synthetic construct
MGATSNWFDP

SEQ ID NO: 441 - protein, 5 aa, synthetic construct
GSVMH

SEQ ID NO: 442 - protein, 19 aa, synthetic construct
RIRSKANSYATAYAASVTG

SEQ ID NO: 443 - protein, 8 aa, synthetic construct
GSYDYFDY

SEQ ID NO: 444 - protein, 5 aa, synthetic construct
GSSMH

SEQ ID NO: 445 - protein, 19 aa, synthetic construct
RIRDKANSYATAYAASVKG

SEQ ID NO: 446 - protein, 10 aa, synthetic construct
HEWDLDAFDI

SEQ ID NO: 447 - protein, 5 aa, synthetic construct
SYYWS

SEQ ID NO: 448 - protein, 16 aa, synthetic construct
RIYTSGSTNYNPSLKS

SEQ ID NO: 449 - protein, 15 aa, synthetic construct
AGSSGSSYYYYGMDV

SEQ ID NO: 450 - protein, 5 aa, synthetic construct
SYYMH

SEQ ID NO: 451 - protein, 17 aa, synthetic construct
IINPSGGSTSYAQKFQG

SEQ ID NO: 452 - protein, 10 aa, synthetic construct
GEEWELLGDY

SEQ ID NO: 453 - protein, 7 aa, synthetic construct
TSGMCVS

SEQ ID NO: 454 - protein, 16 aa, synthetic construct
LIYWDDDKYYSTSLKT

SEQ ID NO: 455 - protein, 10 aa, synthetic construct
MRRGIEAFDI

SEQ ID NO: 456 - protein, 7 aa, synthetic construct
TSGMCVS

SEQ ID NO: 457 - protein, 16 aa, synthetic construct
LIYWDDDKYYSTSLKT

SEQ ID NO: 458 - protein, 10 aa, synthetic construct
MRRGIEAFDI

SEQ ID NO: 459 - protein, 5 aa, synthetic construct
SYSMN

SEQ ID NO: 460 - protein, 17 aa, synthetic construct
SISSSSDYIHYADSVRG

SEQ ID NO: 461 - protein, 7 aa, synthetic construct
SELGFDY

SEQ ID NO: 462 - protein, 7 aa, synthetic construct
TSGMCVS

SEQ ID NO: 463 - protein, 16 aa, synthetic construct
LIYWDDDKYYSTSLKT

SEQ ID NO: 464 - protein, 10 aa, synthetic construct
MRRGIEAFDI

SEQ ID NO: 465 - protein, 5 aa, synthetic construct
DYYWT

SEQ ID NO: 466 - protein, 16 aa, synthetic construct
YIYYSGNTNYNPSLKS

SEQ ID NO: 467 - protein, 13 aa, synthetic construct
EGWMGVTPIAFDF

SEQ ID NO: 468 - protein, 16 aa, synthetic construct
RSSQSLVYSDGNTYLN

SEQ ID NO: 469 - protein, 7 aa, synthetic construct
KVSNRDS

SEQ ID NO: 470 - protein, 9 aa, synthetic construct
MQGTHWPIT

SEQ ID NO: 471 - protein, 14 aa, synthetic construct
TGSSSNIGAGYDVH

SEQ ID NO: 472 - protein, 7 aa, synthetic construct
GNSNRPS

SEQ ID NO: 473 - protein, 9 aa, synthetic construct
QSLDTSHVV

SEQ ID NO: 474 - protein, 11 aa, synthetic construct
SGDKLGAKYAS

SEQ ID NO: 475 - protein, 7 aa, synthetic construct
QDTKRPS

SEQ ID NO: 476 - protein, 9 aa, synthetic construct
QAWDSSTVV

SEQ ID NO: 477 - protein, 11 aa, synthetic construct
SGRELGNKYAY

SEQ ID NO: 478 - protein, 7 aa, synthetic construct
EDYKRPS

SEQ ID NO: 479 - protein, 9 aa, synthetic construct
QAWDSSTVI

SEQ ID NO: 480 - protein, 11 aa, synthetic construct
RASQSISSWLA

SEQ ID NO: 481 - protein, 7 aa, synthetic construct
KASSLES

SEQ ID NO: 482 - protein, 9 aa, synthetic construct
QQYNSYSPT

SEQ ID NO: 483 - protein, 11 aa, synthetic construct
GGDNIGSKSVH

SEQ ID NO: 484 - protein, 7 aa, synthetic construct
DDSDRPS

SEQ ID NO: 485 - protein, 11 aa, synthetic construct
QVWDSSSDHVV

SEQ ID NO: 486 - protein, 13 aa, synthetic construct
SGGSSNIGTNSVN

SEQ ID NO: 487 - protein, 7 aa, synthetic construct
YNNQRPS

SEQ ID NO: 488 - protein, 11 aa, synthetic construct
AAWDDSLNGWV

SEQ ID NO: 489 - protein, 11 aa, synthetic construct
RASQAIRNDLG

SEQ ID NO: 490 - protein, 7 aa, synthetic construct
AASSFQS

SEQ ID NO: 491 - protein, 9 aa, synthetic construct
LQDYRYPYT

SEQ ID NO: 492 - protein, 14 aa, synthetic construct
TGTSSDVGSYNRVS

SEQ ID NO: 493 - protein, 7 aa, synthetic construct
EVSNRPS

SEQ ID NO: 494 - protein, 10 aa, synthetic construct
SLYTGSSTVV

SEQ ID NO: 495 - protein, 14 aa, synthetic construct
TGTSSDVGSYNRVS

SEQ ID NO: 496 - protein, 7 aa, synthetic construct
EVSNRPS

SEQ ID NO: 497 - protein, 10 aa, synthetic construct
SLYTGSSTVV

SEQ ID NO: 498 - protein, 14 aa, synthetic construct
TGSSSNIGAGYDVH

SEQ ID NO: 499 - protein, 7 aa, synthetic construct
ANSNRPS

SEQ ID NO: 500 - protein, 10 aa, synthetic construct
QSYDSSLSVV

SEQ ID NO: 501 - protein, 11 aa, synthetic construct
QASQDISNYLN

SEQ ID NO: 502 - protein, 7 aa, synthetic construct
DASNLEA

SEQ ID NO: 503 - protein, 8 aa, synthetic construct
QQYDNLLT

SEQ ID NO: 504 - protein, 13 aa, synthetic construct
SGSSSNIGSHAVN

SEQ ID NO: 505 - protein, 7 aa, synthetic construct
VNNQRPS

SEQ ID NO: 506 - protein, 11 aa, synthetic construct
AAWDDSLNGPV

SEQ ID NO: 507 - protein, 14 aa, synthetic construct
TGSSSNIGAGYDVD

SEQ ID NO: 508 - protein, 7 aa, synthetic construct
GNSNRPS

SEQ ID NO: 509 - protein, 11 aa, synthetic construct
QSYDNSLSGSV

SEQ ID NO: 510 - protein, 11 aa, synthetic construct
RASQGISSYLV

SEQ ID NO: 511 - protein, 7 aa, synthetic construct
AASNLQS

SEQ ID NO: 512 - protein, 9 aa, synthetic construct
QQFNSYPFT

SEQ ID NO: 513 - protein, 12 aa, synthetic construct
TLSSGYSHYKVD

SEQ ID NO: 514 - protein, 12 aa, synthetic construct
VGTGGIVGSKGD

SEQ ID NO: 515 - protein, 13 aa, synthetic construct
GADYGSGSHFVYV

SEQ ID NO: 516 - protein, 11 aa, synthetic construct
QASQDISNYLN

SEQ ID NO: 517 - protein, 7 aa, synthetic construct
DASNLET

SEQ ID NO: 518 - protein, 9 aa, synthetic construct
QQYDDLLIT

SEQ ID NO: 519 - protein, 11 aa, synthetic construct
RASQGISSYLA

SEQ ID NO: 520 - skipped

SEQ ID NO: 521 - protein, 7 aa, synthetic construct
AASTLQS

SEQ ID NO: 522 - protein, 9 aa, synthetic construct
QQFNSYPFT

SEQ ID NO: 523 - protein, 11 aa, synthetic construct
SGDKLGDKYAC

SEQ ID NO: 524 - protein, 7 aa, synthetic construct
QDSKRPS

SEQ ID NO: 525 - protein, 9 aa, synthetic construct
QAWDSSTVV

SEQ ID NO: 526 - protein, 11 aa, synthetic construct
RASQVISNYLA

SEQ ID NO: 527 - protein, 7 aa, synthetic construct
AASSLQS

SEQ ID NO: 528 - protein, 9 aa, synthetic construct
LQHNDYPFT

SEQ ID NO: 529 - protein, 14 aa, synthetic construct
TGSSSNIGAGYDVH

SEQ ID NO: 530 - protein, 7 aa, synthetic construct
GYSNRPS

SEQ ID NO: 531 - protein, 11 aa, synthetic construct
QSFDISLTGWV

SEQ ID NO: 532 - protein, 14 aa, synthetic construct
TGSSSNIGAGYDVH

SEQ ID NO: 533 - protein, 7 aa, synthetic construct
GYSNRPS

SEQ ID NO: 534 - protein, 11 aa, synthetic construct
QSFDISLTGWV

SEQ ID NO: 535 - protein, 11 aa, synthetic construct
RASQGISIWLA

SEQ ID NO: 536 - protein, 7 aa, synthetic construct
AASSLQS

SEQ ID NO: 537 - protein, 9 aa, synthetic construct
QQANSFPLT

SEQ ID NO: 538 - protein, 14 aa, synthetic construct
TGSSSNIGAGYDVH

SEQ ID NO: 539 - protein, 7 aa, synthetic construct
GYSNRPS

SEQ ID NO: 540 - protein, 11 aa, synthetic construct
QSFDISLTGWV

SEQ ID NO: 541 - protein, 13 aa, synthetic construct
SGSSSNIGSNTVN

SEQ ID NO: 542 - protein, 7 aa, synthetic construct
NNDQRPS

SEQ ID NO: 543 - protein, 11 aa, synthetic construct
AAWDDSLNGWV

SEQ ID NO: 544 - protein, 114 aa, synthetic construct
EVQLVQSGTEVKKPGESLKISCKGSGYSFTSYWIGWVRQMPGKGLEWMGNIYPGDSDTRYSPSFQGHVTISADKSISTAYLQWSSLKASDTAMYYCARRGGGYWGQGTLVTVSS

SEQ ID NO: 545 - protein, 112 aa, synthetic construct
DVVMTQSPLSLPVTLGQPASISCRSSQSLVYSDGNTYLNWFQQRPGQSPRRLIYKVSNRDSGVPDRFSGSGSGTDFTLKISRVEADDVGVYYCMQGTHWPITFGQGTRLEIK

SEQ ID NO: 546 - protein, 119 aa, synthetic construct
EVQLVQSGAEVKKPGESLKISCKGSGYSFTSYWIGWVRQMPGKGLECMGIIYPGDSDTRYSPSFQGQVTISADKSISTAYLQWSSLKASDTAMYYCARHEGAYWYFDLWGRGTLVTVSS

SEQ ID NO: 547 - protein, 109 aa, synthetic construct
QSVLTQPPSVSGAPGQRVTISCTGSSSNIGAGYDVHWYQQLPGTAPKVLIYGNSNRPSGVPDRFSGSKSGTSASLAITGLQAEDEADYYCQSLDTSHVVFGGGTKLTVL

SEQ ID NO: 548 - protein, 117 aa, synthetic construct
QVQLQESGPGLVKPSETLSLTCTVSGGSISSYYWSWIRQPPGKGLEWFGYIYDSGSTNYNPSLKSRVTISVDTSKNQFSLKLSSVTAADTAVYYCARGSYFWYFDLWGRGTLVTVSS

SEQ ID NO: 549 - protein, 106 aa, synthetic construct
SYELTQPPSVSVSPGQTASITCSGDKLGAKYASWYHQKPGQSPVLVIYQDTKRPSGIPERFSGSNSGNTATLTISGTQAMDEADYYCQAWDSSTVVFGGGTKLTVL

SEQ ID NO: 550 - protein, 118 aa, synthetic construct
QVHLQESGPGLVKPSETLSLTCTVSGGSISYYYWNWIRQPPGKGLEWIASIYNSGNINYNPSLKSRVTISVDTSKNQFSLKLTSVTAADTAVYYCARGGYSYGYFDNWGQGILVTVSS

SEQ ID NO: 551 - protein, 106 aa, synthetic construct
SYELTQPPSVSVSPGQTASITCSGRELGNKYAYWYQQKPGQSPVLVIYEDYKRPSGIPERFSGSNSGNTATLTISGTQAMDEADYYCQAWDSSTVIFGGGTKLTVL

SEQ ID NO: 552 - protein, 116 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYFDYWGQGTLVTVSS

SEQ ID NO: 553 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYSPTFGQGTKVEIK

SEQ ID NO: 554 - protein, 120 aa, synthetic construct
QVQLVQPGAEVKKPGASVKVSCKASGYTFTGYYMHWVRQAPGQGLEWMGWINPKSGGTNYAQKFQGRVTMTRDTSISTAYMELTRLRSDDTAVYYCARDQGYGDYVLKHWGQGTLVTVSS

SEQ ID NO: 555 - protein, 108 aa, synthetic construct
SYVLTQPPSVSVAPGQTARITCGGDNIGSKSVHWYQQKPGQAPVPVVYDDSDRPSGIPERFSGSNSGNTATLTISRVEAGDEADYYCQVWDSSSDHVVFGGGTKLTVL

SEQ ID NO: 556 - protein, 120 aa, synthetic construct
QVQLVQSGAEVKKPGASVKVSCKASGYTFIGYYIHWVRQAPGQGLEWMGWINPHSGGTNYAQKFQGRVTMTRDTSISTAYMELSRLRSDDTAIYYCTKEANNWGYAFDIWGQGTMVTVSS

SEQ ID NO: 557 - protein, 110 aa, synthetic construct
QSVLTQPPSASGTPGQRVTISCSGGSSNIGTNSVNWYQQLPGTAPKLLIYYNNQRPSGVPDRFSGSKSGTSASLAISGLQSEDEADYYCAAWDDSLNGWVFGGGTKLTVL

SEQ ID NO: 558 - protein, 117 aa, synthetic construct
QVQLVQSGAEVKKPGASVKVSCKSSGYTFTNYYMHWVRQAPGQGLEWMGIINPSGGSTSYSQKFQGRVTMTRDTSTSTDYMELSSLRSDDTAVYYCARDLGTTTDYWGQGTLVTVSS

SEQ ID NO: 559 - protein, 107 aa, synthetic construct
AIQMTQSPSSLSASVGDRVTITCRASQAIRNDLGWFQQKPGKAPRLLIYAASSFQSGVPSRFSGSGSGTDFTLTISSLQPEDFATYYCLQDYRYPYTFGQGTKLEIK

SEQ ID NO: 560 - protein, 116 aa, synthetic construct
EVQLVESGGGLVQPGGSLRLSCAASGFTFSTYDMHWVRQATGKGLEWVSLIDTAGDTYYPDSVKGRFTISRENAKNSLYLQMNSLRAGDTAVYYCAREGWGYFDYWGQGALVTVSS

SEQ ID NO: 561 - protein, 110 aa, synthetic construct
QSALTQPPSVSGSPGQSVTISCTGTSSDVGSYNRVSWYQQPPGTAPKLMIYEVSNRPSGVPDRFSGSKSGNTASLTISGLQAEDEADYYCSLYTGSSTVVFGGGTKVTVL

SEQ ID NO: 562 - protein, 117 aa, synthetic construct
QVQLLESGPGLVKPSETLSLTCTVSGGSISDYYWSWIRQSPGKGLEWIGYIYYSGSTTYSPSLKSRVTISVDTSKNQFSLKLGSVTAADTAVYYCARDNGDYGFDYWGQGTLVTVSS

SEQ ID NO: 563 - protein, 110 aa, synthetic construct; identical to SEQ ID NO: 561

SEQ ID NO: 564 - protein, 117 aa, synthetic construct
QVQLQQWGAGLLKPSETLSLTCAVYGGSFSDYHWSWIRQPPGKGLEWIGEINHSGSTIYKSSLKSRVTISVDTSKNQFSLKLSSVTAADTAVYYCARVDWANAFDIWGQGTMVTVSS

SEQ ID NO: 565 - protein, 110 aa, synthetic construct
QSVLTQPPSVSGAPGQRVTISCTGSSSNIGAGYDVHWYQQLPGTAPKLLIYANSNRPSGVPDRFSGSKSGTSASLAITGLQAEDEADYYCQSYDSSLSVVFGGGTKLTVL

SEQ ID NO: 566 - protein, 119 aa, synthetic construct
EVQLVESGGGLVQPGGSLKLSCAGSGFTFSGSVMHWVRQASGKGLEWVGRIRSKANSYATAFPASVKGRFTISRDDSKNTAYLQMNSLKTEDTAVYYCTSGSLDVFDFWGQGTMVTVSS

SEQ ID NO: 567 - protein, 106 aa, synthetic construct
DIQMTQSPSSLSASVGDRVTITCQASQDISNYLNWYQQKPGKAPKLLIYDASNLEAGVPSRFSGSGFGTDFTFTISSLHPEDFATYYCQQYDNLLTFGGGTKVEIK

SEQ ID NO: 568 - protein, 122 aa, synthetic construct
QVTLRESGPALVKPTQTLTLTCTFSGFSLSTSGMCVNWIRQPPGKALEWLALIDWDDDKYYSTSLKTRLTISKDTSKNQVVLTMTNMDPVDTATYYCTRGIAAAGSNWFDPWGQGTLVTVSS

SEQ ID NO: 569 - protein, 110 aa, synthetic construct
QSVLTQPPSASGTPGQRVTISCSGSSSNIGSHAVNWYQQLPGTAPKLLIYVNNQRPSGVPDRFSGSKSGTSASLAISGLQSEDEADYYCAAWDDSLNGPVFGGGTKLTVL

SEQ ID NO: 570 - protein, 119 aa, synthetic construct
EVQLVQSGAEVKKPGESLKISCKGSGYSFTGYWIGWVRQMPGKGLEWMGIIYPGDSDTRYSPSFQGQVTISADKSISTAYLQWSSLKASDTAMYYCVRHPRGWWYFDLWGRGTLVTVSS

SEQ ID NO: 571 - protein, 111 aa, synthetic construct
QSVLTQPPSVSGAPGQRVTISCTGSSSNIGAGYDVDWYQQLPGTAPKVLIHGNSNRPSGVPDRFSGSKSGTSASLAITGLQAEDEADYYCQSYDNSLSGSVFGGGTKLTVL

SEQ ID NO: 572 - protein, 121 aa, synthetic construct
EVQLVESGGGLVQPGGSLKLSCAASGFTFSDSSMHWVRQASGKGLEWVGRIRDKANTYATAYAASVKGRFTISRDDSKNTAYLQMNSLKTEDTAVYYCTRHEWDLDAFDIWGQGTMVTVSS

SEQ ID NO: 573 - protein, 107 aa, synthetic construct
DIQLTQSPSFLSASVGDRVTITCRASQGISSYLVWYQQNPGKAPKLLIYAASNLQSGVPSRFSGSGSGTEFTLTISSLQPEDFATYYCQQFNSYPFTFGPGTKVDVK

SEQ ID NO: 574 - protein, 119 aa, synthetic construct
EVQLLESGGDLIQPGGSLRLSCAASGFTFSSYAMSWVRQAPGKGLEWVSAINDRGNSPYYADSVKGRFTISRDNSKNTLFLQMISLRAEDTAVYYCARMGATSNWFDPWGQGTLVTVSS

SEQ ID NO: 575 - protein, 116 aa, synthetic construct
QPVLTQPPSASASLGASVTLTCTLSSGYSHYKVDWYQQRPGKGPRFVMRVGTGGIVGSKGDGIPDRFSVLGSGLNRYLTIKNIQEEDESDYHCGADYGSGSHFVYVFGGGTKLTVL

SEQ ID NO: 576 - protein, 119 aa, synthetic construct
EVQLVESGGGLVQPGGSLKLSCAASGFTFSGSVMHWVRQASGKGLEWVGRIRSKANSYATAYAASVTGRFTISRDDSKNTAFLQMNSLKTEDTAVYYCSSGSYDYFDYWGQGTLVTVSS

SEQ ID NO: 577 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDDLLITFGQGTRLEIK

SEQ ID NO: 578 - protein, 121 aa, synthetic construct
EVQLVESGGGLVQPGGSLKLSCAASGFSISGSSMHWVRQASGKGLEWVGRIRDKANSYATAYAASVKGRFTVSRDDSKNTAYLQMTSLKTEDTAVYFCTRHEWDLDAFDIWGQGTMVTVSS

SEQ ID NO: 579 - protein, 107 aa, synthetic construct
DIQLTQSPSFLSASVGDRVTITCRASQGISSYLAWYQQKPGKAPKLLIYAASTLQSGVPSRFSGSGSGTEFTLTISSLQPEDFATYYCQQFNSYPFTFGPGTKVDIK

SEQ ID NO: 580 - protein, 123 aa, synthetic construct
QVQLQESGPGLVKPSETLSLTCTVSGGSISSYYWSWIRQPAGKGLEWIGRIYTSGSTNYNPSLKSRVTMSVDTSKNQFSLKLSSVTAADTAEYYCARAGSSGSSYYYYGMDVWGQGTTVTVSS

SEQ ID NO: 581 - protein, 106 aa, synthetic construct
SYELTQPPSVSVSPGQTASITCSGDKLGDKYACWYQQKPGQSPVLVIYQDSKRPSGIPERFSGSNSGNTATLTISGTQAMDEADYYCQAWDSSTVVFGGGTKLTVL

SEQ ID NO: 582 - protein, 119 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGEEWELLGDYWGQGTLVTVSS

SEQ ID NO: 583 - protein, 107 aa, synthetic construct
DIQMTQSPSAMSASVGDRVTITCRASQVISNYLAWFQQKPGKVPKRLIYAASSLQSGVPSRFSGSGSGTEFTLTISSLQPEDFATYYCLQHNDYPFTFGPGTKVDIK

SEQ ID NO: 584 - protein, 120 aa, synthetic construct
QVTLRESGPALVKPTQTLTLTCTFSGFSLSTSGMCVSWIRQPPGKALEWLALIYWDDDKYYSTSLKTRLTISKDTSKNQVVLTMTNMDPVDTATYYCARMRRGIEAFDIWGQGTMVTVFS

SEQ ID NO: 585 - protein, 111 aa, synthetic construct
QSVLTQPPSVSGAPGQRVTISCTGSSSNIGAGYDVHWYQQLPGTAPKLLIFGYSNRPSGVPVRFSGSKSGTSASLAITGLLAEDEADFYCQSFDISLTGWVFGGGTKLTVL

SEQ ID NO: 586 - protein, 120 aa, synthetic construct; identical to SEQ ID NO: 584

SEQ ID NO: 587 - protein, 111 aa, synthetic construct; identical to SEQ ID NO: 585

SEQ ID NO: 588 - protein, 116 aa, synthetic construct
EVLLVESGGGLVKPGGSLRLSCAASGFTFSSYSMNWVRQAPGKGLEWVSSISSSSDYIHYADSVRGRFTISRDNAKNSLYLQMNSLRAEDTAVYYCARSELGFDYWGQGTLVTVSS

SEQ ID NO: 589 - protein, 107 aa, synthetic construct
DIQMTQSPSSVSASVGDRVTITCRASQGISIWLAWYQQKPGKAPKPLIYAASSLQSGVPSRFSGSGSGTDFTLTISSLQPEDFATYYCQQANSFPLTFGGGTKVEIK

SEQ ID NO: 590 - protein, 120 aa, synthetic construct; identical to SEQ ID NO: 584

SEQ ID NO: 591 - protein, 111 aa, synthetic construct; identical to SEQ ID NO: 585

SEQ ID NO: 592 - protein, 121 aa, synthetic construct
QVQLQESGPGLVKPSETLSLTCTVSGDSISDYYWTWIRQSPGKGLEWIGYIYYSGNTNYNPSLKSRVTISVDTSKNQFSLKLSSVTAADTAVYYCTREGWMGVTPIAFDFWGQGTMVTVSS

SEQ ID NO: 593 - protein, 110 aa, synthetic construct
QSVLTQPPSASGTPGQRVTISCSGSSSNIGSNTVNWYQQLPGTAPKLLIFNNDQRPSGVPDRFSGSRSGTSASLAISGLQSEDEADYYCAAWDDSLNGWVFGGGTKLTVL

SEQ ID NO: 594 - protein, 5 aa, synthetic construct
SNGMH

SEQ ID NO: 595 - protein, 17 aa, synthetic construct
SIGSGGTSIYYADSVKG

SEQ ID NO: 596 - protein, 6 aa, synthetic construct
EVTGAT

SEQ ID NO: 597 - protein, 5 aa, synthetic construct
NYAMS

SEQ ID NO: 598 - protein, 17 aa, synthetic construct
AINSDGTSTSYADSVKG

SEQ ID NO: 599 - protein, 14 aa, synthetic construct
WEGDTDSDYKFKDY

SEQ ID NO: 600 - protein, 5 aa, synthetic construct
SKAMH

SEQ ID NO: 601 - protein, 17 aa, synthetic construct
SINSGGAITYYKDSVKG

SEQ ID NO: 602 - protein, 6 aa, synthetic construct
DTDGRT

SEQ ID NO: 603 - protein, 5 aa, synthetic construct
NYAMS

SEQ ID NO: 604 - protein, 17 aa, synthetic construct
RINSGGGTTSYADSVKG

SEQ ID NO: 605 - protein, 11 aa, synthetic construct
HSHPDYSSNYY

SEQ ID NO: 606 - protein, 5 aa, synthetic construct
SYAMG

SEQ ID NO: 607 - protein, 17 aa, synthetic construct
AIMRSGDTTVYVDSVKG

SEQ ID NO: 608 - protein, 20 aa, synthetic construct
GRPVTRTYYSTYTYPHDYDY

SEQ ID NO: 609 - protein, 5 aa, synthetic construct
LNTMA

SEQ ID NO: 610 - protein, 16 aa, synthetic construct
FITNGGTTNYVDSVEG

SEQ ID NO: 611 - protein, 9 aa, synthetic construct
QISQYPYNY

SEQ ID NO: 612 - protein, 5 aa, synthetic construct
PYDMS

SEQ ID NO: 613 - protein, 19 aa, synthetic construct
AIDKGGGAGATYYADSVKG

SEQ ID NO: 614 - protein, 14 aa, synthetic construct
TAGPYSDSTPRVAY

SEQ ID NO: 615 - protein, 5 aa, synthetic construct
SYAMS

SEQ ID NO: 616 - protein, 17 aa, synthetic construct
AINSGGDSTSYADSVEG

SEQ ID NO: 617 - protein, 13 aa, synthetic construct
WSDCSEYGCYDQT

SEQ ID NO: 618 - protein, 5 aa, synthetic construct
RYRMG

SEQ ID NO: 619 - protein, 17 aa, synthetic construct
AITKSGATTYDADSVKG

SEQ ID NO: 620 - protein, 16 aa, synthetic construct
SYHSNWWPKNADEYAY

SEQ ID NO: 621 - protein, 7 aa, synthetic construct
TNYYYWS

SEQ ID NO: 622 - protein, 16 aa, synthetic construct
AIAYSGSTYYSPSLKS

SEQ ID NO: 623 - protein, 16 aa, synthetic construct
GINFYSNYPTLNEYDY

SEQ ID NO: 624 - protein, 5 aa, synthetic construct
GYTVG

SEQ ID NO: 625 - protein, 16 aa, synthetic construct
AIQWSSAYTYSEFVKG

SEQ ID NO: 626 - protein, 17 aa, synthetic construct
RRSRTSWYASTAEAYDY

SEQ ID NO: 627 - protein, 5 aa, synthetic construct
SYVVG

SEQ ID NO: 628 - protein, 17 aa, synthetic construct
RISWSGARTYYADSVKG

SEQ ID NO: 629 - protein, 14 aa, synthetic construct
DLKWEVLLSEHFEY

SEQ ID NO: 630 - protein, 5 aa, synthetic construct
TYRMG

SEQ ID NO: 631 - protein, 17 aa, synthetic construct
GISWTGQTTYYAASVKG

SEQ ID NO: 632 - protein, 16 aa, synthetic construct
ETNNLGRWDRPSEYDY

SEQ ID NO: 633 - protein, 5 aa, synthetic construct
SYVVG

SEQ ID NO: 634 - protein, 17 aa, synthetic construct
RISWSGARTYYADSVEG

SEQ ID NO: 635 - protein, 14 aa, synthetic construct
DLKWEVLLSEHYEY

SEQ ID NO: 636 - protein, 5 aa, synthetic construct
AYRMG

SEQ ID NO: 637 - protein, 17 aa, synthetic construct
GITWRGQTTYYAASVKG

SEQ ID NO: 638 - protein, 16 aa, synthetic construct
ETNGLGRWDRPSEYDY

SEQ ID NO: 639 - protein, 5 aa, synthetic construct
SYAMG

SEQ ID NO: 640 - protein, 17 aa, synthetic construct
AISWNGGSRYYVESVEG

SEQ ID NO: 641 - protein, 16 aa, synthetic construct
SRIYTNYVARSYEYDD

SEQ ID NO: 642 - protein, 5 aa, synthetic construct
SYAMG

SEQ ID NO: 643 - protein, 17 aa, synthetic construct
AISWSGLSTYYVESVQG

SEQ ID NO: 644 - protein, 16 aa, synthetic construct
SRIYTNYVARSYEYDD

SEQ ID NO: 645 - protein, 5 aa, synthetic construct
SWTMA

SEQ ID NO: 646 - protein, 16 aa, synthetic construct
AITATGARTSSAESEG

SEQ ID NO: 647 - protein, 16 aa, synthetic construct
TGIASWDPGMADKYTY

SEQ ID NO: 648 - protein, 5 aa, synthetic construct
SWTMA

SEQ ID NO: 649 - protein, 16 aa, synthetic construct
AITATGARTSSAESEG

SEQ ID NO: 650 - protein, 16 aa, synthetic construct
TEIAGWDPGMGDKYPY

SEQ ID NO: 651 - protein, 5 aa, synthetic construct
SYAMG

SEQ ID NO: 652 - protein, 17 aa, synthetic construct
VIMRSGDTTAYVDSVKG

SEQ ID NO: 653 - protein, 20 aa, synthetic construct
GRPVTRTYYHTYTYPHDYDY

SEQ ID NO: 654 - protein, 5 aa, synthetic construct
SYAMG

SEQ ID NO: 655 - protein, 17 aa, synthetic construct
AIMRSGGTTVYVNSVKG

SEQ ID NO: 656 - protein, 20 aa, synthetic construct
GRPVSRTSYYTYTYPHDYDY

SEQ ID NO: 657 - protein, 5 aa, synthetic construct
GYTVG

SEQ ID NO: 658 - protein, 16 aa, synthetic construct
AIQWSSAYTYSEFVKG

SEQ ID NO: 659 - protein, 17 aa, synthetic construct
QRSRTSWYASTAEAYDY

SEQ ID NO: 660 - protein, 5 aa, synthetic construct
RYIMG

SEQ ID NO: 661 - protein, 17 aa, synthetic construct
AISWSGLSTHYADSVQG

SEQ ID NO: 662 - protein, 15 aa, synthetic construct
DRHWETRLLPREYDY

SEQ ID NO: 663 - protein, 5 aa, synthetic construct
IHGMG

SEQ ID NO: 664 - protein, 17 aa, synthetic construct
AIRWSDGTTIHADSVKG

SEQ ID NO: 665 - protein, 21 aa, synthetic construct
TSNPQFGRNWYTVAAGVEYGY

SEQ ID NO: 666 - protein, 5 aa, synthetic construct
INAMG

SEQ ID NO: 667 - protein, 16 aa, synthetic construct
AITSGGSTAYADSVKG

SEQ ID NO: 668 - protein, 14 aa, synthetic construct
DRDPITSWYVRGDY

SEQ ID NO: 669 - protein, 5 aa, synthetic construct
RYEMA

SEQ ID NO: 670 - protein, 17 aa, synthetic construct
HISSSGLTTTYADSVKG

SEQ ID NO: 671 - protein, 16 aa, synthetic construct
NVDGSAWSTLGAGMDY

SEQ ID NO: 672 - protein, 115 aa, synthetic construct
QVQLVESGGGLVRPGGSLRLSCAASGFTFSSNGMHWFRQAPGKGLEWVSSIGSGGTSIYYADSVKGRFTISRDNAKNTLYLQMNSLKSDDTAVYYCSKEVTGATRGQGTQVTVSS

SEQ ID NO: 673 - protein, 123 aa, synthetic construct
QVQLVESGGGLVQPGGSLRLSCAASGFTFSNYAMSWVRQAPGKGLEWVSAINSDGTSTSYADSVKGQFTISRDNAKNTLYLQMNSLKPEDTAVYYCTKWEGDTDSDYKFKDYWGQGTQVTVSS

SEQ ID NO: 674 - protein, 115 aa, synthetic construct
QVQLVESGGGLVQPGGSLRLSCAASGFTFSSKAMHWFRQAPGKGLEWVSSINSGGAITYYKDSVKGRFTVSRDNAKNILYLEMNSLKPEDTALYYCATDTDGRTRGQGTQVTVSS

SEQ ID NO: 675 - protein, 121 aa, synthetic construct
QVQLVESGGGLVQPGGSLRLSCAASGFTFSNYAMSWIRQGPERGFEWVSRINSGGGTTSYADSVKGRFTISRDNAKNTLYLQMNSLKPEDTAVYYCAKHSHPDYSSNYYRPGQGTQVTVSS

SEQ ID NO: 676 - protein, 129 aa, synthetic construct
EVQLVESGGGLVQAGGSLKLSCAASGGTFSSYAMGWFRQAPGKEREFVAAIMRSGDTTVYVDSVKGRFTVSRDNAKNTVYLQMNSLKPEDAAVYYCAAGRPVTRTYYSTYTYPHDYDYWGQGTQVTVSS

SEQ ID NO: 677 - protein, 117 aa, synthetic construct
QVQLVESGGGLVQAGGSLRVSCAASGRIPSLNTMAWYRQAPGKQREFVAFITNGGTTNYVDSVEGRFSISRDNTKNAVDLQMNSLKPEDTGVYYCNVQISQYPYNYWGQGTQVTVSS

SEQ ID NO: 678 - protein, 125 aa, synthetic construct
QVQLVESGGGLVQPGGSLRLSCAASGFTFEPYDMSWFRQAPGKGPEWVSAIDKGGGAGATYYADSVKGRFTISRDNAKKTLYLQMKSLKPEDTAVYYCAITAGPYSDSTPRVAYRGEGTQVTVSS

SEQ ID NO: 679 - protein, 122 aa, synthetic construct
EVQLVESGGGLVQPGGSLRLSCAASGFTFSSYAMSWVRQAPGKGLEWVSAINSGGDSTSYADSVEGRFTISRDNAKNTLYLQMNSLKPDDTAVYYCAKWSDCSEYGCYDQTTTEGTQVTVSS

SEQ ID NO: 680 - protein, 125 aa, synthetic construct
QVQLVESGGGLVQAGGSLRLSCAASGRTFSRYRMGWFRQAPGKEREFVAAITKSGATTYDADSVKGRFTISRDNAKNTLYLQMNSLKPEDTAVYYCAASYHSNWWPKNADEYAYWGQGTQVTVSS

SEQ ID NO: 681 - protein, 126 aa, synthetic construct
QVQLQESGPGLVKPSQTLSLTCTVSGGSITTNYYYWSWIRQPPGKGLEWMGAIAYSGSTYYSPSLKSRTSISRDTSKNQFTLQLSSVTPEDTAVYYCASGINFYSNYPTLNEYDYWGQGTQVTVSS

SEQ ID NO: 682 - protein, 125 aa, synthetic construct
QLQLVESGGGLVQAGGSLRLSCAASGLTFSGYTVGWFRQAPGKEREFVAAINWSSAYTYSEFVKGRFTISRDNAKNTVYLQMSSLKPEDTAVYYCAARRSRTSWYASTAEAYDYWGQGTQVTVSS

SEQ ID NO: 683 - protein, 123 aa, synthetic construct
QVQLVESGGGLVQAGNSLRLSCAASGRTFSSYVVGWFRQAPGKEREFVGRISWSGARTYYADSVKGRFTISRDNAKNTVYLQMNSLKPEDTAVYYCAADLKWEVLLSEHFEYWGQGTQVTVSS

SEQ ID NO: 684 - protein, 125 aa, synthetic construct
QVQLVESGGGLVQPGGSLRLSCATSEHTFGTYRMGWFRQAPGKEREFVAGISWTGQTTYYAASVKGRFAISKDNAKNTVYLQMNTLKSDDTAVYYCAVETNNLGRWDRPSEYDYWGQGTQVTVSS

SEQ ID NO: 685 - protein, 123 aa, synthetic construct
QVQLEESGGGLVQAGGSLRLSCAASGRTFSSYVVGWFRQAPGKEREFVGRISWSGARTYYADSVEGRFTISRDNAKNTVYLQMNSLKPEDTAVYYCAADLKWEVLLSEHYEYWGQGTQVTVSS

SEQ ID NO: 686 - protein, 125 aa, synthetic construct
QVQLVESGGGLVQAGGSLRLSCAASGRIFSAYRMGWFRQAPGKEREFVAGITWRGQTTYYAASVKGRFTISKDNAKKTVYLQMNSLKPEETAVYYCAVETNGLGRWDRPSEYDYWGQGTQVTVSS

SEQ ID NO: 687 - protein, 125 aa, synthetic construct
QVQLVESGGGLVQAGGSLRLSCAASGRTFSSYAMGWFRQAPGKEREFVSAISWNGGSRYYVESVEGRFTISRDNAKNTVYLQMNSLKPEDTAVYYCAASRIYTNYVARSYEYDDWGQGTQVTVSS

SEQ ID NO: 688 - protein, 125 aa, synthetic construct
QVQLVESGGGLVQAGGSLRLSCAASGRTFSSYAMGWFRQAPGKEREFVSAISWSGLSTYYVESVQGRFTISRDNAKNTVYLQMNSLKPEDTAVYYCAASRIYTNYVARSYEYDDWGQGTQVTVSS

SEQ ID NO: 689 - protein, 124 aa, synthetic construct
QVQLVESGGGLVQLGDSLRLSCAASGRGFSSWTMAWFRQALGKRREFVAAITATGARTCSAESEGRFTISRDNAKNMVFLQMNSLKAEDTAVYYCAATGIASWDPGMADKYTYWGQGTQVTVSS

SEQ ID NO: 690 - protein, 124 aa, synthetic construct
QVQLVESGGGLVQLGDSLRLSCAASGRGFSSWTMAWFRQALGKRREFVAAITATGARTCSAESEGRFTISRDNAKNMVFLQMNSLKAEDTAVYYCAATEIAGWDPGMGDKYPYWGQGTQVTVSS

SEQ ID NO: 691 - protein, 129 aa, synthetic construct
QVQLVESGGGLVQAGGSLKLSCAASGGTFSSYAMGWFRQAPGKEREFVAVIMRSGDTTAYVDSVKGRFTVSRDNAKNTVYLQMNSLKPEDAAVYFCAAGRPVTRTYYHTYTYPHDYDYWGQGTQVTVSS

SEQ ID NO: 692 - protein, 129 aa, synthetic construct
QVQLVESGGGLVQAGGSLKLSCAASGGTFSSYAMGWFRQAPGKEREFVAAIMRSGGTTVYVNSVKGRFTVSRDNAKNTVYLQMNSLKPEDAAVYYCAAGRPVSRTSYYTYTYPHDYDYWGQGTQVSVSS

SEQ ID NO: 693 - protein, 125 aa, synthetic construct
QLQLVESGGGLVQAGGSLRLSCAASGLTFSGYTVGWFRQAPGKEREFVAAINWSSAYTYSEFVKGRFTISRDNAKNTVYLQMSSLKPEDTAVYYCAAQRSRTSWYASTAEAYDYWGQGTQVTVSS

SEQ ID NO: 694 - protein, 124 aa, synthetic construct
QVQLVESGGGLVQAGGSLRLSCAASGRTFSRYIMGWFRQATGKEREFVAAISWSGLSTHYADSVQGRFTISRDNAENTVYLQMNSLKPEDTAVYYCAADRHWETRLLPREYDYWGQGTQVTVSS

SEQ ID NO: 695 - protein, 130 aa, synthetic construct
QLQLVESGGGLVQAGGSLRLSCAASGRTFGIHGMGWFRQAPGKEREFAAAIRWSDGTTIHADSVKGRLTISRDNAKNTVYLQMNSLKPEDTAVYYCAATSNPQFGRNWYTVAAGVEYGYWGQGTQVTVSS

SEQ ID NO: 696 - protein, 122 aa, synthetic construct
QLQLVESGGGLVQPGGSLRLSCAASGSIFSINAMGWYRQAPGKQRELVAAITSGGSTAYADSVKGRFTISRDNAKNTVYLQMNSLKPEDTAVYYCNADRDPITSWYVRGDYWGQGTQVTVSS

SEQ ID NO: 697 - protein, 125 aa, synthetic construct
QVQLVESGGGLVQTGASLRLSCAASGRTFGRYEMAWFRQAPGKEREFVAHISSSGLTTTYADSVKGRFTISRGNALNTVYLQMNSLKPEDTAVYYCAANVDGSAWSTLGAGMDYWGKGTLVTISS

SEQ ID NO: 698 - protein, 447 aa, synthetic construct
QVQLVHSGPEVKKPGASVKVSCKASGYTFTDYYMHWVRQAPGQGLEWMGWINPDSGGTNYAQKFQGRVTMTRDTSISTAYMELSRLRSDDTAVYHCAREGATSNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 699 - protein, 354 aa, synthetic construct
QVQLVESGGGLVQAGGSLRLSCAASGRTFSRYRMGWFRQAPGKEREFVAAITKSGATTYDADSVKGRFTISRDNAKNTLYLQMNSLKPEDTAVYYCAASYHSNWWPKNADEYAYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 700 - protein, 449 aa, synthetic construct
QVQLQESGPGLVKPSETLSLTCTVSGGSISSYDWSWIRQSAGKGLEWIGRIYTSGSTIYNPSLKSRVTMSVDTSKNEISLKLSSVTAADTAFYYCAKDRGNYPHDAFDIWGQGTMITVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 701 - protein, 354 aa, synthetic construct
QVQLVESGGGLVQAGGSLRLSCAASGRTFSSYAMGWFRQAPGKEREFVSAISWSGLSTYYVESVQGRFTISRDNAKNTVYLQMNSLKPEDTAVYYCAASRIYTNYVARSYEYDDWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 702 - protein, 214 aa, synthetic construct
DIQLTQSPSFLSASVGDRVTITCRASQGISSFLAWYQQKPGKAPKLLIYAASTLQSGVPSRFSGSGSGTEFTLTISSLQPEDFATYYCQQLNGYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 703 - protein, 450 aa, synthetic construct
QVHLVQSGAEVKKPGASVKVSCEASGYTFTDYYIHWLRQAPGQGLEWMGWINPNSGGTHYAQKFQGRVTMTRDTSISTAYMELSRLSSDDTAVYYCAREGGWNYFSGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 704 - protein, 354 aa, synthetic construct
QVQLVESGGGLVQAGGSLRLSCAASGRIFSAYRMGWFRQAPGKEREFVAGITWRGQTTYYAASVKGRFTISKDNAKKTVYLQMNSLKPEETAVYYCAVETNGLGRWDRPSEYDYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 705 - protein, 214 aa, synthetic construct
DIQLTQSPSFLSASVGDRVTITCRASQGISSYLAWYQQQPGKPPKLLIYAASTLHSGVPSRFSGSGSGTEFTLTISSLQPEDFATYYCQQLNRYPYTCGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 706 - protein, 450 aa, synthetic construct; identical to SEQ ID NO: 703

SEQ ID NO: 707 - protein, 354 aa, synthetic construct; identical to SEQ ID NO: 701

SEQ ID NO: 708 - protein, 214 aa, synthetic construct; identical to SEQ ID NO: 705

SEQ ID NO: 709 - protein, 450 aa, synthetic construct
QVHLVQSGAEVKKPGASVKVSCKASGYTFIDYYMHWVRQAPGQGLEWMGWINPNSGGTHYSQKFQGRVTMTWDTSISTAYMELSRLTSDDTAVYYCAREGDYISSSAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 710 - protein, 354 aa, synthetic construct; identical to SEQ ID NO: 699

SEQ ID NO: 711 - protein, 214 aa, synthetic construct
DIQLTQSPSFLSASVGDRVTITCRASQGISSYLAWYQQKPGKAPKVLIYAASTLQSGVPSRFSGSGSGTEFTLTISSLLPEDFATYFCQQLHRYPYTFGQGAKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 712 - protein, 448 aa, synthetic construct
QVQLQESGPGLVKPSETLSLTCTVSGGSISSYYWSWIRQSAGKELEWIGRIYTSGSTIYNPSLKSRVTMSVDPSKNQFSLKLSSVTAADTAVYYCANSRSYPHDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 713 - protein, 354 aa, synthetic construct; identical to SEQ ID NO: 704

SEQ ID NO: 714 - protein, 214 aa, synthetic construct
DIQLTQSPSFLSASVGYRVTITCRASQGISSYLAWYQQKPGKAPQLLIYAASTLQSGVPSRFSGRGSGTEFTLTISSLQPEDFATYYCQQLNGYPFTFGPGTKVNIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 715 - protein, 445 aa, synthetic construct
QVQLVESGGGVVQPGRSLRLSCAASGFTFSSYGMHWVRQAPGKGLEWVAVIWNDGSNKYYADSVKGRFTISRDNSKNTLYLQMNSLRAEDTAVYYCARGELFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 716 - protein, 344 aa, synthetic construct
QVQLVESGGGLVRPGGSLRLSCAASGFTFSSNGMHWFRQAPGKGLEWVSSIGSGGTSIYYADSVKGRFTISRDNAKNTLYLQMNSLKSDDTAVYYCSKEVTGATRGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 717 - protein, 213 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 718 - protein, 445 aa, synthetic construct; identical to SEQ ID NO: 715

SEQ ID NO: 719 - protein, 352 aa, synthetic construct
QVQLVESGGGLVQPGGSLRLSCAASGFTFSNYAMSWVRQAPGKGLEWVSAINSDGTSTSYADSVKGQFTISRDNAKNTLYLQMNSLKPEDTAVYYCTKWEGDTDSDYKFKDYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 720 - protein, 213 aa, synthetic construct; identical to SEQ ID NO: 717

SEQ ID NO: 721 - protein, 450 aa, synthetic construct
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAREELTGLYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 722 - protein, 354 aa, synthetic construct; identical to SEQ ID NO: 699

SEQ ID NO: 723 - protein, 211 aa, synthetic construct
EIVLTQSPGTLSLSPGERATLSCRASQSINSNYLAWYQQKPGQAPRLLIYGASSRATGIPDRFSGSGSGTDFTLTISRLEPEDFAVYYCQQYDSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 724 - protein, 448 aa, synthetic construct
QVQLVQSGAEVKKPGASAKVSCKASGYTFTDYYLHWVRQAPGQGLEWMGWINPNNGGTHYAQKFQGRVTMTRDTSISTAYMELSRLRSDDTAVYYCAREMGGTTAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 725 - protein, 344 aa, synthetic construct; identical to SEQ ID NO: 716

SEQ ID NO: 726 - protein, 214 aa, synthetic construct
DIQMTQSPSSLSASVGDRVTITCRASQGIGNYLAWFQQRPGKAPKSLIYAASSLQSGVPSKFSGSGSGTDFTLTISSLQPADFAIYYCQQYNIYPYTFGPGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 727 - protein, 448 aa, synthetic construct; identical to SEQ ID NO: 724

SEQ ID NO: 728 - protein, 354 aa, synthetic construct; identical to SEQ ID NO: 699

SEQ ID NO: 729 - protein, 214 aa, synthetic construct; identical to SEQ ID NO: 726

SEQ ID NO: 730 - protein, 448 aa, synthetic construct
QVQLVQSGAEVKKPGASVKVSCKASGYTFTDYYIHWVRQAPGQGLEWMGWINPDSGGTHYAQKFQGRVTMTRDTSISTVYMDLSRLRSDDTAVYYCAREGEGLASFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 731 - protein, 354 aa, synthetic construct; identical to SEQ ID NO: 699

SEQ ID NO: 732 - protein, 214 aa, synthetic construct
DIQMTQSPSSLSASVGNRVTITCRASQGINNYLAWFQQKPGKAPKSLIYAASSLQSGVPSKFSGSGSGTDFTLTISSLQPEDFATYYCQQYNNYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 733 - protein, 449 aa, synthetic construct
QITLKESGPTLVKPTQTLTLTCTFFGFSLNTGGVGVGWIRQPPGKALEWLTLIYWNDDKRYSPSLKSRLTITKDTSKNQVVLTMTNMDPVDTATYYCAHTLRWYHGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 734 - protein, 355 aa, synthetic construct
QVQLQESGPGLVKPSQTLSLTCTVSGGSITTNYYYWSWIRQPPGKGLEWMGAIAYSGSTYYSPSLKSRTSISRDTSKNQFTLQLSSVTPEDTAVYYCASGINFYSNYPTLNEYDYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 735 - protein, 216 aa, synthetic construct
QTVVTQEPSFSVSPGGTVTLTCGLTSGSVSTSYYPSWYQQTPGQAPRTLIYSTYTRSSGVPDRFSGSILGNKAALTITGAQADDESDYYCVLYMGSGIWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 736 - protein, 353 aa, synthetic construct
QVQLVESGGGLVQAGGSLRLSCAASGGPFSGYVMGWFRQVSGKEREFVAGITGISSTYFADSVKGRFTISRDNAKNMVYLQMNSLKPEDTAVYYCAAVNSFGVIPTSPNGMDYWGKGTLVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 737 - protein, 449 aa, synthetic construct
QVTLRESGPALVKPTQTLTLTCTFSGFSLSTSGMCVSWIRQPPGKALEWLALIYWDDDKYYSTSLKTRLTISKDTSKNQVVLTMTNMDPVDTATYYCARMRRGIEAFDIWGQGTMVTVFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 738 - protein, 217 aa, synthetic construct
QSVLTQPPSVSGAPGQRVTISCTGSSSNIGAGYDVHWYQQLPGTAPKLLIFGYSNRPSGVPVRFSGSKSGTSASLAITGLLAEDEADFYCQSFDISLTGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 739 - protein, 353 aa, synthetic construct; identical to SEQ ID NO: 736

SEQ ID NO: 740 - protein, 443 aa, synthetic construct
EVQLVQSGTEVKKPGESLKISCKGSGYSFTSYWIGWVRQMPGKGLEWMGNIYPGDSDTRYSPSFQGHVTISADKSISTAYLQWSSLKASDTAMYYCARRGGGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 741 - protein, 219 aa, synthetic construct
DVVMTQSPLSLPVTLGQPASISCRSSQSLVYSDGNTYLNWFQQRPGQSPRRLIYKVSNRDSGVPDRFSGSGSGTDFTLKISRVEADDVGVYYCMQGTHWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 742 - protein, 352 aa, synthetic construct
QVQLVESGGGLVQAGGSLRLSCAAPGRYAVGWFRQAPGKEYEFVAAITVSGGSKYYEDSVKGRFAISRDNAKNTVYLQMNSLKAEDTAVYYCAARTPKFGSGWYNRRGEYDYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 743 - protein, 445 aa, synthetic construct
EVLLVESGGGLVKPGGSLRLSCAASGFTFSSYSMNWVRQAPGKGLEWVSSISSSSDYIHYADSVRGRFTISRDNAKNSLYLQMNSLRAEDTAVYYCARSELGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 744 - protein, 214 aa, synthetic construct
DIQMTQSPSSVSASVGDRVTITCRASQGISIWLAWYQQKPGKAPKPLIYAASSLQSGVPSRFSGSGSGTDFTLTISSLQPEDFATYYCQQANSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 745 - protein, 353 aa, synthetic construct
QVQLVESGGGLAQAGGSLRLSCAASGRTFSNDDMAWFRQAPGKEREFVAHIDRNAASTNYADSVKGRFTISRDNAKNTVYLQMTRLKPEDTAVYYCAADPDHFGTTWYQQYTYWGPGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 746 - protein, 452 aa, synthetic construct
QVQLQESGPGLVKPSETLSLTCTVSGGSISSYYWSWIRQPAGKGLEWIGRIYTSGSTNYNPSLKSRVTMSVDTSKNQFSLKLSSVTAADTAEYYCARAGSSGSSYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 747 - protein, 212 aa, synthetic construct
SYELTQPPSVSVSPGQTASITCSGDKLGDKYACWYQQKPGQSPVLVIYQDSKRPSGIPERFSGSNSGNTATLTISGTQAMDEADYYCQAWDSSTVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 748 - protein, 353 aa, synthetic construct; identical to SEQ ID NO: 745

SEQ ID NO: 749 - protein, 447 aa, synthetic construct
QVHLQESGPGLVKPSETLSLTCTVSGGSISYYYWNWIRQPPGKGLEWIASIYNSGNINYNPSLKSRVTISVDTSKNQFSLKLTSVTAADTAVYYCARGGYSYGYFDNWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 750 - protein, 212 aa, synthetic construct
SYELTQPPSVSVSPGQTASITCSGRELGNKYAYWYQQKPGQSPVLVIYEDYKRPSGIPERFSGSNSGNTATLTISGTQAMDEADYYCQAWDSSTVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 751 - protein, 353 aa, synthetic construct
EVQLVESGGGLVQPGGSLRLSCAASGFTLDYYAIGWFRQAPGKEREGVSCISSSDRSTWYADSVKGRFTISRDNAKNTVYLQMHSLKPEDTAVYYCATPCYSDYDPEGYEYDYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 752 - protein, 452 aa, synthetic construct; identical to SEQ ID NO: 746

SEQ ID NO: 753 - protein, 212 aa, synthetic construct; identical to SEQ ID NO: 747

SEQ ID NO: 754 - protein, 355 aa, synthetic construct
QVQLVESGGGLVQAGGSLRLSCAASGRTFGTYAMGWFRQAPGKERDFVARISNSGAFTHYADSVKGRFTISRDNAKNTVYLHMNSLENGDTAAYYCVATRGTNSAYYTRSTMYEYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 755 - protein, 452 aa, synthetic construct; identical to SEQ ID NO: 746

SEQ ID NO: 756 - protein, 212 aa, synthetic construct; identical to SEQ ID NO: 747

SEQ ID NO: 757 - protein, 355 aa, synthetic construct; identical to SEQ ID NO: 754

SEQ ID NO: 758 - protein, 449 aa, synthetic construct
QVQLVQPGAEVKKPGASVKVSCKASGYTFTGYYMHWVRQAPGQGLEWMGWINPKSGGTNYAQKFQGRVTMTRDTSISTAYMELTRLRSDDTAVYYCARDQGYGDYVLKHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 759 - protein, 214 aa, synthetic construct
SYVLTQPPSVSVAPGQTARITCGGDNIGSKSVHWYQQKPGQAPVPVVYDDSDRPSGIPERFSGSNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 760 - protein, 355 aa, synthetic construct; identical to SEQ ID NO: 754

SEQ ID NO: 761 - protein, 445 aa, synthetic construct
EVQLVESGGGLVQPGGSLRLSCAASGFTFSTYDMHWVRQATGKGLEWVSLIDTAGDTYYPDSVKGRFTISRENAKNSLYLQMNSLRAGDTAVYYCAREGWGYFDYWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 762 - protein, 216 aa, synthetic construct
QSALTQPPSVSGSPGQSVTISCTGTSSDVGSYNRVSWYQQPPGTAPKLMIYEVSNRPSGVPDRFSGSKSGNTASLTISGLQAEDEADYYCSLYTGSSTVVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 763 - protein, 355 aa, synthetic construct; identical to SEQ ID NO: 754

SEQ ID NO: 764 - protein, 447 aa, synthetic construct; identical to SEQ ID NO: 749

SEQ ID NO: 765 - protein, 212 aa, synthetic construct; identical to SEQ ID NO: 750

SEQ ID NO: 766 - protein, 355 aa, synthetic construct; identical to SEQ ID NO: 754

SEQ ID NO: 767 - protein, 449 aa, synthetic construct; identical to SEQ ID NO: 737

SEQ ID NO: 768 - protein, 217 aa, synthetic construct; identical to SEQ ID NO: 738

SEQ ID NO: 769 - protein, 355 aa, synthetic construct; identical to SEQ ID NO: 754

SEQ ID NO: 770 - protein, 449 aa, synthetic construct
QVQLVQSGAEVKKPGASVKVSCKASGYTFIGYYIHWVRQAPGQGLEWMGWINPHSGGTNYAQKFQGRVTMTRDTSISTAYMELSRLRSDDTAIYYCTKEANNWGYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 771 - protein, 216 aa, synthetic construct
QSVLTQPPSASGTPGQRVTISCSGGSSNIGTNSVNWYQQLPGTAPKLLIYYNNQRPSGVPDRFSGSKSGTSASLAISGLQSEDEADYYCAAWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 772 - protein, 352 aa, synthetic construct
QVQLQESGGGLVQAGGSLRLSCAASGTIFSIDVMAYYRQAPGKQRELVAAISSGGSLNYRDSVKGRFTISRDNAKNAVYLQMNSLKPDDTAVYYCYANIRTSPVTTRPMGNYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 773 - protein, 449 aa, synthetic construct; identical to SEQ ID NO: 758

SEQ ID NO: 774 - protein, 214 aa, synthetic construct; identical to SEQ ID NO: 759

SEQ ID NO: 775 - protein, 353 aa, synthetic construct; identical to SEQ ID NO: 736

SEQ ID NO: 776 - protein, 449 aa, synthetic construct; identical to SEQ ID NO: 770

SEQ ID NO: 777 - protein, 216 aa, synthetic construct; identical to SEQ ID NO: 771

SEQ ID NO: 778 - protein, 355 aa, synthetic construct
EVQLVESGGGLVQAGGSLRLSCAASGLTFRNYAMAWFRQAPGKEREFVAGITWGGGLTHYVDSVKGRFTISRDNGKNTVYLQMNSLKSEDTAVYYCAGKTQGSTWYHLTPDGYDYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 779 - protein, 352 aa, synthetic construct; identical to SEQ ID NO: 719

SEQ ID NO: 780 - protein, 345 aa, synthetic construct
QVQLVESGGGLVQAGGSLRLSCAASRSISSFNEMAWYRQAPGEQRELVATIVSTVGFTNYADSVKGRFTISRDNAKNTVYLQMNSLKAEDTAVYYCNARRISTDYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 781 - protein, 445 aa, synthetic construct; identical to SEQ ID NO: 761

SEQ ID NO: 782 - protein, 356 aa, synthetic construct
QVQLQESGGALVQPGGSLRLSCVVSGFTSDYFAIGWFRQAPGKEREGVSCIGSSDGSTIYSDSVKGRFTISRDNAKNTVYLQMNSLKPEDTGVYYCALDRGRRTIIGYACYATMKYWGKGTPVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 783 - protein, 350 aa, synthetic construct
QVQLVESGGGLVQPGGSLRLSCAASGFTFSNYAMSWIRQGPERGFEWVSRINSGGGTTSYADSVKGRFTISRDNAKNTLYLQMNSLKPEDTAVYYCAKHSHPDYSSNYYRPGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 784 - protein, 356 aa, synthetic construct; identical to SEQ ID NO: 782

SEQ ID NO: 785 - protein, 354 aa, synthetic construct
QVQLVESGGGLVQPGGSLRLSCAASGFTFEPYDMSWFRQAPGKGPEWVSAIDKGGGAGATYYADSVKGRFTISRDNAKKTLYLQMKSLKPEDTAVYYCAITAGPYSDSTPRVAYRGEGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 786 - protein, 352 aa, synthetic construct
EVQLVESGGGLVQPGGSLRLSCAASGFTFQNYAMSWLRKAPGEGLEWVSVINSGGGSTLYADSVKGRFTISRDNAKNTLYLQMHSLKSEDTAVYFCAKRRDDSTFGSLLYTHRGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 787 - protein, 354 aa, synthetic construct; identical to SEQ ID NO: 785

SEQ ID NO: 788 - protein, 352 aa, synthetic construct; identical to SEQ ID NO: 786

SEQ ID NO: 789 - protein, 354 aa, synthetic construct; identical to SEQ ID NO: 704

SEQ ID NO: 790 - protein, 353 aa, synthetic construct
QVQLVESGGGLVQAGGSLRLSCAASGRAFSRYSMGWFRQAPGKEREFVAANYWSNGWTDYADTVKGRFSISRDNAKNTVYLQMNSLKPEDTAVYYCAGRSSSIPSADVVGYDYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 791 - protein, 352 aa, synthetic construct; identical to SEQ ID NO: 719

SEQ ID NO: 792 - protein, 353 aa, synthetic construct; identical to SEQ ID NO: 790

SEQ ID NO: 793 - protein, 354 aa, synthetic construct; identical to SEQ ID NO: 785

SEQ ID NO: 794 - protein, 352 aa, synthetic construct; identical to SEQ ID NO: 742

SEQ ID NO: 795 - protein, 354 aa, synthetic construct; identical to SEQ ID NO: 704

SEQ ID NO: 796 - protein, 352 aa, synthetic construct; identical to SEQ ID NO: 742

SEQ ID NO: 797 - protein, 354 aa, synthetic construct; identical to SEQ ID NO: 701

SEQ ID NO: 798 - protein, 352 aa, synthetic construct
QVQLVESGGGLVQAGGSLRLSCAASARIFNSYAVAWFRQTPGKEREFVASISWSGGSTNYADSVKGRFTISRDNAKNTVFLQMNSLKPADTAVYYCAADRNYYPSVLGKYTYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 799 - protein, 352 aa, synthetic construct; identical to SEQ ID NO: 719

SEQ ID NO: 800 - protein, 352 aa, synthetic construct; identical to SEQ ID NO: 798

SEQ ID NO: 801 - protein, 344 aa, synthetic construct
QVQLVESGGGLVQPGGSLRLSCAASGFTFSSKAMHWFRQAPGKGLEWVSSINSGGAITYYKDSVKGRFTVSRDNAKNILYLEMNSLKPEDTALYYCATDTDGRTRGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 802 - protein, 352 aa, synthetic construct; identical to SEQ ID NO: 798

SEQ ID NO: 803 - protein, 354 aa, synthetic construct; identical to SEQ ID NO: 785

SEQ ID NO: 804 - protein, 352 aa, synthetic construct; identical to SEQ ID NO: 798

SEQ ID NO: 805 - protein, 355 aa, synthetic construct; identical to SEQ ID NO: 734

SEQ ID NO: 806 - protein, 352 aa, synthetic construct; identical to SEQ ID NO: 798

SEQ ID NO: 807 - protein, 354 aa, synthetic construct; identical to SEQ ID NO: 701

SEQ ID NO: 808 - protein, 355 aa, synthetic construct; identical to SEQ ID NO: 778

SEQ ID NO: 809 - protein, 344 aa, synthetic construct; identical to SEQ ID NO: 801

SEQ ID NO: 810 - protein, 355 aa, synthetic construct; identical to SEQ ID NO: 778

SEQ ID NO: 811 - protein, 350 aa, synthetic construct; identical to SEQ ID NO: 783

SEQ ID NO: 812 - protein, 355 aa, synthetic construct; identical to SEQ ID NO: 778

SEQ ID NO: 813 - protein, 354 aa, synthetic construct; identical to SEQ ID NO: 785

SEQ ID NO: 814 - protein, 355 aa, synthetic construct; identical to SEQ ID NO: 778

SEQ ID NO: 815 - protein, 354 aa, synthetic construct; identical to SEQ ID NO: 704

SEQ ID NO: 816 - protein, 355 aa, synthetic construct; identical to SEQ ID NO: 778

SEQ ID NO: 817 - protein, 354 aa, synthetic construct; identical to SEQ ID NO: 701

SEQ ID NO: 818 - protein, 355 aa, synthetic construct; identical to SEQ ID NO: 778

SEQ ID NO: 819 - protein, 353 aa, synthetic construct
QVQLVESGGGLVQLGDSLRLSCAASGRGFSSWTMAWFRQALGKRREFVAAITATGARTSSAESEGRFTISRDNAKNMVFLQMNSLKAEDTAVYYCAATGIASWDPGMADKYTYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 820 - protein, 355 aa, synthetic construct; identical to SEQ ID NO: 778

SEQ ID NO: 821 - protein, 358 aa, synthetic construct
QVQLVESGGGLVQAGGSLKLSCAASGGTFSSYAMGWFRQAPGKEREFVAAIMRSGGTTVYVNSVKGRFTVSRDNAKNTVYLQMNSLKPEDAAVYYCAAGRPVSRTSYYTYTYPHDYDYWGQGTQVSVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 822 - protein, 355 aa, synthetic construct; identical to SEQ ID NO: 778

SEQ ID NO: 823 - protein, 353 aa, synthetic construct
QVQLVESGGGLVQAGGSLRLSCAASGRTFSRYIMGWFRQATGKEREFVAAISWSGLSTHYADSVQGRFTISRDNAENTVYLQMNSLKPEDTAVYYCAADRHWETRLLPREYDYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 824 - protein, 353 aa, synthetic construct; identical to SEQ ID NO: 745

SEQ ID NO: 825 - protein, 350 aa, synthetic construct; identical to SEQ ID NO: 783

SEQ ID NO: 826 - protein, 353 aa, synthetic construct; identical to SEQ ID NO: 745

SEQ ID NO: 827 - protein, 346 aa, synthetic construct
QVQLVESGGGLVQAGGSLRVSCAASGRIPSLNTMAWYRQAPGKQREFVAFITNGGTTNYVDSVEGRFSISRDNTKNAVDLQMNSLKPEDTGVYYCNVQISQYPYNYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 828 - protein, 353 aa, synthetic construct; identical to SEQ ID NO: 745

SEQ ID NO: 829 - protein, 354 aa, synthetic construct; identical to SEQ ID NO: 785

SEQ ID NO: 830 - protein, 348 aa, synthetic construct
EVQLVESGGGLVQPGGSLRLFCAASRSIFNINIMGWYRQAPGKQREWLASITSGGSTFYADSVKDRFTISRDSVKNTVYLQMNSLKPEDTAAYYCNRYRSVPLNTVYPWGQGTLVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 831 - protein, 344 aa, synthetic construct; identical to SEQ ID NO: 716

SEQ ID NO: 832 - protein, 348 aa, synthetic construct; identical to SEQ ID NO: 830

SEQ ID NO: 833 - protein, 354 aa, synthetic construct; identical to SEQ ID NO: 704

SEQ ID NO: 834 - skipped

SEQ ID NO: 835 - protein, 348 aa, synthetic construct; identical to SEQ ID NO: 830

SEQ ID NO: 836 - protein, 354 aa, synthetic construct; identical to SEQ ID NO: 701

SEQ ID NO: 837 - protein, 344 aa, synthetic construct
EVQLVESGGGLVQPGGSLTLSCTASGFRFSDRSMGWYRQAPGKQRELVAAITVGGSTNYADSVKGRFTISRDNAKNTVYLQMNSLKPEDTALYYCNRRYPPDDYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 838 - protein, 344 aa, synthetic construct; identical to SEQ ID NO: 716

SEQ ID NO: 839 - protein, 344 aa, synthetic construct; identical to SEQ ID NO: 837

SEQ ID NO: 840 - protein, 354 aa, synthetic construct; identical to SEQ ID NO: 701

SEQ ID NO: 841 - protein, 353 aa, synthetic construct
QVQLVESGGGLVQAGGSLRLSCLASGRTSNYFMGWFRQAPGKEREFVAAISRSGGGTWYADSVKGRFAISRDNAKNTVNLQMNSLKREDTAVYYCAADRRTGTSVYTVDEYDYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 842 - protein, 352 aa, synthetic construct; identical to SEQ ID NO: 719

SEQ ID NO: 843 - protein, 353 aa, synthetic construct; identical to SEQ ID NO: 841

SEQ ID NO: 844 - protein, 354 aa, synthetic construct; identical to SEQ ID NO: 704

SEQ ID NO: 845 - protein, 353 aa, synthetic construct
QVQLVESGGGLVQAGGSLRLSCAASGRTFRTYAMGWFRQAPGKDREVVGIAAWKVASTYYVDYGDSVRGRFTISRDDAKSTVYLQMNNLMPEDTAVYYCAAGAFRTDWRSYAYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 846 - protein, 354 aa, synthetic construct; identical to SEQ ID NO: 701

SEQ ID NO: 847 - protein, 350 aa, synthetic construct
QVQLVESGGGAVQAGGSLRLSCAASGLTSRHYVMAWFRQAPGIEREFVASIWWGGSTYYADSVKDRFTISRDNAENTVYLQMNSLKPEDTAVYRCAVGTKIGTMGPRYDYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 848 - protein, 354 aa, synthetic construct; identical to SEQ ID NO: 701

SEQ ID NO: 849 - protein, 353 aa, synthetic construct; identical to SEQ ID NO: 751

SEQ ID NO: 850 - protein, 354 aa, synthetic construct
QLQLVESGGGLVQAGGSLRLSCAASGLTFSGYTVGWFRQAPGKEREFVAAINWSSAYTYSEFVKGRFTISRDNAKNTVYLQMSSLKPEDTAVYYCAAQRSRTSWYASTAEAYDYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 851 - protein, 355 aa, synthetic construct; identical to SEQ ID NO: 754

SEQ ID NO: 852 - protein, 354 aa, synthetic construct; identical to SEQ ID NO: 699

SEQ ID NO: 853 - protein, 355 aa, synthetic construct; identical to SEQ ID NO: 754

SEQ ID NO: 854 - protein, 354 aa, synthetic construct; identical to SEQ ID NO: 704

SEQ ID NO: 855 - protein, 345 aa, synthetic construct; identical to SEQ ID NO: 780

SEQ ID NO: 856 - protein, 344 aa, synthetic construct; identical to SEQ ID NO: 716

SEQ ID NO: 857 - protein, 345 aa, synthetic construct; identical to SEQ ID NO: 780

SEQ ID NO: 858 - protein, 352 aa, synthetic construct; identical to SEQ ID NO: 719

SEQ ID NO: 859 - protein, 345 aa, synthetic construct; identical to SEQ ID NO: 780

SEQ ID NO: 860 - protein, 344 aa, synthetic construct; identical to SEQ ID NO: 801

SEQ ID NO: 861 - protein, 345 aa, synthetic construct; identical to SEQ ID NO: 780

SEQ ID NO: 862 - protein, 350 aa, synthetic construct; identical to SEQ ID NO: 783

SEQ ID NO: 863 - protein, 345 aa, synthetic construct; identical to SEQ ID NO: 780

SEQ ID NO: 864 - protein, 354 aa, synthetic construct; identical to SEQ ID NO: 785

SEQ ID NO: 865 - protein, 345 aa, synthetic construct; identical to SEQ ID NO: 780

SEQ ID NO: 866 - protein, 354 aa, synthetic construct; identical to SEQ ID NO: 699

SEQ ID NO: 867 - protein, 345 aa, synthetic construct; identical to SEQ ID NO: 780

SEQ ID NO: 868 - protein, 355 aa, synthetic construct; identical to SEQ ID NO: 734

SEQ ID NO: 869 - protein, 345 aa, synthetic construct; identical to SEQ ID NO: 780

SEQ ID NO: 870 - protein, 354 aa, synthetic construct; identical to SEQ ID NO: 701

SEQ ID NO: 871 - protein, 349 aa, synthetic construct
QVQLQESGGGLVQAGGSLRLSCAASGSIASINAMGWYRQAPGKQRDFVARIQLSGSTNYAHSVRGRFTISRDNAKNTVYLQMNNLKPEDTAVYVCAASNTGSLYGLRNYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 872 - protein, 344 aa, synthetic construct; identical to SEQ ID NO: 801

SEQ ID NO: 873 - protein, 354 aa, synthetic construct
QVQLVESGGGLVQAGGSLRLSCAASGRTFSSYVMGWFRQAPGQEREFVAAIMRNGIMTYYADSVKGRFTISRDNAKNTVYLQMNTLKPDDTAVYYCAAASRVGVSISQSSSYTSWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 874 - protein, 344 aa, synthetic construct; identical to SEQ ID NO: 716

SEQ ID NO: 875 - protein, 354 aa, synthetic construct; identical to SEQ ID NO: 873

SEQ ID NO: 876 - protein, 352 aa, synthetic construct; identical to SEQ ID NO: 719

SEQ ID NO: 877 - protein, 354 aa, synthetic construct; identical to SEQ ID NO: 873

SEQ ID NO: 878 - protein, 344 aa, synthetic construct; identical to SEQ ID NO: 801

SEQ ID NO: 879 - protein, 344 aa, synthetic construct
EVQLVESGGGLVQAGGSLRLSCAASGSKFSFNAMGWYRQTPGKQRELVAIIINGDMINYADSVKGRFTISRDNAKNTMYLQMNSLKPEDTAVYYCKMTRYVYDYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 880 - protein, 344 aa, synthetic construct; identical to SEQ ID NO: 801

SEQ ID NO: 881 - protein, 344 aa, synthetic construct; identical to SEQ ID NO: 879

SEQ ID NO: 882 - protein, 447 aa, synthetic construct
QVQLVQSGAEVKKPGASVRVSCKASGYTFTSYYIHWVRQAPGQGLEWMGIINPSGGSTSYAQKFQGRVTMTRDTSTTTVYMELSSLRSEDTAVYYCAKSNWAYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 883 - protein, 352 aa, synthetic construct; identical to SEQ ID NO: 719

SEQ ID NO: 884 - protein, 214 aa, synthetic construct
SSELTQDPAVSVALGQTVRITCQGDSLSSYYAGWYQQKPGQAPVLVIYGKNNRPSGIPDRFSGSSSEDTASLTITGAQAEDEADYYCNSRDSSDNHLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 885 - protein, 352 aa, synthetic construct; identical to SEQ ID NO: 786

SEQ ID NO: 886 - protein, 352 aa, synthetic construct; identical to SEQ ID NO: 719

SEQ ID NO: 887 - protein, 345 aa, synthetic construct; identical to SEQ ID NO: 780

SEQ ID NO: 888 - protein, 359 aa, synthetic construct
QLQLVESGGGLVQAGGSLRLSCAASGRTFGIHGMGWFRQAPGKEREFAAAIRWSDGTTIHADSVKGRLTISRDNAKNTVYLQMNSLKPEDTAVYYCAATSNPQFGRNWYTVAAGVEYGYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 889 - skipped

SEQ ID NO: 890 - protein, 343 aa, synthetic construct
QLQLVESGGGLVQPGGSLRLSCAASGFTFSSYGMSWVRQAPGKGLEWVSYISAGGGTTTYVDFVKGRFTISRDNAKNMLYLQMNSLKPEDTAVYYCVHAADGSRGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 891 - protein, 354 aa, synthetic construct; identical to SEQ ID NO: 785

SEQ ID NO: 892 - protein, 343 aa, synthetic construct; identical to SEQ ID NO: 890

SEQ ID NO: 893 - protein, 352 aa, synthetic construct
QVQLEESGGGLVQAGGSLRLSCAASGRTFSSYVVGWFRQAPGKEREFVGRISWSGARTYYADSVEGRFTISRDNAKNTVYLQMNSLKPEDTAVYYCAADLKWEVLLSEHYEYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 894 - protein, 356 aa, synthetic construct
QVQLVESGGGLMQAGGSLRLSCALSRGIFSAYAMGWFRQVQGKEREFVSGISRSGSTTDYVDTAKGRFTISRDNARDTVYLQMNSLKPEDTAVYYCAAVTGTFPSSTYHNANAYHHWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 895 - protein, 358 aa, synthetic construct
QVQLVESGGGLVQAGGSLKLSCAASGGTFSSYAMGWFRQAPGKEREFVAVIMRSGDTTAYVDSVKGRFTVSRDNAKNTVYLQMNSLKPEDAAVYFCAAGRPVTRTYYHTYTYPHDYDYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 896 - protein, 353 aa, synthetic construct; identical to SEQ ID NO: 751

SEQ ID NO: 897 - protein, 354 aa, synthetic construct
QLQLVESGGGLVQAGGSLRLSCAASGLTFSGYTVGWFRQAPGKEREFVAAIQWSSAYTYSEFVKGRFTISRDNAKNTVYLQMSSLKPEDTAVYYCAAQRSRTSWYASTAEAYDYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 898 - protein, 352 aa, synthetic construct; identical to SEQ ID NO: 772

SEQ ID NO: 899 - protein, 344 aa, synthetic construct; identical to SEQ ID NO: 716

SEQ ID NO: 900 - protein, 352 aa, synthetic construct; identical to SEQ ID NO: 772

SEQ ID NO: 901 - protein, 344 aa, synthetic construct; identical to SEQ ID NO: 801

SEQ ID NO: 902 - protein, 352 aa, synthetic construct; identical to SEQ ID NO: 772

SEQ ID NO: 903 - protein, 354 aa, synthetic construct; identical to SEQ ID NO: 699

SEQ ID NO: 904 - protein, 345 aa, synthetic construct; identical to SEQ ID NO: 780

SEQ ID NO: 905 - protein, 351 aa, synthetic construct
QVQLEESGGGLVQAGGSLRLSCAASGRTFSSYVVGWFRQAPGKEREFVGRISWSGARTYYADSVEGRFTISRDNAKNTVYLQMNSLKPEDTAVYYCAADLKWEVLLSEHYEYWGQGTQVTVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSP

SEQ ID NO: 906 - protein, 355 aa, synthetic construct; identical to SEQ ID NO: 778

SEQ ID NO: 907 - protein, 354 aa, synthetic construct; identical to SEQ ID NO: 897

SEQ ID NO: 908 - protein, 216 aa, synthetic construct; identical to SEQ ID NO: 762

SEQ ID NO: 909 - protein, 447 aa, synthetic construct; identical to SEQ ID NO: 882

SEQ ID NO: 910 - protein, 9 aa, synthetic construct
PLAPDKTHT

SEQ ID NO: 911 - protein, 4 aa, synthetic construct
PLAP

SEQ ID NO: 912 - protein, 5 aa, synthetic construct
DKTHT

SEQ ID NO: 913 - protein, 7 aa, synthetic construct
EKSYGPP

SEQ ID NO: 914 - protein, 20 aa, synthetic construct
GGGGSGGGGSGGGGSGGGGS

SEQ ID NO: 915 - protein, 9 aa, synthetic construct
PAPNLLGGP

SEQ ID NO: 916 - protein, 6 aa, synthetic construct
GGGGSG

SEQ ID NO: 917 - protein, 5 aa, synthetic construct
GGSGG

SEQ ID NO: 918 - protein, 10 aa, synthetic construct
GGGGSGGGGS

SEQ ID NO: 919 - protein, 9 aa, synthetic construct
GGSGGGGSG

SEQ ID NO: 920 - protein, 10 aa, synthetic construct
GGSGGGGSGS

SEQ ID NO: 921 - protein, 13 aa, synthetic construct
GGSGGGGSGGGGS

SEQ ID NO: 922 - protein, 14 aa, synthetic construct
GGGGSGGGGSGGGG

SEQ ID NO: 923 - protein, 15 aa, synthetic construct
GGGGSGGGGSGGGGS

SEQ ID NO: 924 - protein, 27 aa, synthetic construct
RADAAAAGGGGSGGGGSGGGGSGGGGS

SEQ ID NO: 925 - protein, 6 aa, synthetic construct
ASTKGP

SEQ ID NO: 926 - protein, 13 aa, synthetic construct
ASTKGPSVFPLAP

SEQ ID NO: 927 - protein, 5 aa, synthetic construct
TVAAP

SEQ ID NO: 928 - protein, 6 aa, synthetic construct
RTVAAP

SEQ ID NO: 929 - protein, 12 aa, synthetic construct
TVAAPSVFIFPP

SEQ ID NO: 930 - protein, 13 aa, synthetic construct
RTVAAPSVFIFPP

SEQ ID NO: 931 - protein, 16 aa, synthetic construct
AKTTPKLEEGEFSEAR

SEQ ID NO: 932 - protein, 17 aa, synthetic construct
AKTTPKLEEGEFSEARV

SEQ ID NO: 933 - protein, 9 aa, synthetic construct
AKTTPKLGG

SEQ ID NO: 934 - protein, 10 aa, synthetic construct
SAKTTPKLGG

SEQ ID NO: 935 - protein, 6 aa, synthetic construct
SAKTTP

SEQ ID NO: 936 - protein, 6 aa, synthetic construct
RADAAP

SEQ ID NO: 937 - protein, 9 aa, synthetic construct
RADAAPTVS

SEQ ID NO: 938 - protein, 12 aa, synthetic construct
RADAAAAGGPGS

SEQ ID NO: 939 - protein, 18 aa, synthetic construct
SAKTTPKLEEGEFSEARV

SEQ ID NO: 940 - protein, 5 aa, synthetic construct
ADAAP

SEQ ID NO: 941 - protein, 12 aa, synthetic construct
ADAAPTVSIFPP

SEQ ID NO: 942 - protein, 6 aa, synthetic construct
QPKAAP

SEQ ID NO: 943 - protein, 13 aa, synthetic construct
QPKAAPSVTLFPP

SEQ ID NO: 944 - protein, 6 aa, synthetic construct
AKTTPP

SEQ ID NO: 945 - protein, 13 aa, synthetic construct
AKTTPPSVTPLAP

SEQ ID NO: 946 - protein, 6 aa, synthetic construct
AKTTAP

SEQ ID NO: 947 - protein, 13 aa, synthetic construct
AKTTAPSVYPLAP

SEQ ID NO: 948 - protein, 15 aa, synthetic construct
GENKVEYAPALMALS

SEQ ID NO: 949 - protein, 15 aa, synthetic construct
GPAKELTPLKEAKVS

SEQ ID NO: 950 - protein, 15 aa, synthetic construct
GHEAAAVMQVQYPAS

SEQ ID NO: 951 - protein, 5 aa, synthetic construct
SCDKT

SEQ ID NO: 952 - protein, 6 aa, synthetic construct
KSCDKT